SEQUENCE LISTING (10 sequences)

SEQ ID NO: 1 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): GalNAc-morpholino-thymidine analog 3
  modified_base (2)..(2): GalNAc-morpholino-thymidine analog 3
  modified_base (3)..(3): GalNAc-morpholino-thymidine analog 3
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-OMe-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  misc_feature (20)..(21): /note=“phosphorothiate linkage”
  modified_base (21)..(21): 2′-OMe-nucleotide
  misc_feature (21)..(22): /note=“phosphorothiate linkage”
  modified_base (22)..(22): 2′-deoxy-fluoro-nucleotide
tttaucguacguaccgucguau

SEQ ID NO: 2 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): GalNAc-morpholino-thymidine analog 3
  modified_base (2)..(2): GalNAc-morpholino-thymidine analog 3
  modified_base (3)..(3): GalNAc-morpholino-thymidine analog 3
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-deoxy-fluoro-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  modified_base (22)..(22): 2′-deoxy-fluoro-nucleotide
  modified_base (23)..(23): 2′-OMe-nucleotide
  modified_base (24)..(24): 2′-deoxy-fluoro-nucleotide
  modified_base (25)..(25): Cyclohexane-morpholino-thymidine analog 4
  modified_base (26)..(26): Cyclohexane-morpholino-thymidine analog 4
tttaacaguguucuugcucuauaatt

SEQ ID NO: 3 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): GalNAc-morpholino-thymidine analog 7
  modified_base (2)..(2): GalNAc-morpholino-thymidine analog 7
  modified_base (3)..(3): GalNAc-morpholino-thymidine analog 7
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-deoxy-fluoro-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  modified_base (22)..(22): 2′-deoxy-fluoro-nucleotide
  modified_base (23)..(23): 2′-OMe-nucleotide
  modified_base (24)..(24): 2′-deoxy-fluoro-nucleotide
  modified_base (25)..(25): Cyclohexane-morpholino-thymidine analog 4
  modified_base (26)..(26): Cyclohexane-morpholino-thymidine analog 4
tttaacaguguucuugcucuauaatt

SEQ ID NO: 4 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): Piperidine-morpholino-thymidine analog 1
  modified_base (2)..(2): Piperidine-morpholino-thymidine analog 1
  modified_base (3)..(3): Piperidine-morpholino-thymidine analog 1
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-deoxy-fluoro-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  modified_base (22)..(22): 2′-deoxy-fluoro-nucleotide
  modified_base (23)..(23): 2′-OMe-nucleotide
  modified_base (24)..(24): 2′-deoxy-fluoro-nucleotide
  modified_base (25)..(25): Cyclohexane-morpholino-thymidine analog 4
  modified_base (26)..(26): Cyclohexane-morpholino-thymidine analog 4
tttaacaguguucuugcucuauaatt

SEQ ID NO: 5 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): Guanosine-morpholino-thymidine analog 1
  modified_base (2)..(2): Guanosine-morpholino-thymidine analog 1
  modified_base (3)..(3): Guanosine-morpholino-thymidine analog 1
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-deoxy-fluoro-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  modified_base (22)..(22): 2′-deoxy-fluoro-nucleotide
  modified_base (23)..(23): 2′-OMe-nucleotide
  modified_base (24)..(24): 2′-deoxy-fluoro-nucleotide
  modified_base (25)..(25): Cyclohexane-morpholino-thymidine analog 4
  modified_base (26)..(26): Cyclohexane-morpholino-thymidine analog 4
tttaacaguguucuugcucuauaatt

SEQ ID NO: 6 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): Guanosine-morpholino-thymidine analog 2
  modified_base (2)..(2): Guanosine-morpholino-thymidine analog 2
  modified_base (3)..(3): Guanosine-morpholino-thymidine analog 2
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-deoxy-fluoro-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  modified_base (22)..(22): 2′-deoxy-fluoro-nucleotide
  modified_base (23)..(23): 2′-OMe-nucleotide
  modified_base (24)..(24): 2′-deoxy-fluoro-nucleotide
  modified_base (25)..(25): Cyclohexane-morpholino-thymidine analog 4
  modified_base (26)..(26): Cyclohexane-morpholino-thymidine analog 4
tttaacaguguucuugcucuauaatt

SEQ ID NO: 7 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): Guanosine-morpholino-thymidine analog 3
  modified_base (2)..(2): Guanosine-morpholino-thymidine analog 3
  modified_base (3)..(3): Guanosine-morpholino-thymidine analog 3
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-deoxy-fluoro-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  modified_base (22)..(22): 2′-deoxy-fluoro-nucleotide
  modified_base (23)..(23): 2′-OMe-nucleotide
  modified_base (24)..(24): 2′-deoxy-fluoro-nucleotide
  modified_base (25)..(25): Cyclohexane-morpholino-thymidine analog 4
  modified_base (26)..(26): Cyclohexane-morpholino-thymidine analog 4
tttaacaguguucuugcucuauaatt

SEQ ID NO: 8 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(1): 2′-deoxy-fluoro-nucleotide
  misc_feature (1)..(2): /note=“phosphorothiate linkage”
  modified_base (2)..(2): 2′-deoxy-fluoro-nucleotide
  misc_feature (2)..(3): /note=“phosphorothiate linkage”
  modified_base (3)..(3): 2′-OMe-nucleotide
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-deoxy-fluoro-nucleotide
  modified_base (13)..(13): 2′-OMe-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  misc_feature (19)..(20): /note=“phosphorothiate linkage”
  modified_base (20)..(20): dT
  misc_feature (20)..(21): /note=“phosphorothiate linkage”
  modified_base (21)..(21): dT
auacgacgguacguacgautt

SEQ ID NO: 9 - RNA, 23 nt, Artificial Sequence
  source: /note=“Description of Artificial Sequence Synthetic oligonucleotide”
  modified_base (1)..(1): 2′-OMe-nucleotide
  misc_feature (1)..(2): /note=“phosphorothiate linkage”
  modified_base (2)..(2): 2′-deoxy-fluoro-nucleotide
  misc_feature (2)..(3): /note=“phosphorothiate linkage”
  modified_base (3)..(3): 2′-OMe-nucleotide
  modified_base (4)..(4): 2′-deoxy-fluoro-nucleotide
  modified_base (5)..(5): 2′-OMe-nucleotide
  modified_base (6)..(6): 2′-deoxy-fluoro-nucleotide
  modified_base (7)..(7): 2′-OMe-nucleotide
  modified_base (8)..(8): 2′-deoxy-fluoro-nucleotide
  modified_base (9)..(9): 2′-OMe-nucleotide
  modified_base (10)..(10): 2′-deoxy-fluoro-nucleotide
  modified_base (11)..(11): 2′-OMe-nucleotide
  modified_base (12)..(12): 2′-OMe-nucleotide
  modified_base (13)..(13): 2′-OMe-nucleotide
  modified_base (14)..(14): 2′-deoxy-fluoro-nucleotide
  modified_base (15)..(15): 2′-OMe-nucleotide
  modified_base (16)..(16): 2′-deoxy-fluoro-nucleotide
  modified_base (17)..(17): 2′-OMe-nucleotide
  modified_base (18)..(18): 2′-deoxy-fluoro-nucleotide
  modified_base (19)..(19): 2′-OMe-nucleotide
  modified_base (20)..(20): 2′-deoxy-fluoro-nucleotide
  modified_base (21)..(21): 2′-OMe-nucleotide
  misc_feature (21)..(22): /note=“phosphorothiate linkage”
  modified_base (22)..(22): 2′-OMe-nucleotide
  misc_feature (22)..(23): /note=“phosphorothiate linkage”
  modified_base (23)..(23): 2′-OMe-nucleotide
uuauagagcaagaacacuguuuu

SEQ ID NO: 10 - DNA, 16 bp, Artificial Sequence
  source: /note=“Description of Artificial Sequence Synthetic oligonucleotide”
  modified_base (1)..(16): dT
tttttttttttttttt

(19) United States
(12) Patent Application Publication
(10) Pub. No.: US 2024/0092819 A1
(43) Pub. Date: Mar. 21, 2024

(54) NOVEL LIGANDS FOR ASIALOGLYCOPROTEIN RECEPTOR

(71) Applicant: SANOFI, Paris (FR)

(72) Inventors: Bettina ELSHORST, Frankfurt am Main (DE); Gerhard HESSLER, Frankfurt am Main (DE); Armin HOFMEISTER, Frankfurt am Main (DE); Ziyu LI, Frankfurt am Main (DE); Christoph PÖVERLEIN, Frankfurt am Main (DE); Herman SCHREUDER, Frankfurt am Main (DE); Gernot ZECH, Frankfurt am Main (DE)

(21) Appl. No.: 18/248,988

(22) PCT Filed: Oct. 19, 2021
(86) PCT No.: PCT/EP21/78980

§ 371 Date: Apr. 13, 2023

(30) Foreign Application Priority Data
Oct. 20, 2020 (EP) 20306243.5

(51) Int. Cl. C07H 19/16; C07D 211/56; C07H 19/207; C07H 21/00; C12N 15/113

(52) U.S. Cl. CPC C07H 19/16; C07D 211/56; C07H 19/207; C07H 21/00; C12N 15/1138; C12N 2310/14; C12N 2310/33; C12N 2310/351; C12N 2320/32

(57) ABSTRACT

The present disclosure provides novel piperidine- and guanosine-derived ligands that bind specifically to asialoglycoprotein receptor (ASGPR) and nucleotide analogs conjugated thereto that can be incorporated into oligonucleotides, including double-stranded oligonucleotides such as siRNAs.

### SEQUENCE LISTING

Nucleic acid sequences are disclosed in the present specification that serve as references. The same sequences are also presented in a sequence listing formatted according to standard requirements for the purpose of patent matters. In case of any sequence discrepancy with the standard sequence listing, the sequences described in the present specification shall be the reference.

### BACKGROUND

The concept of using synthetic oligonucleotides to control gene expression dates back to the late 1970s when targeted gene silencing using a short synthetic oligonucleotide was first demonstrated (Stephenson et al., Proc Natl Acad Sci. USA (1978) 75:285-88). Subsequent to Stephenson's discovery, elucidation of the RNA interference pathway for modulation of gene expression and the role of siRNAs in the process has vastly expanded scientists' understanding of posttranscriptional gene expression control in eukaryotic cells.

Synthetic oligonucleotides include single stranded oligonucleotides such as antisense oligonucleotides (“ASOs”), antimiRs or antagomiRs; and double stranded oligonucleotides such as small interfering RNAs (siRNAs). ASOs and siRNAs both work by binding a target RNA through Watson-Crick base pairing, but their mechanisms of action are different. In antisense technology, ASOs form a DNA-RNA duplex with the target RNA and inhibit mRNA translation by a blocking mechanism or cause RNase H-dependent degradation of the targeted RNA. In RNA interference technology, siRNAs bind to the RNA-induced silencing complex (“RISC”), where one strand (the “passenger strand” or “sense strand”) is displaced and the remaining strand (the “guide strand” or “antisense strand”) cooperates with the RISC to bind a complementary RNA (the target RNA). Once bound, the target RNA is cleaved by the RNA endonuclease Argonaute (AGO) in the RISC and then further degraded by RNA exonucleases.

Key challenges in the development of oligonucleotide therapeutics, e.g., siRNA therapeutics, include (i) poor stability of the compounds, (ii) low efficiency of in vivo delivery to target cells, and (iii) side effects such as “off target” gene silencing and unintended immunostimulation. Among these, the most significant obstacle is the targeted delivery and subsequent cellular uptake of siRNAs. To overcome some of these obstacles, researchers have attempted various chemical modifications of the oligonucleotide, including (i) sugar modifications, (ii) internucleotide linkage modifications, and (iii) nucleobase modifications. While these chemical modifications have led to enhanced stability and reduced immunogenicity of the siRNAs, these modifications are still insufficient to deliver these large, negatively charged macromolecules across the negatively charged phospholipid bilayer of the cell membrane and into the cytoplasm.

To this end, some groups have used N-acetylgalactosamine (GalNAc) to target the siRNA attached thereto to hepatocytes, which express the GalNAc-binding asialoglycoprotein receptor (ASGPR) and can internalize the ASGPR-bound siRNA-GalNAc conjugate through endocytosis (see, e.g., Nair et al., J Am Chem Soc. (2014) 136:16958-61). ASGPR is a calcium-dependent, carbohydrate-specific, transmembrane C-type lectin primarily expressed on the sinusoidal surface of hepatocytes. It plays a key role in serum glycoprotein turnover by mediating endocytosis and lysosomal degradation of glycoproteins with exposed terminal galactose or GalNAc residues. (Roggenbuck et al., Auto Immun Highlights. (2012) 3(3):119-25; D'Souza et al., J Controlled Release (2015) 203:126-39).

Given the importance of delivering therapeutic siRNAs to target cells in a tissue-specific manner, there remains a need for developing stable molecular moieties that can be readily conjugated to therapeutic agents such as siRNAs and ASOs and bind specifically to molecules that are expressed specifically in targeted tissues.

### SUMMARY

The present disclosure provides a compound of formula (I)

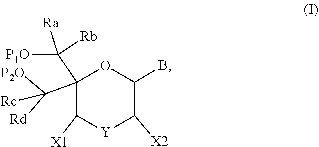

or a pharmaceutically acceptable salt thereof,
wherein:
B is a heterocyclic nucleobase;
P₁ and P₂ are each, independently, H, a reactive phosphorous group, or a protecting group;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is a cell targeting moiety of formula (II) or a protected derivative thereof:

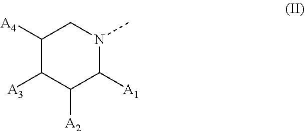

wherein:
R3 targets a mammalian (optionally human) asialoglycoprotein receptor (ASGPR),
A₁, A₂ and A₃ are, independently, H, hydroxy, alkoxy, acyloxy, aryloxy, aroyloxy, alkoxycarbonyl, aryloxycarbonyl, oxo (═O), or a (C1-C20) alkyl group, unsubstituted or optionally substituted by one or more groups selected from OH, a (C3-C8) cycloalkyl group, a (C3-C14) heterocycle, a (C6-C14) aryl group, a (C5-C14) heteroaryl group, —O—Z5, —N(Z5)(Z6), —S—Z5, —CN, —C(=M)-O—Z5, —O—C(=M)-Z5, —C(=M)-N(Z5)(Z6), and —N(Z5)-C(=M)-Z6, wherein:
M is O or S,
each of Z5 and Z6 is, independently, H, a (C1-C6) alkyl group, or a (C6-C14) aryl group, wherein both alkyl and aryl groups are either unsubstituted or optionally substituted by one or more groups selected from halogen, amino, hydroxy, thiol, cyano, alkyl, alkoxy, aryloxy, acyloxy, aroyloxy, carboxy, alkoxycarbonyl, aryloxycarbonyl and arylalkoxycarbonyl;
A₄ is —N(R4)₂, —NH—C(═O)—R4, or

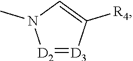

wherein:
D2 and D3 are N, O, or S;
R4 is H or a (C1-C20) alkyl group, unsubstituted or optionally substituted by one or more groups selected from halogen, amino, hydroxy, thiol, cyano, alkyl, alkoxycarbonyl, aryloxycarbonyl, alkoxy, aryloxy, acyloxy, aroyloxy and carboxy; and
each of X1, X2, Ra, Rb, Re, and Rd independently is H or a —(C1-C6) alkyl group.

In another aspect, the present disclosure provides a compound of formula (I)

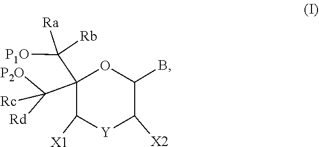

or a pharmaceutically acceptable salt thereof,
wherein:
B is a heterocyclic nucleobase;
P₁ and P₂ are each, independently, H, a reactive phosphorous group, or a protecting group;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is a cell targeting moiety of formula (IVA) or (IVB) or a protected derivative thereof:

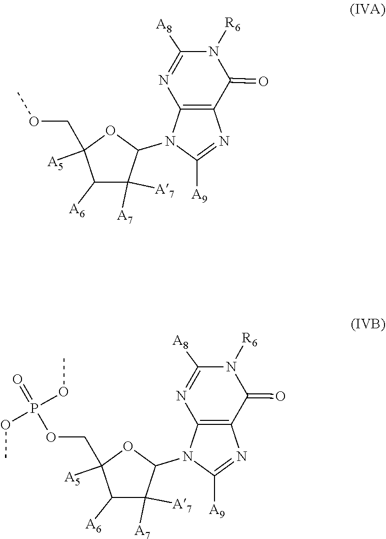

wherein:
R3 targets a mammalian (optionally human) asialoglycoprotein receptor (ASGPR),
R6 is H or a (C1-C6) alkyl group, unsubstituted or optionally substituted by one or more groups selected from halogen, amino, hydroxy, thiol, alkyl, alkoxy, aryloxy, carboxy, alkoxycarbonyl, and aryloxycarbonyl;
A₅, A₆, A₇, and A′₇ are independently H, hydroxy, alkoxy, acyloxy, aryloxy, aroyloxy, alkoxycarbonyl, aryloxycarbonyl, amino, or a (C1-C20) alkyl group unsubstituted or optionally substituted by one or more groups selected from OH, a (C3-C8) cycloalkyl group, a (C3-C14) heterocycle, a (C6-C14) aryl group, a (C5-C14) heteroaryl group, —O—Z7, —N(Z7)(Z8), —S—Z7, —CN, —C(=Q)-O—Z7, —O—C(=Q)-Z7, —C(=Q)-N(Z7)(Z8), and —N(Z7)-C(=Q)-Z8, wherein:
Q is O or S,
each of Z7 and Z8 independently is H, a (C1-C6) alkyl group, or a (C6-C14) aryl group, both groups unsubstituted or optionally substituted by one or more groups selected from a halogen atom and a (C1-C6) alkyl group;
each of A₈ and A₉ independently is H, halogen, OH (or its tautomeric oxo (═O)), —N(R7)₂, —NHR7, or —NH—C(═O)—R7, wherein R7 is hydrogen or a (C1-C20) alkyl group, unsubstituted or optionally substituted by one or more groups selected from a halogen atom, alkoxy, aryloxy, a (C1-C6) alkyl group, a (C3-C8) cycloalkyl group, a (C3-C14) heterocycle, a (C6-C14) aryl group, a (C5-C14) heteroaryl group; and
each of X1, X2, Ra, Rb, Rc, and Rd independently is H or a —(C1-C6) alkyl group.

In another aspect, the present disclosure provides a compound of formula (III)

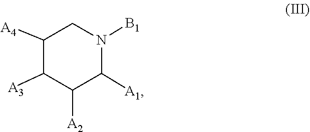

or a pharmaceutically acceptable salt thereof,
wherein:
A1, A2 and A3 are, independently, H, hydroxy, alkoxy, acyloxy, aryloxy, aroyloxy, alkoxycarbonyl, aryloxycarbonyl, oxo (═O), or a (C1-C20) alkyl or alkenyl group, unsubstituted or optionally substituted by one or more groups selected from halogen, hydroxy, a (C3-C8) cycloalkyl group, a (C3-C14) heterocycle, a (C6-C14) aryl group, a (C5-C14) heteroaryl group, —O—Z5, —N(Z5)(Z6), —S—Z5, —CN, —C(=M)-O—Z5, —O—C(=M)-Z5, —C(=M)-N(Z5)(Z6), and —N(Z5)-C(=M)-Z6, wherein:
M is O or S,
each of Z5 and Z6 is, independently, H, a (C1-C6) alkyl group, unsubstituted or optionally substituted by one or more groups selected from halogen, amino, hydroxy, alkoxy, aryloxy, carboxy, alkoxycarbonyl, aryloxycarbonyl, and carbonyloxy;
A4 is —N(R4)2, —N—C(═O)—R4, or

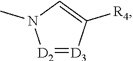

wherein:
D2 and D3 are N, O, or S;
R4 is H or a (C1-C20) alkyl group, unsubstituted or optionally substituted by one or more groups selected from halogen, amino, hydroxy, thiol, cyano, alkyl, alkoxycarbonyl, aryloxycarbonyl, alkoxy, aryloxy, acyloxy, aroyloxy and carboxy;
B1 is H, benzyl ester, -L-R5, or —(CO)-L-R5, wherein:
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —(CO)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy, or haloalkyl, the C2-C25 hydrocarbon chain being optionally substituted with one or more -L′-R5, wherein L′ is a C2-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; and
R5 is H, OH, benzyl, benzyloxy, or a nucleoside, nucleoside analog, nucleotide or nucleotide analog, for example, a nucleoside analog of formula (I).

In yet another aspect, the present disclosure provides a compound of formula (V)

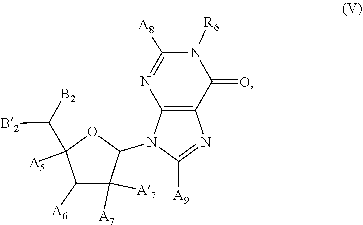

or a pharmaceutically acceptable salt thereof,
wherein:
R6 is H or a (C1-C6) alkyl group, unsubstituted or optionally substituted by one or more groups selected from halogen, amino, hydroxy, thiol, alkoxy, aryloxy, carboxy, alkoxycarbonyl, and aryloxycarbonyl;
each of A5, A6, A7, and A′7 independently is H, hydroxy, alkoxy, acyloxy, aryloxy, aroyloxy, amino, or a (C1-C20) alkyl group unsubstituted or optionally substituted by one or more groups selected from halogen, OH, a (C3-C8) cycloalkyl group, a (C3-C14) heterocycle, a (C6-C14) aryl group, a (C5-C14) heteroaryl group, —O—Z7, —N(Z7)(Z8), —S—Z7, —CN, —C(=Q)-O—Z7, —O—C(=Q)-Z7, —C(=Q)-N(Z7)(Z8), and —N(Z7)-C(=Q)-Z8, wherein:
Q is O or S,
each of Z7 and Z8 independently is H or a (C1-C6) alkyl group, unsubstituted or optionally substituted by one or more groups selected from a halogen atom and a (C1-C6) alkyl group;
each of A8 and A9 independently is H, halogen, OH (or its tautomeric oxo (═O)), —N(R7)2, —NHR7, or —N—C(═O)—R7, wherein R7 is hydrogen or a (C1-C20) alkyl group, unsubstituted or optionally substituted by one or more groups selected from a halogen atom, alkoxy, aryloxy, a (C1-C6) alkyl group, a (C3-C8) cycloalkyl group, a (C3-C14) heterocycle, a (C6-C14) aryl group, a (C5-C14) heteroaryl group;
each of B2 and B′2 independently is —H, —OH, —OR8, —COOH, —C(O)—NR8R′8, —NH₂, —NHR8, —NH—C(O)—R8, —O—P(O)(OH)₂, —O—P(O)(OR8)(OR′8) or a (C1-C6) alkyl optionally substituted by —OH, wherein R8 and R′8 are independently H or -L-R9, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxy, or haloalkyl, the C1-C25 hydrocarbon chain being optionally substituted with one or more -L′-R9, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re), —O—C(O)—(Re), —C(O)—O—(Re), or —O—C(O)—O—;
R9 is H, OH, benzyl, benzyloxy, or a nucleoside, a nucleoside analog, or a nucleotide or a nucleotide analog, for example, a nucleoside analog of formula (I), and wherein when B₂ is CH₂OH, B₂′ is OH, A₅ is H, A₆ is OH, A₇ is H, A₇′ is OH, A₉ is H, and R₆ is H, A₈ is not NH₂.

In yet another aspect, the present disclosure provides an oligonucleotide comprising one or more compounds of formula (VI):

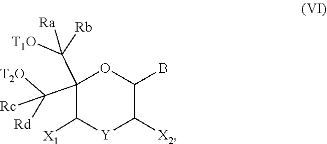

or a pharmaceutically acceptable salt thereof.
wherein:
B is a heterocyclic nucleobase;
one of T₁ and T₂ is an internucleoside linking group linking the compound of formula (VI) to the oligomeric compound and the other of T₁ and T₂ is H, a protecting group, a phosphorus moiety, or an internucleoside linking group linking the compound of formula (VI) to the oligomeric compound;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxy or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is an ASGPR-binding cell targeting moiety of formula (II), (IVA) or (IVB); and each of X1, X2, Ra, Rb, Rc, and Rd independently is H or a —(C1-C6) alkyl group.

The present disclosure also provides a method of delivering an oligonucleotide to liver (hepatic) cells in a human subject in need thereof, comprising administering (e.g., through subcutaneous or intravenous injection or injection through the hepatic portal vein) to the subject an oligonucleotide of the present disclosure.

The present disclosure also refers to use of an oligonucleotide of the present description for the manufacture of a medicament to treat a human subject in need thereof.

The present disclosure also provides an oligonucleotide as described herein for use in treating a human subject in need thereof.

The present disclosure further provides a method of preparing a liver-targeting therapeutic agent (e.g., a protein, a peptide, a peptide mimetic, a small molecule, or an oligonucleotide), comprising reacting a therapeutic moiety and a compound of the present description to allow conjugation of the compound to the therapeutic moiety, thereby generating a liver-targeting therapeutic agent.

The present disclosure further provides a method of delivering a therapeutic agent (e.g., a protein, a peptide, a peptide mimetic, a small molecule, or a polynucleotide) to liver (hepatic) cells in a human subject in need thereof, comprising administering to the subject a therapeutic moiety conjugated to a compound of the present description.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 depicts a scheme for the synthesis of compounds 2, 3, and 23.

FIG. 2 depicts a scheme for the synthesis of compound 30.

FIG. 3 depicts a scheme for the synthesis of compound 37.

FIG. 4 depicts a scheme for the synthesis of compound 47.

FIG. 5 depicts a scheme for the synthesis of compound 58.

FIG. 6 depicts a scheme for the synthesis of compounds 71, 72, and 73.

FIG. 7 depicts a scheme for the synthesis of precursors for simplified piperidine-derived ASGPR binding molecules.

FIG. 8 depicts a scheme for the synthesis of linker precursors.

FIG. 9 depicts a scheme for the synthesis of compounds 112, 117, 119, 120, and 121.

FIG. 10 depicts a scheme for the synthesis of compounds 128, 129, 131, and 132.

FIG. 11 depicts a scheme for the synthesis of compounds 138 and 140.

FIG. 12 depicts a scheme for the synthesis of compounds 146, 147, 148, 153, and 154.

FIG. 13 depicts a scheme for the synthesis of compounds 160, 161, and 162.

FIG. 14 depicts a scheme for the synthesis of compounds 180 and 181.

FIG. 15 depicts a scheme for the synthesis of piperidine precursors for trimerization.

FIG. 16 depicts a scheme for the synthesis of targeted nucleotide precursor 218 (pre-lsT1).

FIG. 17 depicts a scheme for the synthesis of targeted nucleotide precursor 230 (pre-lsT2).

FIG. 18 depicts a scheme for the synthesis of targeted nucleotide precursor 246 (pre-lsT3).

FIG. 19 depicts a scheme for the synthesis of targeted nucleotide precursor 249 (pre-lpT1).

FIG. 20A depicts a scheme for the synthesis of targeted nucleosides 254 and 258.

FIG. 20B depicts a scheme for the synthesis of targeted nucleoside 260.

FIG. 21A depicts a scheme for the synthesis of trimeric targeted nucleotides 261 and 262.

FIG. 21B depicts a scheme for the synthesis of trimeric targeted nucleotides 263, 264, and 265.

FIG. 22 depicts a scheme for the synthesis of trimeric ASGPR-binder 267.

FIG. 23 depicts a scheme for the synthesis of trimeric ASGPR-binder 268.

FIG. 24 depicts a scheme for the synthesis of trimeric ASGPR-binder 269.

FIG. 25A is a graph showing relative TTR protein serum levels at blood sampling time points before and after subcutaneous (s.c.) dosing of siRNA 1-0 (negative control), siRNA 1-1 (positive control), and siRNA1-3 as indicated. Ordinate: TTR serum level relative to pre-dosing+/−SEM. Abscissa: days post-subcutaneous dosing

FIG. 25B is a graph showing relative TTR protein serum levels at blood sampling time points before and after subcutaneous dosing of siRNA1-0 (negative control), siRNA1-2 (positive control), siRNA1-4, siRNA1-5, and siRNA1-6 as indicated. Ordinate: TTR serum level relative to pre-dosing+/−SEM. Abscissa: days post-subcutaneous dosing

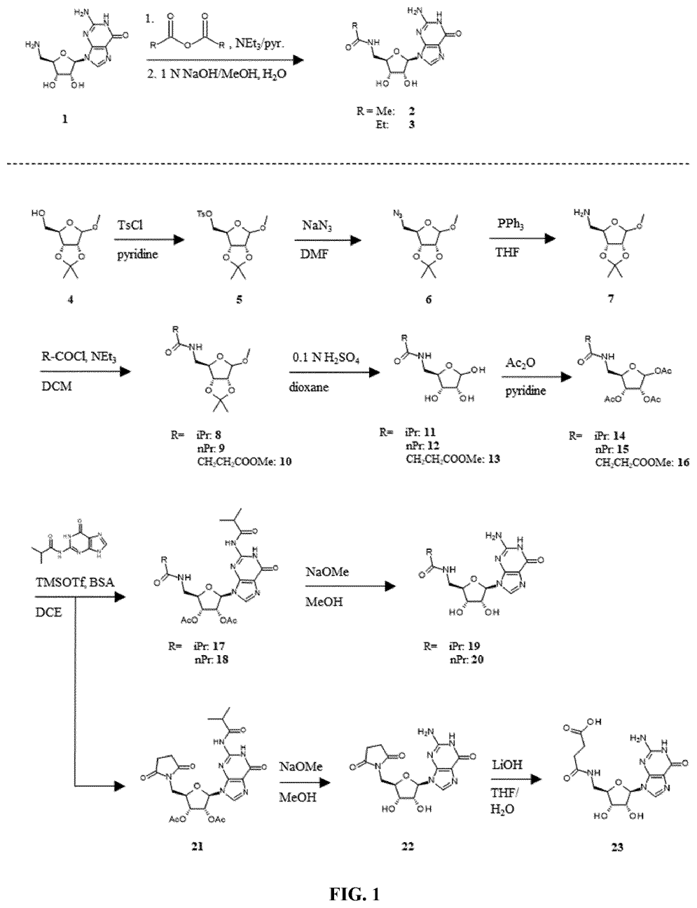

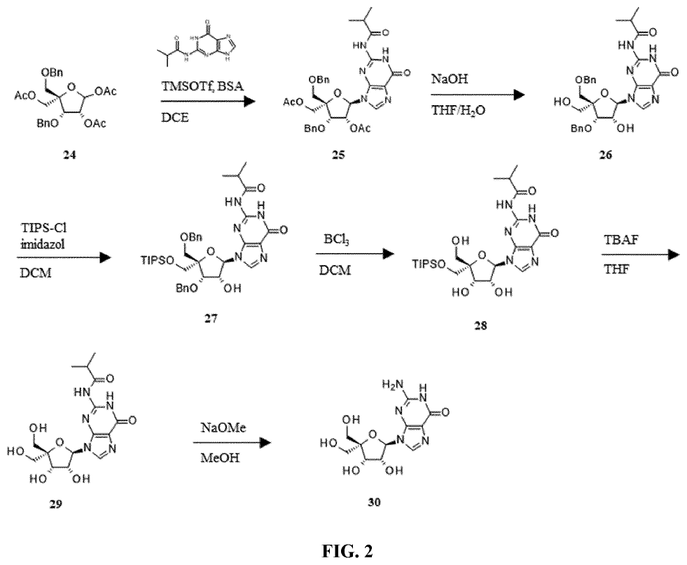

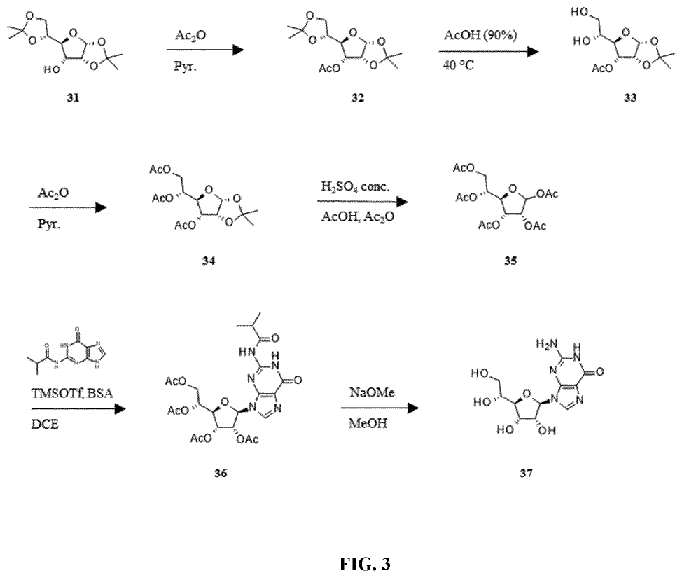

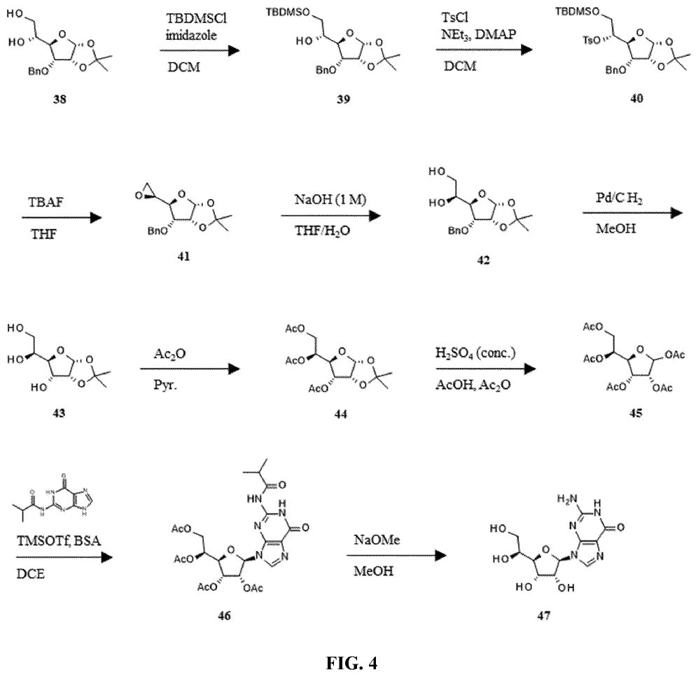

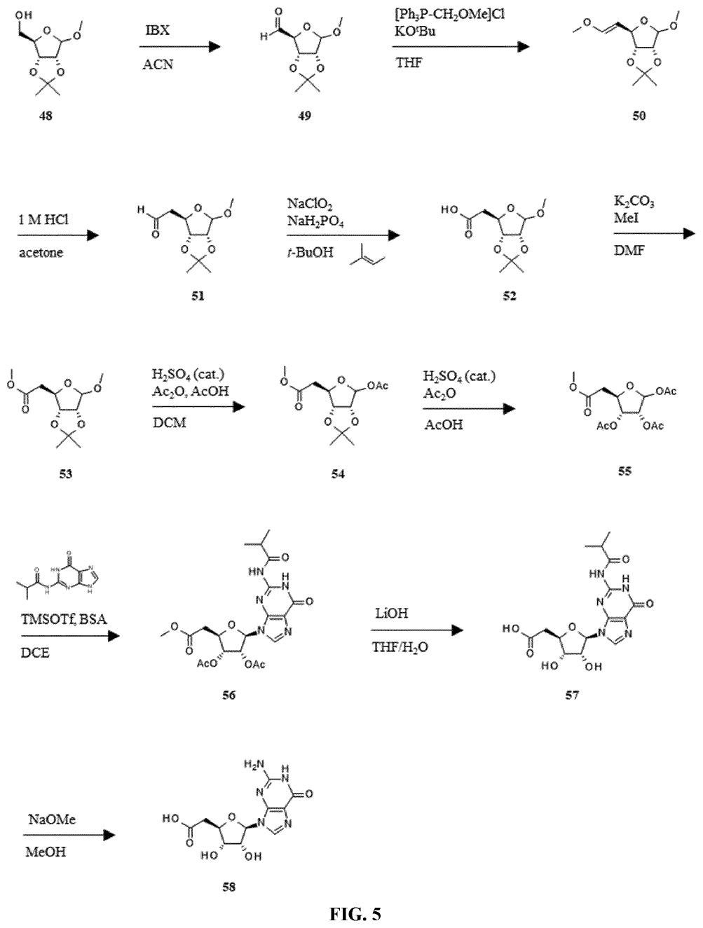

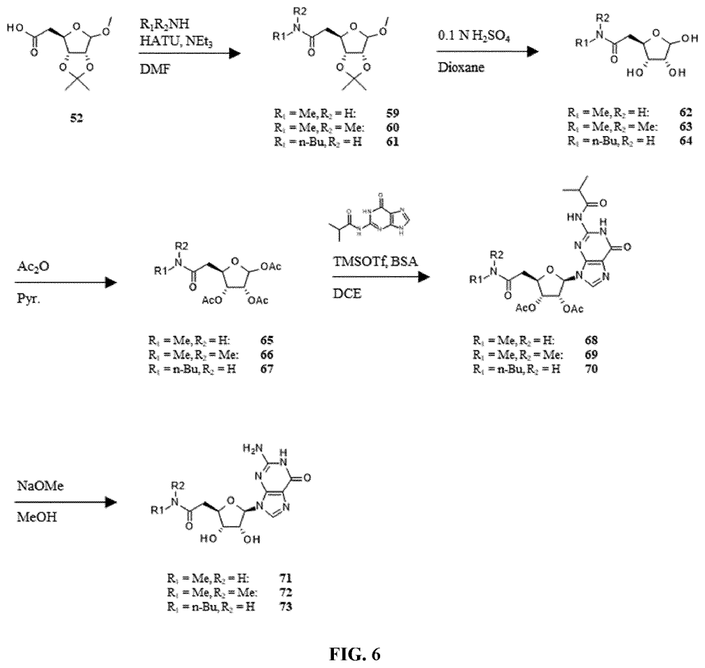

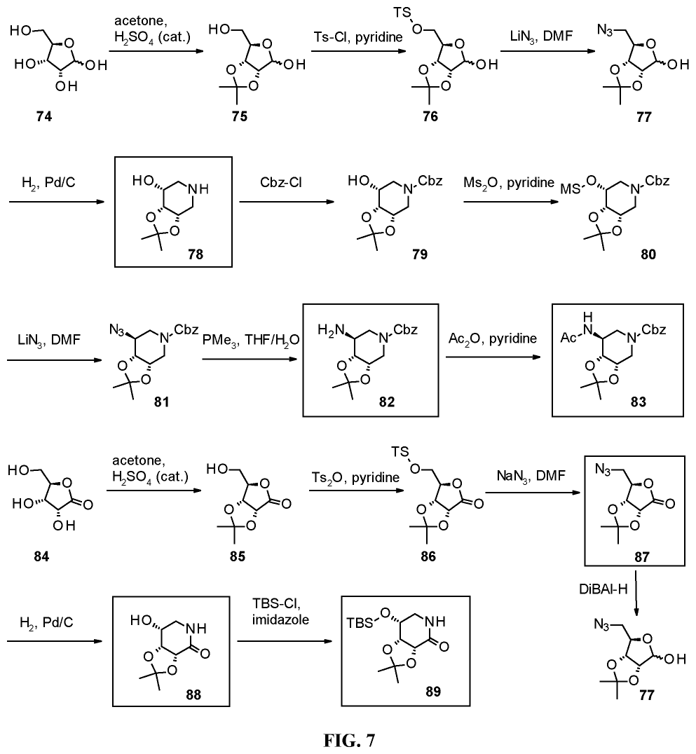

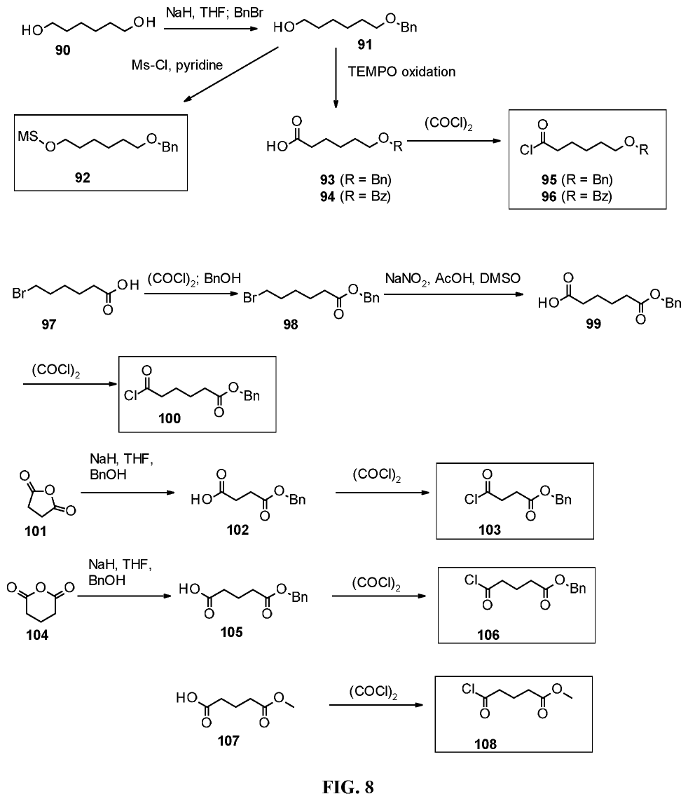

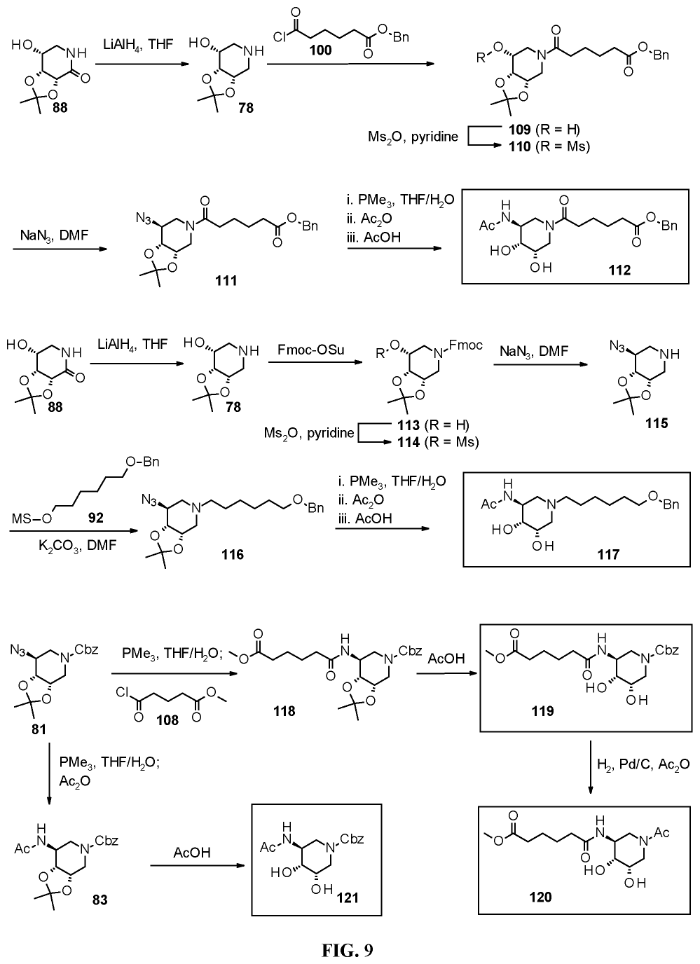

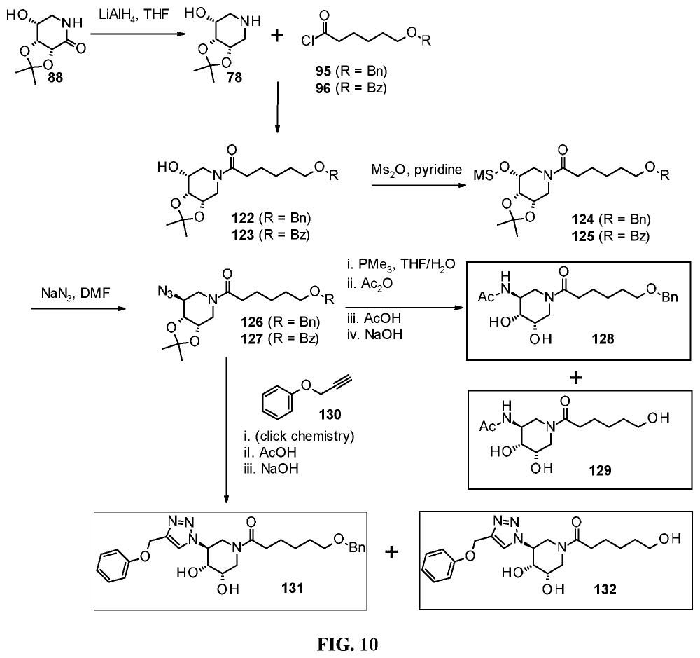

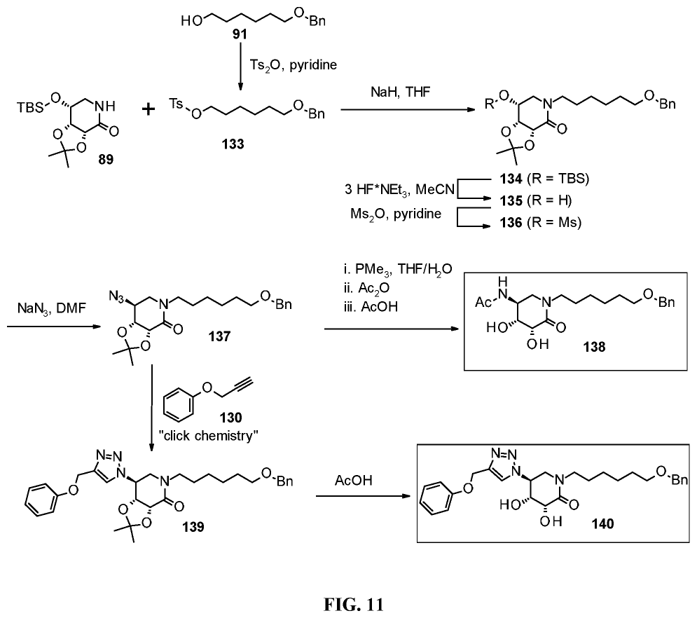

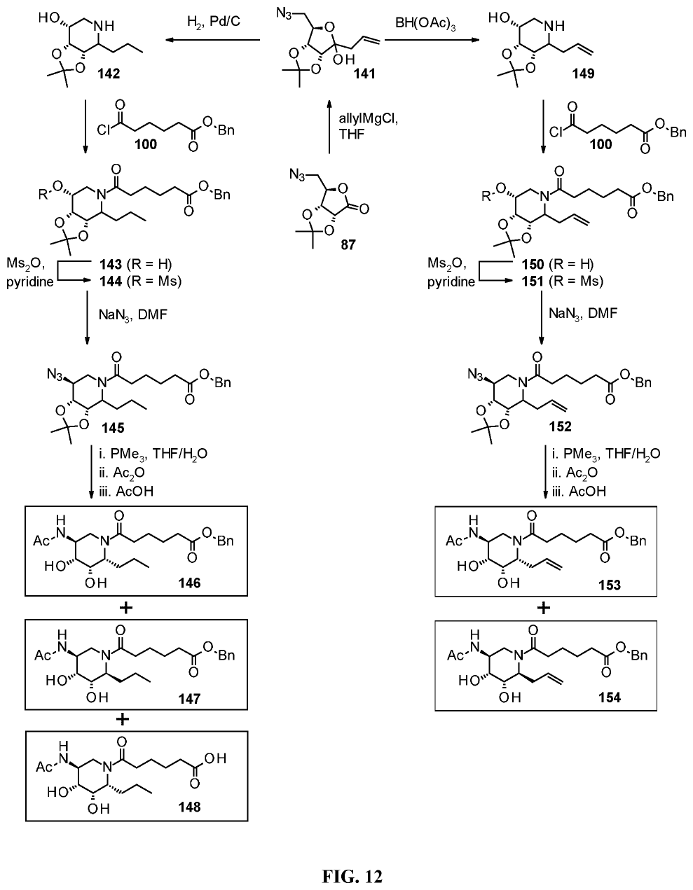

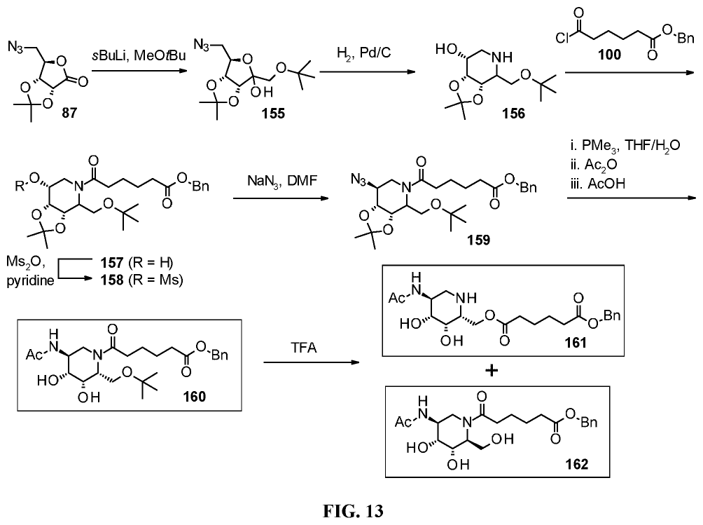

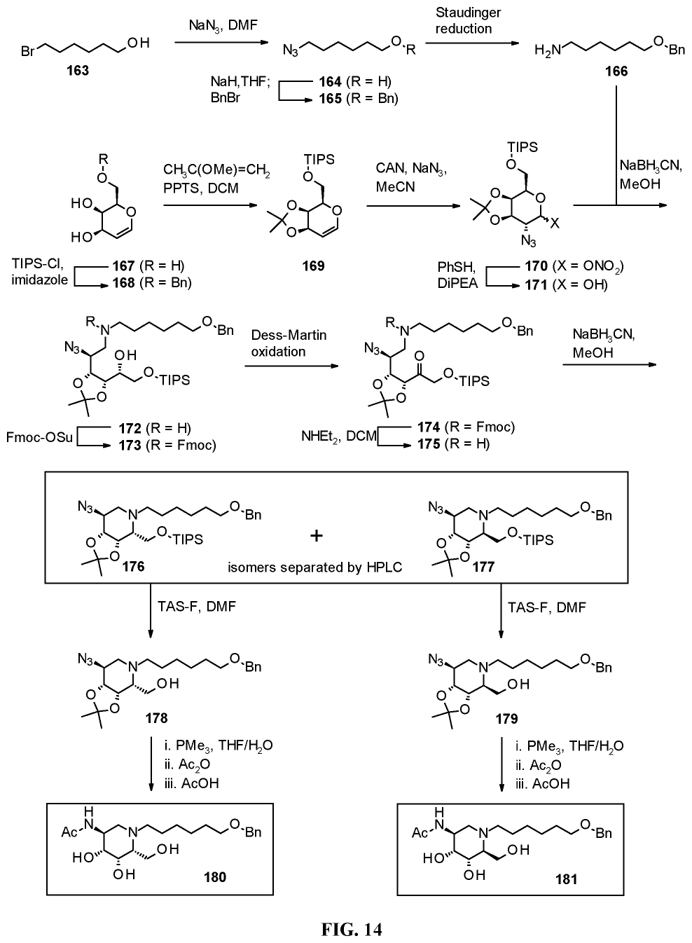

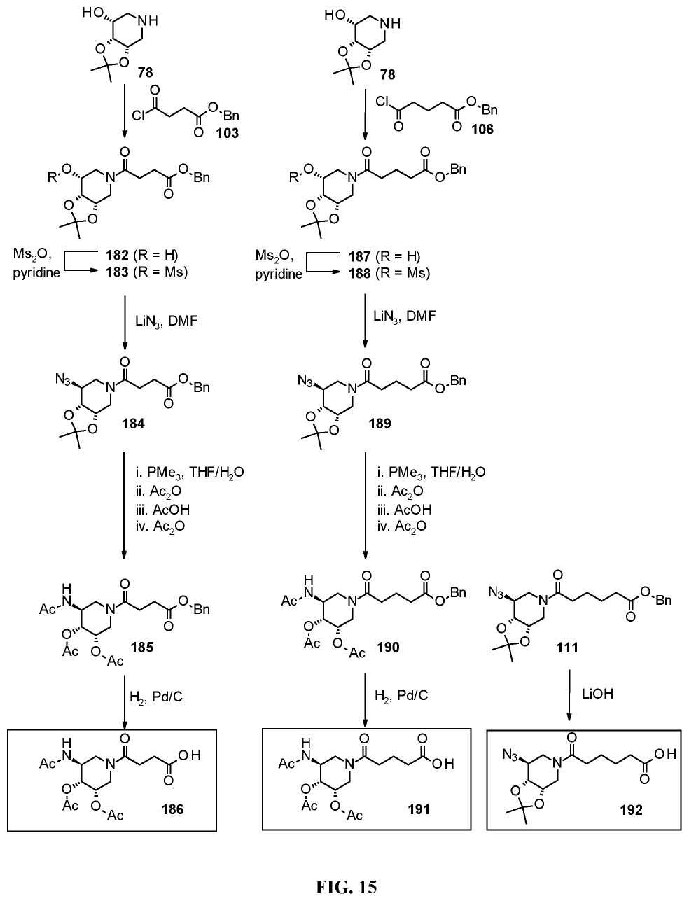

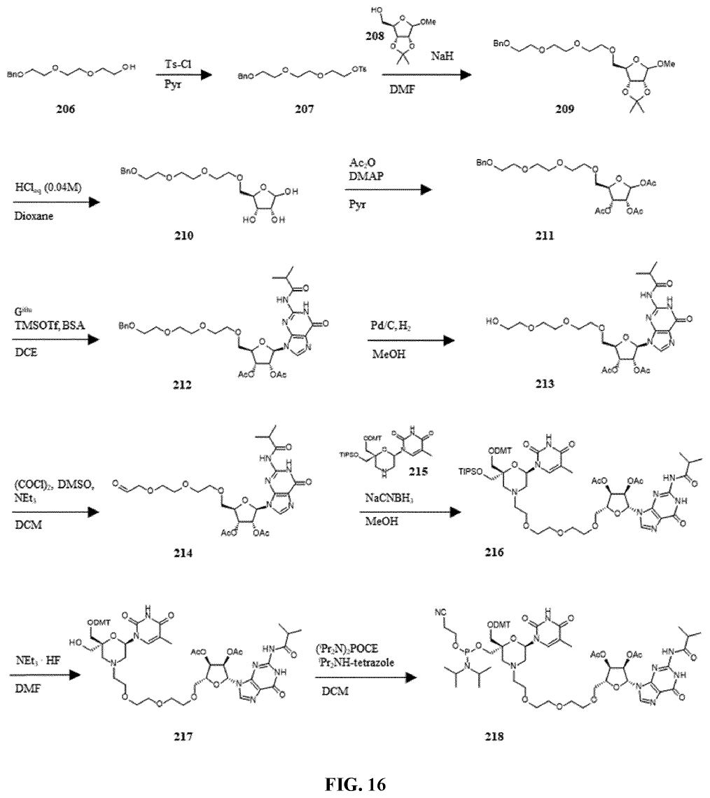

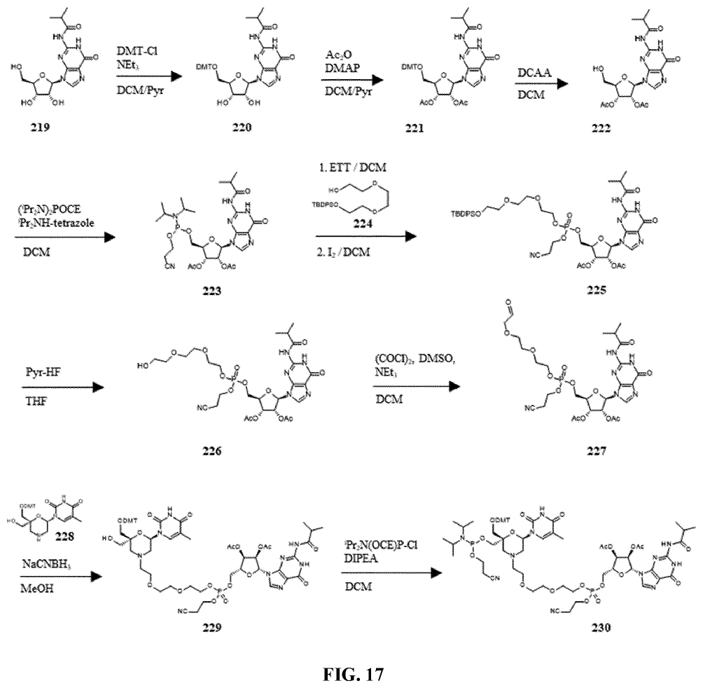

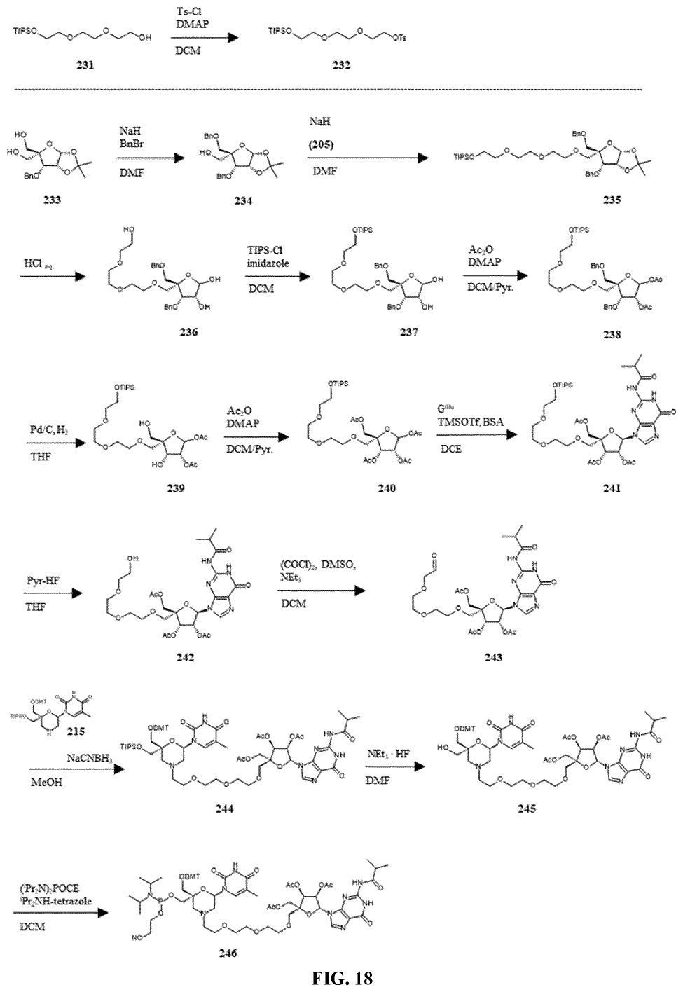

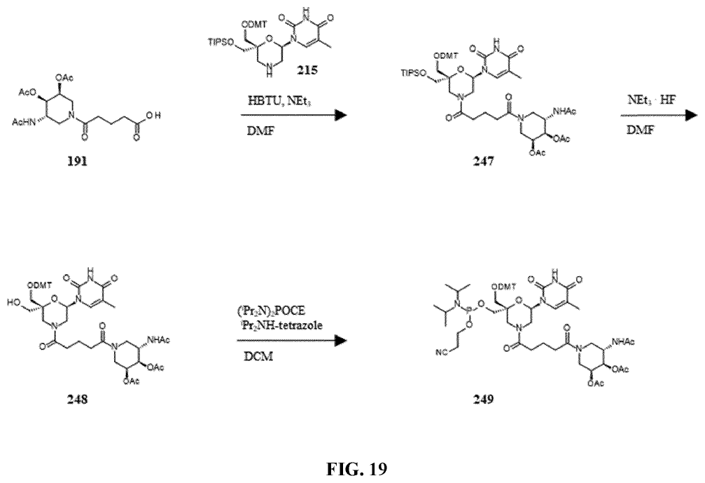

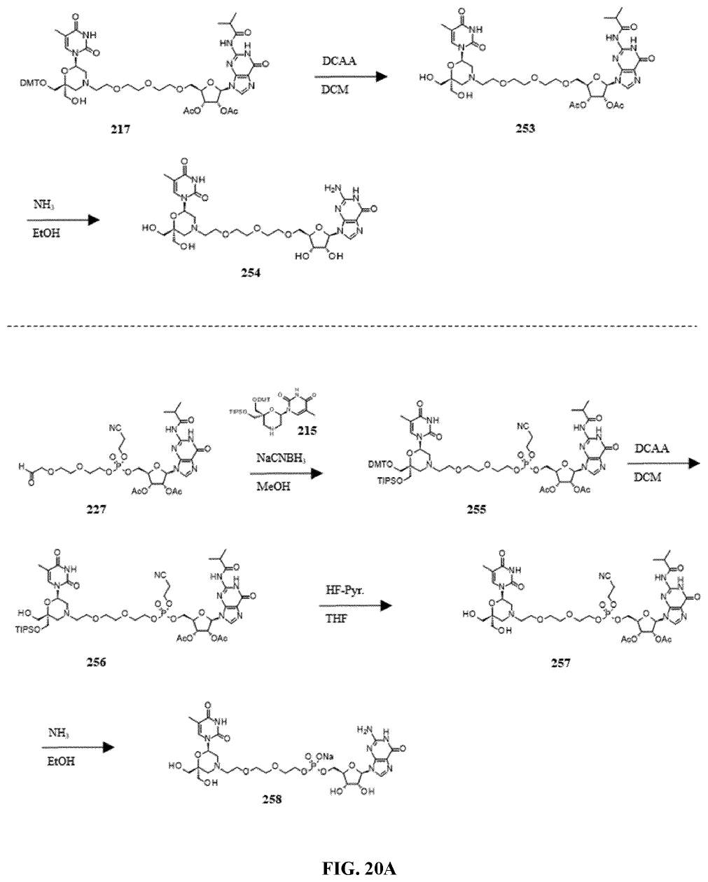

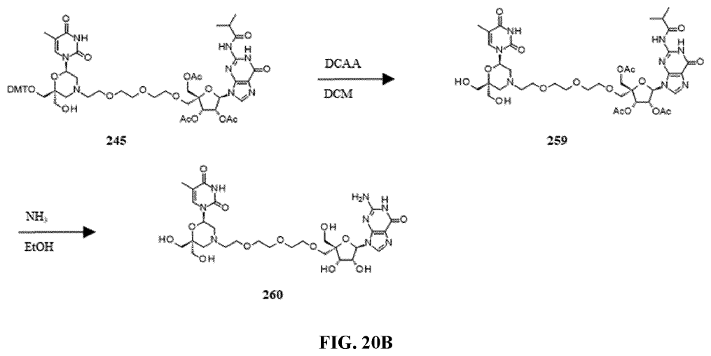

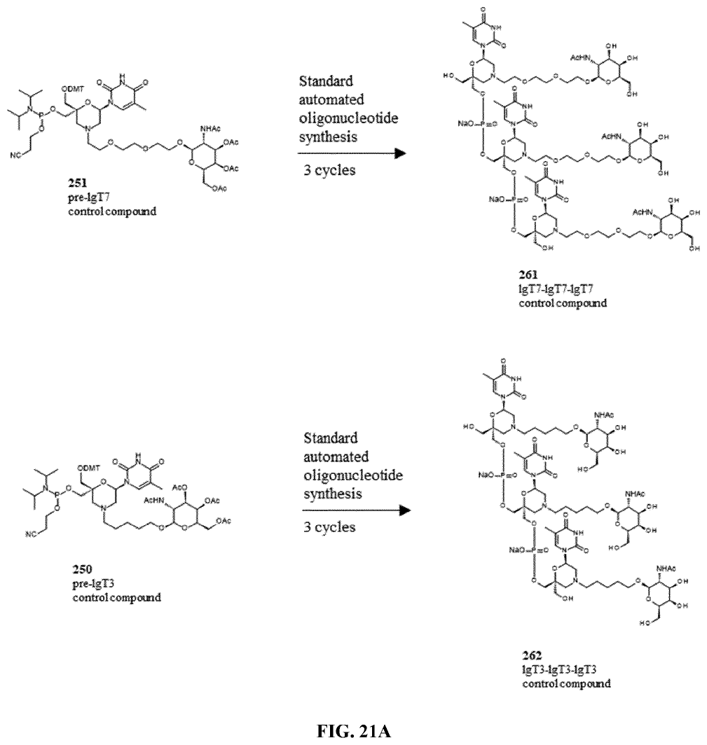

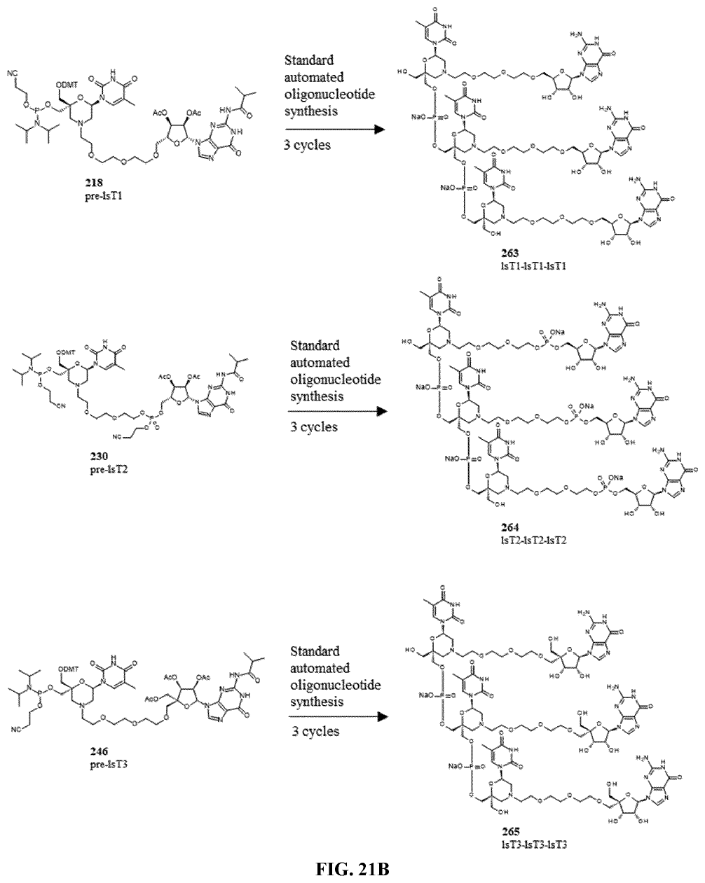

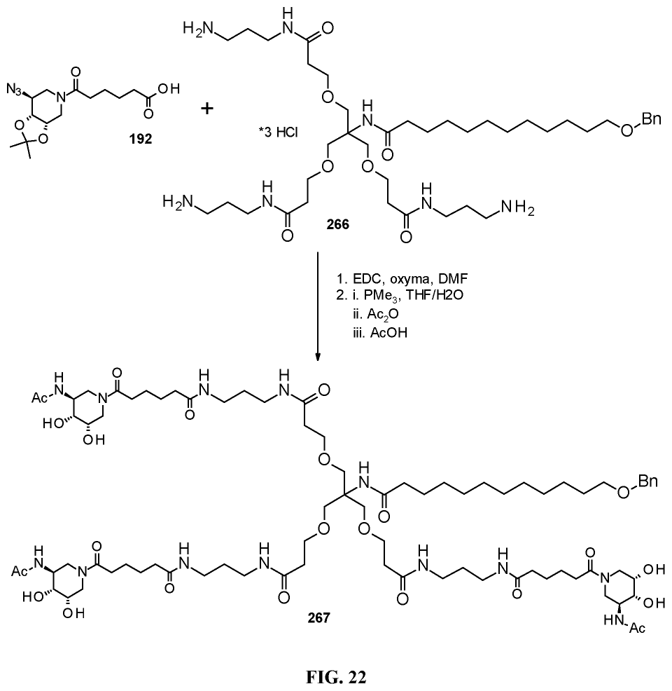

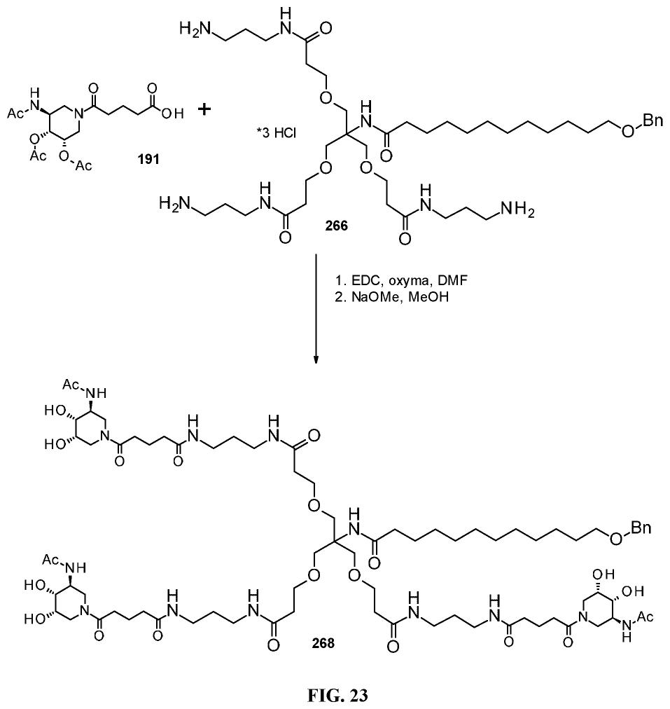

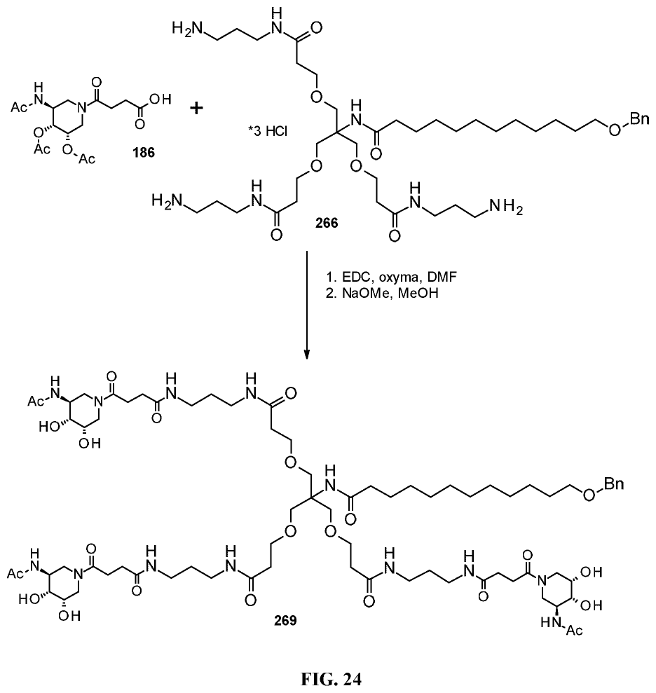

### DETAILED DESCRIPTION

The present disclosure provides novel ligands for asialoglycoprotein receptor (ASGPR), such as human ASGPR. These ASGPR-binding ligands or their chemically protected analogs are piperidine or guanosine derivatives listed in Tables C, D, E, F, G, H, J, K, L, and M, or described in Examples 1-25, and can be conjugated to therapeutic nucleic acid molecules and target them to tissues that express ASGPR, such as the liver. For example, the present ASGPR ligands can be conjugated to nucleotides or to nucleotide analogs that are incorporated into therapeutic oligonucleotides, including double-stranded oligonucleotides such as dsRNAs (e.g., siRNAs) and single-stranded oligonucleotides such as antisense oligonucleotides. Oligonucleotides containing these ASGPR-targeted nucleotide analogs exhibit superior biological activity, including efficient delivery and uptake by specific cells or tissue, e.g., hepatocytes, exceptional in vivo potency, and remarkable in vitro stability. These ASGPR-targeted oligonucleotides may be useful for silencing (e.g., reducing or eradicating) the expression of a target gene. In particular embodiments, this invention encompasses specific piperidine and guanosine-derived ASGPR-binding ligands and nucleotide analogs conjugated thereto for incorporation into double-stranded RNAs (dsRNAs), e.g., siRNAs, that can hybridize to messenger RNAs (mRNAs) of interest so as to reduce or block the expression of target genes of interest.

All technical and scientific terms used herein are the same as those commonly used by those ordinary skilled in the art to which the present invention pertains unless defined specifically otherwise.

An “alkyl group” or a “hydrocarbon chain” refers to a group of 1-20, 1-18, 1-16, 1-12, 1-10, preferably 1-8, more preferably 1-6 unsubstituted or substituted hydrogen-saturated carbons connected in linear, branched, or cyclic fashion, including the combination in linear, branched, and cyclic connectivity. Non-limiting examples include methyl, ethyl, propyl, isopropyl, butyl, tert-butyl, and pentyl.

“Cycloalkyl” refers to a monocyclic or polycyclic non-aromatic radical that contains carbon and hydrogen, and may be saturated, or partially unsaturated. Cycloalkyl groups include groups having from 3 to 10 ring atoms (e.g., C₃-C₁₀ cycloalkyl). Whenever it appears herein, a numerical range such as “3 to 10” refers to each integer in the given range; e.g., “3 to 10 carbon atoms” means that the cycloalkyl group may consist of 3 carbon ring atoms, 4 carbon ring atoms, 5 carbon ring atoms, etc., up to and including 10 carbon ring atoms. In some embodiments, it is a C₃-C₈ cycloalkyl radical. In some embodiments, it is a C₃-C₅ cycloalkyl radical. Examples of cycloalkyl group include, but are not limited to, cyclopropyl, cyclobutyl, cyclopentyl, cyclopentenyl, cyclohexyl, cyclohexenyl, cycloheptyl, cyclooctyl, cyclononyl, cyclodecyl, and norbornyl. The term “cycloalkyl” also refers to spiro-connected ring systems, in which the cycloalkyl rings share one carbon atom.

“Heterocycloalkyl” refers to a 3-to 18-membered nonaromatic ring (e.g., C3-C18 heterocycloalkyl) radical that comprises two to twelve ring carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heterocycloalkyl group may consist of 3 ring atoms, 4 ring atoms, etc., up to and including 18 ring atoms. In some embodiments, it is a C₅-C₁₀ heterocycloalkyl. In some embodiments, it is a C₄-C₁₀ heterocycloalkyl. In some embodiments, it is a C3-C10 heterocycloalkyl. The heterocycloalkyl radical may be a monocyclic, bicyclic, tricyclic or tetracyclic ring system, which may include fused or bridged ring systems. The heteroatoms in the heterocycloalkyl radical may be optionally oxidized. One or more nitrogen atoms, if present, may optionally be quaternized. The heterocycloalkyl radical may be partially or fully saturated. The heterocycloalkyl may be attached to the rest of the molecule through any atom of the ring(s). Examples of such heterocycloalkyl radicals include, but are not limited to, 6,7-dihydro-5H-cyclopenta[b]pyridine, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl group is aziridinyl, azetidinyl, pyrrolidinyl, piperidinyl, homopiperidinyl, morpholinyl, thiomorpholinyl, piperazinyl, tetrahydrofuranyl, tetrahydrothienyl, dihydrooxazolyl, tetrahydropyranyl, tetrahydrothiopyranyl, indolinyl, tetrahydroquinolyl, tetrahydroisoquinolin and benzoxazinyl, preferably dihydrooxazolyl and tetrahydrofuranyl.

“Halogen” refers to any of halogen atoms fluorine (F), chlorine (Cl), bromine (Br), or iodine (I). A particular example of such halo groups is fluorine.

“Amino” refers to unsubstituted amino and substituted amino groups, for example, primary amines, secondary amines, tertiary amines and quaternary amines. Specifically, “amino” refers to NRₐR_{b}, wherein Rₐ and R_{b}, both directly connected to the N, can be independently selected from hydrogen, deuterium, hydroxy, cyano, formyl, alkyl, haloalkyl, alkenyl, haloalkenyl, alkynyl, haloalkynyl, alkoxycarbonyl, alkoxy, haloalkoxy, thioalkoxy, halothioalkoxy, alkanoyl (acyl), haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, a nitrogen protective group, (CO)-alkyl, (CO)—O-alkyl or S(O)ₙR_{c} (n=0 to 2, R_{c} is directly connected to S), wherein R_{c} is independently selected from hydrogen, deuterium, amino, hydroxy, thiol, alkyl, haloalkyl, aryl, heteroaryl, alkenyl, haloalkenyl, alkynyl, haloalkynyl, alkoxy, haloalkoxy, thioalkoxy and halothioalkoxy.

“Aryl” refers to an unsubstituted or substituted C₆-C₁₄ aromatic hydrocarbon. For example, aryl can be phenyl, napthyl, or fluorenyl.

“Heteroaryl” refers to a C₆-C₁₄ aromatic hydrocarbon having one or more heteroatoms, such as N, O, or S. The heteroaryl can be substituted or unsubstituted. Examples of a heteroaryl include, but are not limited to, azepinyl, acridinyl, benzimidazolyl, benzindolyl, 1,3-benzodioxolyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzofurazanyl, benzothiazolyl, benzothienyl, benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some embodiments, the heteroaryl can be dithiazinyl, furyl, imidazolyl, indolyl, isoquinolinyl, isoxazolyl, oxadiazolyl (e.g., (1,3,4)-oxadiazolyl, or (1,2,4)-oxadiazolyl), oxazolyl, pyrazinyl, pyrazolyl, pyrazyl, pyridazinyl, pyridinyl, pyrimidinyl, pyrimidyl, pyrrolyl, quinolinyl, tetrazolyl, thiazolyl, thienyl, triazinyl, (1,2,3)-triazolyl, (1,2,4)-triazolyl, 1,3,4-oxadiazolyl, 1,2,4-oxadiazolyl, 1,2,4-triazolyl, 1,3,4-thiadiazolyl, 5-amino-1,2,4-oxadiazolyl, 5-amino-1,3,4-oxadiazolyl, 5-amino-1,3,4-oxadiazolyl, 3-methyl-1,2,4-oxadiazolyl, 5-methyl-1,2,4-oxadiazolyl, 5-(trifluoromethyl)-1,2,4-oxadiazolyl, 5-(methylamino)-1,2,4-oxadiazolyl, 5-(aminomethyl)-1,2,4-oxadiazolyl, 5-(aminomethyl)-1,3,4-oxadiazolyl, 5-amino-4-cyanooxazolyl, 5,6-dichloro-1H-indolyl, 5,6-difluoro-1H-indolyl, 5-chloro-1H-indolyl, 5,6-dibromo-1H-indolyl, 5-fluoro-1H-indolyl, 5-methoxy-1H-indolyl, 7-fluoro-1H-indolyl, 6-cyano-1H-indolyl, 5-cyano-1H-indolyl, 4-fluoro-1H-indolyl, 5,6-difluoro-1H-indolyl, 6-fluoro-1H-indolyl, or 5,7-difluoro-1h-indolyl.

The substituent on the aryl or heteroaryl group can be alkyl (e.g., C1-C6 alkyl), alkoxy (e.g., C1-C6 alkoxy), amino, cyano, halo (e.g., fluoro, bromo, and chloro), alkylamino (e.g., C1-C6 alkylamino), methyleneamino, nitro, or hydroxyl. The heteroaryl group can have two, three, or four substituents.

“Heterocycle” refers to an unsubstituted or substituted C₆-C₁₄ cyclic hydrocarbon having one or more heteroatoms, such as N, O, or S.

“Alkoxy” refers to an alkyl connected to an oxygen atom (—O-alkyl).

“Aryloxy” refers to an aryl connected to an oxygen atom (—O-aryl).

“Carbonyl” refers to (CO)—, wherein (CO) indicates that the oxygen is connected to the carbon with a double bond.

“Alkanoyl” or “acyl” refers to an alkyl connected to a carbonyl group [—(CO)-alkyl].

“Aroyl” refers to an aryl connected to a carbonyl group [—(CO)-aryl].

“Carboxy” refers to a carboxylic acid group [—(CO)—OH].

“Alkoxycarbonyl” refers to a carboxylic acid ester group [—(CO)—O-alkyl], wherein the alkyl may be further substituted, for example, by an aryl group.

“Aryloxycarbonyl” refers to a carboxylate ester group [—(CO)—O-aryl], wherein the aryl may be further substituted, for example, by an alkyl or aryl group.

“Arylalkoxycarbonyl” refers to a carboxylate ester group [—(CO)—O-alkyl aryl], wherein the aryl may be further substituted, for example, by an alkyl or aryl group.

“Carbonyloxy” refers to an alkanoyl (or acyl) connected to an oxygen atom [—O—(CO)-alkyl].

“Aroyloxy” refers to an aroyl connected to an oxygen atom [—O—(CO)-aryl].

The terms “alkyl”, “cycloalkyl”, “alkenyl”, “alkynyl”, “aryl”, “heteroaryl”, and “heterocyclyl” may also refer to the corresponding “alkylene”, “cycloalkylene”, “alkenylene”, “alkynylene”, “arylene”, “heteroarylene”, and “heterocyclene”, respectively, which are formed by the removal of two hydrogen atoms.

The term “heterocyclic nucleobase” refers to any nitrogen-containing heterocyclic moiety capable of forming Watson-Crick-type hydrogen bonds and stacking interactions in pairing with a complementary nucleobase or nucleobase analog (i.e., derivatives of nucleobases) when that nucleobase is incorporated into a polymeric structure.

Unless otherwise specified, the term “heterocyclic nucleobase” refers herein to an optionally substituted, nitrogen-containing heterocyclic group that can be attached to an optionally substituted ribose ring, optionally substituted deoxyribose ring, optionally substituted dioxane ring, or to an optionally substituted morpholino ring, according to the present disclosure. In some embodiments, the heterocyclic nucleobase can be selected from an optionally substituted purine-base or an optionally substituted pyrimidine-base. The term “purine-base” is used herein in its ordinary sense as understood by those skilled in the art, and includes its tautomers. Similarly, the term “pyrimidine-base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g., 7-methylguanine), theobromine, caffeine, uric acid and isoguanine. Examples of pyrimidine-bases include, but are not limited to, cytosine, thymine, uracil, 5,6-dihydrouracil and 5-alkylcytosine (e.g., 5-methylcytosine). Other nonlimiting examples of heterocyclic nucleobases include diaminopurine, 8-oxo-N6 alkyladenine (e.g., 8-oxo-Ne methyladenine), 7-deazaxanthine, 7-deazaguanine, 7-deazaadenine, N4,N4-ethanocytosin, N6,N6-ethano-2,6-diaminopurine, 5-halouracil (e.g., 5-fluorouracil and 5-bromouracil), pseudoisocytosine, isocytosine, isoguanine, 1,2,4-triazole-3-carboxamides and other heterocyclic nucleobases described in U.S. Pat. Nos. 5,432,272 and 7,125,855, which are incorporated herein by reference disclosing additional heterocyclic bases. In some embodiments, a heterocyclic nucleobase can be optionally substituted with an amine- or an enol protecting group(s).

The term “protecting group” as used herein refers to any atom or group of atoms that is added to a molecule in order to prevent existing groups in the molecule from undergoing unwanted chemical reactions. A “protecting group” may be a labile chemical moiety that is known in the art to protect reactive groups, such as hydroxyl, amino and thiol groups, against undesired or untimely reactions during chemical synthesis. Protecting groups are typically used selectively and/or orthogonally to protect sites during reactions at other reactive sites and can then be removed to leave the unprotected group as it is or available for further reactions.

Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art.

A non-limiting list of protecting groups include benzyl; substituted benzyl; alkylcarbonyls (acetyl or isobutyryl), arylcarbonyls, alkoxycarbonyls and aryloxycarbonyls (e.g., t-butoxycarbonyl (BOC)); arylalkylcarbonyls and arylalkoxycarbonyls (e.g., benzyloxy carbonyl); substituted methyl ether (e.g. methoxymethyl ether); substituted ethyl ether; a substituted benzyl ether; tetrahydropyranyl ether; silylethers (e.g., trimethylsilyl-, triethylsilyl-, triisopropylsilyl-, t-butyldimethylsilyl-, tri-isopropylsilyloxymethyl-, [2-(trimethylsilyl)ethoxy]methyl- or t-butyldiphenylsilyl-); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Preferred protecting groups are selected from a group comprising acteyl (Ac), benzyl (Bzl), isobutyryl (iBu), phenylacetyl, dimethoxytrityl (DMT), methoxytrityl (MMT), triphenylmethyl (Trt), N,N-dimethylformamidine, and 2-cyanoethyl (CE). Unless indicated otherwise, the abbreviations for any protecting groups, amino acids, and other compounds are in accordance with their common usage, recognized abbreviations, or the IUPAC-IUB Commission on Biochemical Nomenclature (see, Biochem. 11:942-944 (1972)).

As used herein, a “reactive phosphorus group” refers to a phosphorus-containing group comprised in a nucleotide unit or in a nucleotide analog unit and which may react with a hydroxyl group or an amine group comprised in another molecule, and especially in another nucleotide unit or in another nucleotide analog, through a nucleophilic attack reaction. Generally, such a reaction, followed by an oxidation step, generates a phosphate ester-type intemucleoside linkage linking the first nucleotide unit or the first nucleotide analog unit to the second nucleotide unit or to the second nucleotide analog unit.

In some embodiments, a reactive phosphorus group can be selected from the group consisting of phosphoramidite, H-phosphonate, alkyl-phosphonate, phosphate or phosphate mimics include but not limited to: natural phosphate, phosphorothioate, phosphorodithioate, borano phosphate, borano thiophosphate, phosphonate, halogen substituted phosphonates and phosphates, phosphoramidates, phosphodiester, phosphotriester, thiophosphodiester, thiophosphotriester, diphosphates and triphosphates. Protecting groups at the nucleotide or nucleotide analog encompass hydroxyl-, amine- and phosphoramidite protecting groups, which may be selected from a group comprising acetyl (Ac), benzyl (Bzl), benzyl (Bn), isobutyryl (iBu), phenylacetyl, benzyloxymethyl acetal (BOM), beta-methoxyethoxymethyl ether (MEM), methoxymethylether (MOM), p-methoxybenzyl ether (PMB), methylthiomethyl ether, pivaloyl (Piv), tetrahydropyranyl (THP), triphenylmethyl (Trt), methoxytrityl [(4-methoxyohenyl)diphenylmethyl] (MMT), dimethoxytrityl, [bis-(4-methoxyphenyl)phenylmethyl (DMT), trimethylsilyl ether (TMS), tert-butyldimethylsilyl ether (TBDMS), tri-iso-propylsilyloxymethyl ether (TOM), tri-isopropylsilyl ether (TIPS), methyl ethers, ethoxyethyl ethers (EE) N,N-dimethylformamidine and 2-cynaonethyl (CE).

### I. Nucleotide Modifications

As used herein, the term “nucleotide” includes naturally occurring or modified nucleotides, or a surrogate replacement moiety. A modified nucleotide, also referred to herein as a “nucleotide analog,” is a non-naturally occurring nucleotide. One of ordinary skill in the art would understand that guanine, cytosine, adenine, uracil, or thymine in a nucleotide may be replaced by other moieties without substantially altering the base-pairing properties of the modified nucleotide. For example, a nucleotide comprising inosine as its base may base-pair with nucleotides containing adenine, cytosine, or uracil. Hence, nucleotides containing uracil, guanine, or adenine may be replaced in the nucleotide sequences of the present disclosure by a nucleotide containing, for example, inosine. Sequences comprising such replacement moieties are included as embodiments of the present disclosure. A modified nucleotide may also be a nucleotide whose ribose moiety is replaced with a non-ribose moiety. As used herein, the terms “nucleoside” and “nucleoside analog” respectively refer to a nucleotide and nucleotide analog without its phosphate groups.

A nucleotide analog of the present disclosure may comprise any modification known in the art, including, for example, end modifications, base modifications, sugar modifications/replacements, and backbone modifications.

End modifications may include, for example, 5′ end modifications (e.g., phosphorylation, conjugation, and inverted linkages) and 3′ end modifications (e.g., conjugation, DNA nucleotides, and inverted linkages).

Base modifications may include, e.g., replacement with stabilizing bases, destabilizing bases, or bases that base-pair with an expanded repertoire of partners; removal of bases (abasic modifications of nucleotides); or conjugation with bases.

Modifications to the sugar group may include chemical modifications at the 2′-carbon atom or the 2′-hydroxy group of the ribose ring, e.g., 2′-desoxy-2′-F (fluoro), 2′-OMe (methoxy), and 2′-O-methoxyethyl modifications. Although the majority of sugar alterations are localized at the 2′-position, modifications at other positions such as the 4′-position are also allowed (Leydler et al., Antisense Res Dev. (1995) 5:161-74).

Other chemical modifications of the sugar group may include linking the 2′-oxygen and 4′-carbon of the ribose scaffold in a nucleoside, creating a so-called locked nucleic acid (“LNA”). LNAs, also referred to as bicyclic nucleic acids, have been shown to have increased RNA-binding affinity (Koshin et al., Tetrahedron (1998) 54:3607-30; Prakash et al., Chem Biodivers. (2011) 8:1616-41), which leads to a significant increase in the melting temperature of the resulting double stranded oligonucleotides. However, fully LNA-modified oligomers longer than eight nucleotides tend to aggregate. Contrasting with the rigid nature of the LNA modification, the highly flexible unlocked nucleic acid (“UNA”) modification may also be incorporated in the nucleotide analogs described herein. UNA nucleosides do not have the C2′-C3′-bond of the ribose sugar. Due to their open chain structure, UNAs are not conformationally restrained and have been used to modulate oligonucleotide flexibility (Mangos et al., J Am Chem Soc. (2003) 125:654-61). UNA inserts can reduce duplex melting temperature (Tm) by 5° C.-10° C. per insert in some cases. Further, UNA inserts can facilitate antisense strand selection by a RISC, and UNA modifications to the seed region of an siRNA guide strand can reduce off-target events (Vaish et al., Nucleic Acids Res. (2011) 39:1823-32). UNA- and LNA-containing siRNAs have been reported by Bramsen et al., Nucleic Acids Research (2010) 38(17):5761-73).

Further, expanded sugar ring systems, including six-membered morpholino ring systems, where the ribose moiety of a nucleoside is replaced by a morpholine ring, may also be incorporated in the nucleotide analogs described herein. Morpholino-based nucleosides form internucleotide linkages within oligonucleotides containing them through the nitrogen atom of the morpholine subunit. Phosphorodiamidate morpholino-based oligonucleotides (“PMOs”) have been used in antisense technology (Corey et al., Genome Biology (2001) 2(5): reviews 1015.1-1015; Partridge et al., Antisense Nucleic Acid Drug Dev. (1996) 6:169-75). Examples of morpholino subunits are also disclosed in U.S. Pat. Nos. 5,034,506; 5,166,315; 5,185,444; 5,698,685; and U.S. Patent Publication US2016US/0186174.

A nucleotide or nucleotide analog of the present disclosure may be conjugated to a cell targeting moiety. Such a nucleotide or nucleotide analog is referred to as a “targeted nucleotide”. A nucleotide or nucleotide analog that is not conjugated to a cell targeting moiety is referred to as a “non-targeted nucleotide”.

Building blocks of nucleotide or nucleotide analogs of the present disclosure, targeted and non-targeted, which are used for the syntheses of oligonucleotides and the incorporation of such nucleotides into an oligonucleotide are called nucleotide precursors. These targeted or non-targeted nucleotide precursors show specific chemical modifications, necessary for the automated oligonucleotide synthesis. Common functionalizations are reactive phosphorous groups, e.g., phosphoramidites as well as specific protecting groups, as e.g., DMT-protecting groups.

Internucleotide linkages constitute the backbone of a nucleic acid molecule. An internucleotide linking group refers to a chemical group linking two adjacent nucleoside residues in a nucleic acid molecule, which encompasses (i) a chemical group linking two adjacent nucleoside residues, (ii) a chemical group linking a nucleoside residue with an adjacent nucleoside analog residue and (iii) a chemical group linking a first nucleoside analog residue with a second nucleoside analog residue, wherein the nucleoside analog residues may be identical or may be distinct. The terms “internucleoside linkage,” “internucleoside linking group,” “internucleotide linkage,” or “internucleotide linking group” are used interchangeably herein and refer to any linker or linkage between two nucleoside (i.e., a heterocyclic base moiety and a sugar moiety) units, as is known in the art, including but not limited to, phosphate, analogs of phosphate, phosphorothioate, phosphonate, guanidium, hydroxylamine, hydroxylhydrazinyl, amide, carbamate, alkyl, and substituted alkyl linkages.

Backbone modifications may include chemical modifications performed on internucleotide linkages by replacing the 3′-5′ phosphodiester linkage with more stable moieties to reduce susceptibility to nuclease degradation. A widely used modification is a partial or complete replacement of the phosphodiester backbone with phosphorothioate linkages, in which a sulfur atom is used in place of a non-bridging oxygen atom. Backbone modifications may also include modification or replacement of the phosphodiester linkages with one or more phosphorodithioates, phosphotriesters, methyl and other alkyl phosphonates, phosphinates, or phosphoramidates. An alternative backbone modification that confers increased stability to nucleic acids is the boranophosphate linkage. In boranophosphate oligonucleotides, the non-bridging phosphodiester oxygen is replaced with an isoelectronic borane (—BH₃) moiety.

Non-targeted nucleotide precursors, found in the present disclosure are described by Hofmeister et al. in WO 2019/170731. Examples are listed in Table A. The morpholine-type nucleotide precursors within the (2S,6R)-diastereomeric series are abbreviated with “pre-1” followed by the nucleobase (T, U, C, A or G) and a number, which specifies the substituent at the morpholine nitrogen. The analogues (2R,6R)-diastereoisomers are abbreviated with an additional “b.” The abbreviations for the corresponding nucleotides within an oligonucleotide sequence are built by the same rules, but without the “pre” and are also shown in Table A.

TABLE A

| | | | Name in | |
|---|---|---|---|---|
| | | Precursor | oligo- | Stereo- |
| Entry | Structure | name | sequence | chemistry |
| 1 | 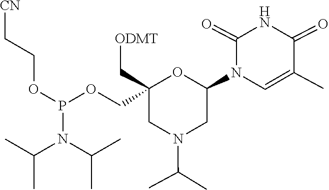 | pre-1T3 | 1T3 | (2S,6R) |
| 2 | 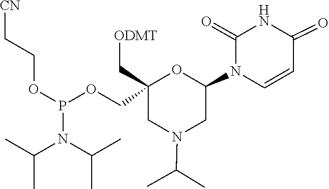 | pre-1U3 | 1U3 | (2S,6R) |
| 3 | 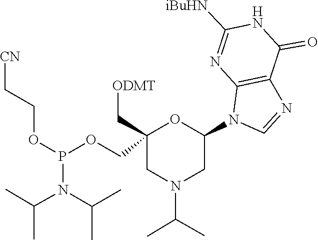 | pre-1G3 | 1G3 | (2S,6R) |
| 4 | 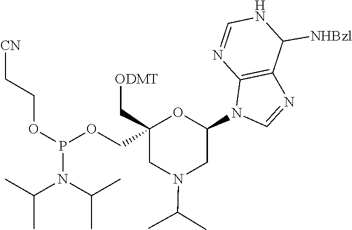 | pre-1A3 | 1A3 | (2S,6R) |
| 5 | 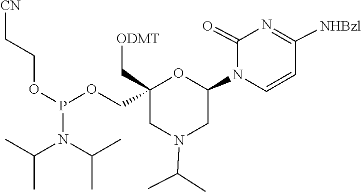 | pre-1C3 | 1C3 | (2S,6R) |
| 6 | 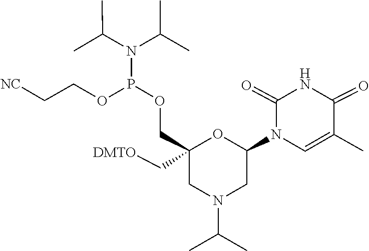 | pre-1T3b | 1T3b | (2R,6R) |
| 7 | 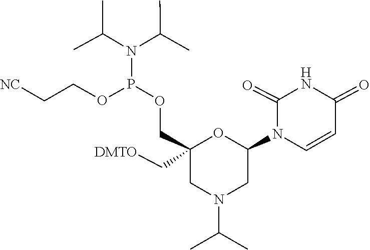 | pre-1U3b | 1U3b | (2R,6R) |
| 8 | 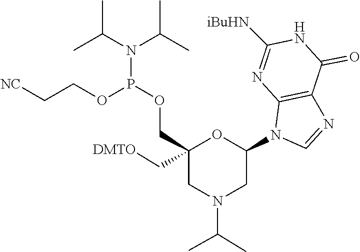 | pre-1G3b | 1G3b | (2R,6R) |
| 9 | 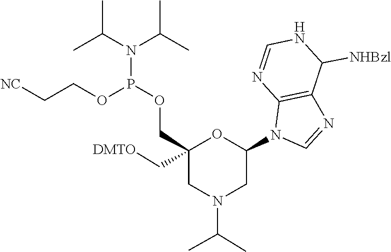 | pre-1A3b | 1A3b | (2R,6R) |
| 10 | 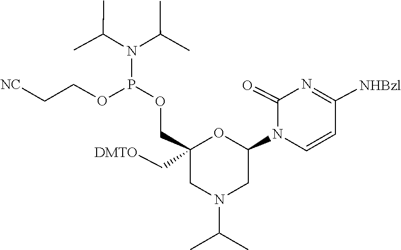 | pre-1C3b | 1C3b | (2R,6R) |
| 11 | 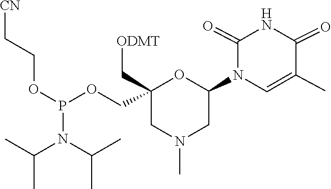 | pre-1T2 | 1T2 | (2S,6R) |
| 12 | 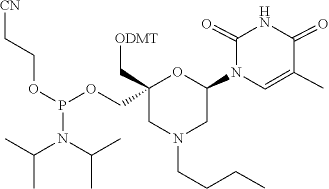 | pre-1T6 | 1T6 | (2S,6R) |
| 13 | 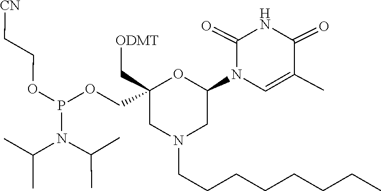 | pre-1T7 | 1T7 | (2S,6R) |
| 14 | 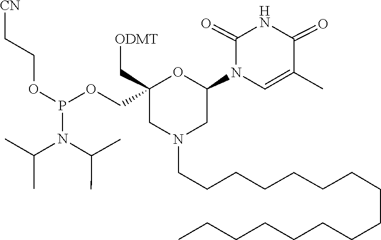 | pre-1T8 | 1T8 | (2S,6R) |
| 15 | 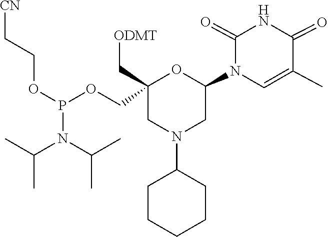 | pre-1T4 | 1T4 | (2S,6R) |
| 16 | 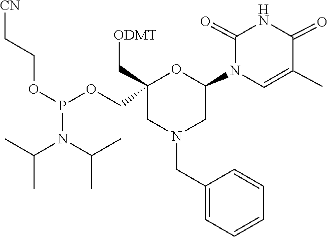 | pre-1T5 | 1T5 | (2S,6R) |
| 17 | 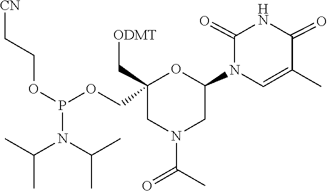 | pre-1T9 | 1T9 | (2S,6R) |
| 18 | 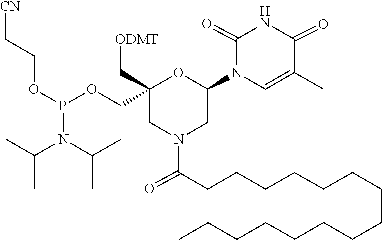 | pre-1T10 | 1T10 | (2S,6R) |
| 19 | 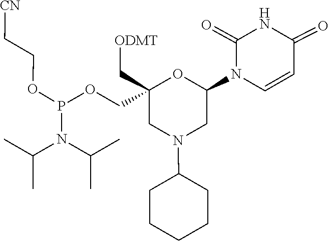 | pre-1U4 | 1U4 | (2S,6R) |
| 20 | 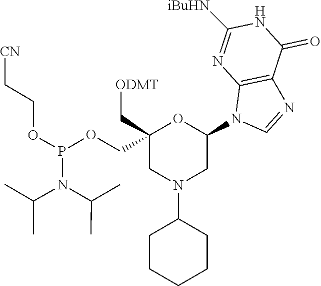 | pre-1G4 | 1G4 | (2S,6R) |
| 21 | 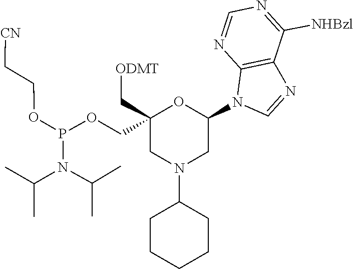 | pre-1A4 | 1A4 | (2S,6R) |
| 22 | 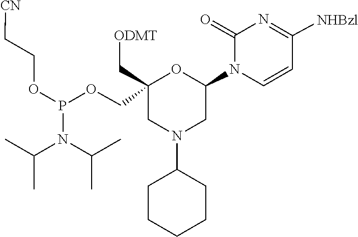 | pre-1C4 | 1C4 | (2S,6R) |
| 23 | 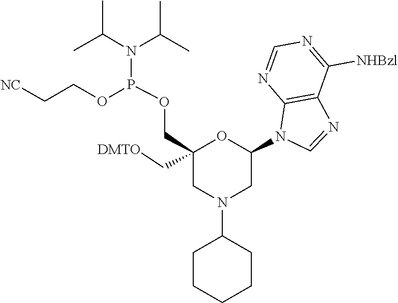 | pre-1A4b | 1A4b | (2R,6R) |
| 24 | 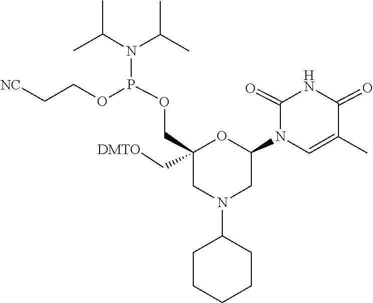 | pre-1T4b | 1T4b | (2R,6R) |

Based on the same scaffold, targeted nucleotide precursors are described in the same application (Hofmeister et al. WO 2019/170731). Using a GalNAc-residue for ASGPR-targeting, the precursor molecules are abbreviated with “pre-lg”, followed by the nucleobase and a number, which specifies the linker between the morpholine nitrogen and the GalNAc-residue. Examples are shown in Table B.

TABLE B

| | | | Name in | |
|---|---|---|---|---|
| | | Precursor | oligo- | Stereo- |
| Entry | Structure | name | sequence | chemistry |
| 1 | 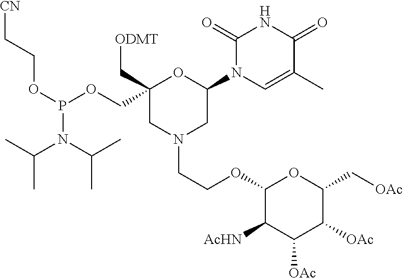 | pre-lgT9 | lgT9 | (2S, 6R) |
| 2 | 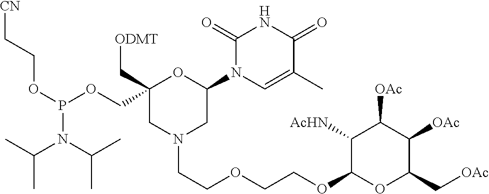 | pre-lgT8 | lgT8 | (2S, 6R) |
| 3 | 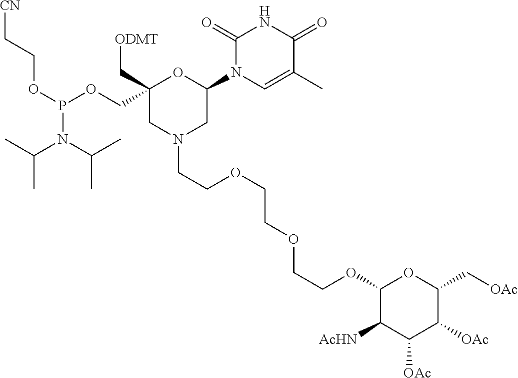 | pre-lgT7 | lgT7 | (2S, 6R) |
| 4 | 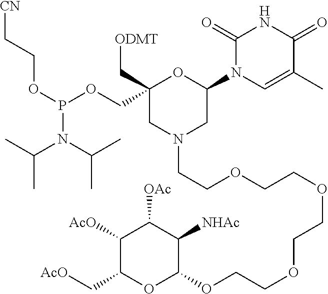 | pre-lgT6 | lgT6 | (2S, 6R) |
| 5 | 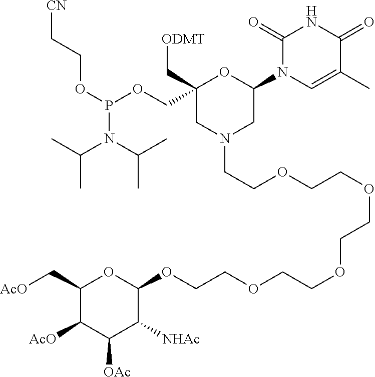 | pre-lgT5 | lgT5 | (2S, 6R) |
| 6 | 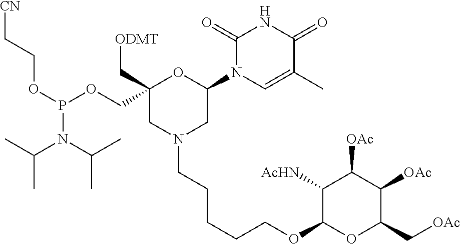 | pre-lgT3 | lgT3 | (2S, 6R) |
| 7 | 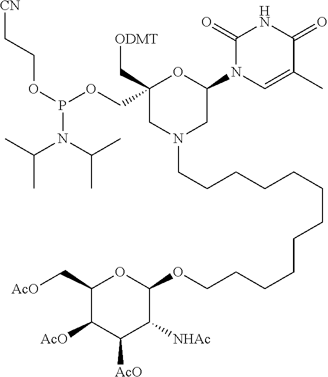 | pre-lgT4 | lgT4 | (2S, 6R) |
| 8 | 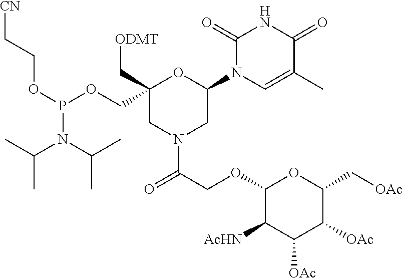 | pre-lgT12 | lgT12 | (2S, 6R) |
| 9 | 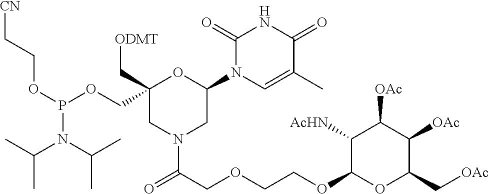 | pre-lgT11 | lgT11 | (2S, 6R) |
| 10 | 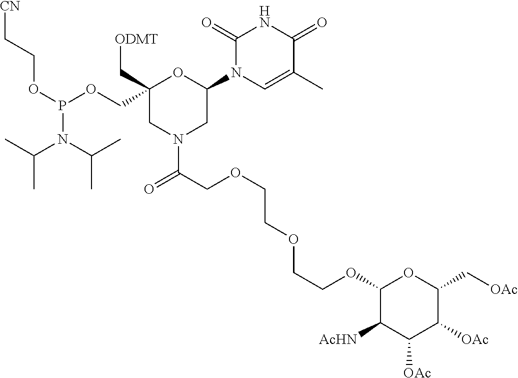 | pre-lgT10 | lgT10 | (2S, 6R) |
| 11 | 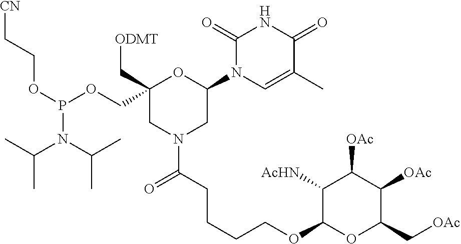 | pre-lgT1 | lgT1 | (2S, 6R) |
| 12 | 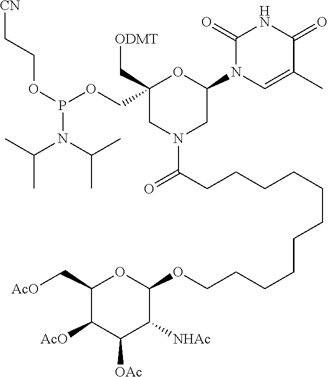 | pre-lgT2 | lgT2 | (2S, 6R) |

In some embodiments, a nucleotide analog precursor of the present disclosure is a compound of general formula (I):

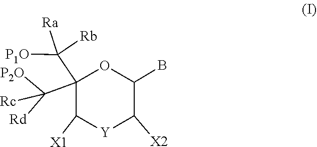

wherein:
B is a heterocyclic nucleobase;
P₁ and P₂ are each, independently, H, a reactive phosphorous group, or a protecting group;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is an ASGPR-binding cell targeting moiety or a protected form thereof, wherein the cell targeting moiety may be piperidine, a piperidine-derived ligand, guanosine, or a guanosine-derived ligand that specifically binds to ASGPR; and
each of X1, X2, Ra, Rb, Rc, and Rd independently is H or a (C1-C6) alkyl group.

As described in the present disclosure, L can be a branched or unbranched linking group. A branched linking group can have 2, 3, 4, or 5 cell targeting moieties or protected forms thereof.

In some embodiments of a compound of formula (I), Y is NR1, wherein R1 is -L-R3, wherein L is a C2-C25 hydrocarbon chain, and R3 is an ASGPR-binding cell targeting moiety or a protected form thereof.

In some embodiments of a compound of formula (I), Y is NR1, wherein R1 is -L-R3, wherein L is a C1-C10 hydrocarbon chain, and R3 is an ASGPR-binding cell targeting moiety or a protected form thereof.

In some embodiments of a compound of formula (I), Y is N—C(═O)—R1, wherein R1 is -L-R3, wherein L is a C2-C25 hydrocarbon chain, and R3 is an ASGPR-binding cell targeting moiety or a protected form thereof.

In some embodiments of a compound of formula (I), Y is N—C(═O)—R1, wherein R1 is -L-R3, wherein L is a C1-C10 hydrocarbon chain, and R3 is an ASGPR-binding cell targeting moiety or a protected form thereof.

In some embodiments of a compound of formula (I), Y is NR1, wherein R1 is -L-R3, wherein L is a C2-C25 hydrocarbon chain optionally terminated by —C(O)—, and R3 is an ASGPR-binding cell targeting moiety or a protected form thereof.

In some embodiments of a compound of formula (I), Y is NR1, wherein R1 is -L-R3, wherein L is a C1-C10 hydrocarbon chain optionally terminated by —C(O)—, and R3 is an ASGPR-binding cell targeting moiety or a protected form thereof.

In some embodiments of a compound of formula (I), Y is N—C(═O)—R1, wherein R1 is -L-R3, wherein L is a C2-C25 hydrocarbon chain optionally terminated by —C(O)—, and R3 is an ASGPR-binding cell targeting moiety or a protected form thereof.

In some embodiments of a compound of formula (I), Y is N—C(═O)—R1, wherein R1 is -L-R3, wherein L is a C1-C10 hydrocarbon chain optionally terminated by —C(O)—, and R3 is an ASGPR-binding cell targeting moiety or a protected form thereof.

In some embodiments of a compound of formula (I), Y is NR1, wherein R1 is -L-R3, wherein L is a C2-C25 hydrocarbon chain optionally interrupted by one or more —O—, and R3 is an ASGPR-binding cell targeting moiety or a protected form thereof.

In some embodiments of a compound of formula (I), Y is NR1, wherein R1 is -L-R3, wherein L is a C2-C10 hydrocarbon chain optionally interrupted by one or more —O—, and R3 is an ASGPR-binding cell targeting moiety or a protected form thereof.

### II. ASGPR Ligands

A nucleotide or nucleotide analog of the present disclosure may be conjugated to one or more ligands targeting specific cells or tissue. Such a ligand is also called a “cell targeting moiety.” As used herein, a “cell targeting ligand or moiety” refers to a molecular group that ensures efficient delivery of an oligonucleotide, e.g. dsRNA, attached thereto to a target cell or tissue by increasing (i) affinity of the dsRNA for the target receptor (e.g., target protein) or cells expressing the target receptor; (ii) uptake of the dsRNA by the target cells; and/or (iii) ability of the dsRNA to be appropriately processed once it has entered into the target cell, including efficient intracellular release of the dsRNA, e.g., by facilitating translocation of the dsRNA from transport vesicles into the cytoplasm. Thus, a cell targeting moiety is used to direct and/or deliver an oligonucleotide to a particular cell, tissue, organ, etc. A cell targeting moiety attached to a nucleotide, a nucleotide analog, or to an oligonucleotide imparts to the nucleotide, nucleotide analog, or oligonucleotide characteristics such that the nucleotide, nucleotide analog, or oligonucleotide is preferentially recognized, bound, internalized, processed, activated, etc. by the targeted cell type(s) relative to non-targeted cell types. Accordingly, compounds comprising a cell targeting moiety preferentially interact with and are taken up by the targeted cell type(s). In some embodiments, a cell targeting moiety may be chemically protected using protection groups well known in the art.

As used herein, “target cells” or “targeted cells” refer to cells of interest. The cells may be found in vitro, in vivo, ex vivo, or in the tissue or organ of an organism. The organism may be an animal, preferably a mammal, more preferably a human, and most preferably a human patient. In particular embodiments, the target cell is a hepatocyte.

### II.1 Piperidine-Derived Cell Targeting Ligands

A cell targeting moiety of the present disclosure may be a piperidine-derived ligand that specifically binds to ASGPR. In some embodiments, the piperidine-derived ASGPR-binding ligand is a moiety of formula (II) or a compound of formula (III)

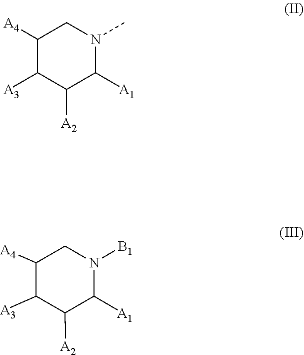

wherein:
A₁, A₂, and A₃ are, independently, H, hydroxy, alkoxy, acyloxy, aryloxy, aroyloxy, alkoxycarbonyl, aryloxycarbonyl, oxo (═O), or a (C1-C20) alkyl or alkenyl group, unsubstituted or optionally substituted by one or more groups selected from OH, a (C3-C8) cycloalkyl group, a (C3-C14) heterocycle, a (C6-C14) aryl group, a (C5-C14) heteroaryl group, —O—Z5, —N(Z5)(Z6), —S—Z5, —CN, —C(=M)-O—Z5, —O—C(=M)-Z5, —C(=M)-N(Z5)(Z6), and —N(Z5)-C(=M)-Z6, wherein:
   M is O or S,
   each of Z5 and Z6 is, independently, H, a (C1-C6) alkyl group, or a (C6-C14) aryl group, wherein both the alkyl and aryl groups may be either unsubstituted or optionally substituted by one or more groups selected from halogen, amino, hydroxy, thiol, cyano, alkyl, alkoxy, aryloxy, acyloxy, aroyloxy, carboxy, alkoxycarbonyl, aryloxycarbonyl and arylalkoxycarbonyl;
A₄ is —N(R4)₂, —NH—C(═O)—R4, or

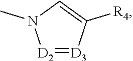

wherein:
D2 and D3 are N, O, or S;
R4 is H or a (C1-C20) alkyl group, unsubstituted or optionally substituted by one or more groups selected from halogen, amino, hydroxy, thiol, cyano, alkyl, alkoxy, aryloxy, acyloxy, aroyloxy, carboxy, alkoxycarbonyl, aryloxycarbonyl, and arylalkoxycarbonyl;
B₁ is H, benzylester, -L-R5, or —(CO)-L-R5, wherein:
   L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxy, or haloalkyl, the C1-C25 hydrocarbon chain being optionally substituted with one or more -L′-R5, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; and
   R5 is H, OH, benzyl, benzyloxy, a nucleoside, a nucleoside analog, a nucleotide, or a nucleotide analog.

The cell targeting moieties of formula (II) or of formula (III) also consist of specific objects of the present disclosure.

As described in the present disclosure, L can be a branched or unbranched linking group. A branched linking group can have 2, 3, 4, or 5 cell targeting moieties.

In some embodiments of a moiety of formula (II) or a compound of formula (III), A1 is H, oxo (═O), or a (C1-C6) alkyl or alkenyl group optionally substituted by hydroxy or alkoxy.

In some embodiments of a moiety of formula (II) or a compound of formula (III), A1 is a (C1-C6) alkyl group optionally substituted by —O—C(=M)-Z5, wherein M is O and Z5 is a (C1-C6) alkyl group optionally substituted by an alkoxycarbonyl or arylalkoxycarbonyl group.

In some embodiments of a moiety of formula (II) or a compound of formula (III), A1 is a (C1-C6) alkyl group optionally substituted by —O—C(=M)-Z5, wherein M is O and Z5 is a (C1-C6) alkyl group optionally substituted by a benzyl ester group.

In some embodiments of a moiety of formula (II) or a compound of formula (III), A2 and A3 are hydroxy or acyloxy.

In some embodiments of a moiety of formula (II) or a compound of formula (III), A2 and A3 are acetoxy.

In some embodiments of a moiety of formula (II) or a compound of formula (III), A4 is —NH—C(═O)—R4, wherein R4 is a (C1-C6) alkyl group optionally substituted by a carboxy or alkoxycarbonyl group.

In some embodiments of a moiety of formula (II) or a compound of formula (III), A4 is —NH—C(═O)—R4, wherein R4 is a (C1-C6) alkyl group optionally substituted by a methyl ester group.

In some embodiments of a moiety of formula (II) or a compound of formula (III), A4 is

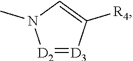

wherein D2 and D3 are N, and R4 is a (C1-C6) alkyl group, optionally substituted by an alkoxy or aryloxy group.

In some embodiments of a moiety of formula (II) or a compound of formula (III), A4 is

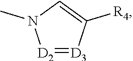

wherein D2 and D3 are N, and R4 is a (C1-C6) alkyl group, optionally substituted by a phenoxy group.

In some embodiments of a compound of formula (III), B1 is H.

In some embodiments of a compound of formula (III), B1 is a benzyloxy carbonyl group.

In some embodiments of a compound of formula (III), L is a C1-C6 hydrocarbon chain.

In some embodiments of a compound of formula (III), L is a C1-C6 hydrocarbon chain optionally terminated by —C(O)—.

In some embodiments of a compound of formula (III), R5 is H, OH, benzyl, or benzyloxy.

In some embodiments of a compound of formula (III), L is a C1-C6 hydrocarbon chain optionally terminated by —C(O)—, and R5 is H, OH, benzyl, or benzyloxy.

In some embodiments of a compound of formula (III), A1 is H, (═O), or a (C1-C6) alkyl or alkenyl group optionally substituted by hydroxy, alkoxy, or aryloxy.

In some embodiments of a compound of formula (III), A1 is a (C1-C6) alkyl group optionally substituted by —O—C(=M)-Z5, wherein M is O and Z5 is a (C1-C6) alkyl group optionally substituted by an alkoxycarbonyl or arylalkoxycarbonyl group.

In some embodiments of a compound of formula (III), A1 is a (C1-C6) alkyl group optionally substituted by —O—C(=M)-Z5, wherein M is O and Z5 is a (C1-C6) alkyl group substituted by a benzyl ester group.

In some embodiments of a compound of formula (III), A2 and A3 are hydroxy.

In some embodiments of a compound of formula (III), A4 is —NH—C(═O)—R4, wherein R4 is a (C1-C6) alkyl group optionally substituted by a carboxy, alkoxycarbonyl, or aryloxycarbonyl group.

In some embodiments of a compound of formula (III), A4 is —NH—C(═O)—R4, wherein R4 is a (C1-C6) alkyl group substituted by a methyl ester group.

In some embodiments of a compound of formula (III), A4 is

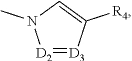

wherein D2 and D3 are N, and R4 is a (C1-C6) alkyl group, optionally substituted by an alkoxy or aryloxy group.

In some embodiments of a compound of formula (III), A4 is

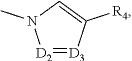

wherein D2 and D3 are N, and R4 is a (C1-C6) alkyl group, substituted by a phenoxy group.

In some embodiments of a compound of formula (III), B1 is H or a benzyl ester group.

Exemplary piperidine-derived ASGPR-binding ligands of formula (III) are shown in Table C below:

TABLE C

| Entry | Structure |
|---|---|
| 1 | 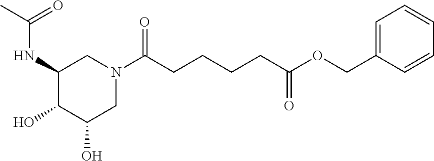 [SMILES: CC(=O)N[C@H]1CN(C(=O)CCCCC(=O)OCc2ccccc2)C[C@H](O)[C@@H]1O] |
| 2 | 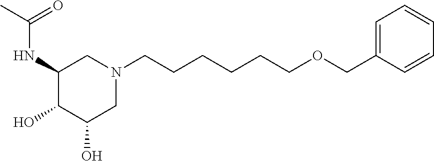 [SMILES: CC(=O)N[C@H]1CN(CCCCCCOCc2ccccc2)C[C@H](O)[C@@H]1O] |
| 3 | 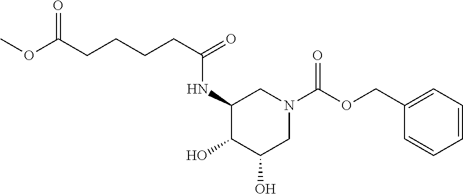 [SMILES: COC(=O)CCCCC(=O)N[C@H]1CN(C(=O)OCc2ccccc2)C[C@H](O)[C@@H]1O] |
| 4 | 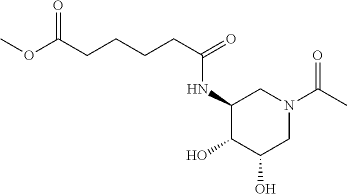 [SMILES: COC(=O)CCCCC(=O)N[C@H]1CN(C(C)=O)C[C@H](O)[C@@H]1O] |
| 5 | 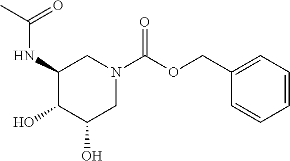 [SMILES: CC(=O)N[C@H]1CN(C(=O)OCc2ccccc2)C[C@H](O)[C@@H]1O] |
| 6 | 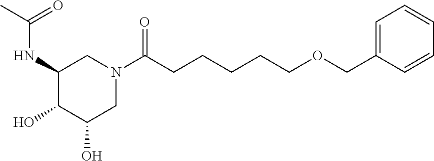 [SMILES: CC(=O)N[C@H]1CN(C(=O)CCCCCOCc2ccccc2)C[C@H](O)[C@@H]1O] |
| 7 | 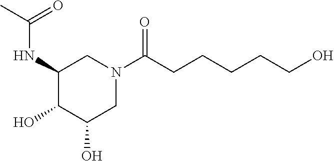 [SMILES: CC(=O)N[C@H]1CN(C(=O)CCCCCO)C[C@H](O)[C@@H]1O] |
| 8 | 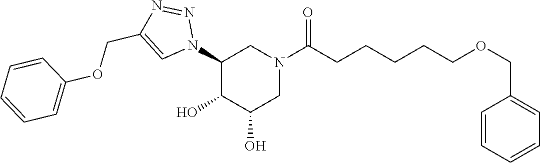 [SMILES: O=C(CCCCCOCc1ccccc1)N1C[C@H](O)[C@H](O)[C@@H](n2cc(COc3ccccc3)nn2)C1] |
| 9 | 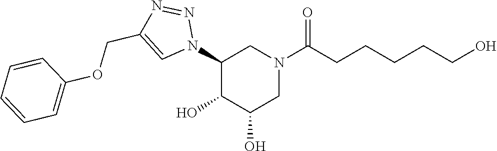 [SMILES: O=C(CCCCCO)N1C[C@H](O)[C@H](O)[C@@H](n2cc(COc3ccccc3)nn2)C1] |
| 10 | 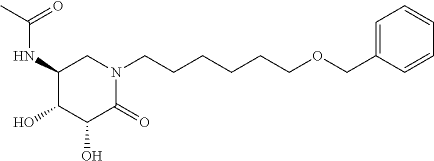 [SMILES: CC(=O)N[C@H]1CN(CCCCCCOCc2ccccc2)C(=O)[C@H](O)[C@@H]1O] |
| 11 | 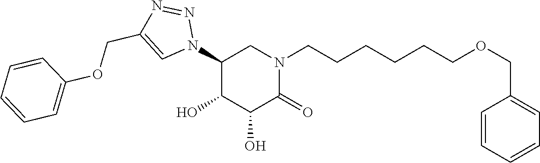 [SMILES: O=C1[C@H](O)[C@H](O)[C@@H](n2cc(COc3ccccc3)nn2)CN1CCCCCCOCc1ccccc1] |
| 12 | 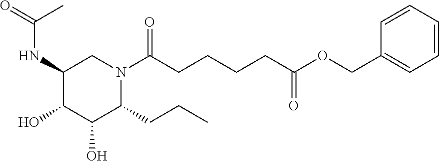 [SMILES: CCC[C@@H]1[C@H](O)[C@H](O)[C@@H](NC(C)=O)CN1C(=O)CCCCC(=O)OCc1ccccc1] |
| 13 | 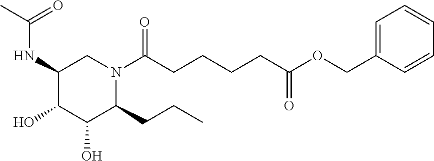 [SMILES: CCC[C@H]1[C@H](O)[C@H](O)[C@@H](NC(C)=O)CN1C(=O)CCCCC(=O)OCc1ccccc1] |
| 14 | 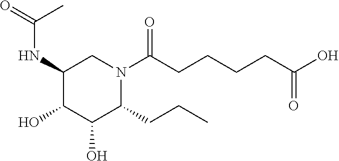 [SMILES: CCC[C@@H]1[C@H](O)[C@H](O)[C@@H](NC(C)=O)CN1C(=O)CCCCC(=O)O] |
| 15 | 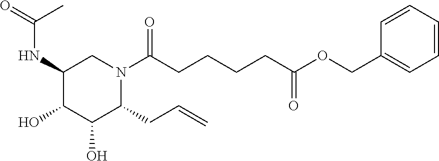 [SMILES: C=CC[C@@H]1[C@H](O)[C@H](O)[C@@H](NC(C)=O)CN1C(=O)CCCCC(=O)OCc1ccccc1] |
| 16 | 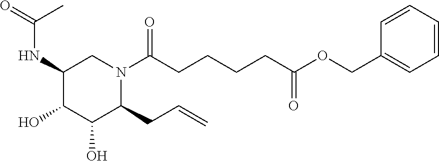 [SMILES: C=CC[C@H]1[C@H](O)[C@H](O)[C@@H](NC(C)=O)CN1C(=O)CCCCC(=O)OCc1ccccc1] |
| 17 | 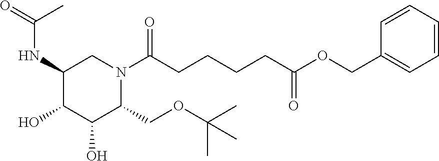 [SMILES: CC(=O)N[C@H]1CN(C(=O)CCCCC(=O)OCc2ccccc2)[C@H](COC(C)(C)C)[C@H](O)[C@@H]1O] |
| 18 | 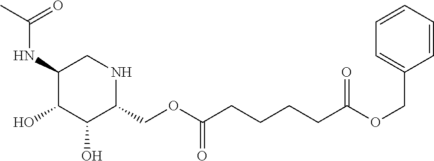 [SMILES: CC(=O)N[C@H]1CN[C@H](COC(=O)CCCCC(=O)OCc2ccccc2)[C@H](O)[C@@H]1O] |
| 19 | 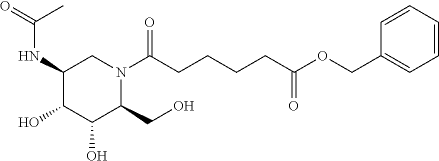 [SMILES: CC(=O)N[C@H]1CN(C(=O)CCCCC(=O)OCc2ccccc2)[C@@H](CO)[C@H](O)[C@@H]1O] |
| 20 | 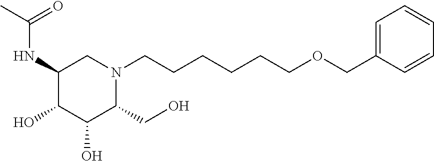 [SMILES: CC(=O)N[C@H]1CN(CCCCCCOCc2ccccc2)[C@H](CO)[C@H](O)[C@@H]1O] |
| 21 | 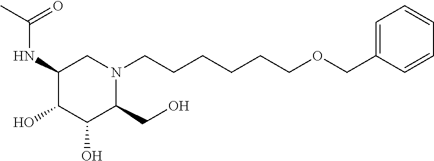 [SMILES: CC(=O)N[C@H]1CN(CCCCCCOCc2ccccc2)[C@@H](CO)[C@H](O)[C@@H]1O] |

### II.2 Guanosine-Derived Cell Targeting Ligands

A cell targeting moiety of the present disclosure may be a guano sine-derived ligand that specifically binds to ASGPR. In some embodiments, the guano sine-derived ASGPR-binding ligand is a moiety of formula (IVA) or (IVB) or a compound of formula (V)

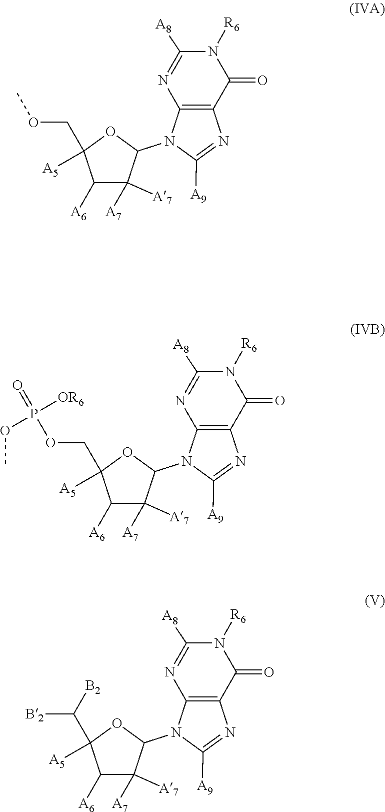

wherein:
each R6 is H or a (C1-C6) alkyl group, unsubstituted or optionally substituted by one or more groups selected from halogen, amino, hydroxy, thiol, alkyl, alkoxy, aryloxy, carboxy, alkoxycarbonyl and aryloxycarbonyl;
A₅, A₆, A₇, and A′₇ are independently H, hydroxy, alkoxy, acyloxy, aryloxy, aroyloxy, alkoxycarbonyl, aryloxycarbonyl, amino, or a (C1-C20) alkyl group unsubstituted or optionally substituted by one or more groups selected from OH, a (C3-C8) cycloalkyl group, a (C3-C14) heterocycle, a (C6-C14) aryl group, a (C5-C14) heteroaryl group, —O—Z7, —N(Z7)(Z8), —S—Z7, —CN, —C(=Q)-O—Z7, —O—C(=Q)-Z7, —C(=Q)-N(Z7)(Z8), and —N(Z7)-C(=Q)-Z8, wherein:
Q is O or S,
each of Z7 and Z8 independently is H, a (C1-C6) alkyl group, or a (C6-C14) aryl group, wherein both the alkyl and aryl groups can be either unsubstituted or optionally substituted by one or more groups selected from a halogen atom and a (C1-C6) alkyl group;
each of A₈ and A₉ independently is H, halogen, OH (or its tautomeric oxo (═O)), —N(R7)₂, —NHR7, or —NH—C(═O)—R7, wherein R7 is hydrogen or a (C1-C20) alkyl group, unsubstituted or optionally substituted by one or more groups selected from a halogen atom, alkoxy, aryloxy, a (C1-C6) alkyl group, a (C3-C8) cycloalkyl group, a (C3-C14) heterocycle, a (C6-C14) aryl group, a (C5-C14) heteroaryl group;
each of B2 and B′2 independently is —H, —R8, —OH, —OR8, —COOH, —C(O)—NR8R′8, —NH₂, —NHR8, —NH—C(O)—R8, —O—P(O)(OH)₂, —O—P(O)(OR8)(OR′8) or a (C1-C6) alkyl optionally substituted by —OH, wherein R8 and R′8 are independently H or -L-R9, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxy, or haloalkyl, the C1-C25 hydrocarbon chain being optionally substituted with one or more -L′-R9, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R9 is H, OH, benzyl, benzyloxy, a nucleoside, a nucleoside analog, a nucleotide or a nucleotide analog.

As described in the present disclosure, L can be a branched or unbranched linking group. A branched linking group can have 2, 3, 4, or 5 cell targeting moieties.

The cell targeting moieties of formula (IVA), IVB) and (V) consist of specific objects of the present disclosure.

In some embodiments of a moiety of formula (IVA) or (IVB) or a compound of formula (V), A5 is H or a (C1-C6) alkyl group, optionally substituted by one or more hydroxy or acyloxy groups.

In some embodiments of a moiety of formula (IVA) or (IVB) or a compound of formula (V), A6 and A7 are hydroxy.

In some embodiments of a moiety of formula (IVA) or (IVB) or a compound of formula (V), A6 and A7 are acyloxy, for example acetoxy.

In some embodiments of a moiety of formula (IVA) or (IVB) or a compound of formula (V), A′7 is a H or (C1-C6) alkyl group, for example methyl.

In some embodiments of a moiety of formula (IVA) or (IVB) or a compound of formula (V), A5 is H or a (C1-C6) alkyl group, optionally substituted by one or more hydroxy or acyloxy, for example acetoxy.

In some embodiments of a moiety of formula (IVA) or (IVB) or a compound of formula (V), A8 is H, halogen, e.g., Cl, OH, or oxo (═O).

In some embodiments of a moiety of formula (IVA) or (IVB) or a compound of formula (V), A8 is —N(R7)₂, wherein R7 is H or a (C1-C6) alkyl group.

In some embodiments of a moiety of formula (IVA) or (IVB) or a compound of formula (V), A8 is —N(R7)₂ or —NHR7, wherein R7 is a (C1-C6) alkyl group, for example methyl.

In some embodiments of a moiety of formula (IVA) or (IVB) or a compound of formula (V), A8 is —NH—C(═O)—R7, wherein R7 is a (C1-C6) alkyl group, for example methyl, ethyl, or isopropyl.

In some embodiments of a moiety of formula (IVA) or (IVB) or a compound of formula (V), A9 is H, OH, oxo (═O) or NH₂.

In some embodiments of a moiety of formula (IVA) or (IVB) or a compound of formula (V), R6 is a H or a (C1-C6) alkyl group, for example methyl.

In some embodiments of a compound of formula (V), B₂ is CH₂OH, B₂′ is OH, A₅ is H, A₆ is OH, A₇ is H, A₇′ is OH, A₉ is H, R₆ is H and A₈ is not NH₂.

In some embodiments of a compound of formula (V), each of B2 and B′2 independently is H, OH, —NH₂, or —COOH.

In some embodiments of a compound of formula (V), B2 is —NH—C(O)—R8, —C(O)—NR8R′8, or —C(O)—NHR8, wherein R8 and R′8 are independently H or -L-R9, wherein L is a C1-C6 hydrocarbon chain optionally terminated by —C(O).

In some embodiments of a compound of formula (V), B2 is —NH—C(O)—R8, —C(O)—NR8R′8, or —C(O)—NHR8, wherein R8 and R′8 are independently H or -L-R9, wherein L is a C1-C6 hydrocarbon chain optionally terminated by —C(O), and R9 is H, OH or a nucleoside analog.

In some embodiments of a compound of formula (V), B′₂ is H and B₂ is OH.

In some embodiments of a compound of formula (V), B′₂ is H and B₂ is —O—P(O)(OH)₂ or —O—P(O)(OR8)(OR′8), wherein R8 and R′8 are H or -L-R9 and R9 is H or a nucleoside analog.

In some embodiments of a compound of formula (V), B′₂ is H and B₂ is —NH₂.

In some embodiments of a compound of formula (V), B′₂ is H and B₂ is —NH—C(O)—R8, wherein R8 is -L-R9, wherein L is a C1-C6 hydrocarbon chain and R9 is H.

In some embodiments of a compound of formula (V), B′₂ is H and B₂ is —NH—C(O)—R8, wherein R8 is -L-R9, wherein L is a C1-C6 hydrocarbon chain and R9 is OH.

In some embodiments of a compound of formula (V), B′₂ is —OH and B₂ is (C1-C6)-alkyl substituted by OH.

In some embodiments of a compound of formula (V), B′₂ is H and B₂ is COOH.

In some embodiments of a compound of formula (V), B′₂ is H and B₂ is —C(O)—NHR8, wherein R8 is -L-R9, wherein L is a C1-C6 hydrocarbon chain, for example methyl or butyl.

In some embodiments of a compound of formula (V), B′₂ is H and B₂ is —C(O)—NR8R′8, wherein R8 and R′8 are -L-R9, wherein L is a C1-C6 hydrocarbon chain, for example methyl.

In some embodiments of a compound of formula (V), B′2 is H and B₂ is OR8, wherein R8 is L-R9 and R9 is a nucleoside analog.

In some embodiments of a compound of formula (V), A5 is H or a (C1-C6) alkyl group, optionally substituted by one or more hydroxy.

In some embodiments of a compound of formula (V), A6 and A7 are hydroxy.

In some embodiments of a compound of formula (V), A′7 is H or a (C1-C6) alkyl group.

In some embodiments of a compound of formula (V), A8 is H, halogen, or hydroxy or its corresponding oxo (═O) tautomer.

In some embodiments of a compound of formula (V), A8 is —N(R7)₂ or —NHR7, wherein R7 is H or a (C1-C6) alkyl group.

In some embodiments of a compound of formula (V), A8 is —NH—C(═O)—R7, wherein R7 is a (C1-C6) alkyl group.

In some embodiments of a compound of formula (V), A9 is H, OH or its corresponding oxo (═O) tautomer, or NH₂.

In some embodiments of a compound of formula (V), R6 is a H or a (C1-C6) alkyl group.

Exemplary guano sine-derived ASGPR binding ligands of formula (V), wherein R9 may be a nucleoside analog, are shown in Table D below:

TABLE D

| Entry | Structure |
|---|---|
| 1 | 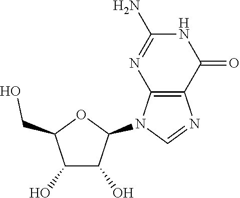 [SMILES: Nc1nc2c(ncn2[C@@H]2O[C@H](CO)[C@@H](O)[C@H]2O)c(=O)[nH]1] |
| 2 | 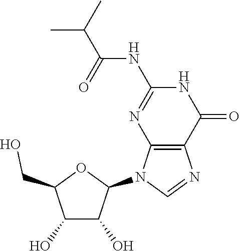 [SMILES: CC(C)C(=O)Nc1nc2c(ncn2[C@@H]2O[C@H](CO)[C@@H](O)[C@H]2O)c(=O)[nH]1] |
| 3 | 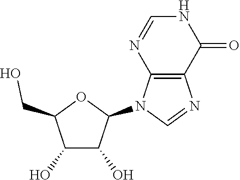 [SMILES: O=c1[nH]cnc2c1ncn2[C@@H]1O[C@H](CO)[C@@H](O)[C@H]1O] |
| 4 | 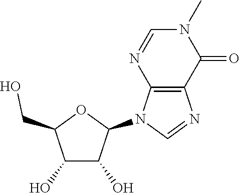 [SMILES: Cn1cnc2c(ncn2[C@@H]2O[C@H](CO)[C@@H](O)[C@H]2O)c1=O] |
| 5 | 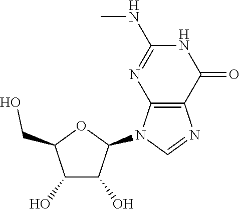 [SMILES: CNc1nc2c(ncn2[C@@H]2O[C@H](CO)[C@@H](O)[C@H]2O)c(=O)[nH]1] |
| 6 | 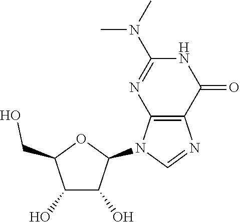 [SMILES: CN(C)c1nc2c(ncn2[C@@H]2O[C@H](CO)[C@@H](O)[C@H]2O)c(=O)[nH]1] |
| 7 | 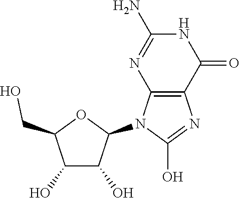 [SMILES: Nc1nc2c(nc(O)n2[C@@H]2O[C@H](CO)[C@@H](O)[C@H]2O)c(=O)[nH]1] |
| 8 | 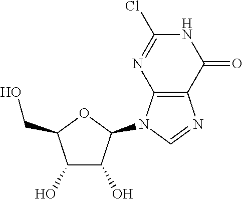 [SMILES: O=c1[nH]c(Cl)nc2c1ncn2[C@@H]1O[C@H](CO)[C@@H](O)[C@H]1O] |
| 9 | 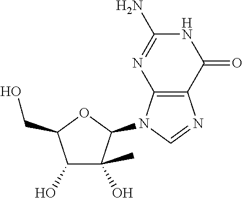 [SMILES: C[C@@]1(O)[C@H](O)[C@@H](CO)O[C@H]1n1cnc2c(=O)[nH]c(N)nc21] |
| 10 | 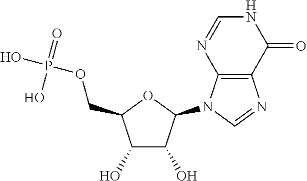 [SMILES: O=c1[nH]cnc2c1ncn2[C@@H]1O[C@H](COP(=O)(O)O)[C@@H](O)[C@H]1O] |
| 11 | 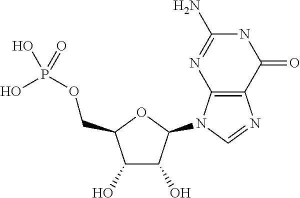 [SMILES: Nc1nc2c(ncn2[C@@H]2O[C@H](COP(=O)(O)O)[C@@H](O)[C@H]2O)c(=O)[nH]1] |
| 12 | 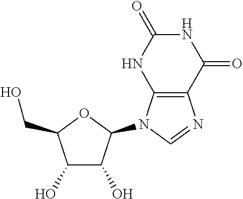 [SMILES: O=c1[nH]c(=O)c2ncn([C@@H]3O[C@H](CO)[C@@H](O)[C@H]3O)c2[nH]1] |
| 13 | 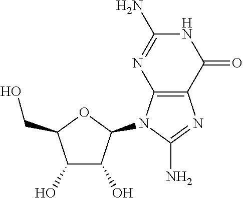 [SMILES: Nc1nc2c(nc(N)n2[C@@H]2O[C@H](CO)[C@@H](O)[C@H]2O)c(=O)[nH]1] |
| 14 | 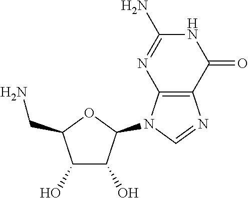 [SMILES: NC[C@H]1O[C@@H](n2cnc3c(=O)[nH]c(N)nc32)[C@H](O)[C@@H]1O] |
| 15 | 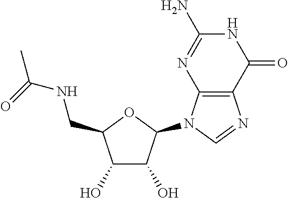 [SMILES: CC(=O)NC[C@H]1O[C@@H](n2cnc3c(=O)[nH]c(N)nc32)[C@H](O)[C@@H]1O] |
| 16 | 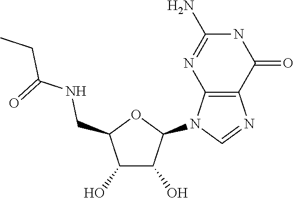 [SMILES: CCC(=O)NC[C@H]1O[C@@H](n2cnc3c(=O)[nH]c(N)nc32)[C@H](O)[C@@H]1O] |
| 17 | 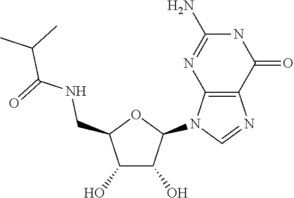 [SMILES: CC(C)C(=O)NC[C@H]1O[C@@H](n2cnc3c(=O)[nH]c(N)nc32)[C@H](O)[C@@H]1O] |
| 18 | 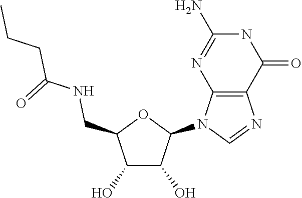 [SMILES: CCCC(=O)NC[C@H]1O[C@@H](n2cnc3c(=O)[nH]c(N)nc32)[C@H](O)[C@@H]1O] |
| 19 | 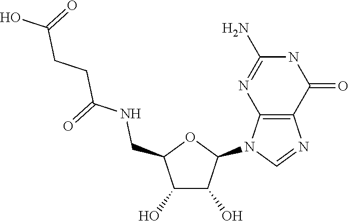 [SMILES: Nc1nc2c(ncn2[C@@H]2O[C@H](CNC(=O)CCC(=O)O)[C@@H](O)[C@H]2O)c(=O)[nH]1] |
| 20 | 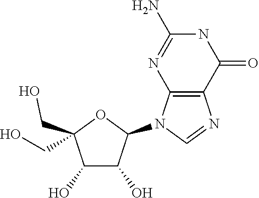 |
| 21 | 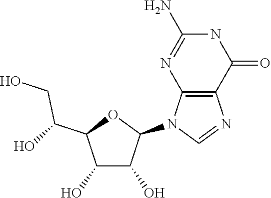 [SMILES: Nc1nc2c(ncn2[C@@H]2O[C@H]([C@H](O)CO)[C@@H](O)[C@H]2O)c(=O)[nH]1] |
| 22 | 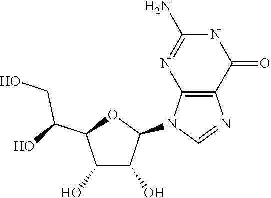 [SMILES: Nc1nc2c(ncn2[C@@H]2O[C@H]([C@@H](O)CO)[C@@H](O)[C@H]2O)c(=O)[nH]1] |
| 23 | 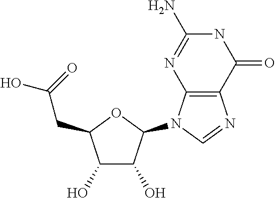 [SMILES: Nc1nc2c(ncn2[C@@H]2O[C@H](CC(=O)O)[C@@H](O)[C@H]2O)c(=O)[nH]1] |
| 24 | 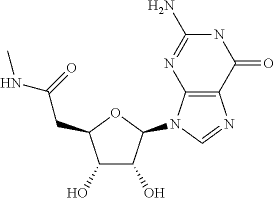 [SMILES: CNC(=O)C[C@H]1O[C@@H](n2cnc3c(=O)[nH]c(N)nc32)[C@H](O)[C@@H]1O] |
| 25 | 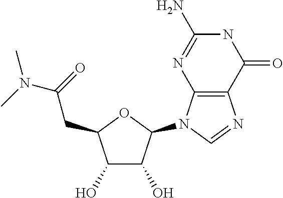 [SMILES: CN(C)C(=O)C[C@H]1O[C@@H](n2cnc3c(=O)[nH]c(N)nc32)[C@H](O)[C@@H]1O] |
| 26 | 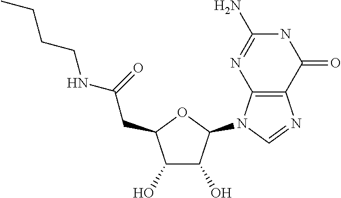 [SMILES: CCCCNC(=O)C[C@H]1O[C@@H](n2cnc3c(=O)[nH]c(N)nc32)[C@H](O)[C@@H]1O] |
| 27 | 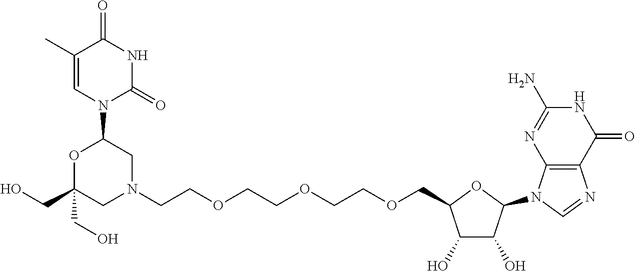 |
| 28 | 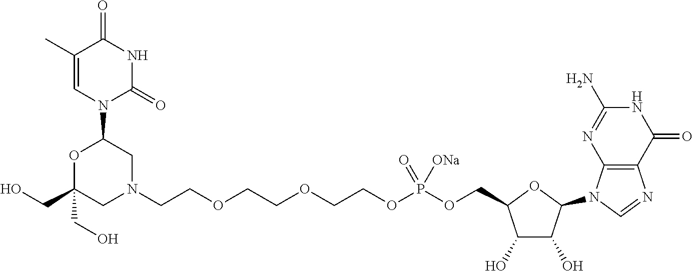 |
| 29 | 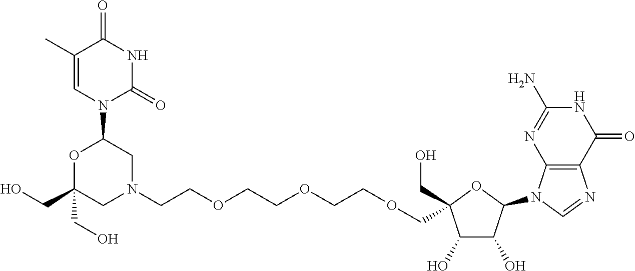 |

### II. 3 Trimeric ASGPR-Binders

Exemplary trimeric ASGPR-binding molecules comprising 3 cell-targeting moieties of formula (II) are shown in Table E below:

TABLE E

| Entry | Structure |
|---|---|
| 1 | 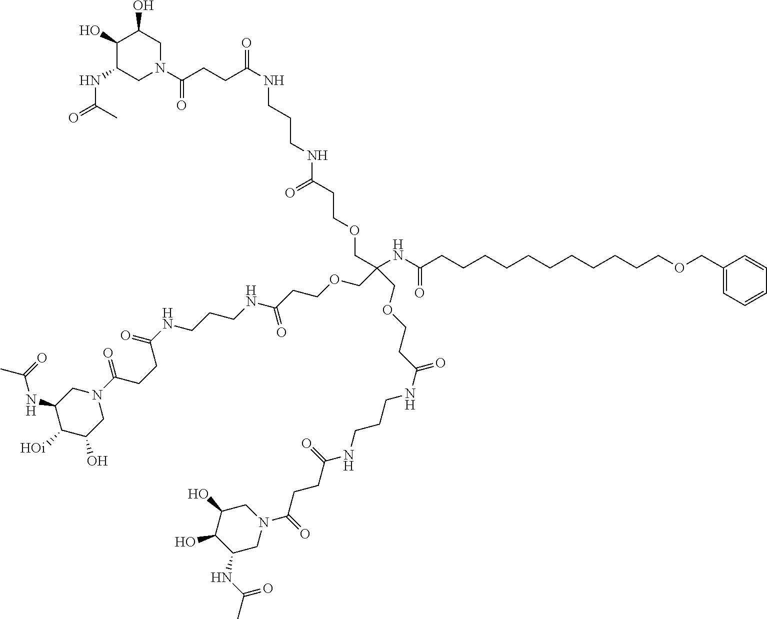 |
| 2 | 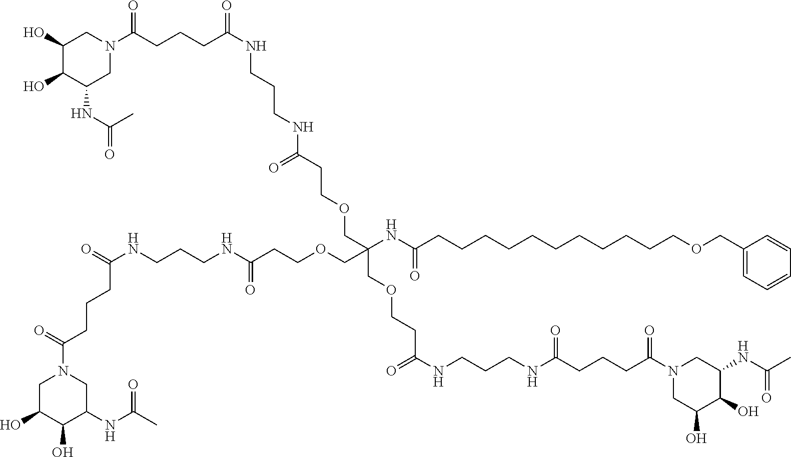 [SMILES: CC(=O)NC1CN(C(=O)CCCC(=O)NCCCNC(=O)CCOCC(COCCC(=O)NCCCNC(=O)CCCC(=O)N2C[C@H](NC(C)=O)[C@@H](O)[C@@H](O)C2)(COCCC(=O)NCCCNC(=O)CCCC(=O)N2C[C@H](NC(C)=O)[C@@H](O)[C@@H](O)C2)NC(=O)CCCCCCCCCCCOCc2ccccc2)C[C@H](O)[C@@H]1O] |
| 3 | 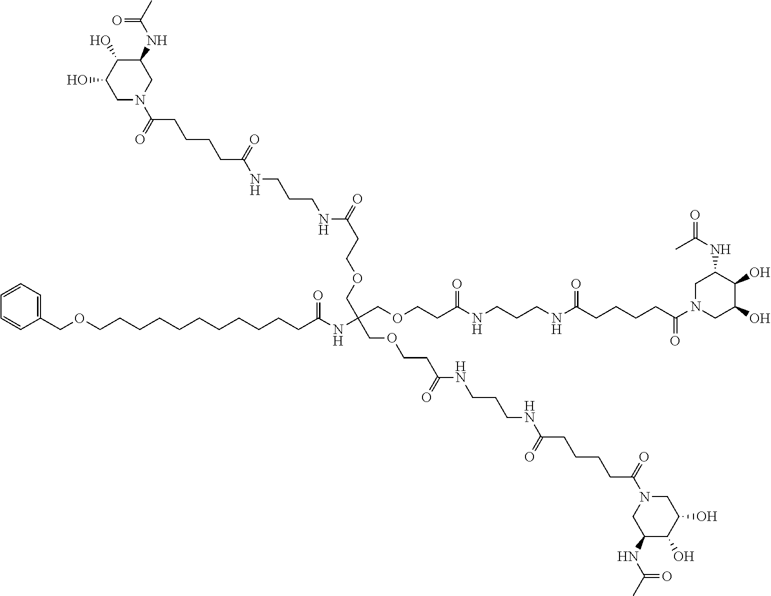 [SMILES: CC(=O)N[C@H]1CN(C(=O)CCCCC(=O)NCCCNC(=O)CCOCC(COCCC(=O)NCCCNC(=O)CCCCC(=O)N2C[C@H](NC(C)=O)[C@@H](O)[C@@H](O)C2)(COCCC(=O)NCCCNC(=O)CCCCC(=O)N2C[C@H](NC(C)=O)[C@@H](O)[C@@H](O)C2)NC(=O)CCCCCCCCCCCOCc2ccccc2)C[C@H](O)[C@@H]1O] |

In some embodiments, a nucleotide analog precursor of formula (I) described herein may be conjugated to one or more ASGPR-binding moieties of formulae (II), (IVA), or (IVB), directly or via a linker. In some embodiments, a nucleotide analog precursor of formula (I) described herein may be conjugated to one, two, three, or four ASGPR-binding moieties of formulae (II), (IVA), or (IVB), directly or via a linker. In particular embodiments, a nucleotide analog precursor of formula (I) described herein may be linked to three ASGPR-binding moieties of formulae (II), (IVA), or (IVB), directly or via a linker. In particular embodiments, a nucleotide analog precursor of formula (I) described herein may be the nucleotide analog in a compound of formula (III) or a compound of formula (V).

As described herein, in an ASGPR-targeted nucleotide analog precursor of formula (I), the ASGPR-binding ligand, e.g., a moiety of formula (II), (IVA), or (IVB), is directly and covalently bound to the nitrogen atom of the morpholino group. In particular embodiments, the ASGPR-binding ligand is covalently bound to the nitrogen atom of the morpholino group via a linker group.

Exemplary nucleotide precursors of formula (I) conjugated directly or via a linker to an ASGPR-binding moiety of formula (II), (IVA), or (IVB) are shown in Table F below. ASGPR-targeted nucleotide analog precursors are abbreviated as described above, but with an “lp” or “ls” instead of the “lg.”

TABLE F

| | | | Name in |
|---|---|---|---|
| | | Precursor | oligo- |
| Entry | Structure | name | sequence |
| 1 | 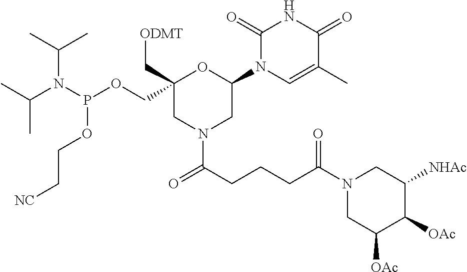 | pre-1pT1 | lpT1 |
| 2 | 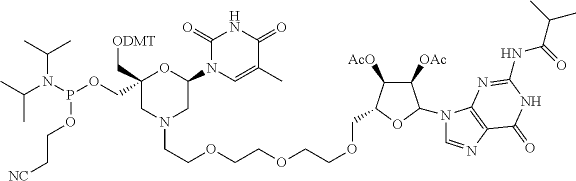 | pre-lsT1 | lsT1 |
| 3 | 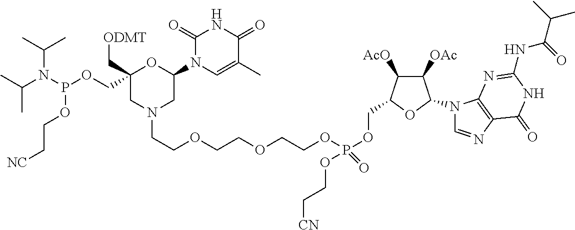 | pre-lsT2 | lsT2 |
| 4 | 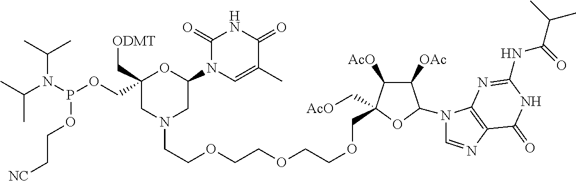 | pre-lsT3 | lsT3 |

Another aspect of this invention pertains to ASGPR-targeted oligonucleotides comprising one or more targeted nucleotide analogs derived from precursor compounds having the structure of formula (I) optimized for delivery to specific cells or tissue, e.g., hepatocytes. Compounds of formula (I) disclosed herein are nucleotide analog precursors, which, in the process of the oligonucleotide synthesis, convert to monomer units of oligomeric compounds, particularly as monomer units of oligonucleotides, including as monomer units of double-stranded RNA (“dsRNA”) oligomers, and especially as monomer units of siRNAs. Incorporation of ASGPR-targeted nucleotide analog precursors of formula (I) described herein into an oligonucleotide leads to the corresponding monomer units of the oligonucleotides described herein as compounds of formula (VI).

An ASGPR-targeted oligonucleotide of the present disclosure comprises one or more compounds of formula (VI):

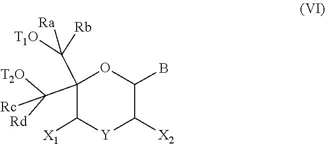

wherein:
B is a heterocyclic nucleobase;
one of T₁ and T₂ is an internucleoside linking group linking the compound of formula (VI) to the oligomeric compound and the other of T₁ and T₂ is H, a protecting group, a phosphorus moiety, or an internucleoside linking group linking the compound of formula (VI) to the oligomeric compound;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxy, or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is an ASGPR-binding cell targeting moiety of formula (II), (IVA) or (IVB); and each of X1, X2, Ra, Rb, Rc, and Rd independently is H or a —(C1-C6) alkyl group.

As described in the present disclosure, L can be a branched or unbranched linking group. A branched linking group can have 2, 3, 4, or 5 cell targeting moieties.

In some embodiments of a compound of formula (VI), Y is NR1, wherein R1 is -L-R3, wherein L is a C2-C25 hydrocarbon chain, and R3 is an ASGPR-binding cell targeting moiety of formula (II), (IVA) or (IVB).

In some embodiments of a compound of formula (VI), Y is NR1 and L is a C1-C10 hydrocarbon chain.

In some embodiments of a compound of formula (VI), Y is NR1 and L is a C1-C10 hydrocarbon chain optionally terminated by —C(O)—.

In some embodiments of a compound of formula (VI), Y is NR1, wherein R1 is -L-R3, wherein L is a C1-C10 hydrocarbon chain, and R3 is an ASGPR-binding cell targeting moiety of formula (II), (IVA) or (IVB).

In some embodiments of a compound of formula (VI), Y is N—C(═O)—R1 and L is a C1-C10 hydrocarbon chain optionally terminated by —C(O)—.

In some embodiments of a compound of formula (VI), Y is N—C(═O)—R1, wherein R1 is -L-R3, wherein L is a C2-C25 hydrocarbon chain, and R3 is an ASGPR-binding cell targeting moiety of formula (II), (IVA) or (IVB).

In some embodiments of a compound of formula (VI), Y is N—C(═O)—R1, wherein R1 is -L-R3, wherein L is a C1-C10 hydrocarbon chain, and R3 is an ASGPR-binding cell targeting moiety of formula (II), (IVA) or (IVB).

In some embodiments of a compound of formula (VI), Y is NR1, wherein R1 is -L-R3, wherein L is a C2-C25 hydrocarbon chain optionally terminated by —C(O)—, and R3 is an ASGPR-binding cell targeting moiety of formula (II), (IVA) or (IVB).

In some embodiments of a compound of formula (VI), Y is NR1, wherein R1 is -L-R3, wherein L is a C1-C10 hydrocarbon chain optionally terminated by —C(O)—, and R3 is an ASGPR-binding cell targeting moiety of formula (II), (IVA) or (IVB).

In some embodiments of a compound of formula (VI), Y is N—C(═O)—R1, wherein R1 is -L-R3, wherein L is a C2-C25 hydrocarbon chain optionally terminated by —C(O)—, and R3 is an ASGPR-binding cell targeting moiety of formula (II), (IVA) or (IVB).

In some embodiments of a compound of formula (VI), Y is N—C(═O)—R1, wherein R1 is -L-R3, wherein L is a C1-C10 hydrocarbon chain optionally terminated by —C(O)—, and R3 is an ASGPR-binding cell targeting moiety of formula (II), (IVA) or (IVB).

In some embodiments of a compound of formula (VI), Y is NR1, wherein R1 is -L-R3, wherein L is a C2-C25 hydrocarbon chain optionally interrupted by one or more —O—, and R3 is an ASGPR-binding cell targeting moiety of formula (II), (IVA) or (IVB).

In some embodiments of a compound of formula (VI), Y is NR1, wherein R1 is -L-R3, wherein L is a C2-C10 hydrocarbon chain optionally interrupted by one or more —O—, and R3 is an ASGPR-binding cell targeting moiety of formula (II), (IVA) or (IVB).

Exemplary trimeric oligonucleotides of formula (VI) in which every nucleotide is an ASGPR-targeted nucleotide analog can be understood as trivalent ASGPR-binders and therefore functional analogs to structures shown in Table E. Examples are shown in Table G below.

TABLE G

| Entry | Structure/sequence |
|---|---|
| 1 | 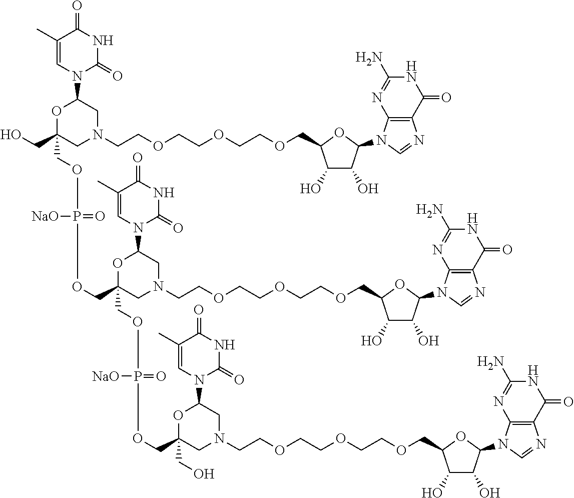 |
| 2 | 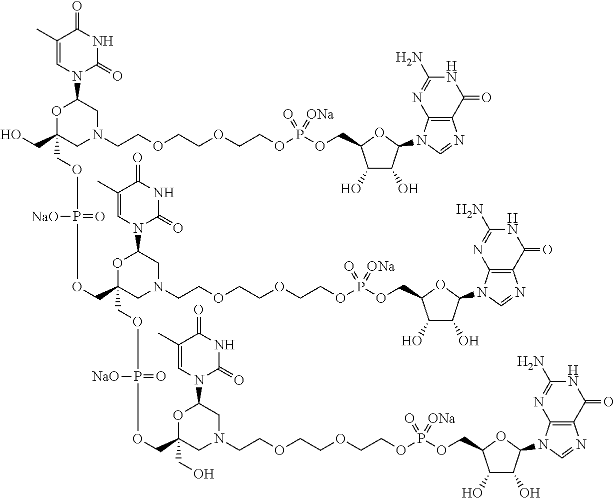 |
| 3 | 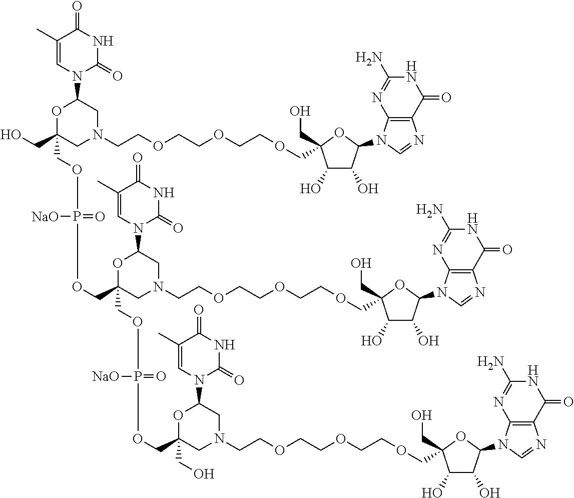 |
| 4 | 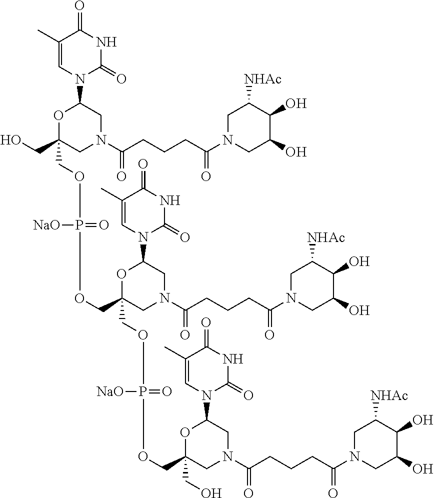 |

In some embodiments, ASGPR-targeted oligonucleotide according to the present disclosure is a single-stranded oligonucleotide, e.g., an ASO.

In some embodiments, an ASGPR-targeted oligonucleotide according to the present disclosure is an antisense oligonucleotide that targets a human mRNA.

In some other embodiments, an ASGPR-targeted oligonucleotide according to the present disclosure is a double-stranded oligonucleotide, e.g., an siRNA.

In some other embodiments, an ASGPR-targeted oligonucleotide according to the present disclosure is a double-stranded interfering RNA that targets a human mRNA and comprises a sense strand and an antisense strand.

In some embodiments, ASGPR-targeted oligonucleotides according to the present disclosure comprise one or more non-targeted nucleotides or nucleotide analogs and one or more ASGPR-targeted nucleotide analogs of formula (VI).

In some embodiments of an ASGPR-targeted oligonucleotide according to the present disclosure, either being a single-stranded or a double-stranded oligonucleotide, an oligonucleotide strand thereof comprises one or more ASGPR-targeted nucleotide analogs of formula (VI) which may be located at various locations within the oligonucleotide strand, e.g. internally and/or at the 3′ end or 5′ end thereof.

As used herein, a nucleotide analog refers to a compound that functions as a nucleotide in terms of being able to be incorporated into the phosphate backbone of a nucleic acid molecule, and/or being able to form a base pair with another nucleotide.

In some embodiments of an ASGPR-targeted oligonucleotide according to the present disclosure, either being a single-stranded or a double-stranded oligonucleotide, an oligonucleotide strand thereof comprises one or more ASGPR-targeted nucleotide analogs of formula (VI) which are located either at the 5′ end or at the 3′ end, or at both ends, of the oligonucleotide strand.

In some embodiments of an ASGPR-targeted oligonucleotide according to the present disclosure, either being a single-stranded or a double-stranded oligonucleotide, an oligonucleotide strand thereof comprises from 1 to 10 ASGPR-targeted nucleotide analogs of formula (VI) which are located either at the 5′ end or at the 3′ end of the strand, or at one or more other locations within the strand.

In some embodiments, the ASGPR-targeted oligonucleotide further comprises from 1 to 10 non-targeted nucleotide analogs which may be located at various locations within the oligonucleotide strand, e.g. internally and/or at the 3′ end or 5′ end thereof.

In some embodiments of an ASGPR-targeted oligonucleotide according to the present disclosure, either being a single-stranded or a double-stranded oligonucleotide, an oligonucleotide strand thereof comprises (A) one or more ASGPR-targeted nucleotide analogs of formula (VI) which are located either at the 3′ end or at the 5′ end, or at both ends, of the oligonucleotide strand and (B) one or more non-targeted nucleotide analogs which are located either at the 3′ end or at the 5′ end, or at both ends, of the oligonucleotide strand, with the ASGPR-targeted nucleotide analogs of formula (VI) and the non-targeted nucleotide analogs being located at distinct positions within the oligonucleotide strand.

In some embodiments of an ASGPR-targeted oligonucleotide according to the present disclosure, either being a single-stranded or a double-stranded oligonucleotide, an oligonucleotide strand thereof comprises from 1 to 10 ASGPR-targeted nucleotide analogs of formula (VI) which are located either at the 3′ end, or at the 5′ end of the strand. In some embodiments, the ASGPR-targeted oligonucleotide further comprises from 1 to 10 non-targeted nucleotide analogs which are located at the opposite end of the oligonucleotide strand. Thus, according to these embodiments, the number of ASGPR-targeted nucleotide analogs of formula (VI) at the selected end of the oligonucleotide strand may be 1, 2, 3, 4, 5, 6, 7, 8, 9 or 10. According to some of these embodiments, the number of non-targeted nucleotide analogs at the selected end of the oligonucleotide strand, if present, may be 1, 2, 3, 4, 5, 6, 7, 8, 9 or 10.

In particular embodiments, the one or more ASGPR-targeted nucleotide analogs of formula (VI) are linked, one to the other so as to form a continuous chain of these targeted nucleotide analogs at the selected end of the oligonucleotide strand.

In particular embodiments, the one or more ASGPR-targeted nucleotide analogs of formula (VI) are located at the 5′ end of a strand of an ASGPR-targeted oligonucleotide, either being single-stranded or double-stranded oligonucleotide. In some of these embodiments, the 5′ end nucleotide is an ASGPR-targeted nucleotide analog of formula (VI).

In some embodiments of an ASGPR-targeted oligonucleotide according to the present disclosure, either being a single-stranded or a double-stranded oligonucleotide, an oligonucleotide strand thereof comprises one or more non-targeted nucleotide analogs either at the 3′ end or at the 5′ end thereof, and especially at an end opposite to the end comprising one or more targeted nucleotide analogs of formula (VI).

In particular embodiments, the one or more non-targeted nucleotide analogs are linked, one to the other so as to form a continuous chain of these non-targeted nucleotide analogs at the selected end of the oligonucleotide strand.

In particular embodiments, the one or more non-targeted nucleotide analogs are located at the 3′ end of an oligonucleotide strand of an ASGPR-targeted oligonucleotide.

Thus, the present disclosure encompasses single-stranded ASGPR-targeted oligonucleotides comprising (i) one or more ASGPR-targeted nucleotide analogs of formula (VI), preferably from 1 to 10 ASGPR-targeted nucleotide analogs of formula (VI), which may be consecutive in the oligonucleotide chain and which are located at the 5′ end of the single-stranded targeted oligonucleotides. In some of these embodiments, the single-stranded targeted oligonucleotides further comprise (ii) one or more non-targeted nucleotide analogs, e.g., from 1 to 10 non-targeted nucleotide analogs which may be consecutive in the oligonucleotide chain and which are located at the 3′ end of the single-stranded targeted oligonucleotides.

Illustrations of single-stranded targeted oligonucleotides comprising (i) three targeted nucleotide analogs of formula (VI) at the 5′ end thereof and (ii) two non-targeted nucleotide analogs at the 3′ end thereof are disclosed in the examples herein.

The present disclosure also encompasses double-stranded oligonucleotides wherein (i) a first strand is a targeted oligonucleotide comprising one or more targeted nucleotide analogs of formula (VI) and one or more non-targeted nucleotides or nucleotide analogs, as described above, and wherein (ii) a second strand is another targeted oligonucleotide comprising one or more targeted nucleotide analogs of formula (VI) and one or more non-targeted nucleotides or nucleotide analogs.

The present disclosure further encompasses double-stranded oligonucleotides wherein (i) a first strand is a targeted oligonucleotide comprising one or more targeted nucleotide analogs of formula (VI) and one or more non-targeted nucleotides or nucleotide analogs, as described above, and (ii) a second strand is a non-targeted oligonucleotide that does not comprise any targeted nucleotides or nucleotide analogs.

### IV. Double-Stranded RNAs

An important aspect of the present disclosure relates to double-stranded ribonucleic acid (dsRNA) molecules, especially siRNAs, comprising a nucleotide analog conjugated to an ASGPR-targeted moiety, wherein the nucleotide analog has a structure shown in formula (VI). As used herein, the term “double-stranded RNA” or “dsRNA” refers to an oligoribonucleotide molecule comprising a duplex structure having two anti-parallel and substantially complementary nucleic acid strands. The two strands forming the duplex structure may be different portions of one larger RNA molecule, or they may be on separate RNA molecules. When the two strands are on separate RNA molecules, the dsRNA structure may function as small interfering RNA (siRNA). Where the two strands are part of one larger molecule and are connected by an uninterrupted chain of nucleotides between the 3′-end of a first strand and the 5′-end of a second strand, the connecting RNA chain is referred to as a “hairpin loop” and the RNA molecule may be termed “short hairpin RNA,” or “shRNA.” The RNA strands may have the same or a different number of nucleotides. In addition to the duplex structure, a dsRNA may comprise overhangs of one or more (e.g., 1, 2 or 3) nucleotides.

As used herein, the term “polynucleotide” refers to a polymeric form of nucleotides of at least 10 bases in length, either ribonucleotides or deoxyribonucleotides or a modified form of either type of nucleotide, or a combination thereof. The term includes single and double stranded forms.

As used herein, the term “oligonucleotide” refers to a polymeric form of nucleotides of no more than 50 bases in length, either ribonucleotides or deoxyribonucleotides or a modified form of either type of nucleotide, or a combination thereof. The term includes single and double stranded forms.

A “dsRNA” may include naturally occurring ribonucleotides, and/or chemically modified analogs thereof. A dsRNA of the present disclosure may comprise one or more modifications that could enhance its cellular uptake, affinity for the target sequence, inhibitory activity, and/or stability. Further, a dsRNA of the present disclosure may include one or more modified nucleotides known in the art, including, without limitation, 2′-O-methyl modified nucleotides, 2′-deoxy-2′-fluoro modified nucleotides, 2′-deoxy modified nucleotides, 2′-O-methoxyethyl modified nucleotides, modified nucleotides comprising alternate internucleotide linkages such as thiophosphates and phosphorothioates, phosphotriester modified nucleotides, modified nucleotides terminally linked to a cholesterol derivative or lipophilic moiety, peptide nucleic acids (PNAs; see, e.g., Nielsen et al., Science (1991) 254:1497-500), constrained ethyl (cEt) modified nucleotides, inverted deoxy modified nucleotides, inverted dideoxy modified nucleotides, locked nucleic acid modified nucleotides (LNA), unlocked nucleic acid modified nucleotides (UNA), abasic modifications of nucleotides, 2′-amino modified nucleotides, 2′-alkyl modified nucleotides, morpholino-modified nucleotides, phosphoramidate modified nucleotides, modified nucleotides comprising modifications at other sites of the sugar or base of an oligonucleotide, and non-natural base-containing modified nucleotides.

In some embodiments, at least one of the one or more modified nucleotides is a 2′-O-methyl nucleotide, a 5′-phosphorothioate nucleotide, or a terminal nucleotide linked to a cholesterol derivative, a lipophilic group, or any other cell targeting moiety. The incorporation of 2′-O-methyl, 2′-O-ethyl, 2′-O-propyl, 2′-O-alkyl, 2′-O-aminoalkyl, or 2′-deoxy-2′-fluoro (i.e., 2′-fluoro) groups in nucleosides or nucleotides of an oligonucleotide may confer enhanced hybridization properties and/or enhanced nuclease stability to the oligonucleotide. Further, oligonucleotides containing phosphorothioate backbones (e.g., a phosphorothioate linkage between two consecutive nucleotides at one or more positions of the dsRNA) may have enhanced nuclease stability. In some embodiments, the dsRNA may contain nucleotides with a modified ribose, such as locked nucleic acid (LNA) units.

In some embodiments, a dsRNA of the present disclosure comprises one or more 2′-O-methyl nucleotides and one or more 2′-fluoro nucleotides. In some embodiments, the dsRNA comprises two or more 2′-O-methyl nucleotides and two or more 2′-fluoro nucleotides. In some embodiments, the dsRNA comprises two or more 2′-O-methyl nucleotides (OMe) and two or more 2′-fluoro nucleotides (F) in an alternating pattern, e.g., the pattern OMe-F—OMe-F or the pattern F—OMe-F—OMe. In some embodiments, the dsRNA comprises up to 10 contiguous nucleotides that are each a 2′-O-methyl nucleotide. In some embodiments, the dsRNA comprises up to 10 contiguous nucleotides that are each a 2′-fluoro nucleotide. In some embodiments, the dsRNA comprises two or more 2′-fluoro nucleotides at the 5′- or 3′-end of the antisense strand.

As used herein, “dsRNAs” are not limited to those with ribose-containing nucleotides. A dsRNA herein encompasses a double-stranded polynucleotide (e.g., oligonucleotide) molecule where the ribose moiety in some or all of its nucleotides has been replaced by another moiety, so long as the resultant double-stranded molecule can inhibit the expression of a target gene by RNA interference. The dsRNA may also include one or more, but not more than 60% (e.g., not more than 50%, 40%, 30%, 20%, or 10%) deoxyribonucleotides or chemically modified analogs thereof.

In some embodiments, a nucleotide or nucleotide analog of the present disclosure may be linked to an adjacent nucleotide or nucleotide analog through a linkage between the 3′-carbon of the sugar moiety of the first nucleotide and the 5′-carbon of the sugar moiety of the second nucleotide (herein referred to as a 3′-5′ internucleotide linkage). In another aspect, a nucleotide or nucleotide analog of the present disclosure may be linked to an adjacent nucleotide or nucleotide analog through a linkage between the 2′-carbon of the sugar moiety of the first nucleotide and the 5′-carbon of the sugar moiety of the second nucleotide (herein referred to as a 2′-5′ internucleotide linkage).

As used herein, the term “internucleotide linking group” encompasses phosphorus and non-phosphorus containing internucleotide linking groups.

In some embodiments of a dsRNA of the present disclosure, the internucleotide backbone linkage is a phosphorus-containing internucleotide linking group, e.g., phosphodiesters, phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, and 2′-5′ linked analogs thereof.

In some embodiments, a dsRNA of the present disclosure comprises one or more phosphorothioate groups. In some embodiments, a dsRNA of the present disclosure comprises two or more, three or more, four or more, five or more, six or more, seven or more, eight or more, nine or more, or 10 or more phosphorothioate groups. In some embodiments, the dsRNA does not comprise any phosphorothioate group.

In some embodiments, a dsRNA of the present disclosure comprises one or more phosphotriester groups. In some embodiments, the dsRNA comprises two or more, three or more, four or more, five or more, six or more, seven or more, eight or more, nine or more, or 10 or more phosphotriester groups. In some embodiments, the dsRNA does not comprise any phosphotriester group.

In some embodiments of a dsRNA of the present disclosure, the internucleotide backbone linkage is a non-phosphodiester linkage, e.g., a phosphorothioate, phosphorodithioate, alkyl-phosphonate and phosphoramidate backbone linking groups.

In some embodiments, dsRNAs of the of the present disclosure comprise one or more internucleoside linking groups that do not contain a phosphorus atom. Such oligonucleotides include, but are not limited to, those that are formed by short chain alkyl or cycloalkyl internucleoside linking groups, mixed heteroatom and alkyl or cycloalkyl internucleoside linking groups, or one or more short chain heteroatomic or heterocyclic internucleoside linking groups. These include those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above phosphorus-containing internucleotide linkages include U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Representative U.S. patents that teach the preparation of the above non-phosphorus containing internucleoside linking group include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In some embodiments, dsRNAs of the present disclosure comprise one or more neutral internucleoside linking groups that are non-ionic. Neutral internucleoside linking groups encompass nonionic linking groups comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook Eds. ACS Symposium Series 580; Chapters 3 and 4, (pp. 40-65)). Further neutral internucleoside linking groups encompass nonionic linkages comprising mixed N, O, S and CH₂ component parts.

A dsRNA of the present disclosure comprises a sense strand comprising a sense sequence and an antisense strand comprising an antisense sequence, wherein the sense and antisense sequences are substantially or fully complementary to each other. Unless otherwise indicated, the term “complementary” refers herein to the ability of a polynucleotide comprising a first contiguous nucleotide sequence, under certain conditions, e.g., physiological conditions, to hybridize to and form a duplex structure with another polynucleotide comprising a second contiguous nucleotide sequence. This may include base-pairing of the two polynucleotides (e.g., two oligonucleotides) over the entire length of the first or second contiguous nucleotide sequence; in this case, the two nucleotide sequences are considered “fully complementary” to each other. For example, in a case where a dsRNA comprises a first oligonucleotide 21 nucleotides in length and a second oligonucleotide 23 nucleotides in length, and where the two oligonucleotides form 21 contiguous base-pairs, the two oligonucleotides may be referred to as “fully complementary” to each other. Where a first polynucleotide (e.g., oligonucleotide) sequence is referred to as “substantially complementary” to a second polynucleotide sequence, the two sequences may base-pair with each other over 80% or more (e.g., 90% or more) of their length of hybridization, with no more than 20% (e.g., no more than 10%) of mismatching base-pairs (e.g., for a duplex of 20 nucleotides, no more than 4 or no more than 2 mismatched base-pairs). Where two oligonucleotides are designed to form a duplex with one or more single-stranded overhangs, such overhangs shall not be regarded as mismatches for the determination of complementarity. Complementarity of two sequences may be based on Watson-Crick base-pairs and/or non-Watson-Crick base-pairs. As used herein, a polynucleotide which is “substantially complementary to at least part of” an mRNA refers to a polynucleotide which is substantially complementary to a contiguous portion of an mRNA of interest.

In some embodiments, dsRNA is an siRNA where the sense and antisense strands are not covalently linked to each other. In some embodiments, the sense and antisense strands of the dsRNA are covalently linked to each other, e.g., through a hairpin loop (such as in the case of shRNA), or by means other than a hairpin loop (such as by a connecting structure referred to as a “covalent linker”).

### IV.1 Lengths

In some embodiments, each of the sense sequence (in the sense strand) and the antisense sequence (in the antisense strand) is 9-30 nucleotides in length. For example, each sequence can be any of a range of nucleotide lengths having an upper limit of 21, 22, 23, 24, 25, 26, 27, 28, 29, or 30 and an independently selected lower limit of 9, 10, 11, 12, 13, 14, 15, 16, 17, 18, 19, or 20. In some embodiments, the number of nucleotides in each sequence may be 15-25 (i.e., 15 to 25 nucleotides in each sequence), 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, each sequence is greater than 8, 9, 10, 11, 12, 13, 14, 15, 16, 17, 18, 19, 20, 21, 22, 23, 24, 25, 26, 27, 28, 29, or 30 nucleotides in length. In some embodiments, each sequence is less than 21, 22, 23, 24, 25, 26, 27, 28, 29, 30, or 31 nucleotides in length. In some embodiments, each sequence is 9, 10, 11, 12, 13, 14, 15, 16, 17, 18, 19, 20, 21, 22, 23, 24, 25, 26, 27, 28, 29, or 30 nucleotides in length.

In some embodiments, the sense and antisense sequences are each at least 15 and no greater than 25 nucleotides in length. In some embodiments, the sense and antisense sequences are each at least 19 and no greater than 23 nucleotides in length. For example, the sequences are 15, 16, 17, 18, 19, 20, 21, 22, 23, 24, or 25 nucleotides in length.

In some embodiments, the dsRNA has sense and antisense strands of the same length or different lengths. For example, the sense strand may be 1, 2, 3, 4, 5, 6, or 7 nucleotides longer than the antisense strand. Alternatively, the sense strand may be 1, 2, 3, 4, 5, 6, or 7 nucleotides shorter than the antisense strand.

In some embodiments, each of the sense strand and the antisense strand is 9-36 nucleotides in length. For example, each strand can be any of a range of nucleotide lengths having an upper limit of 21, 22, 23, 24, 25, 26, 27, 28, 29, 30, 31, 32, 33, 34, 35, or 36 and an independently selected lower limit of 9, 10, 11, 12, 13, 14, 15, 16, 17, 18, 19, or 20. In some embodiments, the number of nucleotides in each strand may be 15-25, 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, each strand is greater than 8, 9, 10, 11, 12, 13, 14, 15, 16, 17, 18, 19, 20, 21, 22, 23, 24, 25, 26, 27, 28, 29, or 30 nucleotides in length. In some embodiments, each strand is less than 20, 21, 22, 23, 24, 25, 26, 27, 28, 29, 30, 31, 32, 33, 34, 35, 36, or 37 nucleotides in length. In some embodiments, each strand is 9, 10, 11, 12, 13, 14, 15, 16, 17, 18, 19, 20, 21, 22, 23, 24, 25, 26, 27, 28, 29, 30, 31, 32, 33, 34, 35, or 36 nucleotides in length.

In some embodiments, the sense and antisense strands are each at least 15 and no greater than 25 nucleotides in length. In some embodiments, the sense and antisense strands are each at least 19 and no greater than 23 nucleotides in length. For example, the strands are 19, 20, 21, 22, or 23 nucleotides in length.

In some embodiments, the sense strand may have 21, 22, 23, or 24 nucleotides, including any modified nucleotides, while the antisense strand may have 21 nucleotides, including any modified nucleotides; in certain embodiments, the sense strand may have a sense sequence having 17, 18, or 19 nucleotides, while the antisense strand may have an antisense sequence having 19 nucleotides.

### IV.2 Overhangs

In some embodiments, a dsRNA of the present disclosure comprises one or more overhangs at the 5′-end, 3′-end, or both ends of one or both of the sense and antisense strands. In some embodiments, the one or more overhangs improve the deliverability, inhibitory activity, and/or stability of the dsRNA.

“Overhang” refers herein to the unpaired nucleotide(s) that protrude from the duplex structure of a dsRNA when a 3′ end of a first strand of the dsRNA extends beyond the 5′ end of a second strand, or vice versa. “Blunt end” means that there are no unpaired nucleotides at that end of the dsRNA, i.e., no nucleotide overhang. A “blunt-ended” dsRNA is a dsRNA that is double-stranded over its entire length, i.e., no nucleotide overhang at either end of the duplex molecule. Chemical caps or non-nucleotide chemical moieties conjugated to the 3′ end and/or the 5′ end of a dsRNA are not considered herein in determining whether a dsRNA has an overhang or not.

In some embodiments, an overhang comprises one or more, two or more, three or more, or four or more nucleotides. For example, the overhang may comprise 1, 2, 3, or 4 nucleotides.

In some embodiments, an overhang of the present disclosure comprises one or more nucleotides (e.g., ribonucleotides or deoxyribonucleotides, naturally occurring or chemically modified analogs thereof). In some embodiments, the overhang comprises one or more thymines or chemically modified analogs thereof. In certain embodiments, the overhang comprises one or more thymines.

In some embodiments, the dsRNA comprises an overhang located at the 3′-end of the antisense strand. In some embodiments, the dsRNA comprises a blunt end at the 5′-end of the antisense strand. In some embodiments, the dsRNA comprises an overhang located at the 3′-end of the antisense strand and a blunt end at the 5′-end of the antisense strand. In some embodiments, the dsRNA comprises an overhang located at the 3′-end of the sense strand. In some embodiments, the dsRNA comprises a blunt end at the 5′-end of the sense strand. In some embodiments, the dsRNA comprises an overhang located at the 3′-end of the sense strand and a blunt end at the 5′-end of the sense strand. In some embodiments, the dsRNA comprises overhangs located at the 3′-end of both the sense and antisense strands of the dsRNA.

In some embodiments, the dsRNA comprises an overhang located at the 5′-end of the antisense strand. In some embodiments, the dsRNA comprises a blunt end at the 3′-end of the antisense strand. In some embodiments, the dsRNA comprises an overhang located at the 5′-end of the antisense strand and a blunt end at the 3′-end of the antisense strand. In some embodiments, the dsRNA comprises an overhang located at the 5′-end of the sense strand. In some embodiments, the dsRNA comprises a blunt end at the 3′-end of the sense strand. In some embodiments, the dsRNA comprises an overhang located at the 5′-end of the sense strand and a blunt end at the 3′-end of the sense strand. In some embodiments, the dsRNA comprises overhangs located at both the 5′-end of the sense and antisense strands of the dsRNA.

In some embodiments, the dsRNA comprises an overhang located at the 3′-end of the antisense strand and an overhang at the 5′-end of the antisense strand. In some embodiments, the dsRNA comprises an overhang located at the 3′-end of the sense strand and an overhang at the 5′-end of the sense strand.

In some embodiments, the dsRNA has two blunt ends.

In some embodiments, the overhang is the result of the sense strand being longer than the antisense strand. In some embodiments, the overhang is the result of the antisense strand being longer than the sense strand. In some embodiments, the overhang is the result of sense and antisense strands of the same length being staggered. In some embodiments, the overhang forms a mismatch with the target mRNA. In some embodiments, the overhang is complementary to the target mRNA.

In some embodiments, the dsRNA comprises a modified ribonucleoside such as a deoxyribonucleoside, including, for example, deoxyribonucleoside overhang(s), and one or more deoxyribonucleosides within the double-stranded portion of a dsRNA. However, it is self-evident that under no circumstances is a double-stranded DNA molecule encompassed by the term “dsRNA.”

In some embodiments, the dsRNA comprises two or more, three or more, four or more, five or more, six or more, seven or more, eight or more, nine or more, or 10 or more different modified nucleotides described herein. In some embodiments, the dsRNA comprises up to two contiguous modified nucleotides, up to three contiguous modified nucleotides, up to four contiguous modified nucleotides, up to five contiguous modified nucleotides, up to six contiguous modified nucleotides, up to seven contiguous modified nucleotides, up to eight contiguous modified nucleotides, up to nine contiguous modified nucleotides, or up to 10 contiguous modified nucleotides. In some embodiments, the contiguous modified nucleotides are the same modified nucleotide. In some embodiments, the contiguous modified nucleotides are two or more, three or more, four or more, five or more, six or more, seven or more, eight or more, nine or more, or ten or more different modified nucleotides.

As used herein, the term “antisense strand” in a dsRNA refers to the strand of the dsRNA containing a sequence that is substantially complementary to a target sequence. The other strand in the dsRNA is the “sense strand”.

In some embodiments, targeted nucleotide analogs of formula (VI) are located at the 5′-end, at the 3′-end, or both at the 5′-end and at the 3′-end of a nucleic acid strand of a dsRNA, such as the 5′-end or at the 3′-end of a nucleic acid strand of an siRNA. In particular embodiments, targeted nucleotide analogs of formula (VI) are located at the 5′-end, at the 3′-end, or both at the 5′-end and at the 3′-end of the sense strand of an siRNA. In some other embodiments, targeted nucleotide analogs of formula (VI) are located at the 5′-end, at the 3′-end, or both at the 5′-end and at the 3′-end of the antisense strand of an siRNA.

In some embodiments, targeted nucleotide analogs of formula (VI) are exclusively located at the 5′-end of a nucleic acid strand of a dsRNA, such as exclusively located at the 5′-end of a nucleic acid strand of a siRNA. In particular embodiments, targeted nucleotide analogs of formula (VI) are located at the 5′-end of the sense strand of an siRNA.

In some embodiments, targeted nucleotide analogs of formula (VI) are located both at the 5′-end of the sense strand of an siRNA and at the 5′-end of the antisense strand of the siRNA. In some other embodiments, targeted nucleotide analogs of formula (VI) are located both at the 3′-end of the sense strand of an siRNA and at the 3′-end of the antisense strand of the siRNA. In still other embodiments, targeted nucleotide analogs of formula (VI) are located both at the 5′-end of the sense strand of an siRNA and at the 3′-end of the antisense strand of the siRNA, or at the 3′-end of the sense strand of an siRNA and at the 5′-end of the antisense strand of the siRNA.

In certain embodiments, targeted nucleotide analogs of formula (VI) are located (i) both at the 5′-end and at the 3′-end of the sense strand of an siRNA and (ii) are located at the 5′-end of the antisense strand of the siRNA. In certain other embodiments, targeted nucleotide analogs of formula (VI) are located (i) both at the 5′-end and at the 3′-end of the sense strand of an siRNA and (ii) are located at the 3′-end of the antisense strand of the siRNA.

In some embodiments, 2 to 10 (e.g., 2 to 5) targeted nucleotide analogs of formula (VI) are present in an oligonucleotide. As used herein, 2 to 10 nucleotide analogs of formula (VI) encompass 2, 3, 4, 5, 6, 7, 8, 9 and 10 nucleotide analogs of formula (VI).

In particular embodiments, targeted nucleotide analogs of formula (VI) are located in an overhang of a dsRNA, such as of an siRNA. For example, the targeted nucleotide analogs of formula (VI) are located in an overhang, such as the 5′-overhang of the sense strand of an siRNA.

The present disclosure also describes an siRNA comprising:
a sense strand comprising (i) one or more ASGPR-targeted nucleotide analogs of formula (VI), especially from 1 to 10 ASGPR-targeted nucleotide analogs of formula (VI) which are located at the 5′ end thereof and (ii) one or more non-targeted nucleotide analogs, especially from 1 to 10 non-targeted nucleotide analogs which are located at the 3′ end thereof, and
an antisense strand, which is either a non-targeted oligonucleotide or an ASGPR-targeted oligonucleotide.

The present disclosure also describes an siRNA comprising:
a sense strand comprising (i) one or more ASGPR-targeted nucleotide analogs of formula (VI), especially from 1 to 10 ASGPR-targeted nucleotide analogs of formula (VI) which are located at the 3′ end thereof, and (ii) one or more non-targeted nucleotide analogs, especially from 1 to 10 non-targeted nucleotide analogs which are located at the 5′ end thereof, and
an antisense strand, which is either a non-targeted oligonucleotide or an ASGPR-targeted oligonucleotide.

The present disclosure further describes an siRNA comprising:
a sense strand comprising (i) one or more ASGPR-targeted nucleotide analogs of formula (VI), especially from 1 to 10 ASGPR-targeted nucleotide analogs of formula (VI) which are located at the 5′ end thereof and (ii) one or more non-targeted nucleotide analogs, especially from 1 to 10 non-targeted nucleotide analogs which are located at the 3′ end thereof, and
an antisense strand comprising one or more non-targeted nucleotides or nucleotide analogs, especially from 1 to 10 non-targeted nucleotides or nucleotide analogs.

The present disclosure further describes an siRNA comprising:
a sense strand comprising (i) one or more ASGPR-targeted nucleotide analogs of formula (VI), especially from 1 to 10 ASGPR-targeted nucleotide analogs of formula (VI) which are located at the 5′ end thereof and (ii) one or more non-targeted nucleotide analogs, especially from 1 to 10 non-targeted nucleotide analogs which are located at the 3′ end thereof, and
an antisense strand that may or may not include such nucleotide analogs.

Within the scope of the present disclosure, the “percentage identity” between two sequences of nucleic acids means the percentage of identical nucleotides residues between the two sequences to be compared, obtained after optimal alignment, this percentage being purely statistical and the differences between the two sequences being distributed randomly along their length. The comparison of two nucleic acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981), by means of the local homology algorithm of Neddleman and Wunsch (1970), by means of the similarity search method of Pearson and Lipman (1988)), or by means of computer software using these algorithms (GAP, BESTFIT, FASTA and TFASTA in the Wisconsin Genetics Software Package, Genetics Computer Group, 575 Science Dr., Madison, WI, or by the comparison software BEAST NR or BEAST P). The percentage identity between two nucleic acid sequences is determined by comparing the two optimally-aligned sequences in which the nucleic acid sequence to compare can have additions or deletions compared to the reference sequence for optimal alignment between the two sequences. Percentage identity is calculated by determining the number of positions at which the nucleotide residue is identical between the two sequences, preferably between the two complete sequences, dividing the number of identical positions by the total number of positions in the alignment window and multiplying the result by 100 to obtain the percentage identity between the two sequences.

As intended herein, nucleotide sequences having at least 70% nucleotide identity with a reference sequence encompass those having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the reference sequence.

As used herein, the term “introducing into a cell” means facilitating uptake or absorption into the cell, as would be understood by one of ordinary skill in the art. Absorption or uptake of dsRNA can occur through unaided diffusive or active cellular processes, or by auxiliary agents or devices. The meaning of this term is not to be limited to a cell in vitro; a dsRNA may also be “introduced into a cell”, wherein the cell is part of a living organism. In such an instance, introduction into the cell will include delivery to the organism. For example, for in vivo delivery, dsRNA can be injected into a tissue site or administered systemically. In vivo delivery can also be mediated by a beta-glucan delivery system (See, e.g., Tesz et al., Biochem J. (2011) 436(2):351-62). In vitro introduction into a cell includes methods known in the art such as electroporation and lipofection. Further approaches are described herein below or known in the art.

As used herein, the terms “inhibit the expression of” or “inhibiting expression of” insofar as they refer to a target gene, refer to the at least partial suppression of the expression of the target gene, as manifested by a reduction of the amount of mRNA transcribed from the target gene. As used herein, the term “inhibiting” is used interchangeably with “reducing”, “silencing”, “downregulating”, “suppressing”, “knock-down” and other similar terms, and include any level of inhibition. The degree of inhibition is usually expressed in terms of (((mRNA in control cells)−(mRNA in treated cells))/(mRNA in control cells))*100%. Alternatively, the degree of inhibition may be given in terms of a reduction of a parameter that is functionally linked to a target gene transcription, e.g., the amount of protein encoded by the target gene which is secreted by a cell, or the number of cells displaying a certain phenotype, e.g., apoptosis. In principle, target gene silencing may be determined in any cell expressing the target, either constitutively or by genomic engineering, and by any appropriate assay. However, when a reference is needed in order to determine whether a given dsRNA inhibits the expression of the target gene by a certain degree and therefore is encompassed by the present disclosure, the assays provided in the Examples below shall serve as such a reference.

As used herein, in the context of a target gene expression, the terms “treat”, “treatment” and the like refer to relief from or alleviation of pathological processes mediated by the expression of a target gene. In the context of the present disclosure, insofar as it relates to any of the other conditions recited herein below (other than pathological processes mediated by target expression), the terms “treat”, “treatment”, and the like refer to relieving or alleviating one or more symptoms associated with such condition.

As used herein, the terms “prevent” or “delay progression of” (and grammatical variants thereof) with respect to a disease or disorder relate to prophylactic treatment of a disease, e.g., in an individual suspected to have the disease, or at risk for developing the disease. Prevention may include, but is not limited to, preventing or delaying onset or progression of the disease and/or maintaining one or more symptoms of the disease at a desired or sub-pathological level. As used herein, the terms “therapeutically effective amount” and “prophylactically effective amount” refer to an amount that provides a therapeutic benefit in the treatment, prevention, or management of pathological processes mediated by target gene expression, or an overt symptom of pathological processes mediated by the expression of a target gene. The specific amount that is therapeutically effective can be readily determined by an ordinary medical practitioner, and may vary depending on factors such as the type and stage of pathological processes mediated by the target gene expression, the patient's medical history and age, and the administration of other therapeutic agents that inhibit biological processes mediated by the target gene.

As used herein, the term “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

### V. Synthesis Methods

### V.1 Preparation of Compounds of Formula (I)

In some embodiments, compounds of formula (I) may be prepared according to the methods described in WO 2019/170731, which is incorporated in its entirety herein by reference. In some other embodiments, compounds of formula (I) may be prepared according to the detailed methods illustrated in Examples 1-25 of the present disclosure.

### V.2 Preparation of Oligonucleotides Comprising Compounds of Formula (VI)

Oligonucleotides of the present invention such as those comprising one or more compounds of formula (VI) may be chemically synthesized using protocols known in the art. See, e.g., Caruthers et al., Methods in Enzymology (1992) 211:3-19; Thompson et al., International PCT Publication No. WO 99/54459; Wincott et al., 1995, Nucleic Acids Res., 23:2677-2684; Wincott et al., 1997, Methods Mol. Bio., 74:59; Brennan et al., 1998, Biotechnol Bioeng., 61:33-45; and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end. In certain embodiments, oligonucleotides comprising compounds of formula (II) are synthesized, deprotected, and analyzed according to methods described in U.S. Pat. Nos. 6,995,259; 6,686,463; 6,673,918; 6,649,751; 6,989,442; and 7,205,399. In a non-limiting synthesis example, small scale syntheses are conducted on a 394 Applied Biosystems, Inc./Thermo Fischer Scientific Inc. synthesizer.

Alternatively, oligonucleotides comprising one or more compounds of formula (VI) can be synthesized separately and joined together post synthesis, for example, by ligation (Moore et al., 1992, Science 256:9923; Draper et al., International PCT Publication No. WO 93/23569; Shabarova et al., 1991, Nucleic Acids Research 19:4247; Bellon et al., 1997, Nucleosides & Nucleotides, 16:951; Bellon et al., 1997, Bioconjugate Chem., 8:204), or by hybridization following synthesis and/or deprotection. Various modified oligonucleotides according to the present disclosure may also be synthesized using the teachings of Scaringe et al., U.S. Pat. Nos. 5,889,136; 6,008,400; and 6,111,086.

V.3 Preparation of Modified dsRNAs

dsRNAs of the present disclosure may be chemically/physically linked to one or more ligands, moieties or conjugates. In some embodiments, the dsRNA is conjugated/attached to one or more ligands via a linker. Any linker known in the art may be used, including, for example, multivalent branched linkers. Conjugating a ligand to a dsRNA may alter its distribution, enhance its cellular absorption and/or targeting to a particular tissue and/or uptake by one or more specific cell types (e.g., liver cells), and/or enhance the lifetime of the dsRNA agent. In some embodiments, a hydrophobic ligand is conjugated to the dsRNA to facilitate direct permeation across the cellular membrane and/or uptake by the cells (e.g., liver cells).

In some embodiments of a dsRNA conjugate, one or more nucleotides may comprise a targeting moiety-bearing group, such as one or more nucleotides comprise a targeting moiety bearing group wherein a targeting moiety is covalently linked to the nucleotide backbone, possibly via a linking group. According to these embodiments, one or more nucleotides of a dsRNA are conjugated to a targeting moiety-bearing group comprising a targeting moiety and wherein the targeting moiety may be, a ligand (e.g., a cell penetrating moiety or agent) that enhances intracellular delivery of the compositions.

Ligand-conjugated dsRNAs and ligand-molecule bearing sequence-specific linked nucleosides and nucleotides of the present disclosure may be assembled by any method known in the art, including, for example, by assembly on a suitable DNA synthesizer utilizing standard nucleotide precursors, or nucleotide or nucleoside conjugate precursors that already bear the linking moiety, ligand-nucleotide, or nucleoside-conjugated precursors that already bear the ligand molecule, or non-nucleoside ligand-bearing building blocks.

Ligand-conjugated dsRNAs of the present disclosure may be synthesized by any method known in the art, including, for example, by the use of a dsRNA bearing a pendant reactive functionality such as that derived from the attachment of a linking molecule onto the dsRNA. In some embodiments, this reactive oligonucleotide may be reacted directly with commercially available ligands, ligands that are synthesized bearing any of a variety of protecting groups, or ligands that have a linking moiety attached thereto. In some embodiments, the methods facilitate the synthesis of ligand-conjugated dsRNA by the use of nucleoside monomers that have been appropriately conjugated with ligands and that may further be attached to a solid support material. In some embodiments, a dsRNA bearing an aralkyl ligand attached to the 3′-end of the dsRNA is prepared by first covalently attaching a monomer building block to a controlled-pore-glass support via an aminoalkyl group; then, nucleotides are bonded via standard solid-phase synthesis techniques to the monomer building-block bound to the solid support. The monomer building-block may be a nucleoside or other organic compound that is compatible with solid-phase synthesis.

The present disclosure also relates to a method of preparing a liver-targeting therapeutic agent, comprising mixing a therapeutic moiety and a compound of any one of claims 1-44 to allow conjugation of the compound to the therapeutic moiety, thereby generating a liver-targeting therapeutic agent.

### VI. Compositions

Certain aspects of the present disclosure relate to compositions (e.g., pharmaceutical compositions) comprising a dsRNA as described herein. In some embodiments, the composition (e.g., pharmaceutical composition) further comprises a pharmaceutically acceptable carrier. In some embodiments, the composition (e.g., pharmaceutical composition) is useful for treating a disease or disorder associated with the expression or activity of the targeted gene.

Compositions (e.g., pharmaceutical compositions) of the present disclosure are formulated based upon the mode of delivery, including, for example, compositions formulated for delivery to the liver via parenteral delivery.

The compositions (e.g., pharmaceutical composition) of the present disclosure may be administered in dosages sufficient to inhibit expression of the targeted gene. In some embodiments, a suitable dose of a dsRNA is in the range of 0.01 mg/kg-400 mg/kg body weight of the recipient.

One of ordinary skill in the art will appreciate that certain factors may influence the dosage and timing required to effectively treat a subject, including, but not limited to, severity of the disease or disorder, previous treatments, the general health and/or age of the subject, and one or more other diseases being present. Moreover, treatment of a subject with a therapeutically effective amount of a pharmaceutical composition can include a single treatment or a series of treatments. Estimates of effective dosages and in vivo half-lives for dsRNAs as disclosed herein may be made using conventional methodologies or on the basis of in vivo testing using an appropriate animal model.

dsRNA molecules of the present disclosure can be formulated in a pharmaceutically acceptable carrier or diluent. Pharmaceutically acceptable carriers can be liquid or solid, and may be selected with the planned manner of administration in mind so as to provide for the desired bulk, consistency, and other pertinent transport and chemical properties. Any known pharmaceutically acceptable carrier or diluent may be used, including, for example, water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), calcium salts (e.g., calcium sulfate, calcium chloride, calcium phosphate, etc.) and wetting agents (e.g., sodium lauryl sulfate).

dsRNA molecules of the present disclosure can be formulated into compositions (e.g., pharmaceutical compositions) containing the dsRNA admixed, encapsulated, conjugated, or otherwise associated with other molecules, molecular structures, or mixtures of nucleic acids. For example, a composition comprising one or more dsRNAs as described herein can contain other therapeutic agents such as other lipid lowering agents (e.g., statins). In some embodiments, the composition (e.g., pharmaceutical composition) further comprises a delivery vehicle (as described herein).

VII. Vectors and dsRNA Delivery

A dsRNA of the present disclosure may be delivered directly or indirectly. In some embodiments, the dsRNA is delivered directly by administering a composition (e.g., pharmaceutical composition) comprising the dsRNA to a subject. In some embodiments, the dsRNA is delivered indirectly by administering one or more vectors described herein.

A dsRNA of the present disclosure may be delivered by any method known in the art, including, for example, by adapting a method of delivering a nucleic acid molecule for use with a dsRNA (see e.g., Akhtar et al., Trends Cell Biol. (1992) 2(5):139-44; WO 94/02595), or via additional methods known in the art (see e.g, Kanasty et al., Nature Materials (2013) 12:967-77; Wittrup, A. and Lieberman, J. (2015) Nature Reviews Genetics 16: 543-552; Whitehead et al., Nature Reviews Drug Discovery (2009) 8:129-38; Gary et al., (2007) 121 (1-2): 64-73; Wang. J. et al. (2010) AAPSJ. 12(4): 492-503; Draz, M. et al. (2014) Theranostics 4(9): 872-892; Wan, C. et al. (2013) Drug Deliv. And Transl. Res. 4(1): 74-83; Erdmann, V. A. and Barciszewski, J. (eds.) (2010) “RNA Technologies and Their Applications”, Springer-V erlag Berlin Heidelberg, DOI 10.1007/978-3-642-12168-5; Xu, C. and Wang, J. (2Q\5) Asian Journal of Pharmaceutical Sciences 10(1): 1-12).

In some embodiments, a dsRNA of the present disclosure is delivered by a delivery vehicle comprising the dsRNA. In some embodiments, the delivery vehicle is a liposome, lipoplex, complex, or nanoparticle.

### VII.1 Liposomal Formulations

Liposomes are unilamellar or multilamellar vesicles which have a membrane formed from a lipophilic material and an aqueous interior. In some embodiments, a liposome is a vesicle composed of amphiphilic lipids arranged in a spherical bilayer or bilayers. The aqueous portion contains the composition to be delivered. Cationic liposomes possess the advantage of being able to fuse to the cell wall. Advantages of liposomes include, e.g., liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes. For example, engineered cationic liposomes and sterically stabilized liposomes can be used to deliver the dsRNA. See, e.g., Podesta et al. (2009) Methods Enzymol. 464, 343-54; U.S. Pat. No. 5,665,710.

### VII.2 Nucleic Acid-Lipid Particles

In some embodiments, a dsRNA of the present disclosure is fully encapsulated in a lipid formulation, e.g., to form a nucleic acid-lipid particle, e.g., a SPLP, pSPLP, or SNALP. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. Nucleic acid-lipid particles, e.g., SNALPs, typically contain a cationic lipid, a non-cationic lipid, cholesterol and a lipid that prevents aggregation of the particle and increases circulation time (e.g., a PEG-lipid conjugate). SNALPs and SPLPs are useful for systemic applications, as they exhibit extended circulation lifetimes following intravenous (i.v.) injection and accumulate at distal sites (e.g., sites physically separated from the administration site). SPLPs include “pSPLP”, which include an encapsulated condensing agent-nucleic acid complex as set forth in PCT Publication No. WO 00/03683.

In some embodiments, dsRNAs when present in the nucleic acid-lipid particles are resistant in aqueous solution to degradation with a nuclease. Nucleic acid-lipid particles and their methods of preparation are disclosed in, e.g., U.S. Pat. Nos. 5,976,567; 5,981,501; 6,534,484; 6,586,410; and 6,815,432; and PCT Publication No. WO 96/40964.

In some embodiments, the nucleic acid-lipid particles comprise a cationic lipid. Any cationic lipid or mixture thereof known in the art may be used. In some embodiments, the nucleic acid-lipid particles comprise a non-cationic lipid. Any non-cationic lipid or mixture thereof known in the art may be used. In some embodiments, the nucleic acid-lipid particle comprises a conjugated lipid (e.g., to prevent aggregation). Any conjugated lipid known in the art may be used.

### VII.3 Additional Formulations for Delivery

Factors that are important to consider in order to successfully deliver a dsRNA molecule in vivo include: (1) biological stability of the delivered molecule, (2) preventing nonspecific effects, and (3) accumulation of the delivered molecule in the target tissue. The nonspecific effects of a dsRNA can be minimized by local administration, for example by direct injection or implantation into a tissue or topically administering the preparation. For administering a dsRNA systemically for the treatment of a disease, the dsRNA may be modified or alternatively delivered using a drug delivery system; both methods act to prevent the rapid degradation of the dsRNA by endo and exo-nucleases in vivo. Modification of the RNA or the pharmaceutical carrier may also permit targeting of the dsRNA composition to the target tissue and avoid undesirable off-target effects. As described above, dsRNA molecules may be modified by chemical conjugation to lipophilic groups such as cholesterol to enhance cellular uptake and prevent degradation. In some embodiments, the dsRNA is delivered using drug delivery systems such as a nanoparticle, a dendrimer, a polymer, liposomes, or a cationic delivery system. Positively charged cationic delivery systems facilitate binding of a dsRNA molecule (negatively charged) and also enhance interactions at the negatively charged cell membrane to permit efficient uptake of a dsRNA by the cell. Cationic lipids, dendrimers, or polymers can either be bound to a dsRNA, or induced to form a vesicle or micelle (See e.g., Kim S. H. et al. (2008) Journal of Controlled Release 129(2): 107-116) that encases a dsRNA. The formation of vesicles or micelles further prevents degradation of the dsRNA when administered systemically. Methods for making and administering cationic-dsRNA complexes are known in the art. In some embodiments, a dsRNA forms a complex with cyclodextrin for systemic administration.

VIII. Methods of Using dsRNA

Certain aspects of the present disclosure relate to methods for inhibiting the expression of a targeted gene in a mammal comprising administering an effective amount of one or more dsRNAs of the present disclosure, one or more vectors of the present disclosure, or a composition (e.g., pharmaceutical composition) of the present disclosure comprising one or more dsRNAs of the present disclosure. Certain aspects of the present disclosure relate to methods of treating and/or preventing one or more target gene-mediated diseases or disorders comprising administering one or more dsRNAs of the present disclosure and/or one or more vectors of the present disclosure and/or a composition (e.g., pharmaceutical composition) comprising one or more dsRNAs of the present disclosure. In some embodiments, downregulating target gene expression in a subject alleviates one or more symptoms of a targeted gene-mediated disease or disorder in the subject.

The present disclosure further relates to a method of delivering an oligonucleotide to liver (hepatic) cells in a human subject in need thereof, comprising administering to the subject an oligonucleotide as described herein.

In some embodiments of the said method, the administration is through intravenous or subcutaneous injection or injection through the hepatic portal vein.

The present disclosure also pertains to the use an oligonucleotide as described herein for the manufacture of a medicament to treat a human subject in need thereof. In some of these embodiments, the said oligonucleotide as described herein is for use in treating a human subject in need thereof. This disclosure also concerns a method of delivering a therapeutic agent to liver (hepatic) cells in a human subject in need thereof, comprising administering to the subject a therapeutic moiety conjugated to a compound as described herein, especially of a compound of formula (I) or of formula (II) described herein, and even more specifically an oligonucleotide comprising one or more of the said compound(s).

The present disclosure further relates to the use of a compound as described herein, especially of a compound of formula (I) or of formula (II) described herein, and even more specifically an oligonucleotide comprising one or more of the said compound(s), for the manufacture of a medicament that targets a therapeutic agent to liver (hepatic) cells in a human subject in need thereof.

The present disclosure also pertains to the compound as described herein, especially of a compound of formula (I) or of formula (II) described herein, and even more specifically an oligonucleotide comprising one or more of the said compound(s), for use in delivering a therapeutic agent to liver (hepatic) cells in a human subject in need thereof.

In some embodiments of the above uses or methods, the therapeutic agent is a protein, a peptide, a peptide mimetic, a small molecule, or a polynucleotide.

In some embodiments, expression of the target gene in the subject is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100% after treatment as compared to pretreatment levels. In some embodiments, expression of the target gene is inhibited by at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 25 fold, at least about 50 fold, at least about 75 fold, or at least about 100 fold after treatment as compared to pretreatment levels. In some embodiments, the target gene is inhibited in the liver of the subject.

In some embodiments, the subject is human. In some embodiments, the subject has or has been diagnosed with a target gene-mediated disorder or disease. In some embodiments, the subject is suspected to have a target gene-mediated disorder or disease. In some embodiments, the subject is at risk for developing a target gene-mediated disorder or disease.

As it is understood from the content of the present disclosure, a dsRNA as described herein has its main characteristics lying in the presence of one or more nucleotide analogs of formula (II) comprised therein, which nucleotide analogs of formula (IV) possess specific structural features of the “sugar-like” group thereof. A dsRNA as described herein is generally conceived for targeting a selected nucleic acid sequence comprised in a target nucleic acid of interest. Especially, embodiments of a dsRNA described herein consisting of siRNAs comprise an antisense strand that specifically hybridizes with a nucleic acid sequence comprised in a target nucleic acid of interest. A dsRNA or composition (e.g., pharmaceutical composition) described herein may be for use in the treatment of target gene-mediated disorder or disease. In particular, a dsRNA or composition (e.g., pharmaceutical composition) described herein, and especially a dsRNA comprising one or more targeted nucleotide analogs, and especially one or more ASGPR-targeted nucleotide analogs of formula (IV), may be for use in the treatment of target gene-mediated disorder or disease wherein liver-targeting is needed.

Certain aspects of the present disclosure also relate to a method of delivery of nucleic acids to hepatocytes comprising contacting the hepatocyte with a dsRNA described herein.

A dsRNA or composition (e.g., pharmaceutical composition) described herein may be administered by any means known in the art, including, without limitation, oral or parenteral routes, including intravenous, intramuscular, subcutaneous, pulmonary, transdermal, and airway (aerosol) administration. Typically, when treating a mammal with hyperlipidemia, the dsRNA molecules are administered systemically via parenteral means. In some embodiments, the dsRNAs and/or compositions are administered by subcutaneous administration. In some embodiments, the dsRNAs and/or compositions are administered by intravenous administration. In some embodiments, the dsRNAs and/or compositions are administered by pulmonary administration.

A treatment or preventative effect of a dsRNA is evident when there is a statistically significant improvement in one or more parameters of disease status, or by a failure to worsen or to develop symptoms where they would otherwise be anticipated. For example, a favorable change of at least 10%, 15%, 20%, 25%, 30%, 35%, 40%, 45%, 50% or more in a measurable parameter of disease may be indicative of effective treatment. Efficacy for a given dsRNA or composition comprising the dsRNA may also be judged using an experimental animal model for the given disease or disorder known in the art. When using an experimental animal model, efficacy of treatment is evidenced when a statistically significant reduction in a marker or symptom is observed.

### IX. Kits and Articles of Manufacture

Certain aspects of the present disclosure relate to an article of manufacture or a kit comprising one or more of the dsRNAs, vectors, or compositions (e.g., pharmaceutical compositions) as described herein useful for the treatment and/or prevention of a disease. The article of manufacture or kit may further comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating or preventing the disease and may have a sterile access port (for example the container may be an intravenous solution bag or a vial having a stopper pierceable by a hypodermic injection needle). At least one active agent in the composition is a dsRNA as described herein. The label or package insert indicates that the composition is used for treating a disease. Moreover, the article of manufacture or kit may comprise (a) a first container with a composition contained therein, wherein the composition comprises a dsRNA as described herein; and (b) a second container with a composition contained therein, wherein the composition comprises a second therapeutic agent (e.g., an additional agent as described herein). The article of manufacture or kit in this aspect of the present disclosure may further comprise a package insert indicating that the compositions can be used to treat a particular disease. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and/or user standpoint, including other buffers, diluents, filters, needles, and syringes.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to the manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

### EXAMPLES

In order for the present disclosure to be better understood, the following examples are set forth. These examples are for illustration only and are not to be construed as limiting the scope of the present disclosure in any manner.

Abbreviations used:
AcOH: acetic acid
FA: formic acid
ACN: acetonitrile
DCM: dichloromethane
DMA: dimethylacetamide
DCE: dichloroethane
DMF: dimethylformamide
DMSO: dimethylsulfoxide
EtOAc: ethyl acetate
EtOH: ethanol
Et₂O: diethylether
iPrOH: isopropanol
THF: tetrahydrofuran
MeOH: methanol
NMP: N-methyl-2-pyrrolidone
PE: petroleum ether
Pyr: pyridine
iPr: isopropyl
iBu: isobutyryl
cHex: cyclohexyl
MTB: methyl-tert-butyl
DIPEA: diisopropylethylamine
DMAP: 4-(dimethylamino)-pyridine
DBU: 1,8-Diazabicyclo[5.4.0]undec-7-ene
HBTU: (2-(1H-Benzotriazol-1-yl)-1,1,3,3-tetramethyluronium-hexafluorophosphate)
TBTU: O-(Benzotriazol-1-yl)-N,N,N′,N′-tetramethyluronium tetrafluoroborate
DDTT: 3-((N,N-dimethyl-aminomethylidene)amino)-3H-1,2,4-dithiazole-5-thione
NEt₃: triethylamine
NEM: N-ethyl-morpholine
BSA: N,O-bis-trimethylsilyl acetamide
TMSOTf: trimethylsilyltrifluormethanesulfonate
Ts: p-toluenesulfonyl
Tf: trifluormethanesulfonyl
trifluoromethanesulfonate
TFA: trifluoroacetic acid
DCAA: dichloroacetic acid
TEA: triethylammonium
TIPS: triisopropylsilyl
TBDMS: tert-butyldimethylsilyl
DMT: 4,4′-dimethoxytrityl
Bzl: benzyl
Bn: benzyl
BOM: benzyloxymethyl
Ac: acetyl
ⁱBu: isobutyryl
Boc: tert-butyloxycarbonyl
Fmoc: fluorenylmethyloxycarbonyl
Fmoc-OSu: N-(9-Fluorenylmethoxycarbonyloxy)succinimide
CE: cyanoethyl
CPG: controlled pore glass
T: thymine
U: uracile
C: cytosine
A: adenine
G: guanine
I: hypoxanthinehypoxanthine
T^{BOM}: N-benzyloxymethyl-thymine
U^{BOM}: N-benzyloxymethyl-uracile
U^{Bzl}: N-benzyl-uracile
C^{Bzl}: N-benzyl-cytosine
A^{Bzl}: N-benzyl-adenine
G^{iBu}: N-isobutyryl-guanine
GalNAc: D-N-acetylgalactosamine
FR: flow rate
HPLC: high pressure liquid chromatography
MS-TOF: Mass spectrometry-time of flight
LC-MS: High-pressure liquid chromatography-Mass spectrometry
Rₜ: retention time
RT: room temperature
Hal: halogen
ELSD: evaporative light scattering detector
quant.: quantitative
sat.: saturated
i. vac.: in vacuum
n.d.: not determined
TLC: thin layer chromatography
h: hour
min: minutes
Tm: melting temperature
r: ribonucleotide
d: desoxy-ribonucleotide
m: 2′-OMe-nucleotide
f: 2′-desoxy-fluoro-nucleotide
ss: sense strand
as: antisense strand
ds: double strand
chol: cholesterol
PO: phosphodiester linkage
* or PS: phosphorothiate linkage
mpk: mg/kg
M: molar
#: number, n°
FBS: fetal bovine serum
ATP: adenosine-triphosphate
pre-lB: precursor nucleotide
pre-lgB: targeted precursor nucleotide
lB: nucleotide analog
lgB: targeted nucleotide analog

### Example 1: Synthetic Scheme for the Syntheses of Example Compounds 2, 3 and 23

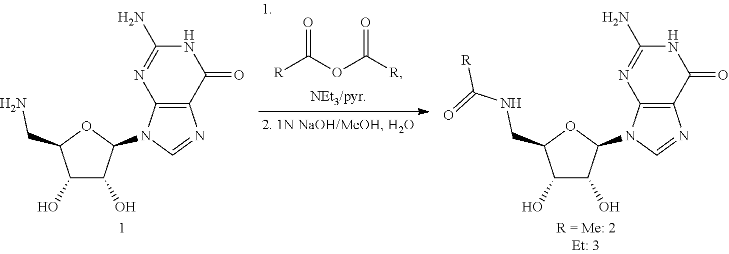

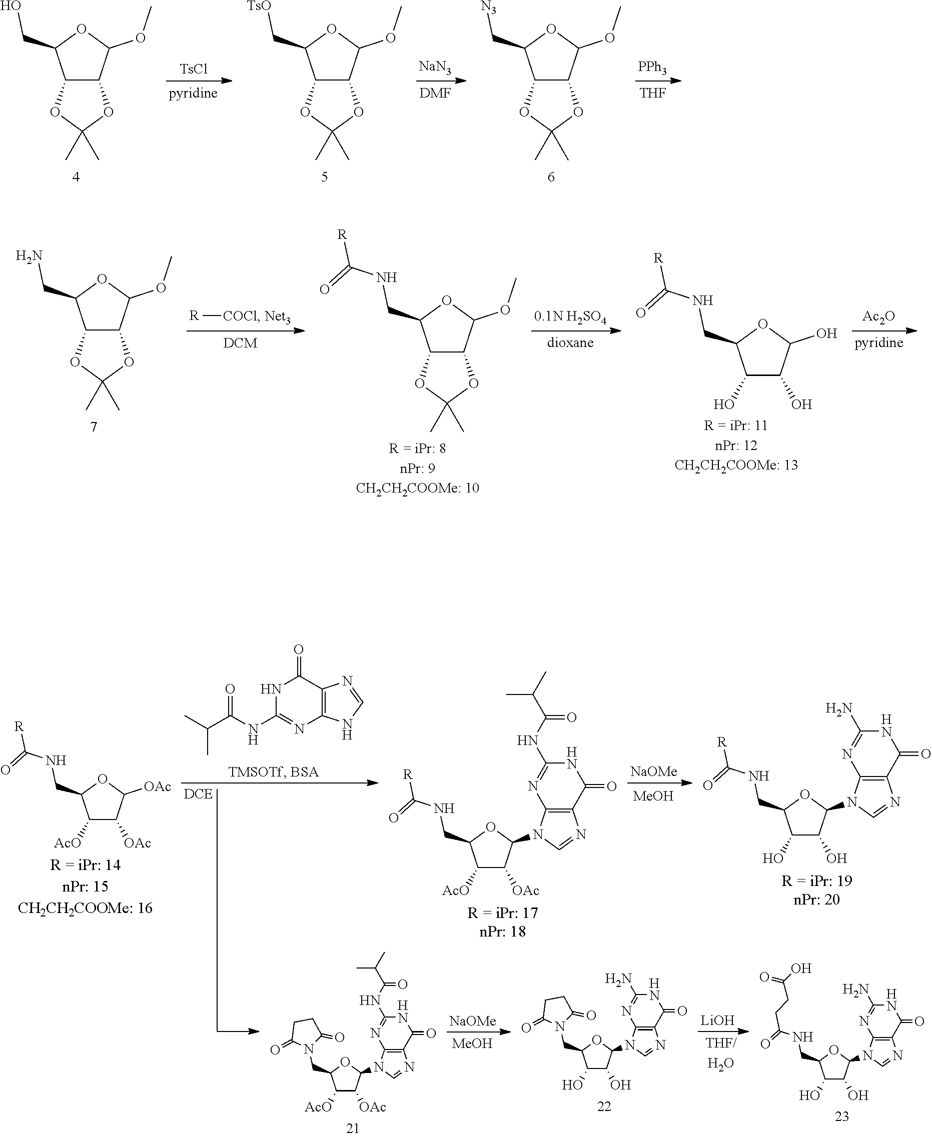

### Example 1.1: Synthesis of N-[[(2R,3S,4R,5R)-5-(2-amino-6-oxo-1H-purin-9-yl)-3,4-dihydroxy-tetrahydrofuran-2-yl]methyl]acetamide (2)

5′-Desoxy-5′-amino-guanosine (1, 100 mg, 0.34 mmol) was dissolved in 1.4 ml pyridine and 89 mg (0.67 mmol) NEt₃ were added at room temperature. After adding 139 mg (1.35 mmol) acetic anhydride, the reaction solution was stirred at room temperature overnight. The solvents were removed in vacuo and the residue was dissolved in 5 ml MeOH/H₂O (1:1). After adding 1 ml (1.0 mmol) of a 1 M NaOH-solution, the reaction mixture was stirred for 2 h at room temperature. The solution was diluted with 2.5 ml H₂O and neutralized with 2 N HCl. After adding 130 mg Amberlite IRN 150 ion exchanger, the mixture was stirred for 15 min. The mixture was filtered and the MeOH evaporated. Lyophilization of the aqueous solution gave 64 mg (58.6%) of the title compound N-[[(2R,3S,4R,5R)-5-(2-amino-6-oxo-1H-purin-9-yl)-3,4-dihydroxy-tetrahydrofuran-2-yl]methyl]acetamide (2) as colorless foam.

LC-MS (Method A):

Rₜ[min] (ELSD-signal): 0.30

MS (calc.: 324.1) (m/z)=325.3 [M+H⁺]

¹H-NMR (600 MHz, DMSO-d₆) δ[ppm]: 10.68, (s, 1H), 8.00 (t, J=5.9 Hz, 1H), 7.91 (s, 1H), 6.52 (br s, 2H), 5.66 (d, J=5.9 Hz, 1H), 5.42 (d, J=6.1 Hz, 1H), 5.15 (br d, J=4.6 Hz, 1H), 4.42 (dd, J=11.2, 5.5 Hz, 1H), 4.02 (m, 1H), 3.83 (m, 1H), 3.44 (dt, J=13.9, 5.7 Hz 1H), 3.21 (dt, J=13.8, 6.2 Hz 1H), 1.81 (s, 3H).

### Example 1.2: Synthesis of N-[[(2R,3S,4R,5R)-5-(2-amino-6-oxo-1H-purin-9-yl)-3,4-dihydroxy-tetrahydrofuran-2-yl]methyl]propanamide (3)

5′-Desoxy-5′-amino-guanosine (1, 100 mg, 0.34 mmol) was dissolved in 1.4 ml pyridine and 89 mg (0.67 mmol) NEt₃ were added at room temperature. After adding 177 mg (1.35 mmol) propionic acid anhydride, the reaction solution was stirred at room temperature overnight. The solvents were removed in vacuo and the residue was dissolved in 5 ml MeOH/H₂O (1:1). After adding 1 ml (1.0 mmol) of a 1 M NaOH-solution, the reaction mixture was stirred for 2 h at room temperature. The MeOH was removed in vacuo and the precipitate collected by filtration. After drying of the precipitate in vacuo, 62 mg (54.4%) of the title compound N-[[(2R,3S,4R,5R)-5-(2-amino-6-oxo-1H-purin-9-yl)-3,4-dihydroxy-tetrahydrofuran-2-yl]-methyl]propanamide (3) were obtained as colorless solid.

LC-MS (Method A):

Rₜ[min] (ELSD-signal): 0.51

MS (calc.: 338.1) (m/z)=339.3 [M+H⁺]

¹H-NMR (600 MHz, DMSO-d₆) δ[ppm]: 10.87 (br s, 1H), 7.92 (t, J=5.9 Hz, 1H), 7.88 (s, 1H), 6.54 (br s, 2H), 5.66 (d, J=6.1 Hz, 1H), 5.42 (br s, 1H), 5.14 (br s, 1H), 4.41 (t, J=5.6 Hz, 1H), 4.02 (t, J=4.4 Hz, 1H), 3.83 (m, 1H), 3.44 (dt, J=13.9, 5.6 Hz, 1H), 3.23 (dt, J=13.9, 6.2 Hz, 1H), 2.09 (q, J=7.5 Hz, 2H), 0.98 (t, J=7.6 Hz, 3H).

### Example 1.3: Synthesis of [(3aR,6R,6aR)-4-methoxy-2,2-dimethyl-3a,4,6,6a-tetrahydrofuro[3,4-d][1,3]dioxol-6-yl]methyl 4-methylbenzenesulfonate (5)

To a mixture of Methyl-2,3-Di-O-isopropylidene-D-riboside (4, 48.5 g, 0.237 mol, 1.0 eq) in pyridine (97 mL) was added TsCl (68 g, 0.356 mol, 1.5 eq) in portions at 0° C. The mixture was stirred at 25° C. for 5 h, to achieve complete conversion. After adding 100 ml of cold water, the mixture was stirred at 25° C. for 1 h. The precipitate was filtered, washed with 2×100 ml cold water and dried in vacuo, which gave 79.6 g (93.5%, crude) of the tosylate [(3aR,6R,6aR)-4-methoxy-2,2-dimethyl-3a,4,6,6a-tetrahydrofuro[3,4-d][1,3]dioxol-6-yl]methyl 4-methyl-benzenesulfonate (5) were isolated as white solid.

¹H-NMR (400 MHz, CDCl₃) δ[ppm]: 7.70-7.78 (m, 2H), 7.29 (d, J=8.0 Hz, 2H), 4.86 (s, 1H), 4.51-4.56 (m, 1H), 4.43-4.49 (m, 1H), 4.24 (t, J=7.2 Hz, 1H), 3.90-3.99 (m, 2H), 3.17 (s, 3H), 2.39 (s, 3H), 1.38 (s, 3H), 1.22 (s, 3H).

### Example 1.4: Synthesis of (3aR,6R,6aR)-6-(azidomethyl)-4-methoxy-2,2-dimethyl-3a,4,6,6a-tetrahydrofuro-[3,4-d][1,3]dioxole;[(3aR,6R,6aR)-4-methoxy-2,2-dimethyl-3a,4,6,6a-tetrahydrofuro[3,4-d][1,3]dioxol-6-yl]methyl 4-methylbenzenesulfonate (6)

A mixture of the tosylate [(3aR,6R,6aR)-4-methoxy-2,2-dimethyl-3a,4,6,6a-tetrahydro-furo[3,4-d][1,3]dioxol-6-yl]methyl 4-methylbenzenesulfonate (5, 36 g, 0.100 mol, 1.0 eq) and NaN₃ (13 g, 0.201 mol, 2.0 eq) in DMF (360 ml) was heated to 120° C. for 4 h. The heating bath was removed and the mixture allowed to reach room temperature. 200 mL of acetone were added and stirring was continued for 30 min. The acetone was removed in vacuo and the remaining solution poured into 1000 ml water. After extraction with 3×1000 ml, the organic layer was dried over anhydrous Na₂SO₄, filtered and concentrated in vacuo. The residue was purified by column chromatography (PE/EtOAc 30:1) to give compound 21.0 g (91.2%) of the desired azide (3aR,6R,6aR)-6-(azidomethyl)-4-methoxy-2,2-dimethyl-3a,4,6,6a-tetrahydrofuro-[3,4-d][1,3]dioxole;[(3aR,6R,6aR)-4-methoxy-2,2-dimethyl-3a,4,6,6a-tetra-hydrofuro[3,4-d][1,3]dioxol-6-yl]methyl 4-methylbenzenesulfonate (6) as colorless oil.

¹H-NMR (400 MHz, CDCl₃) δ[ppm]: 4.93 (s, 1H), 4.49-4.58 (m, 2H), 4.22 (t, J=7.3 Hz, 1H), 3.38 (dd, J=12.5, 7.64 Hz, 1H), 3.31 (s, 3H), 3.20 (dd, J=12.53, 6.8 Hz, 1H), 1.42 (s, 3H), 1.25 (s, 3H).

### Example 1.5: Synthesis of [(3aR,6R,6aR)-4-methoxy-2,2-dimethyl-3a,4,6,6a-tetrahydrofuro[3,4-d][1,3]dioxol-6-yl]methanamine (7)

To a mixture of compound the azide (3aR,6R,6aR)-6-(azidomethyl)-4-methoxy-2,2-dimethyl-3a,4,6,6a-tetrahydrofuro-[3,4-d][1,3]dioxole;[(3aR,6R,6aR)-4-methoxy-2,2-dimethyl-3a,4,6,6a-tetrahydrofuro[3,4-d][1,3]dioxol-6-yl]methyl 4-methylbenzenesulfonate (6, 15 g, 65.4 mmol, 1.0 eq) in THF (75 ml) was added PPh₃ (20.6 g, 78.524 mmol, 1.2 eq) in portions at 25° C. The solution was stirred for 16 hrs to achieve complete conversion. After adding 75 ml H₂O, stirring was continued for 5 h. The mixture was extracted with 2×200 ml DCM and the combined organic layers dried over anhydrous Na₂SO₄. After filtration, the solvent was evaporated in vacuo to give 30 g of the title compound [(3aR,6R,6aR)-4-methoxy-2,2-dimethyl-3a,4,6,6a-tetrahydrofuro[3,4-d][1,3]dioxol-6-yl]methanamine (7) (crude, purity ˜44%) as white solid, which was used without further purification.

### Example 1.6: Synthesis of N-[[(3aR,6R,6aR)-4-methoxy-2,2-dimethyl-3a,4,6,6a-tetrahydrofuro[3,4-d][1,3]-dioxol-6-yl]methyl]-2-methyl-propanamide (8)

The amine [(3aR,6R,6aR)-4-methoxy-2,2-dimethyl-3a,4,6,6a-tetrahydrofuro[3,4-d][1,3]-dioxol-6-yl]methanamine (7, 30 g, crude purity 44%) was dissolved in 43 ml NEt₃ and 173 ml DCM. After adding 2-methyl-propanoyl chlorid (8.4 g, 78.5 mmol) dissolved in 108 ml DCM dropwise at 0° C., the ice bath was removed and the solution stirred at room temperature for 3 h. The solution was diluted with DCM (300 ml) and washed with water (2×150 ml). The combined organic layers were dried over anhydrous Na₂SO₄, filtered and concentrated in vacuo. The residue was purified by flash chromatography (PE/EtOAc 1:1) to give 17.86 g (65.3%, two steps) of the title compound N-[[(3aR,6R,6aR)-4-methoxy-2,2-dimethyl-3a,4,6,6a-tetrahydrofuro[3,4-d][1,3]-dioxol-6-yl]methyl]-2-methyl-propanamide (8) as color-less oil.

¹H-NMR (400 MHz, CDCl₃) δ[ppm]: 6.17 (br s, 1H), 4.91 (s, 1H), 4.45-4.57 (m, 2H), 4.30 (t, J=5.1 Hz, 1H), 3.51 (dt, J=14.1, 6.4 Hz, 1H), 3.34 (s, 3H), 3.26 (dt, J=14.2, 4.5 Hz, 1H), 2.30 (spt, J=6.9 Hz, 1H), 1.40 (s, 3H), 1.23 (s, 3H), 1.09 (d, J=7.0 Hz, 6H).

### Example 1.7: Synthesis of 2-Methyl-N-[[(2R,3S,4R)-3,4,5-trihydroxytetrahydrofuran-2-yl]methyl]propenamide (11)

The starting material N-[[(3aR,6R,6aR)-4-methoxy-2,2-dimethyl-3a,4,6,6a-tetrahydrofuro-[3,4-d][1,3]-dioxol-6-yl]methyl]-2-methyl-propanamide (8, 12.86 g, 47.0 mmol, 1.0 eq) was dissolved in 0.1 N H₂SO₄ (155 ml, 15.5 mmol, 0.33 eq) and dioxane (77 ml). The reaction mixture was refluxed for 2 h, to achieve complete conversion. The reaction was cooled to room temperature and neutralized with Ba(OH)₂·8H₂O. After evaporation of the solvents in vacuo, the residue was co-evaporated three times with 100 ml dioxane, yielding 10.3 g (crude) of the deprotected product 2-Methyl-N-[[(2R,3S,4R)-3,4,5-trihydroxytetrahydrofuran-2-yl]methyl]-propenamide (11) as white solid, which was used without further purification.

### Example 1.8: Synthesis of [(2R,3R,4R)-4,5-diacetoxy-2-[(2-methylpropanoylamino)-methyl]tetrahydrofuran-3-yl] acetate (14)

The ribose derivative 2-Methyl-N-[[(2R,3S,4R)-3,4,5-trihydroxytetrahydrofuran-2-yl]methyl]-propenamide (11, 10.3 g, 47.0 mmol, 1.0 eq) was co-evaporated three times with 100 ml pyridine and dissolved in pyridine (155 ml). After adding 52 ml acetic anhydride at room temperature in one portion, the solution was stirred for 16 h. The reaction solution was concentrated in vacuo and the residue purified by column chromatography (PE/EtOAc 1:1), yielding 14.4 g (88.5%, two steps) of the acetyl-riboside [(2R,3R,4R)-4,5-diacetoxy-2-[(2-methylpropanoylamino)methyl]tetrahydrofuran-3-yl] acetate (14) as yellow oil.

¹H-NMR (400 MHz, CDCl₃) δ[ppm]: 6.14 (s, 1H), 5.80-5.91 (m, 1H), 5.33 (d, J=4.8 Hz, 1H), 5.13-5.20 (m, 1H), 4.23-4.32 (m, 1H), 3.67 (ddd, J=14.3, 6.02, 3.9 Hz, 1H), 3.35-3.46 (m, 1H), 2.30-2.44 (m, 1H), 2.05-2.17 (m, 9H), 1.11-1.22 (m, 6H).

### Example 1.9: Synthesis of [(2R,3R,4R,5R)-4-acetoxy-2-[(2-methylpropanoylamino)-methyl]-5-[2-(2-methyl-propanoyl-amino)-6-oxo-1H-purin-9-yl]tetrahydrofuran-3-yl] acetate (17)

The starting material [(2R,3R,4R)-4,5-diacetoxy-2-[(2-methylpropanoylamino)methyl]-tetrahydrofuran-3-yl] acetate (14, 12 g, 34.7 mmol, 1.0 eq) and compound isobutyryl-guanine (11.5 g, 52.1 mmol, 1.5 eq) were dissolved in DCE (480 ml). At room temperature, BSA (28.3 g, 0.139 mol, 4.0 eq) was added dropwise and the solution was stirred at 95° C. for 2 h. TMSOTf (23 g, 0.104 mol, 3.0 eq) was added at 90° C. and stirring at this temperature was continued for 7 h. The heating bath was removed and the solution cooled to room temperature. After adding 200 ml of water, the mixture was extracted with DCM (3×200 ml) and the combined organic layers were dried over anhydrous Na₂SO₄, filtered and concentrated in vacuo. The residue was purified by flash chromatography (4% MeOH in EtOAc) to give 13.3 g of the guanosine analog (17) (75.7%, purity 66%) as yellow foam. 5 g were purified by reverse flash chromato-graphy (TFA) to give pure [(2R,3R,4R,5R)-4-acetoxy-2-[(2-methylpropanoylamino)methyl]-5-[2-(2-methyl-propanoyl-amino)-6-oxo-1H-purin-9-yl]tetrahydrofuran-3-yl] acetate (17) (3.4 g) as white foam.

¹H-NMR (400 MHz, DMSO-d₆) δ[ppm]: 12.12 (s, 1H), 11.37-11.74 (m, 1H), 8.31 (s, 1H), 8.01-8.13 (m, 1H), 6.05 (d, J=6.5 Hz, 1H), 5.71 (t, J=6.2 Hz, 1H), 5.45 (dd, J=5.7, 3.6 Hz, 1H), 4.18 (td, J=5.9, 3.8 Hz, 1H), 3.63 (dt, J=13.9, 6.1 Hz, 1H), 3.39 (br d, J=2.0 Hz, 1H), 2.79 (spt, J=6.8 Hz, 1H), 2.32-2.45 (m, 1H), 2.08-2.16 (m, 3H), 1.97-2.05 (m, 3H), 1.14 (dd, J=6.9, 2.1 Hz, 6H), 1.00 (t, J=6.48 Hz, 6H).

MS (calc.: 506.2) (m/z)=507.5 [M+H⁺].

### Example 1.10: Synthesis of N-[[(2R,3S,4R,5R)-5-(2-amino-6-oxo-1H-purin-9-yl)-3,4-dihydroxy-tetrahydrofuran-2-yl]methyl]-2-methyl-propanamide (19)

The starting material [(2R,3R,4R,5R)-4-acetoxy-2-[(2-methylpropanoylamino)methyl]-5-[2-(2-methyl-propanoyl-amino)-6-oxo-1H-purin-9-yl]tetrahydrofuran-3-yl] acetate (17, 2 g, 3.95 mmol, 1.0 eq) was dissolved in 0.1 N NaOMe in MeOH solution (13 mL, 1.303 mmol, 0.33 eq) and heated at 60° C. for 5 h. After the solution was cooled to room temperature, the pH was adjusted to pH=6 by the addition of an aqueous 1 N HCl. The precipitate was filtered and triturated with water (20 ml) and acetone (5 ml), yielding 1.12 g (80.6%) of the guanosine analog N-[[(2R,3S,4R,5R)-5-(2-amino-6-oxo-1H-purin-9-yl)-3,4-dihydroxy-tetrahydrofuran-2-yl]methyl]-2-methyl-propanamide (19) as colorless solid.

MS (calc.: 352.1) (m/z)=353.1 [M+H⁺].

¹H-NMR (400 MHz, DMSO-d₆) δ[ppm]: 10.66 (s, 1H), 7.83-8.00 (m, 2H), 6.48 (br s, 2H), 5.67 (d, J=6.1 Hz, 1H), 5.45 (br d, J=5.8 Hz, 1H), 5.16 (br d, J=3.9 Hz, 1H), 4.40 (q, J=5.4 Hz, 1H), 4.02 (br d, J=3.4 Hz, 1H), 3.78-3.90 (m, 1H), 3.41-3.51 (m, 1H), 3.15-3.31 (m, 1H), 2.39 (spt, J=6.9 Hz, 1H), 0.98 (dd, J=6.9, 3.1 Hz, 6H).

### Example 1.11: Synthesis of N-[[(3aR,6R,6aR)-4-methoxy-2,2-dimethyl-3a,4,6,6a-tetrahydrofuro[3,4-d][1,3]-dioxol-6-yl]methyl]butanamide (9)

The starting material [(3aR,6R,6aR)-4-methoxy-2,2-dimethyl-3a,4,6,6a-tetrahydrofuro[3,4-d][1,3]dioxol-6-yl]methanamine (7, 22.5 g, purity 39%, impurity Ph₃PO, 43.6 mmol, 1.0 eq) was acylated with propionyl chloride (5.6 g, 52.3 mmol, 1.2 eq), following the protocol, described for the synthesis of N-[[(3aR,6R,6aR)-4-methoxy-2,2-dimethyl-3a,4,6,6a-tetra-hydrofuro[3,4-d][1,3]-dioxol-6-yl]methyl]-2-methyl-propanamide (8), which gave, after column chromatography (PE/EtOAc 2:1), 17.2 g (purity 69%, impurity Ph₃PO, 100%) of the desired amide N-[[(3aR,6R,6aR)-4-methoxy-2,2-dimethyl-3a,4,6,6a-tetrahydrofuro[3,4-d]-[1,3]-dioxol-6-yl]methyl]butanamide (9) as colorless oil.

### Example 1.12: Synthesis of N-[[(2R,3S,4R)-3,4,5-trihydroxytetrahydrofuran-2-yl]methyl]-butanamide (12)

The ribose derivative N-[[(3aR,6R,6aR)-4-methoxy-2,2-dimethyl-3a,4,6,6a-tetrahydrofuro-[3,4-d][1,3]-dioxol-6-yl]methyl]butanamide (9, 17.2 g, purity 69%, 43.6 mmol, 1.0 eq) was dissolved in 0.1 N H₂SO₄ (140 ml, 14.4 mmol, 0.33 eq) and dioxane (70 ml). After heating under reflux for 2 h, the reaction was cooled to room temperature and the mixture neutralized with Ba(OH)₂·8H₂O (solid). The solution was evaporated in vacuo, the residue diluted with water (100 ml) and washed with EtOAc (3×50 ml). The aqueous layer was concentrated in vacuo and the residue was co-evaporated with dioxane (3×100 ml), yielding the title compound N-[[(2R,3S,4R)-3,4,5-trihydroxytetrahydrofuran-2-yl]methyl]butanamide (12) (10.2 g, purity 94%, 100%) as white solid, which was used without further purification.

### Example 1.13: Synthesis of [(2R,3R,4R)-4,5-diacetoxy-2-[(butanoylamino)methyl]tetra-hydrofuran-3-yl] acetate (15)

Following the protocol, described for the synthesis for [(2R,3R,4R)-4,5-diacetoxy-2-[(2-methylpropanoylamino)methyl]tetrahydrofuran-3-yl] acetate (14), the starting material N-[[(2R,3S,4R)-3,4,5-trihydroxytetrahydrofuran-2-yl]methyl]butanamide (12, 10.2 g, 43.6 mmol, 1.0 eq) was acetylated, to yield, after silicagel chromatography (PE/EtOAc 1:1), 10.1 g (67.3%) of the triacetate [(2R,3R,4R)-4,5-diacetoxy-2-[(butanoylamino)methyl]tetrahydrofuran-3-yl] acetate (15) as yellow oil.

### Example 1.14: Synthesis of [(2R,3R,4R,5R)-4-acetoxy-2-[(butanoylamino)methyl]-5-[2-(2-methylpropanoyl-amino)-6-oxo-1H-purin-9-yl]tetrahydrofuran-3-yl] acetate (18)

The starting compound [(2R,3R,4R)-4,5-diacetoxy-2-[(butanoylamino)methyl]tetrahydro-furan-3-yl] acetate (15, 5 g, 14.5 mmol, 1.0 eq) was glycosylated with isobutyryl-guanine, following the protocol, described for the synthesis of [(2R,3R,4R,5R)-4-acetoxy-2-[(2-methylpropanoylamino)methyl]-5-[2-(2-methyl-propanoyl-amino)-6-oxo-1H-purin-9-yl]tetra-hydrofuran-3-yl] acetate (17). After silicagel chromatography (EtOAc/MeOH 20:1), 4.5 g (61.6%) of the title compound [(2R,3R,4R,5R)-4-acetoxy-2-[(butanoylamino)methyl]-5-[2-(2-methylpropanoyl-amino)-6-oxo-1H-purin-9-yl]tetrahydrofuran-3-yl] acetate (18) were isolated as white foam.

MS (calc.: 506.2) (m/z)=507.3 [M+H⁺].

### Example 1.15: Synthesis of N-[[(2R,3S,4R,5R)-5-(2-amino-6-oxo-1H-purin-9-yl)-3,4-dihydroxy-tetrahydrofuran-2-yl]methyl]butanamide (20)

Following the protocol, described for the synthesis of N-[[(2R,3S,4R,5R)-5-(2-amino-6-oxo-1H-purin-9-yl)-3,4-dihydroxy-tetrahydrofuran-2-yl]methyl]-2-methyl-propanamide (19), the starting material [(2R,3R,4R,5R)-4-acetoxy-2-[(butanoylamino)methyl]-5-[2-(2-methylpro-panoyl-amino)-6-oxo-1H-purin-9-yl]tetrahydrofuran-3-yl] acetate (18, 3.0 g, 5.9 mmol, 1.0 eq) was treated with NaOMe in MeOH at 60° C. for 8 h. After workup as described for (19), 1.46 g (70.2%) of the title compound N-[[(2R,3S,4R,5R)-5-(2-amino-6-oxo-1H-purin-9-yl)-3,4-dihydroxy-tetrahydrofuran-2-yl]methyl]butanamide (20) were isolated as white solid.

MS (calc.: 352.1) (m/z)=353.0 [M+H⁺].

¹H-NMR (400 MHz, DMSO-d₆) δ[ppm]: 10.71 (s, 1H), 8.01-7.92 (m, 2H), 6.52 (br s, 2H), 5.68 (d, J=6.0 Hz, 1H), 5.57-4.89 (m, 2H), 4.42 (t, J=5.6 Hz, 1H), 4.07-3.98 (m, 1H), 3.88-3.78 (m, 1H), 3.45 (td, J=5.7, 13.7 Hz, 1H), 3.30-3.17 (m, 1H), 2.12-2.02 (m, 2H), 1.50 (sxt, J=7.4 Hz, 2H), 0.93-0.74 (m, 3H).

### Example 1.16: Synthesis of Methyl 4-[[(3aR,6R,6aR)-4-methoxy-2,2-dimethyl-3a,4,6,6a-tetrahydrofuro[3,4-d][1,3]-dioxol-6-yl]methylamino]-4-oxo-butanoate (10)

To a solution of the starting compound [(3aR,6R,6aR)-4-methoxy-2,2-dimethyl-3a,4,6,6a-tetrahydrofuro[3,4-d][1,3]dioxol-6-yl]methanamine (7, 21.3 g, crude, 1.0 eq) in DCM (150 ml) was added at 0° C. NEt₃ (36.4 ml) dropwise, followed by a solution of succinylchloride monomethyl ester (7.55 g, 50.3 mmol, 1.2 eq) in DCM (92 ml). After stirring at room temperature for 3 h, the solution was diluted with DCM (200 ml), washed with water (100 ml), dried over anhydrous Na₂SO₄, filtered and concentrated in vacuo. The residue was purified by column chromatography (PE/EtOAc 1:1), yielding 20.0 g (66%, 46% purity) of the title compound Methyl 4-[[(3aR,6R,6aR)-4-methoxy-2,2-dimethyl-3a,4,6,6a-tetrahydrofuro[3,4-d][1,3]-dioxol-6-yl]methylamino]-4-oxo-butanoate (10) as yellow oil.

¹H-NMR (400 MHz, CDCl₃) δ[ppm]: 4.99 (s, 1H), 4.67-4.59 (m, 2H), 4.37 (t, J=5.3 Hz, 1H), 3.73-3.68 (m, 3H), 3.63-3.54 (m, 1H), 3.45-3.40 (m, 3H), 3.39-3.29 (m, 1H), 2.79-2.59 (m, 2H), 2.51-2.44 (m, 2H), 1.48 (s, 3H), 1.32 (s, 3H).

### Example 1.17: Synthesis of Methyl 4-oxo-4-[[(2R,3S,4R)-3,4,5-trihydroxytetrahydrofuran-2-yl]methylamino]-butanoate (13)

To a solution of Methyl 4-[[(3aR,6R,6aR)-4-methoxy-2,2-dimethyl-3a,4,6,6a-tetrahydrofuro-[3,4-d][1,3]-dioxol-6-yl]methylamino]-4-oxo-butanoate (10, 5 g, 7.25 mmol, 1.0 eq) in dioxane (30 ml) was added a 0.1 N H₂SO₄ solution (24 ml, 2.4 mmol, 0.33 eq) in one portion at room temperature. The solution was stirred at 120° C. for 2 h, to achieve complete conversion. The reaction mixture was adjusted with Ba(OH)₂·8H₂O (solid) to pH=7 and filtered. The filtrate was washed with EtOAc (2×30 ml). The aqueous layer separated and concentrated in vacuo to give compound 2.0 g (crude, quant.) of the deprotected ribose derivative Methyl 4-oxo-4-[[(2R,3S,4R)-3,4,5-trihydroxytetrahydrofuran-2-yl]methylamino]-butanoate (13) as yellow oil, which was used without further purification.

### Example 1.18: Synthesis of Methyl 4-oxo-4-[[(2R,3R,4R)-3,4,5-triacetoxytetrahydrofuran-2-yl]methylamino]-butanoate (16)

To a solution of the starting material Methyl 4-oxo-4-[[(2R,3S,4R)-3,4,5-trihydroxytetra-hydrofuran-2-yl]methylamino]-butanoate (13, 2.0 g, 7.6 mmol, 1.0 eq) in pyridine (20 ml) was added Ac₂O (10 ml) dropwise at room temperature. The solution was stirred for 12 h and evaporated in vacuo. The residue was purified by column chromatography (PE/EtOAc 2:1), which gave 1.2 g (41%) of title compound Methyl 4-oxo-4-[[(2R,3R,4R)-3,4,5-triacetoxy-tetrahydrofuran-2-yl]methylamino]-butanoate (16) as yellow oil.

MS (calc.: 389.1) (m/z)=412.2 [M+Na⁺].

¹H-NMR (400 MHz, CDCl₃) δ[ppm]: 6.15-6.11 (m, 1H), 5.41-5.36 (m, 1H), 5.28 (d, J=5.4 Hz, 1H), 5.26-5.23 (m, 1H), 5.23-5.07 (m, 1H), 4.51-4.44 (m, 1H), 3.88-3.67 (m, 4H), 2.73 (s, 3H), 2.18 (s, 3H), 2.13 (s, 3H), 2.08 (s, 3H).

### Example 1.19: Synthesis of [(2R,3R,4R,5R)-4-acetoxy-2-[(2,5-dioxopyrrolidin-1-yl)methyl]-5-[2-(2-methyl-propanoyl-amino)-6-oxo-1H-purin-9-yl]tetrahydrofuran-3-yl] acetate (21)

To a solution of the starting material Methyl 4-oxo-4-[[(2R,3R,4R)-3,4,5-triacetoxy-tetrahydrofuran-2-yl]methylamino]-butanoate (16, 2.8 g, 7.2 mmol, 1.0 eq) and isobutyryl guanine (2.38 g, 10.8 mmol, 1.5 eq) in DCE (110 ml) was added BSA (5.84 g, 28.8 mmol, 4.0 eq) dropwise at room temperature. After stirring at 95° C. for 2 h, TMSOTf (4.8 g, 21.6 mmol, 3.0 eq) was added dropwise at 90° C. and stirring was continued at 90° C. for 12 h. The solution was cooled to room temperature and 150 ml of H₂O were added. The layer were separated and the aqueous layer was extracted with DCM (3×100 ml). The combined organic layers were dried over anhydrous Na₂SO₄, filtered and concentrated in vacuo. The residue was purified by flash chromatography (10% MeOH in EtOAc) and reverse flash chromatography (FA), yielding 1.5 g (40%) of the title compound [(2R,3R,4R,5R)-4-acetoxy-2-[(2,5-dioxopyrrolidin-1-yl)methyl]-5-[2-(2-methyl-propanoyl-amino)-6-oxo-1H-purin-9-yl]tetrahydrofuran-3-yl]acetate (21) as white foam.

MS (calc.: 518.2) (m/z)=519.5 [M+H⁺].

¹H-NMR (400 MHz, CDCl₃) δ[ppm]: 12.06 (br s, 1H), 9.70 (s, 1H), 7.61 (s, 1H), 5.89-5.79 (m, 2H), 5.69 (t, J=5.4 Hz, 1H), 4.64-4.53 (m, 1H), 4.36 (dd, J=9.4, 13.3 Hz, 1H), 3.68 (dd, J=5.7, 13.3 Hz, 1H), 2.74-2.63 (m, 5H), 2.04 (s, 3H), 1.99 (s, 3H), 1.24 (d, J=6.8 Hz, 3H), 1.19 (d, J=6.8 Hz, 3H).

### Example 1.20: Synthesis of 1-[[(2R,3S,4R,5R)-5-(2-amino-6-oxo-1H-purin-9-yl)-3,4-dihydroxy-tetrahydrofuran-2-yl]methyl]pyrrolidine-2,5-dione (22)

To a solution of [(2R,3R,4R,5R)-4-acetoxy-2-[(2,5-dioxopyrrolidin-1-yl)methyl]-5-[2-(2-methyl-propanoyl-amino)-6-oxo-1H-purin-9-yl]tetrahydrofuran-3-yl] acetate (21, 1.2 g, 2.1 mmol, 1.0 eq) in MeOH (6.3 ml) was added a freshly-made 1 M methanolic NaOMe-solution (0.63 ml, 0.63 mmol, 0.33 eq) dropwise at room temperature. After heating for 9 h at 60° C., the solution was allowed to reach room temperature. The mixture was filtered and the filtration residue dried in vacuo, which gave 720 g (85.7%) of the guanosine analog 1-[[(2R,3S,4R,5R)-5-(2-amino-6-oxo-1H-purin-9-yl)-3,4-dihydroxy-tetrahydrofuran-2-yl]methyl]pyrrolidine-2,5-dione (22) as white solid.

MS (calc.: 364.1) (m/z)=365.0 [M+H⁺].

### Example 1.21: Synthesis of 4-[[(2R,3S,4R,5R)-5-(2-amino-6-oxo-1H-purin-9-yl)-3,4-dihydroxy-tetrahydrofuran-2-yl]methylamino]-4-oxo-butanoic acid (23)

To a mixture of 1-[[(2R,3S,4R,5R)-5-(2-amino-6-oxo-1H-purin-9-yl)-3,4-dihydroxy-tetra-hydrofuran-2-yl]methyl]pyrrolidine-2,5-dione (22, 720 mg, 2.0 mmol, 1.0 eq) in a mixed solvent of THF (7 ml) and water (7 ml) was added LiOH·H₂O (80 mg, 1.9 mmol, 1.0 eq) in portions at room temperature. The solution was stirred at 30° C. for 2 h. Evaporation of the solvents in vacuo gave 770 mg of the title compound 4-[[(2R,3S,4R,5R)-5-(2-amino-6-oxo-1H-purin-9-yl)-3,4-dihydroxy-tetrahydrofuran-2-yl]methylamino]-4-oxo-butanoic acid (23) (Li-salt) as yellow solid.

MS (calc.: 382.1) (m/z)=383.1 [M+H⁺].

¹H-NMR (400 MHz, D₂O) δ[ppm]: 7.78 (s, 1H), 5.79 (d, J=5.4 Hz, 1H), 4.63 (br t, J=5.3 Hz, 1H), 4.23 (br t, J=4.5 Hz, 1H), 4.12 (br d, J=4.6 Hz, 1H), 3.56-3.42 (m, 2H), 2.37 (br s, 4H).

### Example 2: Synthetic Scheme for the Synthesis of Example Compound 30

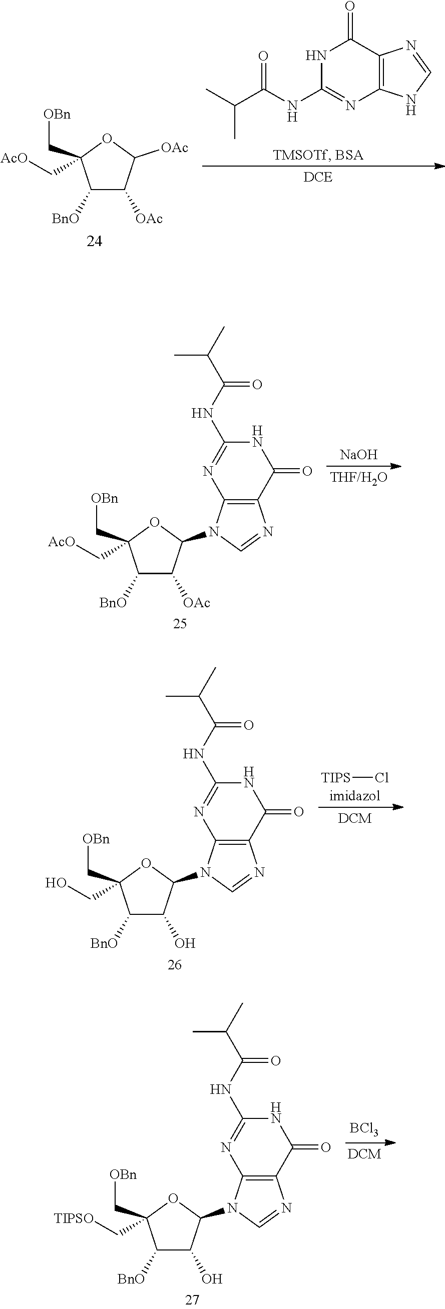

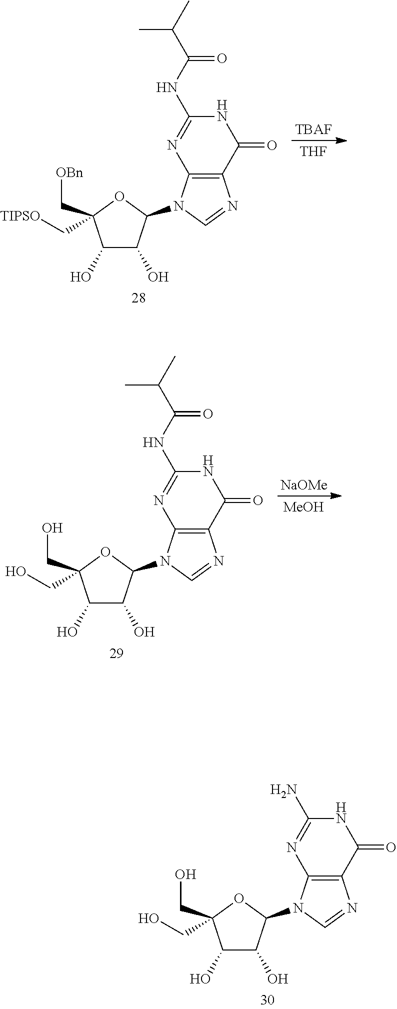

### Example 2.1: Synthesis of [(2S,3S,4R,5R)-4-acetoxy-3-benzyloxy-2-(benzyloxymethyl)-5-[2-(2-methylpro-panoylamino)-6-oxo-1H-purin-9-yl]tetrahydrofuran-2-yl]methyl acetate (25)

To a solution of the starting material [(2S,3S,4R)-4,5-diacetoxy-3-benzyloxy-2-(benzyloxy-methyl)tetrahydrofuran-2-yl]methyl acetate (24, 148.5 g, 0.30 mol) in 6.68 l DCE was added N-isobutyryl-guanine (135 g, 0.61 mol) and BSA (311.85 ml, 1.2 mol) at 15° C. under N₂-atmosphere. The mixture was stirred at 85° C. for 3 h. TMSOTf (183.4 g, 0.90 mol) was added at 85° C. and stirring was continued for 3 h, to achieve complete conversion. The mixture was cooled to room temperature and poured into 6.5 l sat. NaHCO₃-solution. The organic layer was separated and the aqueous phase extracted twice with 5 l DCM. The organic layers were combined and dried over anhydrous Na₂SO₄, filtered and concentrated. The obtained crude product was purified by preparative HPLC (0.1% TFA/ACN), yielding compound [(2S,3S,4R,5R)-4-acetoxy-3-benzyloxy-2-(benzyloxymethyl)-5-[2-(2-methylpro-panoyl-amino)-6-oxo-1H-purin-9-yl]tetrahydrofuran-2-yl]methyl acetate (25) (128 g, 64%) as white solid. 1H-NMR (DMSO-d6, 400 MHz) δ[ppm]: 12.09 (s, 1H), 11.62 (s, 1H), 8.14 (s, 1H), 7.41-7.30 (m, 10H), 6.12 (d, J=6.4 Hz, 1H), 5.90 (t, J₁=J₂=5.6 Hz, 1H), 4.71 (d, J=5.2 Hz, 1H), 4.63-4.55 (m, 4H), 4.34 (d, J=5.6 Hz, 1H), 4.23 (d, J=5.6 Hz, 1H), 3.71-3.66 (m, 2H), 3.18 (d, J=4.8 Hz, 1H), 2.76-2.51 (m, 1H), 2.05 (s, 3H), 1.99 (s, 3H), 1.20-1.12 (s, 6H).

### Example 2.2: Synthesis of N-[9-[(2R,3R,4S,5R)-4-benzyloxy-5-(benzyloxymethyl)-3-hydroxy-5-(hydroxy-methyl)tetrahydrofuran-2-yl]-6-oxo-1H-purin-2-yl]-2-methyl-propan-amide (26)

To a solution of compound [(2S,3S,4R,5R)-4-acetoxy-3-benzyloxy-2-(benzyloxymethyl)-5-[2-(2-methylpro-panoylamino)-6-oxo-1H-purin-9-yl]tetrahydrofuran-2-yl]methyl acetate (25, 72 g, 0.11 mol) in 1.7 l THF/EtOH (4:1) was added dropwise a 1 M NaOH-solution (443 ml) at 0° C. The solution was stirred at this temperature for 1 h to reach complete conversion. The pH was adjusted to 7 by adding an aqueous 1 N HCl and the solvent was removed. The residue was dissolved in H₂O (500 ml) and extracted with 3×500 ml DCM. The organic layers were combined, dried over anhydrous Na₂SO₄ and concentrated to give compound N-[9-[(2R,3R,4S,5R)-4-benzyloxy-5-(benzyloxymethyl)-3-hydroxy-5-(hydroxy-methyl)tetrahydro-furan-2-yl]-6-oxo-1H-purin-2-yl]-2-methyl-propanamide (26) (113 g, quant.) as a colorless solid, which was used in the next step without further purification. 1H-NMR (DMSO-d6, 400 MHz) δ[ppm]: 12.07 (s, 1H), 11.66 (s, 1H), 8.10 (s, 1H), 7.42-7.30 (m, 10H), 5.92 (d, J=6.8 Hz, 1H), 4.99 (s, 1H), 4.87-4.84 (m, 2H), 4.63 (d, J=15.6 Hz, 1H), 4.56 (s, 2H), 4.24 (d, J=4.8 Hz, 1H), 3.69-3.62 (m, 4H), 2.76-2.73 (m, 1H), 1.13-1.04 (m, 7H).

### Example 2.3: Synthesis of N-[9-[(2R,3R,4S,5S)-4-benzyloxy-5-(benzyloxymethyl)-3-hydroxy-5-(triisopropyl-silyloxymethyl)tetrahydrofuran-2-yl]-6-oxo-1H-purin-2-yl]-2-methyl-propanamide (27)

To a solution of N-[9-[(2R,3R,4S,5R)-4-benzyloxy-5-(benzyloxymethyl)-3-hydroxy-5-(hy-droxymethyl)tetrahydrofuran-2-yl]-6-oxo-1H-purin-2-yl]-2-methyl-propanamide (26, 75 g, 133 mmol) in anhydrous DCM (1568 ml) was added imidazole (38 g, 559 mmol) and TIPSCl (35.9 g, 186 mmol) at 0° C. under N₂-atmosphere. After stirring for 12 h between 10 and 15° C., the solution was poured into ice-water (2 l) and extracted with DCM (3×1.5 l). The organic layers were combined and washed with brine (1 l), dried over anhydrous Na₂SO₄, filtered and concentrated. The residue was purified by column chromatography on silica gel (PE/EtOAc 2:1 to EtOAc), yielding 65 g (68%) of the silylether N-[9-[(2R,3R,4S,5S)-4-benzyloxy-5-(benzyloxymethyl)-3-hydroxy-5-(triisopropyl-silyloxymethyl)tetrahydrofuran-2-yl]-6-oxo-1H-purin-2-yl]-2-methyl-propanamide (27) as white foam. 1H-NMR (DMSO-d6, 400 MHz) δ[ppm]: 12.07 (s, 1H), 11.61 (s, 1H), 8.14 (s, 1H), 7.37-7.22 (m, 10H), 5.89 (d, J=6.8 Hz, 1H), 5.72 (d, J=5.6 Hz, 1H), 4.94-4.93 (m, 2H), 4.90-4.53 (m, 3H), 4.19 (d, J=4.4 Hz, 1H), 3.92-3.88 (m, 2H), 3.85-3.71 (m, 2H), 2.78-2.71 (m, 1H), 1.13-1.05 (m, 6H), 1.00-0.94 (m, 21H).

### Example 2.4: Synthesis of N-[9-[(2R,3R,4S,5S)-3,4-dihydroxy-5-(hydroxymethyl)-5-(triisopropylsilyloxy-methyl)tetrahydrofuran-2-yl]-6-oxo-1H-purin-2-yl]-2-methyl-propan-amide (28)

To a solution of N-[9-[(2R,3R,4S,5S)-4-benzyloxy-5-(benzyloxymethyl)-3-hydroxy-5-(triiso-propylsilyloxymethyl)tetrahydrofuran-2-yl]-6-oxo-1H-purin-2-yl]-2-methyl-propanamide (27, 95 g, 0.132 mol) in anhydrous DCM (300 ml) was added BCl₃ (921 ml) at −70° C. under N2-atmosphere. The reaction solution was stirred between −75 and −60° C. for 2 h, when full conversion was detected. To the mixture were added approx. 200 ml of a saturated solution of NH₃ in MeOH. The pH was adjusted to 10-11 and the solvents were removed under reduced pressure. The crude product was purified by column chromatography on silica gel (PE/EtOAc 20:1 to 4:1), yielding the debenzylated product N-[9-[(2R,3R,4S,5S)-3,4-dihydroxy-5-(hydroxymethyl)-5-(triisopropylsilyloxy-methyl)tetrahydrofuran-2-yl]-6-oxo-1H-purin-2-yl]-2-methyl-propanamide (28) (51 g, 71.6%) as yellow solid. 1H-NMR (DMSO-d6, 400 MHz) δ[ppm]: 11.86 (s, 2H), 8.27 (s, 1H), 5.83 (d, J=7.2 Hz, 1H), 5.42 (s, 1H), 5.06 (s, 2H), 4.64 (s, 1H), 4.17 (d, J=4.0 Hz, 1H), 3.89 (d, J=10.8 Hz, 1H), 3.79 (d, J=10.4 Hz, 1H), 3.67 (s, 2H), 2.80-2.73 (m, 1H), 1.17-1.08 (m, 6H), 1.02-0.92 (m, 21H).

### Example 2.5: Synthesis of N-[9-[(2R,3R,4S)-3,4-dihydroxy-5,5-bis(hydroxymethyl)-tetrahydrofuran-2-yl]-6-oxo-1H-purin-2-yl]-2-methyl-propanamide (29)

To a solution of N-[9-[(2R,3R,4S,5S)-3,4-dihydroxy-5-(hydroxymethyl)-5-(triisopropyl-silyloxymethyl)tetrahydrofuran-2-yl]-6-oxo-1H-purin-2-yl]-2-methyl-propanamide (28, 3.2 g, 6.10 mmol, 1.0 eq) in THF (15 ml) was added TBAF (15 ml, 15.0 mmol, 2.5 eq, 1 mol/1 in THF) dropwise at 15° C. The mixture was stirred at this temperature for 12 h, to achieve complete deprotection. The reaction solution was concentrated in vacuo to give compound N-[9-[(2R,3R,4S)-3,4-dihydroxy-5,5-bis(hydroxymethyl)tetrahydrofuran-2-yl]-6-oxo-1H-purin-2-yl]-2-methyl-propanamide (29) (2.5 g, crude) as yellow oil, which was used for the next step without further purification.

### Example 2.6: Synthesis of 2-Amino-9-[(2R,3R,4S)-3,4-dihydroxy-5,5-bis(hydroxymethyl)-tetrahydrofuran-2-yl]-1H-purin-6-one (30)

To a solution of N-[9-[(2R,3R,4S)-3,4-dihydroxy-5,5-bis(hydroxymethyl)tetrahydrofuran-2-yl]-6-oxo-1H-purin-2-yl]-2-methyl-propanamide (29, 2.2 g, 5.74 mmol, 1.0 eq) in MeOH (22 ml) was added a NaOMe-solution (2.8 ml, 2.8 mmol, 0.5 eq, 1 mol/1 in MeOH) dropwise at 15° C. The mixture was stirred at 60° C. for 4 h, to achieve complete conversion. After cooling to room temperature, the precipitate was filtered and the filter cake triturated with MeOH (5 ml). After drying, 1.4 g (78%) of 2-Amino-9-[(2R,3R,4S)-3,4-dihydroxy-5,5-bis(hydroxy-methyl)tetrahydrofuran-2-yl]-1H-purin-6-one (30) were isolated as white solid.

MS (calc.: 313.1) (m/z)=314.1 [M+H⁺].

1H-NMR (DMSO-d6, 400 MHz) δ[ppm]: 10.76 (br s, 1H), 7.92 (s, 1H), 6.48 (br s, 2H), 5.73 (d, J=7.4 Hz, 1H), 5.33 (br s, 1H), 5.09 (br s, 2H), 4.80-4.41 (m, 2H), 4.15 (d, J=5.1 Hz, 1H), 3.65-3.47 (m, 4H).

### Example 3: Synthetic Scheme for the Synthesis of Example Compound 37

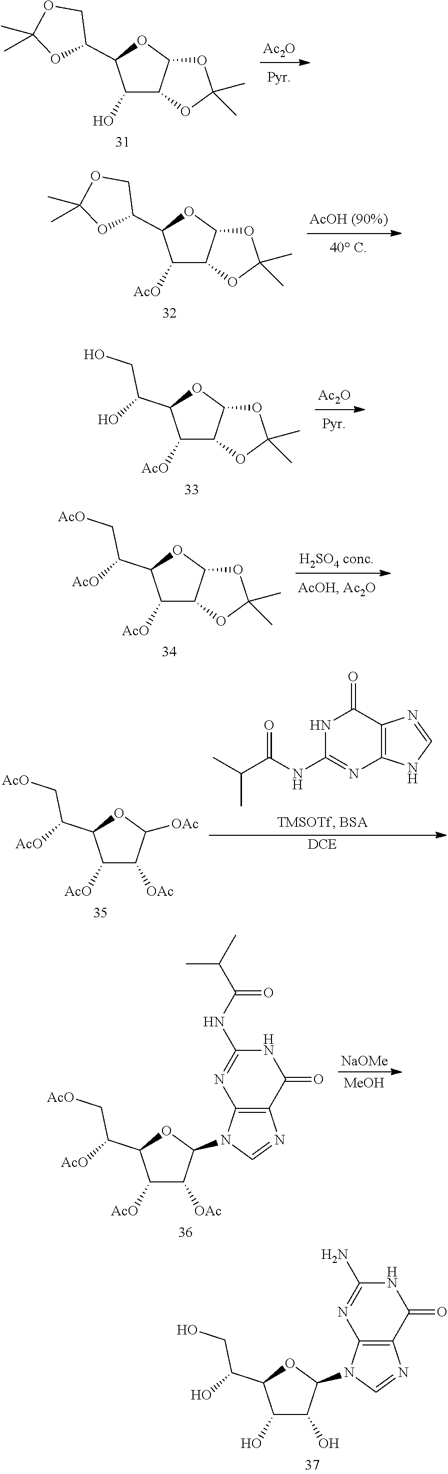

### Example 3.1: Synthesis of [(3aR,5R,6R,6aR)-5-[(4R)-2,2-Dimethyl-1,3-dioxolan-4-yl]-2,2-dimethyl-3a,5,6,6a-tetrahydrofuro[2,3-d][1,3]dioxol-6-yl] acetate (32)

To a solution of the allose derivative (3aR,5S,6R,6aR)-5-[(4R)-2,2-dimethyl-1,3-dioxolan-4-yl]-2,2-dimethyl-3a,5,6,6a-tetrahydrofuro[2,3-d][1,3]dioxol-6-ol (31, 10.0 g, 38.4 mmol, 1.0 eq) in pyridine (25 ml) was added acetic anhydride (25 ml) dropwise at 25° C. The solution was stirred for 12 h to achieve complete conversion. The reaction mixture was concentrated in vacuo and the residue was poured into a mixture of EtOAc (100 ml) and water (100 ml). After the layers were separated, the aqueous layer was extracted with EtOAc (2×100 ml). The combined organic phases were washed with sat. citric acid solution (100 ml) and brine (100 ml), dried over anhydrous Na₂SO₄, filtered and concentrated in vacuo, yielding 13.3 g (crude) of the acetyl protected product [(3aR,5R,6R,6aR)-5-[(4R)-2,2-dimethyl-1,3-dioxolan-4-yl]-2,2-dimethyl-3a,5,6,6a-tetrahydrofuro[2,3-d][1,3]dioxol-6-yl] acetate (32) as yellow solid, which was used without further purification.

### Example 3.2: Synthesis of [(3aR,5R,6R,6aR)-5-[(1R)-1,2-Dihydroxyethyl]-2,2-dimethyl-3a,5,6,6a-tetrahydro-furo[2,3-d][1,3]dioxol-6-yl] acetate (33)

A solution of the diisopropylidene protected starting material [(3aR,5R,6R,6aR)-5-[(4R)-2,2-dimethyl-1,3-dioxolan-4-yl]-2,2-dimethyl-3a,5,6,6a-tetrahydrofuro[2,3-d][1,3]dioxol-6-yl] acetate (32, 13.3 g, 38.4 mmol, 1.0 eq) in 90% AcOH (140 ml) was stirred at 40° C. After 12 h, the reaction mixture was concentrated in vacuo, which gave 13.8 g (crude) of the desired diol [(3aR,5R,6R,6aR)-5-[(1R)-1,2-dihydroxyethyl]-2,2-dimethyl-3a,5,6,6a-tetrahydro-furo[2,3-d][1,3]dioxol-6-yl] acetate (33) as yellow oil, which was used without further purification.

### Example 3.3: Synthesis of [(2R)-2-[(3aR,5R,6R,6aR)-6-Acetoxy-2,2-dimethyl-3a,5,6,6a-tetrahydrofuro[2,3-d]-[1,3]dioxol-5-yl]-2-acetoxy-ethyl] acetate (34)

To a solution of the starting material [(3aR,5R,6R,6aR)-5-[(1R)-1,2-dihydroxyethyl]-2,2-dimethyl-3a,5,6,6a-tetrahydro-furo[2,3-d][1,3]dioxol-6-yl] acetate (33, 13.8 g, 38.4 mmol, 1.00 eq) in pyridine (30 ml) was added acetic anhydride (30 ml) dropwise at 25° C. After 12 h, the solvent was removed in vacuo and the residue was dissolved in EtOAc (100 ml). The organic layer was washed with water (50 ml) and brine (50 ml), dried over anhydrous Na₂SO₄, filtered and concentrated in vacuo. After purification on silica (PE/EtOAc 2:1), 7.5 g (56.4%, three steps) of the title compound [(2R)-2-[(3aR,5R,6R,6aR)-6-acetoxy-2,2-dimethyl-3a,5,6,6a-tetrahydrofuro[2,3-d]-[1,3]dioxol-5-yl]-2-acetoxy-ethyl] acetate (34) were isolated as colorless solid. 1H-NMR (CDCl₃, 400 MHz) δ[ppm]: 5.79 (d, J=3.2 Hz, 1H), 5.30 (ddd, J=3.7, 4.8, 6.7 Hz, 1H), 4.88-4.78 (m, 2H), 4.39 (dd, J=3.7, 12.1 Hz, 1H), 4.27 (ddd, J=2.6, 5.2, 8.1 Hz, 1H), 4.12 (dd, J=6.8, 12.0 Hz, 1H), 2.14 (s, 3H), 2.09 (s, 3H), 2.06 (s, 3H), 1.56 (s, 3H), 1.34 (s, 3H).

### Example 3.4: Synthesis of [(2R)-2-Acetoxy-2-[(2R,3R,4R)-3,4,5-triacetoxytetrahydrofuran-2-yl]ethyl] acetate (35)

The starting material [(2R)-2-[(3aR,5R,6R,6aR)-6-acetoxy-2,2-dimethyl-3a,5,6,6a-tetrahydro-furo[2,3-d]-[1,3]dioxol-5-yl]-2-acetoxy-ethyl] acetate (34, 6.5 g, 18.8 mmol, 1.00 eq) was dissolved in AcOH (35 ml). After adding acetic anhydride (7 ml) and H₂SO₄ (150 mg, cat.) dropwise at 0° C., the mixture was stirred at room temperature for 12 h, to achieve complete conversion. The reaction mixture was diluted with EtOAc (200 ml) and washed with water (100 ml) and brine (100 ml). The organic layer was separated, dried over anhydrous Na₂SO₄, filtered and concentrated in vacuo. Silicagel chromatography (PE/EtOAc 2:1) gave 6.5 g (89.0%) of the peracetylated product [(2R)-2-acetoxy-2-[(2R,3R,4R)-3,4,5-triacetoxy-tetrahydrofuran-2-yl]ethyl] acetate (35) as yellow oil. 1H-NMR (CDCl₃, 400 MHz) δ[ppm]: 6.47-6.14 (m, 1H), 5.55-5.47 (m, 1H), 5.36-5.17 (m, 2H), 4.47-4.29 (m, 2H), 4.17-4.02 (m, 1H), 2.14-2.05 (m, 15H).

### Example 3.5: Synthesis of [(2R)-2-Acetoxy-2-[(2R,3R,4R,5R)-3,4-diacetoxy-5-[2-(2-methylpropanoylamino)-6-oxo-1H-purin-9-yl]tetrahydrofuran-2-yl]ethyl] acetate (36)

Glycosyl donor [(2R)-2-acetoxy-2-[(2R,3R,4R)-3,4,5-triacetoxytetrahydrofuran-2-yl]ethyl] acetate (35, 5.5 g, 14.1 mmol, 1.0 eq) and isobutyryl-guanosine (4.7 g, 21.1 mmol, 1.5 eq) were dissolved in DCE (220 ml). After adding BSA (11.5 g, 56.4 mmol, 4.0 eq) dropwise at room temperature, the mixture was stirred at 95° C. for 2 h. TMSOTf (9.4 g, 242.3 mmol, 3.0 eq) was added dropwise at 90° C. and the solution was stirred at this temperature for 12 h, to achieve complete conversion. After the reaction solution was cooled to room temperature, the mixture was filtered and the filtrate poured into water (100 ml). After extraction with DCM (3×100 ml), the combined organic layers were dried over anhydrous Na₂SO₄, filtered and concentrated in vacuo. The residue was purified by reverse flash chromatography (FA), yielding 4.4 g (57.1%) of the guanosine analog [(2R)-2-acetoxy-2-[(2R,3R,4R,5R)-3,4-diacetoxy-5-[2-(2-methylpropanoylamino)-6-oxo-1H-purin-9-yl]tetrahydrofuran-2-yl]ethyl] acetate (36) as white foam. 1H-NMR (DMSO-d₆, 400 MHz) δ[ppm]: 12.12 (br s, 1H), 11.54 (s, 1H), 8.33 (s, 1H), 6.07 (d, J=7.0 Hz, 1H), 5.81 (t, J=6.6 Hz, 1H), 5.58 (dd, J=3.6, 6.1 Hz, 1H), 5.41 (dt, J=3.1, 5.9 Hz, 1H), 4.40-4.26 (m, 2H), 4.09 (br dd, J=5.7, 12.3 Hz, 1H), 2.78 (td, J=6.8, 13.6 Hz, 1H), 2.14 (s, 3H), 2.08 (s, 3H), 2.03 (s, 3H), 2.00 (s, 3H), 1.14 (d, J=6.7 Hz, 6H).

### Example 3.6: Synthesis of 2-Amino-9-[(2R,3R,4S,5R)-5-[(1R)-1,2-dihydroxyethyl]-3,4-dihydroxy-tetrahydro-furan-2-yl]-1H-purin-6-one (37)

To a solution of the starting material [(2R)-2-acetoxy-2-[(2R,3R,4R,5R)-3,4-diacetoxy-5-[2-(2-methylpropanoylamino)-6-oxo-1H-purin-9-yl]tetrahydrofuran-2-yl]ethyl]acetate (36, 3.0 g, 5.4 mmol, 1.0 eq) in MeOH (18 ml) was added NaOMe (1.8 ml, 1.8 mmol, 0.33 eq, 1 mol/1 in MeOH) dropwise at 15° C. The mixture was stirred at 60° C. for 8 h, to achieve complete conversion. The mixture was filtered and the filter cake dried in vacuo, yielding 1.68 g (98.8%) of the guanosine analog 2-Amino-9-[(2R,3R,4S,5R)-5-[(1R)-1,2-dihydroxyethyl]-3,4-dihydroxy-tetrahydro-furan-2-yl]-1H-purin-6-one (37) as colorless solid.

MS (calc.: 313.1) (m/z)=313.9 [M+H⁺].

1H-NMR (DMSO-d₆, 400 MHz) δ[ppm]: 7.88 (s, 1H), 6.77 (br s, 2H), 5.67 (d, J=7.1 Hz, 1 H), 5.59-5.29 (m, 2H), 5.28-4.95 (m, 1H), 4.83-4.55 (m, 1H), 4.51-4.42 (m, 1H), 4.16 (br dd, J=1.5, 4.8 Hz, 1H), 3.96-3.88 (m, 1H), 3.72-3.61 (m, 1H), 3.47-3.40 (m, 2H).

### Example 4: Synthetic Scheme for the Synthesis of Example Compound 47

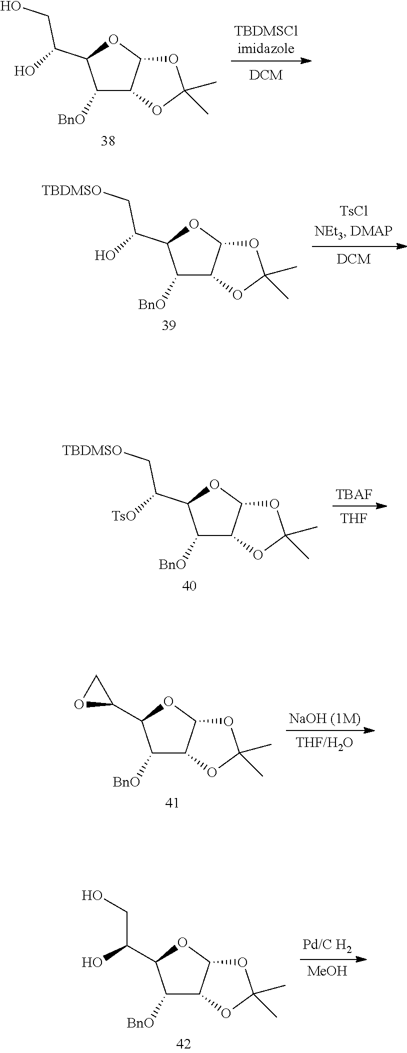

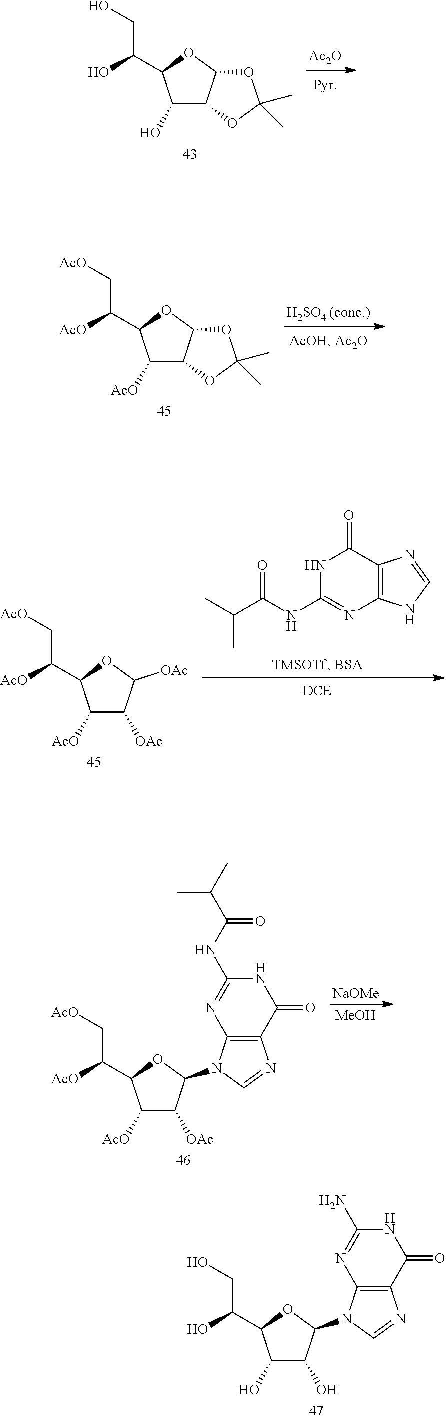

### Example 4.1: Synthesis of (1R)-1-[(3aR,5R,6R,6aR)-6-Benzyloxy-2,2-dimethyl-3a,5,6,6a-tetrahydrofuro[2,3-d][1,3]dioxol-5-yl]-2-[tert-butyl(dimethyl)silyl]oxy-ethanol (39)

To a solution of the diol (1R)-1-[(3aR,5R,6R,6aR)-6-benzyloxy-2,2-dimethyl-3a,5,6,6a-tetrahydrofuro[2,3-d][1,3]dioxol-5-yl]ethane-1,2-diol (38, 6.64 g, 21.4 mmol, 1.0 eq) in DCM (130 ml) was added imidazole (4.36 g, 64.2 mmol, 3.0 eq) and TBDMSCl (3.7 g, 24.6 mmol, 1.15 eq) in portions at room temperature. After stirring for 12 h, the solvent was evaporated in vacuo and the residue was poured into a mixture of EtOAc (100 ml) and water (100 ml). The aqueous layer was extracted with EtOAc (2×100 ml) and the combined organic phases washed with brine (100 ml). The organic solution was dried over anhydrous Na₂SO₄, filtered and concentrated in vacuo. After purification by column chromatography (PE/EA 5:1), 7.0 g (77.8%) of the silylether (1R)-1-[(3aR,5R,6R,6aR)-6-benzyloxy-2,2-dimethyl-3a,5,6,6a-tetrahydrofuro[2,3-d][1,3]dioxol-5-yl]-2-[tert-butyl(dimethyl)silyl]oxy-ethanol (39) were isolated as white solid.

MS (calc.: 424.2) (m/z)=447.3 [M+Na⁺].

1H-NMR (CDCl₃, 400 MHz) δ[ppm]: 7.44-7.29 (m, 5H), 5.75 (d, J=3.7 Hz, 1H), 4.79 (d, J=11.7 Hz, 1H), 4.62 (d, J=11.7 Hz, 1H), 4.57 (t, J=4.1 Hz, 1H), 4.11-4.05 (m, 1H), 3.97 (dd, J=4.4, 8.7 Hz, 1H), 3.95-3.89 (m, 1H), 3.75-3.65 (m, 2H), 2.53 (d, J=3.1 Hz, 1H), 1.64-1.57 (m, 3H), 1.37 (s, 3H), 0.95-0.89 (m, 9H), 0.08 (d, J=0.7 Hz, 6H).

### Example 4.2: Synthesis of [(1R)-1-[(3aR,5S,6R,6aR)-6-Benzyloxy-2,2-dimethyl-3a,5,6,6a-tetrahydrofuro[2,3-d][1,3]dioxol-5-yl]-2-[tert-butyl(dimethyl)silyl]oxy-ethyl] 4-methyl-benzenesulfonate (40)

To a solution of the starting material (1R)-1-[(3aR,5R,6R,6aR)-6-benzyloxy-2,2-dimethyl-3a,5,6,6a-tetrahydrofuro[2,3-d][1,3]dioxol-5-yl]-2-[tert-butyl(dimethyl)silyl]oxy-ethanol (39, 6.6 g, 15.5 mmol, 1.0 eq) in DCM (70 ml) was added NEt₃ (4.7 g, 15.5 mmol, 3.0 eq) and DMAP (1.69 g, 15.5 mmol, 1.0 eq) in portions at room temperature, followed by p-toluenesulfonyl chloride (5.93 g, 31.1 mmol, 2.0 eq). The solution was stirred for 12 h and the solvent was evaporated in vacuo. The residue was dissolved in EtOAc (150 ml) and washed with water (100 ml) and brine (100 ml). After drying over Na₂SO₄ and evaporation of the solvent in vacuo, the crude product was purified by column chromatography (PE/EA 7:1), yielding 7.0 g (77.7%) of the title compound [(1R)-1-[(3aR,5S,6R,6aR)-6-benzyloxy-2,2-dimethyl-3a,5,6,6a-tetrahydrofuro[2,3-d][1,3]dioxol-5-yl]-2-[tert-butyl(dimethyl)silyl]oxy-ethyl] 4-methylbenzenesulfonate (40) as white solid.

1H-NMR (CDCl₃, 400 MHz) δ[ppm]: 7.77 (d, J=8.3 Hz, 2H), 7.39-7.32 (m, 5H), 7.30-7.25 (m, 2H), 5.34 (d, J=3.5 Hz, 1H), 4.88 (dt, J=2.0, 6.5 Hz, 1H), 4.72 (d, J=11.5 Hz, 1H), 4.54 (d, J=11.5 Hz, 1H), 4.47-4.43 (m, 1H), 4.24 (dd, J=2.1, 8.8 Hz, 1H), 3.94-3.89 (m, 1H), 3.85-3.76 (m, 2H), 2.44 (s, 3H), 1.52 (s, 3H), 1.33 (s, 3H), 0.86 (s, 9H), 0.03-0.04 (m, 6H).

### Example 4.3: Synthesis of (3aR,5R,6R,6aR)-6-Benzyloxy-2,2-dimethyl-5-[(2S)-oxiran-2-yl]-3a,5,6,6a-tetra-hydrofuro[2,3-d][1,3]dioxole (41)

The furanose [(1R)-1-[(3aR,5S,6R,6aR)-6-benzyloxy-2,2-dimethyl-3a,5,6,6a-tetrahydrofuro-[2,3-d][1,3]dioxol-5-yl]-2-[tert-butyl(dimethyl)silyl]oxy-ethyl] 4-methylbenzenesulfonate (40, 7 g, 12.1 mmol, 1.00 eq) and TBAF (30 ml, 30 mmol, 2.50 eq, 1 mol/1 in THF) was stirred at room temperature for 12 h, to achieve complete conversion. The solution was concentrated in vacuo and the residue dissolved in EtOAc (150 ml). After washing with water (100 ml) and brine (100 ml), the organic phase was dried over anhydrous Na₂SO₄, filtered and concentrated in vacuo. Purification of the crude product by column chromatography (PE/EA 5:1) gave 4 g (88.8%) of the oxirane (3aR,5R,6R,6aR)-6-benzyloxy-2,2-dimethyl-5-[(2S)-oxiran-2-yl]-3a,5,6,6a-tetra-hydrofuro[2,3-d][1,3]dioxole (41) as colorless oil.

1H-NMR (CDCl₃, 400 MHz) δ[ppm]: 7.34-7.22 (m, 5H), 5.63 (d, J=3.7 Hz, 1H), 4.72 (d, J=12.0 Hz, 1H), 4.55 (d, J=12.0 Hz, 1H), 4.49 (t, J=3.9 Hz, 1H), 3.96 (dd, J=4.0, 9.0 Hz, 1H), 3.72 (dd, J=4.2, 9.0 Hz, 1H), 3.05-2.97 (m, 1H), 2.80 (dd, J=2.7, 5.4 Hz, 1H), 2.74-2.69 (m, 1H), 1.51 (s, 3H), 1.28 (s, 3H).

### Example 4.4: Synthesis of (1S)-1-[(3aR,5R,6R,6aR)-6-Benzyloxy-2,2-dimethyl-3a,5,6,6a-tetrahydrofuro[2,3-d][1,3]dioxol-5-yl]ethane-1,2-diol (42)

The starting material (3aR,5R,6R,6aR)-6-benzyloxy-2,2-dimethyl-5-[(2S)-oxiran-2-yl]-3a,5,6,6a-tetra-hydrofuro[2,3-d][1,3]dioxole (41, 4 g, 13.7 mmol, 1.00 eq) was dissolved in a mixed solvent of THF (80 ml) and H₂O (40 ml). After the addition of a 1 M NaOH-solution (30 ml) at room temperature, the mixture was stirred at 90° C. for 48 h. After the reaction solution was cooled to room temperature, the THF was removed in vacuo and the aqueous layer was adjusted to pH=2-3 adding a 2 M HCl. The aqueous mixture was extracted with EtOAc (3×100 ml) and the combined organic layers dried over anhydrous Na₂SO₄. After evaporation in vacuo, 4.2 g (crude) of the diol (1S)-1-[(3aR,5R,6R,6aR)-6-benzyloxy-2,2-dimethyl-3a,5,6,6a-tetrahydrofuro[2,3-d][1,3]dioxol-5-yl]ethane-1,2-diol (42) were isolated as yellow oil and used without further purification.

1H-NMR (CDCl₃, 400 MHz) δ[ppm]: 7.34-7.23 (m, 5H), 5.70-5.64 (m, 1H), 4.73-4.67 (m, 1H), 4.55-4.46 (m, 2H), 4.04-3.97 (m, 1H), 3.86 (dd, J=4.3, 8.9 Hz, 1H), 3.74-3.60 (m, 3H), 2.42-2.18 (m, 1H), 1.52 (s, 3H), 1.29 (s, 3H).

### Example 4.5: Synthesis of (1S)-1-[(3aR,5R,6R,6aR)-6-Hydroxy-2,2-dimethyl-3a,5,6,6a-tetrahydrofuro[2,3-d][1,3]dioxol-5-yl]ethane-1,2-diol (43)

To a solution of the benzylether (1S)-1-[(3aR,5R,6R,6aR)-6-benzyloxy-2,2-dimethyl-3a,5,6,6a-tetrahydrofuro[2,3-d][1,3]dioxol-5-yl]ethane-1,2-diol (42, 4 g, 13.5 mmol, 1.00 eq) in MeOH (80 ml) was added Pd/C (1 g, 10%) in portions at room temperature under N2-atmosphere. The mixture was stirred at 45° C. under 3.5 bar H2-atmosphere. After 12 h, the mixture was cooled to room temperature. The catalyst was separated by filtration and the filtrate concentrated in vacuo, yielding 2.9 g (97%) of the title compound (1S)-1-[(3aR,5R,6R,6aR)-6-hydroxy-2,2-dimethyl-3a,5,6,6a-tetrahydrofuro[2,3-d][1,3]dioxol-5-yl]ethane-1,2-diol (43) as colorless oil. 1H-NMR (CDCl₃, 400 MHz) δ[ppm]: 5.84 (d, J=3.8 Hz, 1H), 4.65-4.58 (m, 1H), 4.09 (br dd, J=5.3, 8.1 Hz, 1H), 3.86-3.76 (m, 4H), 2.99-2.62 (m, 3H), 1.62-1.56 (m, 3H), 1.40 (s, 3H).

### Example 4.6: Synthesis of [(2S)-2-[(3aR,5R,6R,6aR)-6-Acetoxy-2,2-dimethyl-3a,5,6,6a-tetrahydrofuro[2,3-d][1,3]dioxol-5-yl]-2-acetoxy-ethyl] acetate (44)

To a solution of the starting material (1S)-1-[(3aR,5R,6R,6aR)-6-hydroxy-2,2-dimethyl-3a,5,6,6a-tetrahydrofuro[2,3-d][1,3]dioxol-5-yl]ethane-1,2-diol (43, 2.9 g, 13.1 mmol, 1.00 eq) in pyridine (30 ml) was added acetic anhydride (15 ml) dropwise at room temperature. After stirring for 12 h, the solvent was removed in vacuo and the residue dissolved in EtOAc (100 ml). The organic layer was washed with water (50 ml) and brine (50 ml), dried over anhydrous Na₂SO₄, filtered and concentrated in vacuo. The crude product was purified by column chromatography (PE/EA 4:1); which gave 4 g (88%) of the title compound [(2S)-2-[(3aR,5R,6R,6aR)-6-acetoxy-2,2-dimethyl-3a,5,6,6a-tetrahydrofuro[2,3-d][1,3]dioxol-5-yl]-2-acetoxy-ethyl] acetate (44) as colorless oil.

1H-NMR (CDCl₃, 400 MHz) δ[ppm]: 5.75 (d, J=3.7 Hz, 1H), 5.25-5.14 (m, 1H), 4.77-4.69 (m, 1H), 4.59 (dd, J=4.8, 9.2 Hz, 1H), 4.35-4.19 (m, 2H), 4.14 (dd, J=7.5, 11.8 Hz, 1H), 2.06 (s, 3H), 2.05 (s, 3H), 1.97 (s, 3H), 1.49 (s, 3H), 1.27 (s, 3H).

### Example 4.7: Synthesis of [(2S)-2-Acetoxy-2-[(2R,3R,4R)-3,4,5-triacetoxytetrahydrofuran-2-yl]ethyl] acetate (45)

Following the protocol, described for the synthesis of [(2R)-2-Acetoxy-2-[(2R,3R,4R)-3,4,5-triacetoxytetrahydrofuran-2-yl]ethyl] acetate (35), 4 g (11.5 mmol, 1.0 eq.) of the iso-propylidene protected starting material [(2S)-2-[(3aR,5R,6R,6aR)-6-acetoxy-2,2-dimethyl-3a,5,6,6a-tetrahydrofuro[2,3-d][1,3]dioxol-5-yl]-2-acetoxy-ethyl] acetate (44) were converted to the peracetylated furanose derivative [(2S)-2-acetoxy-2-[(2R,3R,4R)-3,4,5-triacetoxytetra-hydrofuran-2-yl]ethyl] acetate (45). After column chromatography (PE/EtOAc 3:1), 3.4 g (75.5%) of the title compound were isolated colorless oil.

### Example 4.8: Synthesis of [(2S)-2-Acetoxy-2-[(2R,3R,4R,5R)-3,4-diacetoxy-5-[2-(2-methylpropanoylamino)-6-oxo-1H-purin-9-yl]tetrahydrofuran-2-yl]ethyl] acetate (46)

Following the protocol, described for the synthesis of [(2R)-2-Acetoxy-2-[(2R,3R,4R,5R)-3,4-diacetoxy-5-[2-(2-methylpropanoylamino)-6-oxo-1H-purin-9-yl]tetrahydrofuran-2-yl]ethyl] acetate (36), 3.4 g (8.7 mmol, 1.0 eq.) of the starting material [(2S)-2-acetoxy-2-[(2R,3R,4R)-3,4,5-triacetoxytetrahydrofuran-2-yl]ethyl] acetate (45) were glycosylated, which gave 2.0 g (41.6%) of the title compound [(2S)-2-acetoxy-2-[(2R,3R,4R,5R)-3,4-diacetoxy-5-[2-(2-methylpropanoylamino)-6-oxo-1H-purin-9-yl]tetrahydrofuran-2-yl]ethyl] acetate (46) as colorless foam.

MS (calc.: 551.2) (m/z)=552.3 [M+H⁺].

1H-NMR (CDCl₃, 400 MHz) δ[ppm]: 12.44-12.16 (m, 1H), 9.48 (s, 1H), 7.74 (s, 1H), 6.01-5.95 (m, 1H), 5.93-5.85 (m, 2H), 5.39-5.32 (m, 1H), 4.82 (dd, J=4.3, 12.3 Hz, 1H), 4.45-4.37 (m, 2H), 2.27 (s, 3H), 2.18 (s, 3H), 2.14 (s, 3H), 2.04 (s, 3H), 1.31 (d, J=1.6 Hz, 3H), 1.29 (d, J=1.6 Hz, 3H).

### Example 4.9: Synthesis of 2-Amino-9-[(2R,3R,4S,5R)-5-[(1S)-1,2-dihydroxyethyl]-3,4-dihydroxy-tetrahydro-furan-2-yl]-1H-purin-6-one (47)

To a solution of the starting material [(2S)-2-acetoxy-2-[(2R,3R,4R,5R)-3,4-diacetoxy-5-[2-(2-methylpropanoylamino)-6-oxo-1H-purin-9-yl]tetrahydrofuran-2-yl]ethyl]acetate (46, 960 mg, 1.74 mmol, 1.0 eq) in MeOH (10 ml) was added NaOMe (0.87 ml, 0.87 mmol, 0.5 eq, 1 mol/1 in MeOH) dropwise at 15° C. The mixture was heated to 60° C. for 4 h, to achieve complete conversion. The mixture was filtered and the filter cake dried in vacuo, yielding 500 mg (91.7%) of the desired product 2-Amino-9-[(2R,3R,4S,5R)-5-[(1S)-1,2-dihydroxyethyl]-3,4-dihydroxy-tetrahydro-furan-2-yl]-1H-purin-6-one (47) as white solid.

MS (calc.: 313.1) (m/z)=314.1 [M+H⁺].

1H-NMR (D₂O, 400 MHz) δ[ppm]: 7.83 (s, 1H), 5.77 (d, J=6.4 Hz, 1H), 4.68-4.66 (m, 1H), 4.36 (dd, J=2.9, 5.2 Hz, 1H), 4.17-4.13 (m, 1H), 3.84 (ddd, J=2.4, 5.2, 7.5 Hz, 1H), 3.63-3.51 (m, 2H), 3.26 (s, 1H).

### Example 5: Synthetic Scheme for the Synthesis of Example Compound 58

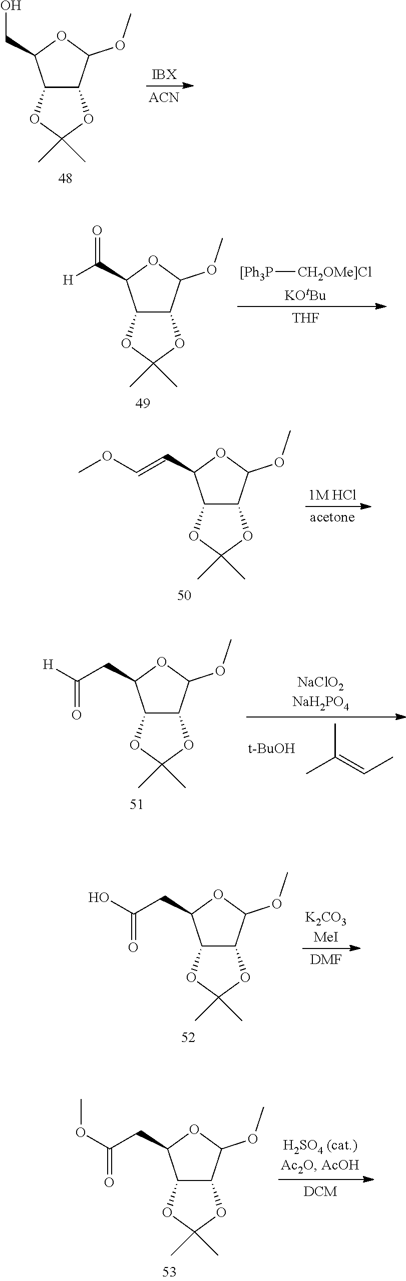

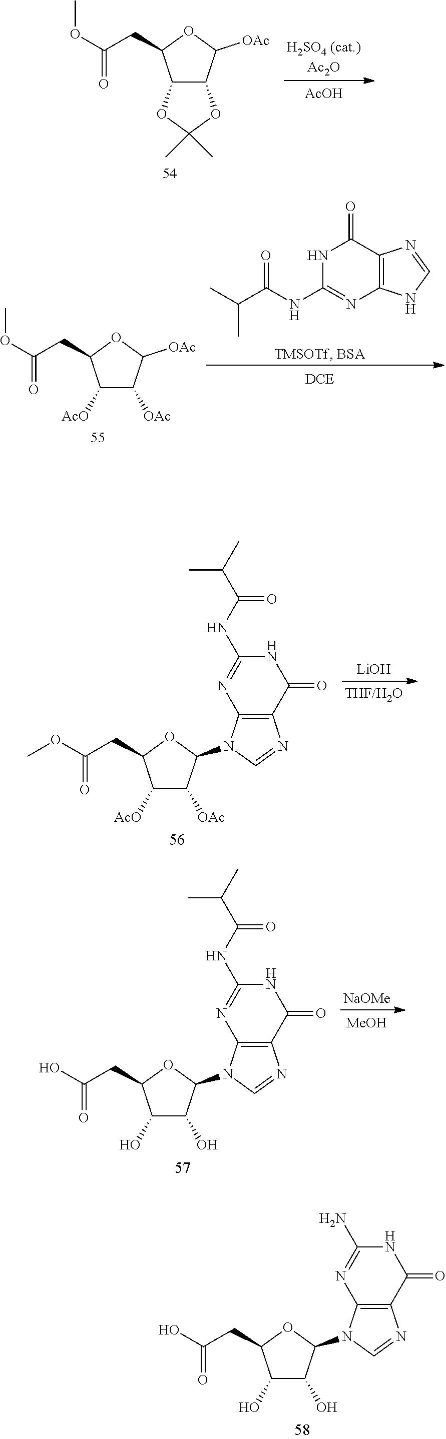

### Example 5.1: Synthesis of (3aR,6S,6aR)-4-Methoxy-2,2-dimethyl-3a,4,6,6a-tetrahydro-furo[3,4-d][1,3]dioxole-6-carbaldehyde (49)

To a solution of the ribose derivative [(3aR,6R,6aR)-4-methoxy-2,2-dimethyl-3a,4,6,6a-tetrahydrofuro[3,4-d][1,3]dioxol-6-yl]methanol (48, 11.2 g, 0.055 mol, 1.0 eq) in ACN (450 ml) was added 2-iodoxybenzoic acid (19.2 g, 0.069 mol, 1.25 eq) in portions at 15° C. After stirring at 90° C. for 3 h, the reaction mixture cooled to room temperature, filtered and the filtrate was concentrated in vacuo. The residue was dissolved in EtOAc (200 ml), washed with sat. Na₂S203 (50 ml) and brine (50 ml). The organic layer was dried over anhydrous Na₂SO₄, filtered and concentrated in vacuo to give 9.7 g (87.3%) of the aldehyde (3aR,6S,6aR)-4-methoxy-2,2-dimethyl-3a,4,6,6a-tetrahydrofuro[3,4-d][1,3]dioxole-6-carbaldehyde (49) as white solid. 1H-NMR (CDCl₃, 400 MHz) δ[ppm]: 9.50 (s, 1H), 5.01 (s, 1H), 4.97 (d, J=5.9 Hz, 1H), 4.42 (d, J=6.1 Hz, 1H), 4.39 (s, 1H), 3.37 (s, 3H), 1.41 (s, 3H), 1.25 (s, 3H).

### Example 5.2: Synthesis of (3aR,6R,6aR)-4-Methoxy-6-[(E)-2-methoxyvinyl]-2,2-dimethyl-3a,4,6,6a-tetrahydro-furo[3,4-d][1,3]dioxole (50)

To a suspension of (methoxymethyl)triphenylphosphonium chloride (40 g, 0.116 mol, 3.0 eq) in THF (580 ml) was added t-BuOK (96 ml, 96.4 mmol, 2.5 eq, 1 mol/1 in THF) dropwise at 0° C. The resulting red-colored mixture was stirred at 0° C. for 1 h, followed by the addition of a solution of (3aR,6S,6aR)-4-methoxy-2,2-dimethyl-3a,4,6,6a-tetrahydrofuro[3,4-d][1,3]-dioxole-6-carbaldehyde (49, 7.8 g, 38.6 mmol, 1.0 eq) in THF (116 ml.) Stirring was continued at 0° C. for 1 h to achieve complete conversion. The mixture was quenched with sat. NaHCO₃ (120 ml) and extracted with methyl-tert. butylether (3×300 ml). The combined organic layers were dried over anhydrous Na₂SO₄, filtered and concentrated in vacuo. The crude product was purified by flash chromatography (PE/EtOAc 5:1), yielding 5.1 g (57.3%) of the Wittig product (3aR,6R,6aR)-4-methoxy-6-[(E)-2-methoxyvinyl]-2,2-dimethyl-3a,4,6,6a-tetrahydro-furo[3,4-d][1,3]dioxole (50) as yellow oil (mixture of E and Z isomers).

### Example 5.3: Synthesis of 2-[(3aR,6R,6aR)-4-Methoxy-2,2-dimethyl-3a,4,6,6a-tetrahydro-furo[3,4-d][1,3]dioxol-6-yl]acetaldehyde (51)

To a mixture of the vinylether (3aR,6R,6aR)-4-methoxy-6-[(E)-2-methoxyvinyl]-2,2-dimethyl-3a,4,6,6a-tetrahydro-furo[3,4-d][1,3]dioxole (50, 5.2 g, 22.6 mmol, 1.0 eq) in acetone (113 ml) was added aqueous 1 N HCl (0.56 ml, 1 mol/1 in water) dropwise at 10° C. After stirring for 2 h, another 0.56 ml of aqueous 1 N HCl were added and stirring was continued at 10° C. for 4 h. The solution was neutralized by the addition of NEt₃ and the solvent was removed in vacuo. The residue was purified by flash chromatography (PE/EA 3:1), which gave 3.9 g (80%) of the aldehyde 2-[(3aR,6R,6aR)-4-methoxy-2,2-dimethyl-3a,4,6,6a-tetrahydrofuro[3,4-d][1,3]diox-ol-6-yl]acetaldehyde (51) as colorless oil. 1H-NMR (CDCl₃, 400 MHz) δ[ppm]: 9.81 (m, 1H), 4.98 (s, 1H), 4.73 (dd, J=8.4, 6.6 Hz, 1H), 4.55-4.66 (m, 2H), 3.33 (s, 3H), 2.62-2.91 (m, 2H), 1.51 (s, 3H), 1.34 (s, 3H).

### Example 5.4: Synthesis of 2-[(3aR,6R,6aR)-4-Methoxy-2,2-dimethyl-3a,4,6,6a-tetrahydro-furo[3,4-d][1,3]dioxol-6-yl]acetic acid (52)

The aldehyde 2-[(3aR,6R,6aR)-4-methoxy-2,2-dimethyl-3a,4,6,6a-tetrahydrofuro[3,4-d][1,3]-dioxol-6-yl]acetaldehyde (51, 4 g, 18.5 mmol, 1.0 eq) was dissolved in a mixed solvent of t-BuOH (184 ml) and 2-methylbut-2-ene (60 ml). After the addition of a solution of NaClO₂ (16.7 g, 0.185 mol, 10.0 eq) in water (18.5 ml) and NaH₂PO₄ (22 g, 0.185 mol, 10.0 eq) in water (18.5 ml) sequentially at 10° C., the mixture was stirred at 10° C. for 16 h, to achieve complete conversion. The reaction mixture was diluted with sat. NH₄Cl (300 ml) and extracted with EtOAc (3×500 ml). The combined organic layers were dried over anhydrous Na₂SO₄, filtered and concentrated in vacuo. The crude product was purified by flash chromatography (EtOAc) yielding 4.1 g (95.4%) of the carboxylic acid 2-[(3aR,6R,6aR)-4-methoxy-2,2-dimethyl-3a,4,6,6a-tetrahydrofuro[3,4-d][1,3]dioxol-6-yl]acetic acid (52) as yellow oil. 1H-NMR (CDCl₃, 400 MHz) δ[ppm]: 4.90 (s, 1H), 4.50-4.66 (m, 3H), 3.23-3.35 (m, 3H), 2.62 (m, 2H), 1.42 (s, 3H), 1.25 (s, 3H).

### Example 5.5: Synthesis of Methyl 2-[(3aR,6R,6aR)-4-methoxy-2,2-dimethyl-3a,4,6,6a-tetrahydrofuro[3,4-d][1,3]dioxol-6-yl]acetate (53)

To a solution of the carboxylic acid 2-[(3aR,6R,6aR)-4-methoxy-2,2-dimethyl-3a,4,6,6a-tetrahydrofuro[3,4-d][1,3]dioxol-6-yl]acetic acid (52, 10 g, 43.1 mmol, 1.0 eq) in DMF (600 ml) was added K₂CO₃ (7.14 g, 51.7 mmol, 1.2 eq) and Mel (9.17 g, 64.6 mmol, 1.5 eq) in portions at 0° C. After stirring for 2 h at this temperature, the mixture was poured into ice-water (1 l) and extracted with methyl tert-butyl ether (3×1 l). The combined organic layers were dried over anhydrous Na₂SO₄, filtered and concentrated in vacuo. After flash chromatography (PE/EtOAc 4:1) 10.0 g (94.3%) of the methylester methyl 2-[(3aR,6R,6aR)-4-methoxy-2,2-dimethyl-3a,4,6,6a-tetrahydrofuro[3,4-d][1,3]dioxol-6-yl]acetate (53) were isolated as color-less oil. 1H-NMR (CDCl₃, 400 MHz) δ[ppm]: 4.97 (s, 1H), 4.59-4.71 (m, 3H), 3.73 (s, 3H), 3.29-3.40 (m, 3H), 2.55-2.76 (m, 2H), 1.50 (s, 3H), 1.33 (s, 3H).

### Example 5.6: Synthesis of Methyl 2-[(3aR,6R,6aR)-4-acetoxy-2,2-dimethyl-3a,4,6,6a-tetra-hydrofuro[3,4-d][1,3]dioxol-6-yl]acetate (54)

To a mixture of the methylglycoside methyl 2-[(3aR,6R,6aR)-4-methoxy-2,2-dimethyl-3a,4,6,6a-tetrahydrofuro[3,4-d][1,3]dioxol-6-yl]acetate (53, 5 g, 20.3 mmol, 1.0 eq) in a mixed solvent of acetic anhydride (17 ml), AcOH (50 ml) and DCM (50 ml) were added 8 drops of H₂SO₄ (conc.) at 10° C. After stirring at this this temperature for 16 h, the mixture was diluted with DCM (100 ml) and neutralized with sat. NaHCO₃-solution (˜200 ml). The organic layer was separated and washed with brine (50 ml), dried over anhydrous Na₂SO₄ and concentrated in vacuo. Flash chromatography (PE/EtOAc 2:1) of the crude product gave 4.3 g (76.9%) of the title compound methyl 2-[(3aR,6R,6aR)-4-acetoxy-2,2-dimethyl-3a,4,6,6a-tetrahydro-furo[3,4-d][1,3]dioxol-6-yl]acetate (54) as white solid. 1H-NMR (CDCl₃, 400 MHz) δ[ppm]: 6.22 (m, 1H), 4.68-4.83 (m, 3H), 3.74 (s, 3H), 2.55-2.79 (m, 2H), 2.10 (s, 3H), 1.52 (s, 3H), 1.36 (s, 3H).

### Example 5.7: Synthesis of Methyl 2-[(2R,3R,4R)-3,4,5-triacetoxytetrahydrofuran-2-yl]-acetate (55)

To a mixture of the starting compound methyl 2-[(3aR,6R,6aR)-4-acetoxy-2,2-dimethyl-3a,4,6,6a-tetrahydrofuro[3,4-d][1,3]dioxol-6-yl]acetate (54, 3.8 g, 13.9 mmol, 1.0 eq) in a mixed solvent of acetic anhydride (19 ml) and AcOH (9.5 ml) was added 10 drops of H₂SO₄ (conc.) at 10° C. After stirring for 16 h at 10° C., the mixture was diluted with EtOAc (100 ml) and washed with sat. NaHCO₃ aqueous solution (2×150 ml), and brine (100 ml), dried over anhydrous Na₂SO₄ and concentrated in vacuo. The crude product was purified by flash chromatography (PE/EtOAc 2:1), yielding 1.78 g (40.5%) of the title compound methyl 2-[(2R,3R,4R)-3,4,5-triacetoxytetrahydrofuran-2-yl]acetate (55) as yellow oil. 1H-NMR (CDCl₃, 400 MHz) δ[ppm]: 6.08 (m, 1H), 5.17-5.31 (m, 2H), 4.44-4.58 (m, 1H), 3.64 (s, 3H), 2.58-2.81 (m, 2H), 2.00-2.07 (m, 9H).

### Example 5.8: Synthesis of Methyl 2-[(2R,3R,4R,5R)-3,4-diacetoxy-5-[2-(2-methylpro-panoylamino)-6-oxo-1H-purin-9-yl]tetrahydrofuran-2-yl]acetate (56)

A solution of the starting compound methyl 2-[(2R,3R,4R)-3,4,5-triacetoxytetrahydrofuran-2-yl]acetate (55, 1.26 g, 4.0 mmol, 1.0 eq), isobutyryl guanine (1.05 g, 4.8 mmol, 1.2 eq) and BSA (3.22 g, 15.8 mmol, 4.0 eq) in DCE (50 ml) was stirred at 100° C. for 2 h, followed by the addition of TMSOTf (2.64 g, 11.9 mmol, 3.0 eq). Stirring was continued for 1 h at 100° C. The solution was cooled to room temperature and diluted with DCM (100 ml). The white precipitate was filtered and the organic solution dried over anhydrous Na₂SO₄. After evaporation of the solvent, the crude product was purified by prep-TLC (EtOAc/MeOH 20:1), yielding 780 mg (41.1%) of the guanosine derivative methyl 2-[(2R,3R,4R,5R)-3,4-diacetoxy-5-[2-(2-methylpropanoylamino)-6-oxo-1H-purin-9-yl]tetrahydrofuran-2-yl]acetate (56) as colorless foam. 1H-NMR (DMSO-d₆, 400 MHz) δ[ppm]: 12.17 (s, 1H), 11.66 (s, 1H), 8.35 (s, 1H), 6.11 (d, J=6.7 Hz, 1H), 5.97 (s, 1H), 5.48 (dd, J=5.6, 3.7 Hz, 1H), 4.45-4.58 (m, 1H), 3.66 (s, 3H), 2.95-3.18 (m, 2H), 2.75-2.91 (m, 1H), 2.19 (s, 3H), 2.08 (s, 3H), 1.20 (br d, J=6.7 Hz, 6H).

### Example 5.9: Synthesis of 2-[(2R,3S,4R,5R)-3,4-Dihydroxy-5-[2-(2-methylpropanoyl-amino)-6-oxo-1H-purin-9-yl]tetrahydrofuran-2-yl]acetic acid (57)

The starting material methyl 2-[(2R,3R,4R,5R)-3,4-diacetoxy-5-[2-(2-methylpropanoyl-amino)-6-oxo-1H-purin-9-yl]tetrahydrofuran-2-yl]acetate (56, 1.61 g, 3.4 mmol, 1.0 eq) was dissolved in a mixed solvent of THF (65 ml) and water (13 ml). LiOH·H₂O (479 mg, 11.4 mmol, 3.4 eq) was added in portions at 0° C. and the reaction was stirred at this temperature for 3 h. The solution was diluted with water (50 ml) and washed with DCM (100 ml). The aqueous layer was separated and neutralized with aqueous 1 N HCl. The precipitate was filtered and the aqueous filtrate washed with DCM (2×100 ml). The combined organic layers were dried over Na₂SO₄ and concentrated in vacuo. The crude product was dissolved in water and DMF (20 ml, v/v=10/1) and purified by reverse flash chromatography (neutral), yielding 1.15 g (62.9%) of the title compound 2-[(2R,3S,4R,5R)-3,4-dihydroxy-5-[2-(2-methylpropanoylamino)-6-oxo-1H-purin-9-yl]tetrahydrofuran-2-yl]acetic acid (57) as yellow foam.

MS (calc.: 381.1) (m/z)=382.0 [M+H⁺].

1H-NMR (D₂O, 400 MHz) δ[ppm]: 8.09 (s, 1H), 5.91 (br d, J=5.0 Hz, 1H), 4.76-4.80 (m, 1H), 4.41 (br s, 1H), 4.20-4.31 (m, 1H), 2.54-2.76 (m, 3H), 1.16 (br d, J=6.9 Hz, 6H).

### Example 5.10: Synthesis of 2-[(2R,3S,4R,5R)-5-(2-Amino-6-oxo-1H-purin-9-yl)-3,4-dihydroxy-tetrahydrofuran-2-yl]acetic acid (58)

To a solution of the starting material 2-[(2R,3S,4R,5R)-3,4-dihydroxy-5-[2-(2-methylpro-panoylamino)-6-oxo-1H-purin-9-yl]tetrahydrofuran-2-yl]acetic acid (57, 650 mg, 1.7 mmol, 1.0 eq) in MeOH (13 ml) was added a NaOMe-solution (1.7 ml, 1.7 mmol, 1.0 eq, 1 mol/1 in MeOH) dropwise at 10° C. After stirring at 60° C. for 10 h, the solvent was removed in vacuo and the residue washed with MeOH (5 ml). After drying in vacuo, 444 mg (87% purity, 72.8%) of the guanosine analog 2-[(2R,3S,4R,5R)-5-(2-amino-6-oxo-1H-purin-9-yl)-3,4-dihydroxy-tetrahydrofuran-2-yl]acetic acid (58) were isolated as yellow solid.

MS (calc.: 311.1) (m/z)=311.9 [M+H⁺].

1H-NMR (D₂O, 400 MHz) δ[ppm]: 7.76 (m, 1H), 5.73 (br d, J=5.5 Hz, 1H), 4.57 (br t, J=5.4 Hz, 1H), 4.21-4.36 (m, 1H), 4.11 (br t, J=4.6 Hz, 1H), 2.39-2.64 (m, 2H).

### Example 6: Synthetic Scheme for the Syntheses of Example Compounds 71, 72, and 73

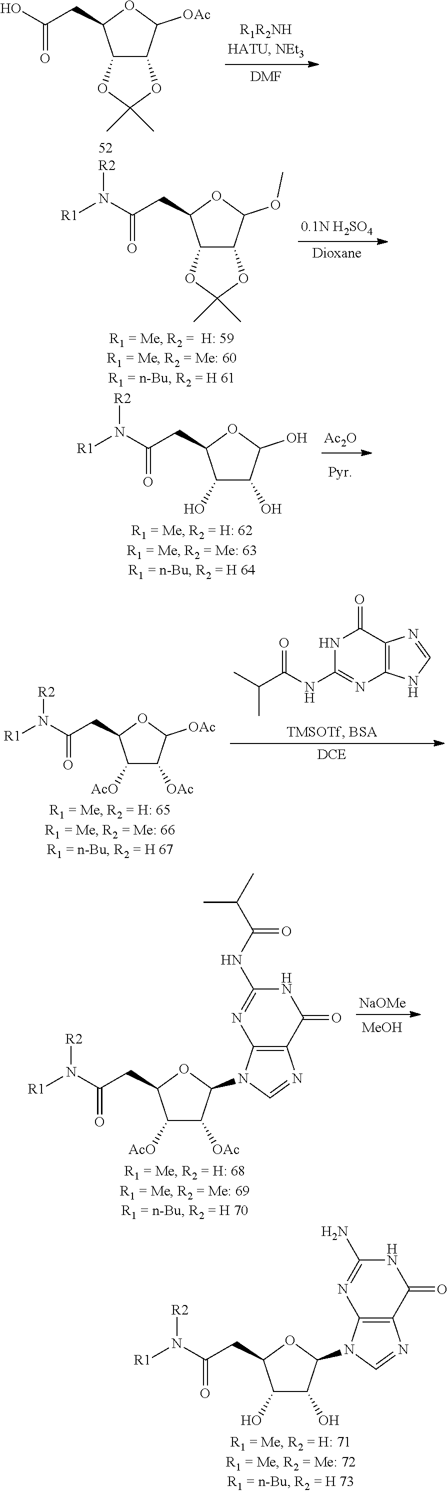

### Example 6.1: Synthesis of 2-[(3aR,6R,6aR)-4-Methoxy-2,2-dimethyl-3a,4,6,6a-tetrahydro-furo[3,4-d][1,3]-dioxol-6-yl]-N-methyl-acetamide (59)

To a solution of the carboxylic acid 2-[(3aR,6R,6aR)-4-Methoxy-2,2-dimethyl-3a,4,6,6a-tetrahydro-furo[3,4-d][1,3]dioxol-6-yl]acetic acid (52, 3 g, 12.9 mmol, 1.0 eq) and MeNH₂·HCl (959 mg, 14.2 mmol, 1.1 eq) in DMF (129 ml) was added HATU (7.37 g, 19.4 mmol, 1.5 eq) and NEt₃ (3.51 g, 27.1 mmol, 2.1 eq) at 10° C. The solution was stirred at 10° C. for 3 h, diluted with EtOAc (500 ml) and washed with water (2×200 ml). The organic layer separated and dried over anhydrous Na₂SO₄, filtered and concentrated in vacuo. Purification of the crude product by flash chromatography (EtOAc) gave 3.54 g (89.5%, purity ˜80%) of the desired amide 2-[(3aR,6R,6aR)-4-methoxy-2,2-dimethyl-3a,4,6,6a-tetrahydrofuro[3,4-d][1,3]-dioxol-6-yl]-N-methyl-acetamide (59) as yellow oil.

1H-NMR (CDCl₃, 400 MHz) δ[ppm]: 6.05 (br s, 1H), 4.90 (s, 1H), 4.44-4.61 (m, 3H), 3.28 (s, 3H), 2.74 (s, 3H), 2.31-2.53 (m, 2H), 1.41 (s, 3H), 1.24 (s, 3H).

### Example 6.2: Synthesis of 2-[(3aR,6R,6aR)-4-Methoxy-2,2-dimethyl-3a,4,6,6a-tetrahydro-furo[3,4-d][1,3]-dioxol-6-yl]-N,N-dimethyl-acetamide (60)

Following the protocol, described for the synthesis of 2-[(3aR,6R,6aR)-4-methoxy-2,2-dimethyl-3a,4,6,6a-tetrahydrofuro[3,4-d][1,3]-dioxol-6-yl]-N-methyl-acetamide (59), 6.0 g (25.8 mmol, 1.0 eq) of the carboxylic acid 2-[(3aR,6R,6aR)-4-Methoxy-2,2-dimethyl-3a,4,6,6a-tetrahydro-furo[3,4-d][1,3]dioxol-6-yl]acetic acid (52) and 2.3 g (28 mmol, 1.1 eq) of dimethylamine-hydrochloride gave 5.6 g (84.8%) of the desired dimethylamide 2-[(3aR,6R,6aR)-4-methoxy-2,2-dimethyl-3a,4,6,6a-tetrahydrofuro[3,4-d][1,3]-dioxol-6-yl]-N,N-dimethyl-acetamide (60) as yellow oil. 1H-NMR (CDCl₃, 400 MHz) δ[ppm]: 4.95 (s, 1H), 4.72-4.65 (m, 2H), 4.60 (d, J=5.9 Hz, 1H), 3.34 (s, 3H), 3.00 (s, 3H), 2.80 (s, 3H), 2.70-2.80 (m, 1H), 2.50-2.60 (m, 1H), 1.49 (s, 3H), 1.31 (s, 3H).

### Example 6.3: Synthesis of 2-[(3aR,6R,6aR)-4-Methoxy-2,2-dimethyl-3a,4,6,6a-tetrahydro-furo[3,4-d][1,3]-dioxol-6-yl]-N-butyl-acetamide (61)

Following the protocol, described for the synthesis of 2-[(3aR,6R,6aR)-4-methoxy-2,2-dimethyl-3a,4,6,6a-tetrahydrofuro[3,4-d][1,3]-dioxol-6-yl]-N-methyl-acetamide (59), 6.0 g (25.8 mmol, 1.0 eq) of the carboxylic acid 2-[(3aR,6R,6aR)-4-Methoxy-2,2-dimethyl-3a,4,6,6a-tetrahydro-furo[3,4-d][1,3]dioxol-6-yl]acetic acid (52) and 2.1 g (28 mmol, 1.1 eq) of n-butylamine gave 7.0 g (93.3%) of the desired n-butylamide 2-[(3aR,6R,6aR)-4-methoxy-2,2-dimethyl-3a,4,6,6a-tetrahydrofuro[3,4-d][1,3]-dioxol-6-yl]-N-butyl-acetamide (61), after silicagel chromatography (PE/Et=Ac 1:1), as yellow oil. 1H-NMR (CDCl₃, 400 MHz) δ[ppm]: 6.06 (br s, 1H), 4.99 (s, 1H), 4.71-4.50 (m, 3H), 3.37 (s, 3H), 3.33-3.25 (m, 2H), 2.60-2.51 (m, 1H), 2.50-2.39 (m, 1H), 1.56-1.46 (m, 5H), 1.42-1.30 (m, 5H), 0.94 (t, J=7.3 Hz, 3H).

### Example 6.4: Synthesis of N-Methyl-2-[(2R,3S,4R)-3,4,5-trihydroxytetrahydrofuran-2-yl]acetamide (62)

The starting material 2-[(3aR,6R,6aR)-4-methoxy-2,2-dimethyl-3a,4,6,6a-tetrahydrofuro[3,4-d][1,3]-dioxol-6-yl]-N-methyl-acetamide (59, 2 g, 8.2 mmol, 1.0 eq) was dissolved in a mixed solvent of 0.1 N H₂SO₄ (27 ml, 2.7 mmol, 0.33 eq) and dioxane (13.5 ml). After stirring for 2 h at 120° C. (oil bath), the reaction mixture was cooled to 10° C. and neutralized with Ba(OH)₂·8H₂O (solid). The mixture was concentrated in vacuo and the residue co-evaporated with dioxane (3×50 ml) to give 3 g (crude) of the deprotected furanose derivative N-methyl-2-[(2R,3S,4R)-3,4,5-trihydroxytetrahydrofuran-2-yl]acetamide (62) as yellow solid, which was used for next step without further purification.

### Example 6.5: Synthesis of N,N-Dimethyl-2-[(2R,3S,4R)-3,4,5-trihydroxytetrahydrofuran-2-yl]acetamide (63)

Following the protocol, described for the synthesis of N-methyl-2-[(2R,3S,4R)-3,4,5-trihydroxytetrahydrofuran-2-yl]acetamide (62), 5.6 g (21.6 mmol, 1.0 eq) of the starting material 2-[(3aR,6R,6aR)-4-methoxy-2,2-dimethyl-3a,4,6,6a-tetrahydrofuro[3,4-d][1,3]-diox-ol-6-yl]-N,N-dimethyl-acetamide (60) were hydrolyzed with 0.1 N H₂SO₄. After neutralization with Ba(OH)₂·8H₂O, the mixture was poured into water (150 ml) and washed with EtOAc (2×50 ml). The aqueous layer was separated and concentrated in vacuo to give the title compound N,N-dimethyl-2-[(2R,3S,4R)-3,4,5-trihydroxytetrahydrofuran-2-yl]acetamide (63) (8 g, crude) as yellow oil, which was used for next step without further purification.

### Example 6.6: Synthesis of N-Butyl-2-[(2R,3S,4R)-3,4,5-trihydroxytetrahydrofuran-2-yl]-acetamide (64)

Following the protocol, described for the synthesis of N-methyl-2-[(2R,3S,4R)-3,4,5-trihydroxytetrahydrofuran-2-yl]acetamide (62), 2-[(3aR,6R,6aR)-4-methoxy-2,2-dimethyl-3a,4,6,6a-tetrahydrofuro[3,4-d][1,3]-dioxol-6-yl]-N-butyl-acetamide (61, 7.0 g, 24.0 mmol, 1.0 eq) was hydrolyzed, yielding the title compound N-butyl-2-[(2R,3S,4R)-3,4,5-trihydroxytetrahydrofuran-2-yl]acetamide (64) (6 g, crude), after working up as described for N,N-dimethyl-2-[(2R,3S,4R)-3,4,5-trihydroxytetrahydrofuran-2-yl]acetamide (63), as yellow oil, which was used in the next step without further purification.

### Example 6.7: Synthesis of [(2R,3R,4R)-4,5-Diacetoxy-2-[2-(methylamino)-2-oxo-ethyl]-tetrahydrofuran-3-yl] acetate (65)

The crude product N-methyl-2-[(2R,3S,4R)-3,4,5-trihydroxytetrahydrofuran-2-yl]acetamide (62, 3 g, 8.2 mmol, purity 52%, 1.0 eq) was co-evaporated with pyridine (3×20 ml) and dissolved in pyridine (20 ml). After adding acetic anhydride (20 ml) dropwise at 10° C., the solution was stirred at this temperature for 16 h, to achieve complete conversion. The reaction mixture was concentrated in vacuo and the residue was purified by flash chromatography (EtOAc), which gave 1.46 g (56.1%, two steps) of the peracetylated product [(2R,3R,4R)-4,5-diacetoxy-2-[2-(methylamino)-2-oxo-ethyl]tetrahydrofuran-3-yl] acetate (65) as yellow oil. 1H-NMR (CDCl₃, 400 MHz) δ[ppm]: 6.02-6.39 (m, 1H), 5.60-5.94 (m, 1H), 5.11-5.33 (m, 2H), 4.37-4.56 (m, 1H), 2.74 (d, J=4.89 Hz, 3H), 2.38-2.60 (m, 2H), 1.96-2.09 (m, 9H).

### Example 6.8: Synthesis of [(2R,3R,4R)-4,5-Diacetoxy-2-[2-(dimethylamino)-2-oxo-ethyl]-tetrahydrofuran-3-yl] acetate (66)

Following the protocol, described for the synthesis of [(2R,3R,4R)-4,5-diacetoxy-2-[2-(methylamino)-2-oxo-ethyl]tetrahydrofuran-3-yl] acetate (65), crude N,N-dimethyl-2-[(2R,3S,4R)-3,4,5-trihydroxytetrahydrofuran-2-yl]acetamide (63, 8 g) were acetylated and gave, after silicagel chromatography (EtOAc) 4.0 g (55.9%, two steps) of the title compound [(2R,3R,4R)-4,5-diacetoxy-2-[2-(dimethylamino)-2-oxo-ethyl]tetrahydrofuran-3-yl] acetate (66) as colorless oil. 1H-NMR (CDCl₃, 400 MHz) δ[ppm]: 6.01-6.34 (m, 1H), 5.19-5.31 (m, 2H), 4.52-4.70 (m, 1H), 2.91-2.96 (m, 3H), 2.83-2.89 (m, 3H), 2.66-2.73 (m, 1H), 2.57-2.65 (m, 1H), 2.04-2.07 (m, 3H), 2.02 (s, 3H), 2.00 (s, 3H).

### Example 6.9: Synthesis of [(2R,3R,4R)-4,5-Diacetoxy-2-[2-(butylamino)-2-oxo-ethyl]-tetrahydrofuran-3-yl] acetate (67)

Following the protocol, described for the synthesis of [(2R,3R,4R)-4,5-diacetoxy-2-[2-(methylamino)-2-oxo-ethyl]tetrahydrofuran-3-yl] acetate (65), crude N-butyl-2-[(2R,3S,4R)-3,4,5-trihydroxytetrahydrofuran-2-yl]acetamide (64, 6 g) were acetylated and gave, after silicagel chromatography (PE/EtOAc 1:1) 6.5 g (75.4%, two steps) of the title compound [(2R,3R,4R)-4,5-diacetoxy-2-[2-(butylamino)-2-oxo-ethyl]tetrahydrofuran-3-yl]acetate (67) as yellow oil. 1H-NMR (DMSO-d₆, 400 MHz) δ[ppm]: 7.84-7.98 (m, 1H), 5.92-6.33 (m, 1H), 5.16-5.36 (m, 2H), 4.40-4.49 (m, 1H), 2.98-3.09 (m, 2H), 2.37-2.49 (m, 2H), 2.01-2.11 (m, 9H), 1.32-1.41 (m, 2H), 1.21-1.31 (m, 2H), 0.83-0.91 (m, 3H).

### Example 6.10: Synthesis of [(2R,3R,4R,5R)-4-Acetoxy-2-[2-(methylamino)-2-oxo-ethyl]-5-[2-(2-methylpropanoylamino)-6-oxo-1H-purin-9-yl]tetrahydrofuran-3-yl] acetate (68)

To a solution of the ribose derivative [(2R,3R,4R)-4,5-diacetoxy-2-[2-(methylamino)-2-oxo-ethyl]tetrahydrofuran-3-yl] acetate (65, 2.58 g, 8.1 mmol, 1.0 eq) and isobutyryl guanine (2.16 g, 9.8 mmol, 1.2 eq) in DCE (103 ml) was added BSA (6.6 g, 32.5 mmol, 4.0 eq) dropwise at 10° C. The reaction solution was stirred at 100° C. for 2 h, followed by the addition of TMSOTf (5.4 g, 24.4 mmol, 3.0 eq). The stirring for another h at 100° C., the reaction solution was cooled to 10° C. and diluted with DCM (100 ml). The organic solution was washed with sat. NaHCO₃ (100 ml) and brine (100 ml), dried over anhydrous Na₂SO₄, filtered and concentrated in vacuo. The crude product was purified by prep-TLC (EtOAc/MeOH 4:1), yielding 1.48 g (38%) of the guanosine analog [(2R,3R,4R,5R)-4-acetoxy-2-[2-(methylamino)-2-oxo-ethyl]-5-[2-(2-methylpropanoylamino)-6-oxo-1H-purin-9-yl]tetrahydrofuran-3-yl] acetate (68) as yellow foam.

MS (calc.: 478.2) (m/z)=479.1 [M+H⁺].

1H-NMR (DMSO-d₆, 400 MHz) δ[ppm]: 12.13 (br s, 1H), 11.66 (br s, 1H), 8.26 (s, 1H), 7.92 (br d, J=4.7 Hz, 1H), 6.01 (d, J=6.5 Hz, 1H), 5.86 (t, J=6.1 Hz, 1H), 5.47 (dd, J=5.8, 3.9 Hz, 1H), 4.39-4.49 (m, 1H), 2.79 (dt, J=13.7, 6.9 Hz, 1H), 2.62-2.73 (m, 2H), 2.57 (d, J=4.5 Hz, 3H), 2.12 (s, 3H), 2.02 (s, 3H), 1.14 (d, J=6.9 Hz, 6H).

### Example 6.11: Synthesis of [(2R,3R,4R,5R)-4-Acetoxy-2-[2-(dimethylamino)-2-oxo-ethyl]-5-[2-(2-methylpropanoyl-amino)-6-oxo-1H-purin-9-yl]tetrahydrofuran-3-yl] acetate (69)

Referring to the protocol described for the synthesis of [(2R,3R,4R,5R)-4-acetoxy-2-[2-(methylamino)-2-oxo-ethyl]-5-[2-(2-methylpropanoylamino)-6-oxo-1H-purin-9-yl]tetra-hydrofuran-3-yl] acetate (68), starting material [(2R,3R,4R)-4,5-diacetoxy-2-[2-(dimethylamino)-2-oxo-ethyl]tetrahydrofuran-3-yl] acetate (66, 3 g, 9.05 mmol, 1.0 eq) was glycosylated with isobutyryl guanine (2.4 g, 10.8 mmol, 1.2 eq). After a reaction time of 3 h at 100° C., the reaction was complete. Working up as described in (68) and purification by reverse flash chromatography (FA) and SFC (neuture-MeOH, REG2S(s,s)), gave 1.8 g (40%) of the guanosine analog [(2R,3R,4R,5R)-4-acetoxy-2-[2-(dimethylamino)-2-oxo-ethyl]-5-[2-(2-methylpropanoylamino)-6-oxo-1H-purin-9-yl]tetrahydrofuran-3-yl] acetate (69) as yellow foam.

MS (calc.: 492.2) (m/z)=493.1 [M+H⁺].

1H-NMR (DMSO-d₆, 400 MHz) δ[ppm]: 12.11 (br s, 1H), 11.66 (br s, 1H), 8.33 (s, 1H), 6.08-6.14 (m, 1H), 6.02-6.07 (m, 1H), 5.43 (dd, J=2.5, 5.4 Hz, 1H), 4.45-4.58 (m, 1H), 3.01-3.10 (m, 1H), 2.89-3.00 (m, 4H), 2.73-2.85 (m, 4H), 2.15 (s, 3H), 2.00 (s, 3H), 1.13 (dd, J=1.7, 6.8 Hz, 6H).

### Example 6.12: Synthesis of [(2R,3R,4R,5R)-4-Acetoxy-2-[2-(butylamino)-2-oxo-ethyl]-5-[2-(2-methylpro-panoyl-amino)-6-oxo-1H-purin-9-yl]tetrahydrofuran-3-yl] acetate (70)

To a solution of the starting compound [(2R,3R,4R)-4,5-diacetoxy-2-[2-(butylamino)-2-oxo-ethyl]tetrahydrofuran-3-yl] acetate (67, 3 g, 8.35 mmol, 1.0 eq) and isobutyryl guanine (2.2 g, 10.0 mmol, 1.2 eq) in DCE (120 ml) was added BSA (6.8 g, 33.4 mmol, 4.0 eq) dropwise at 10° C. The mixture was stirred at 100° C. for 1 h, followed by the addition of TMSOTf (5.5 g, 25.1 mmol, 3.0 eq). After another 3 h at 100° C., the reaction mixture was cooled to 10° C. and poured into water (100 ml). The layers were separated and the aqueous phase extracted with DCM (2×100 ml). The combined organic phases were dried over anhydrous Na₂SO₄, filtered and concentrated in vacuo. The crude product was purified by reverse flash chromatography (FA) and SFC (neuture-MeOH, REG2S(s,s)), which gave 1.5 g (30%) of the guanosine derivative [(2R,3R,4R,5R)-4-acetoxy-2-[2-(butylamino)-2-oxo-ethyl]-5-[2-(2-methylpro-panoylamino)-6-oxo-1H-purin-9-yl]tetrahydrofuran-3-yl] acetate (70) as yellow foam.

MS (calc.: 520.2) (m/z)=521.1 [M+H⁺].

1H-NMR (DMSO-d₆, 400 MHz) δ[ppm]: 11.30-12.37 (2×br s, 2H), 8.26 (s, 1H), 7.89 (t, J=5.5 Hz, 1H), 6.02 (d, J=6.8 Hz, 1H), 5.87 (t, J=6.2 Hz, 1H), 5.47 (dd, J=3.6, 5.6 Hz, 1H), 4.36-4.51 (m, 1H), 3.04 (q, J=6.3 Hz, 2H), 2.83-2.74 (m, 1H), 2.71-2.62 (m, 2H), 2.12 (s, 3H), 2.02 (s, 3H), 1.30-1.39 (m, 2H), 1.18-1.28 (m, 2H), 1.14 (d, J=6.9 Hz, 6H), 0.82 (t, J=7.2 Hz, 3H).

### Example 6.13: Synthesis of 2-[(2R,3S,4R,5R)-5-(2-Amino-6-oxo-1H-purin-9-yl)-3,4-dihydroxy-tetrahydrofuran-2-yl]-N-methyl-acetamide (71)

The starting material [(2R,3R,4R,5R)-4-acetoxy-2-[2-(methylamino)-2-oxo-ethyl]-5-[2-(2-methylpropanoylamino)-6-oxo-1H-purin-9-yl]tetrahydrofuran-3-yl] acetate (68, 930 mg, 1.9 mmol, 1.0 eq) was dissolved in MeOH (18.6 ml) and a NaOMe-solution (0.64 ml, 0.64 mmol, 0.33 eq, 1 mol/1 in MeOH) was added dropwise at 10° C. After stirring for 3 h at 60° C., the solvent was removed in vacuo and the residue washed with MeOH (5 ml), yielding 501 mg (79.5%) of the title compound 2-[(2R,3S,4R,5R)-5-(2-amino-6-oxo-1H-purin-9-yl)-3,4-dihydroxy-tetrahydrofuran-2-yl]-N-methyl-acetamide (71) as white solid.

MS (calc.: 324.1) (m/z)=324.8 [M+H⁺].

1H-NMR (DMSO-d₆, 400 MHz) δ[ppm]: 7.89 (br d, J=4.5 Hz, 1H), 7.85 (s, 1H), 6.88 (br s, 2H), 5.70 (d, J=5.4 Hz, 1H), 4.54 (t, J=5.1 Hz, 1H), 4.17-4.26 (m, 1H), 4.08 (t, J=4.6 Hz, 1H), 2.61 (d, J=4.4 Hz, 3H), 2.43-2.54 (m, 2H).

1H-NMR (D₂O, 400 MHz) δ[ppm]: 7.73 (s, 1H), 5.72 (d, J=4.9 Hz, 1H), 4.62 (br t, J=5.0 Hz, 1H), 4.24-4.35 (m, 1H), 4.13-4.22 (m, 1H), 2.54-2.69 (m, 2H), 2.52 (s, 3H).

### Example 6.14: Synthesis of 2-[(2R,3S,4R,5R)-5-(2-Amino-6-oxo-1H-purin-9-yl)-3,4-dihydroxytetrahydrofuran-2-yl]-N,N-dimethyl-acetamide (72)

The starting material [(2R,3R,4R,5R)-4-acetoxy-2-[2-(dimethylamino)-2-oxo-ethyl]-5-[2-(2-methylpropanoyl-amino)-6-oxo-1H-purin-9-yl]tetrahydrofuran-3-yl] acetate (69, 1.6 g, 3.25 mmol, 1.0 eq) was dissolved in MeOH (30 ml) and 1.62 ml (1.62 mmol, 0.5 eq, 1 mol/1 in MeOH) of a NaOMe-solution were added dropwise at 10° C. After stirring for 4 h at 60° C., the solvent was evaporated in vacuo and the residue triturated with 10 ml MeOH, which gave 0.99 g (90%) of the desired guanosine analog 2-[(2R,3S,4R,5R)-5-(2-amino-6-oxo-1H-purin-9-yl)-3,4-dihydroxy-tetrahydrofuran-2-yl]-N,N-dimethyl-acetamide (72) as brown solid.

MS (calc.: 338.1) (m/z)=339.1 [M+H⁺].

1H-NMR (D₂O, 400 MHz) δ[ppm]: 7.70 (br s, 1H), 5.70 (m, 1H), 4.68 (br s, 1H), 4.34-4.22 (m, 2H), 2.83 (br s, 3H), 2.82-2.73 (m, 2H), 2.71 (br s, 3H).

### Example 6.15: Synthesis of 2-[(2R,3S,4R,5R)-5-(2-Amino-6-oxo-1H-purin-9-yl)-3,4-dihydroxy-tetrahydrofuran-2-yl]-N-butyl-acetamide (73)

Following the protocol, described for the synthesis of 2-[(2R,3S,4R,5R)-5-(2-amino-6-oxo-1H-purin-9-yl)-3,4-dihydroxy-tetrahydrofuran-2-yl]-N,N-dimethyl-acetamide (72), 1.4 g (2.7 mmol, 1.0 eq) of the starting material [(2R,3R,4R,5R)-4-acetoxy-2-[2-(butylamino)-2-oxo-ethyl]-5-[2-(2-methyl-propanoylamino)-6-oxo-1H-purin-9-yl]tetrahydrofuran-3-yl]acetate (70) were treated with NaOMe in MeOH, yielding 800 mg (81.2%) of the title compound 2-[(2R,3S,4R,5R)-5-(2-amino-6-oxo-1H-purin-9-yl)-3,4-dihydroxy-tetrahydrofuran-2-yl]-N-butyl-acetamide (73) as white solid.

MS (calc.: 366.2) (m/z)=366.9 [M+H⁺].

1H-NMR (DMSO-d₆, 400 MHz) δ[ppm]: 7.69-7.84 (m, 2H), 6.81 (br s, 2H), 5.65 (d, J=5.6 Hz, 1H), 4.97-5.59 (m, 1H), 4.51 (t, J=5.3 Hz, 1H), 4.11-4.23 (m, 1H), 4.02 (t, J=4.3 Hz, 1H), 3.02 (q, J=6.3 Hz, 2H), 2.48 (m, 2H), 1.28-1.38 (m, 2H), 1.18-1.26 (m, 2H), 0.82 (t, J=7.3 Hz, 3H).

1H-NMR (D₂O, 400 MHz) δ[ppm]: 7.84 (s, 1H), 5.80 (d, J=5.9 Hz, 1H), 4.93 (t, J=5.5 Hz, 1H), 4.33-4.41 (m, 1H), 4.25-4.32 (m, 1H), 3.13 (td, J=6.7, 13.4 Hz, 1H), 2.93 (td, J=6.4, 13.2 Hz, 1H), 2.58-2.73 (m, 2H), 1.10-1.28 (m, 2H), 0.85-1.01 (m, 2H), 0.60 (t, J=7.3 Hz, 3H).

### Example 7: Synthesis of Precursor for Simplified Piperidine-Derived ASGPR Binders

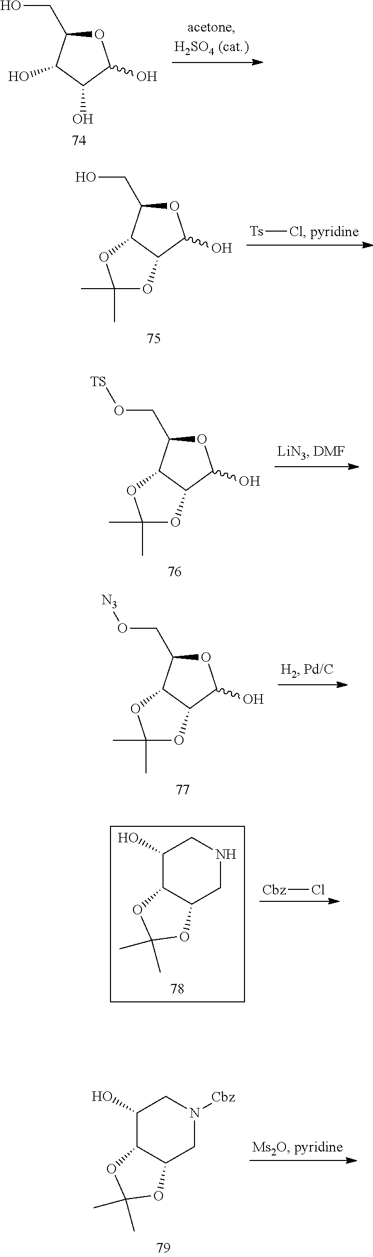

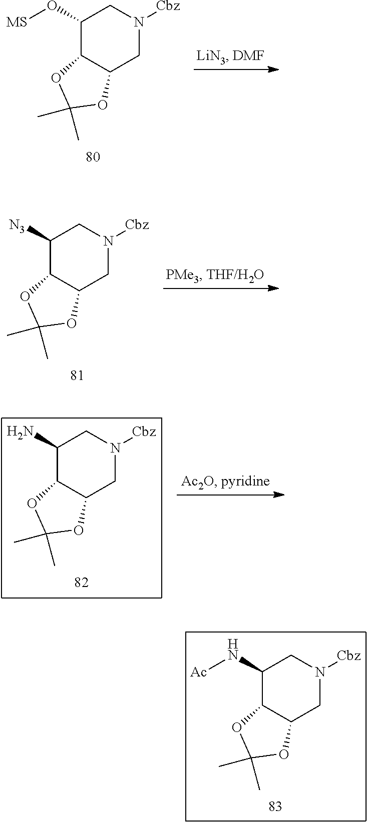

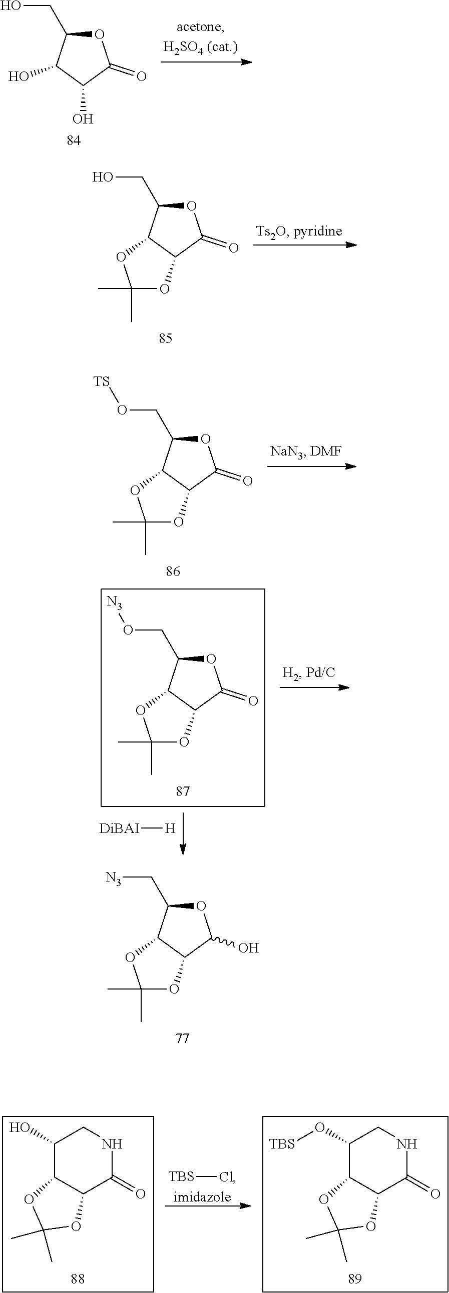

### Example 7.1: Synthesis of (3aR,6R,6aR)-6-(hydroxymethyl)-2,2-dimethyl-3a,4,6,6a-tetrahydrofuro[3,4-d][1,3]dioxol-4-ol (75)

To a solution of D-ribose (74, 50.25 g, 334.71 mmol, 1.00 equiv.) in acetone (500 mL) was added concentrated sulfuric acid (3.50 g, 35.65 mmol, 0.11 equiv.). The reaction mixture was stirred overnight until TLC indicated full conversion of the starting material. Saturated aqueous NaHCO₃-solution (300 mL) and toluene (300 mL) were added and the mixture was concentrated in vacuo to remove the acetone from the mixture.

EtOAc (300 mL), saturated aqueous NaHCO₃-solution (100 mL) and water (50 mL) were added, the layers were separated, and the aqueous layer was re-extracted with EtOAc (3×100 mL). The combined organic layers were washed with saturated aqueous NaCl-solution (100 mL), dried (MgSO₄), filtered and concentrated in vacuo. The crude product was purified by flash chromatography (silica, 0-100% EtOAc in n-heptane) to yield (3aR,6R,6aR)-6-(hydroxymethyl)-2,2-dimethyl-3a,4,6,6a-tetrahydrofuro[3,4-d][1,3]dioxol-4-ol (75, 33.79 g, 177.66 mmol, 53%) as a colorless oil.

### Example 7.2: Synthesis of [(3aR,6R,6aR)-4-hydroxy-2,2-dimethyl-3a,4,6,6a-tetrahydrofuro[3,4-d][1,3]dioxol-6-yl]methyl 4-methylbenzenesulfonate (76)

A solution of (3aR,6R,6aR)-6-(hydroxymethyl)-2,2-dimethyl-3a,4,6,6a-tetrahydrofuro[3,4-d][1,3]dioxol-4-ol (75, 8.02 g, 42.18 mmol, 1.00 equiv.) in anhydrous pyridine (20 mL) was cooled to 0° C. and tosyl chloride (9.85 g, 51.67 mmol, 1.20 equiv.) was added. The reaction mixture was stirred for 1.5 h at 0° C. until LC/MS indicated full conversion of the starting material. EtOAc (300 mL) and aqueous 1 N HCl (150 mL) were added, the layers were separated, and the aqueous layer was re-extracted with EtOAc (2×100 mL). The combined organic layers were washed with saturated aqueous NaHCO₃-solution (2×50 mL), saturated aqueous NaCl-solution (2×50 mL), dried (MgSO₄), filtered and concentrated in vacuo. The crude product was directly used for the next step.

LC-MS (Method D):

Rₜ[min] (UV-signal 220 nm): 1.24

M[g/mol]: 327.0 [M+H−H₂O⁺]

### Example 7.3: Synthesis of (3aR,6R,6aR)-6-(aminomethyl)-2,2-dimethyl-3a,4,6,6a-tetrahydrofuro[3,4-d][1,3]dioxol-4-ol (77)

From[(3aR,6R,6aR)-4-hydroxy-2,2-dimethyl-3a,4,6,6a-tetrahydrofuro[3,4-d][1,3]dioxol-6-yl]methyl 4-methylbenzenesulfonate (76): The crude product [(3aR,6R,6aR)-4-hydroxy-2,2-dimethyl-3a,4,6,6a-tetrahydrofuro[3,4-d][1,3]dioxol-6-yl]methyl 4-methylbenzenesulfonate (76, max. 42.18 mmol, 1.00 equiv.) was dissolved in DMF (10 mL), LiN₃ (2 M in DMF, 60.0 mL, 120 mmol, 2.80 equiv.) was added and the mixture was stirred at 75° C. overnight. EtOAc (300 mL) and water (100 mL) were added, the layers were separated, and the aqueous layer was re-extracted with EtOAc (3×50 mL). The combined organic layers were washed with saturated aqueous NaCl-solution (50 mL), dried (MgSO₄), filtered and concentrated in vacuo. The crude product was purified by flash chromatography (silica, 0-70% EtOAc in n-heptane) to yield (3aR,6R,6aR)-6-(aminomethyl)-2,2-dimethyl-3a,4,6,6a-tetrahydrofuro[3,4-d][1,3]dioxol-4-ol (77, 3.51 g, 16.32 mmol, 39% over two steps) as a colorless oil.

From (3aR,6R,6aR)-6-(aminomethyl)-2,2-dimethyl-6,6a-dihydro-3aH-furo[3,4-d][1,3]dioxol-4-one (87): A solution of (3aR,6R,6aR)-6-(aminomethyl)-2,2-dimethyl-6,6a-dihydro-3aH-furo[3,4-d][1,3]dioxol-4-one (87, 3.05 g, 14.28 mmol, 1.00 equiv.) in anhydrous DCM (50 mL) was cooled to −78° C. and DiBAl-H (1 M in toluene, 24.00 mL, 24.00 mmol, 1.68 equiv.) was added. The reaction mixture was stirred for 30 min at −78° C. until LC/MS indicated full conversion of the starting material. Saturated aqueous Rochelle's salt solution (30 mL) and EtOAc (150 mL) were added, the mixture was stirred for 1 h at r.t., the layers were separated, and the aqueous layer was re-extracted with EtOAc (60 mL). The combined organic layers were washed with saturated aqueous NaCl-solution (50 mL), dried (MgSO₄), filtered and concentrated in vacuo. The crude product (3aR,6R,6aR)-6-(aminomethyl)-2,2-dimethyl-3a,4,6,6a-tetrahydrofuro[3,4-d][1,3]dioxol-4-ol (77, 2.90 g, 13.48 mmol, 94%) was obtained as a colorless oil and used for the next step without further purification.

LC-MS (Method D):

Rₜ[min] (UV-signal 220 nm): 0.80

M[g/mol]: 170.0 [M+H−N₂−H₂O⁺]

### Example 7.4: Synthesis of (3aS,7R,7aR)-2,2-dimethyl-3a,4,5,6,7,7a-hexahydro-[1,3]dioxolo[4,5-c]pyridin-7-ol (78)

(3aR,6R,6aR)-6-(aminomethyl)-2,2-dimethyl-3a,4,6,6a-tetrahydrofuro[3,4-d][1,3]dioxol-4-ol (77, 3.37 g, 15.66 mmol, 1.00 equiv.) was dissolved in THF (50 mL), 10% Pd/C (0.17 g, 0.16 mmol, 0.01 equiv.) was added and the mixture was hydrogenated in an autoclave at r.t. and 4 bar hydrogen gas for 4 d. Since complete piperidine formation was detected by LC/MS, the reaction mixture was filtered. The crude product (3aS,7R,7aR)-2,2-dimethyl-3a,4,5,6,7,7a-hexahydro-[1,3]dioxolo[4,5-c]pyridin-7-ol (78) was directly used for further reactions as stock solution in THF.

LC-MS (Method D):

Rₜ[min] (TIC-signal): 0.14

M[g/mol]: 173.9 [M+H⁺]

### Example 7.5: Synthesis of benzyl (3aS,7R,7aR)-7-hydroxy-2,2-dimethyl-4,6,7,7a-tetrahydro-3aH-[1,3]dioxolo[4,5-c]pyridine-5-carboxylate (79)

To a solution of (3aS,7R,7aR)-2,2-dimethyl-3a,4,5,6,7,7a-hexahydro-[1,3]dioxolo[4,5-c]pyridin-7-ol (78), directly obtained from hydrogenation of (3aR,6R,6aR)-6-(aminomethyl)-2,2-dimethyl-3a,4,6,6a-tetrahydrofuro[3,4-d][1,3]dioxol-4-ol (77, max. 15.66 mmol, 1.00 equiv.), in THF (90 mL), saturated aqueous NaHCO₃-solution (30 mL) and Cbz-Cl (2.67 g, 15.66 mmol, 1.00 equiv.) were added and the reaction mixture was stirred overnight. EtOAc (200 mL), saturated aqueous NaHCO₃-solution (50 mL) and water (30 mL) were added, the layers were separated, and the aqueous layer was re-extracted with EtOAc (2×50 mL). The combined organic layers were washed with saturated aqueous NaCl-solution (50 mL), dried (MgSO₄), filtered and concentrated in vacuo. The crude product was purified by flash chromatography (silica, 0-100% EtOAc in n-heptane) to yield benzyl (3aS,7R,7aR)-7-hydroxy-2,2-dimethyl-4,6,7,7a-tetrahydro-3aH-[1,3]dioxolo[4,5-c]pyridine-5-carboxylate (79, 3.37 g, 10.96 mmol, 70% over two steps) as a colorless oil.

LC-MS (Method D):

Rₜ[min] (UV-signal 220 nm): 1.08

M[g/mol]: 308.0 [M+H⁺]

### Example 7.6: Synthesis of benzyl (3aS,7R,7aS)-2,2-dimethyl-7-methylsulfonyloxy-4,6,7,7a-tetrahydro-3aH-[1,3]dioxolo[4,5-c]pyridine-5-carboxylate (80)

A solution of benzyl (3aS,7R,7aR)-7-hydroxy-2,2-dimethyl-4,6,7,7a-tetrahydro-3aH-[1,3]dioxolo[4,5-c]pyridine-5-carboxylate (79, 5.00 g, 16.27 mmol, 1.00 equiv.) in anhydrous DCM (80 mL) was cooled to 0° C. and pyridine (5.25 mL, 65.07 mmol, 4.00 equiv.) and mesyl anhydride (11.33 g, 65.07 mmol, 4.00 equiv.) were added. The reaction mixture was stirred for 1.5 h at 0° C. until LC/MS indicated full conversion of the starting material. Aqueous 1 N HCl (100 mL) and EtOAc (250 mL) were added, the layers were separated, the organic layer was washed with saturated aqueous NaCl-solution (50 mL), dried (MgSO₄), filtered and concentrated in vacuo. The crude product was purified by flash chromatography (silica, 0-100% EtOAc in n-heptane) to yield benzyl (3aS,7R,7aS)-2,2-dimethyl-7-methylsulfonyloxy-4,6,7,7a-tetrahydro-3aH-[1,3]dioxolo[4,5-c]pyridine-5-carboxylate (80, 3.63 g, 9.41 mmol, 58%) as a colorless oil.

LC-MS (Method D):

Rₜ[min] (UV-signal 220 nm): 1.34

M[g/mol]: 386.0 [M+H⁺]

### Example 7.7: Synthesis of benzyl (3aS,7S,7aR)-7-hydroxy-2,2-dimethyl-4,6,7,7a-tetrahydro-3aH-[1,3]dioxolo[4,5-c]pyridine-5-carboxylate (81)

To a solution of benzyl (3aS,7R,7aS)-2,2-dimethyl-7-methylsulfonyloxy-4,6,7,7a-tetrahydro-3aH-[1,3]dioxolo[4,5-c]pyridine-5-carboxylate (80, 3.63 g, 9.41 mmol, 1.00 equiv.) in DMF (3 mL), LiN₃ (2 M in DMF, 12 mL, 24 mmol, 2.5 equiv.) was added and the mixture was stirred at 100° C. for 2 d. The reaction was stopped since significant amounts of an elimination product were detected by LC/MS. EtOAc (100 mL) and water (50 mL) were added, the layers were separated, and the aqueous layer was re-extracted with EtOAc (3×20 mL). The combined organic layers were washed with saturated aqueous NaCl-solution (30 mL), dried (MgSO₄), filtered and concentrated in vacuo. The crude product was purified by flash chromatography (silica, 0-100% EtOAc in n-heptane) to yield benzyl (3aS,7S,7aR)-7-hydroxy-2,2-dimethyl-4,6,7,7a-tetrahydro-3aH-[1,3]dioxolo[4,5-c]pyridine-5-carboxylate (81, 410 mg, 1.23 mmol, 13%) and recovered benzyl (3aS,7R,7aS)-2,2-dimethyl-7-methylsulfonyloxy-4,6,7,7a-tetrahydro-3aH-[1,3]dioxolo[4,5-c]pyridine-5-carboxylate (80, 1.77 g, 4.58 mmol, 49%) as colorless oils.

LC-MS (Method D):

Rₜ[min] (UV-signal 220 nm): 1.52

M[g/mol]: 305.1 [M+H−N₂⁺]

### Example 7.8: Synthesis of benzyl (3aS,7S,7aR)-7-amino-2,2-dimethyl-4,6,7,7a-tetrahydro-3aH-[1,3]dioxolo[4,5-c]pyridine-5-carboxylate (82)

To a solution of benzyl (3aS,7S,7aR)-7-hydroxy-2,2-dimethyl-4,6,7,7a-tetrahydro-3aH-[1,3]dioxolo[4,5-c]pyridine-5-carboxylate (81, 533 mg, 1.66 mmol, 1.00 equiv.) in THF (10 mL) trimethylphosphane (1 M in THF, 2.50 mL, 2.50 mmol, 1.50 equiv.) was added and the reaction mixture was stirred for 1 h until complete consumption of starting material was monitored by LC/MS. Water (1 mL) was added and the reaction mixture was concentrated in vacuo. The crude product was directly acetylated.

LC-MS (Method D):

Rₜ[min] (UV-signal 220 nm): 0.87

M[g/mol]: 307.1 [M+H⁺]

### Example 7.9: Synthesis of benzyl (3aS,7S,7aR)-7-acetamido-2,2-dimethyl-4,6,7,7a-tetrahydro-3aH-[1,3]dioxolo[4,5-c]pyridine-5-carboxylate (83)

Crude benzyl (3aS,7S,7aR)-7-amino-2,2-dimethyl-4,6,7,7a-tetrahydro-3aH-[1,3]dioxolo[4,5-c]pyridine-5-carboxylate (82, max. 1.66 mmol, 1.00 equiv.) was dissolved in EtOAc (10 mL), pyridine (0.70 mL, 8.65 mmol, 5.20 equiv.) and acetic anhydride (0.80 mL, 8.34 mL, 5.01 equiv.) were added and the reaction mixture was stirred overnight at r.t. LC/MS indicated full acetylation so the crude mixture was concentrated in vacuo and purified by flash chromatography (silica, 0-100% EtOAc in n-heptane) to yield benzyl (3aS,7S,7aR)-7-acetamido-2,2-dimethyl-4,6,7,7a-tetrahydro-3aH-[1,3]dioxolo[4,5-c]pyridine-5-carboxylate (83, 474 mg, 1.36 mmol, 82% over two steps) as a colorless oil.

LC-MS (Method D):

Rₜ[min] (UV-signal 220 nm): 1.08

M[g/mol]: 349.0 [M+H⁺]

### Example 7.10: Synthesis of (3aR,6R,6aR)-6-(hydroxymethyl)-2,2-dimethyl-6,6a-dihydro-3aH-furo[3,4-d][1,3]dioxol-4-one (85)

To a solution of D-ribono-1,4-lactone (84, 20.21 g, 136.45 mmol, 1.00 equiv.) in acetone (400 mL) was added concentrated aqueous HCl (37%, 9.50 mL, 113.77 mmol, 0.83 equiv.). The reaction mixture was stirred overnight until TLC indicated full conversion of the starting material. Solid NaHCO₃ was added, the reaction mixture was filtered and the filter cake was rinsed with acetone (100 mL). The combined filtrates were concentrated in vacuo and (3aR,6R,6aR)-6-(hydroxymethyl)-2,2-dimethyl-6,6a-dihydro-3aH-furo[3,4-d][1,3]dioxol-4-one (85, 25.21 g, 133.97 mmol, 98%) was directly used for the next step as crude product.

### Example 7.11: Synthesis of [(3aR,6R,6aR)-2,2-dimethyl-4-oxo-6,6a-dihydro-3aH-furo[3,4-d][1,3]dioxol-6-yl]methyl 4-methylbenzenesulfonate (86)

A solution of (3aR,6R,6aR)-6-(hydroxymethyl)-2,2-dimethyl-6,6a-dihydro-3aH-furo[3,4-d][1,3]dioxol-4-one (85, 5.22 g, 27.74 mmol, 1.00 equiv.) in anhydrous DCM (50 mL) was cooled to 0° C. and tosyl anhydride (13.38 g, 41.00 mmol, 1.48 equiv.) and pyridine (6.65 mL, 82.22 mmol, 2.96 equiv.) were added. The reaction mixture was stirred for 1.5 h at 0° C. and overnight at r.t. until LC/MS indicated full conversion of the starting material. EtOAc (300 mL) and aqueous 1 N HCl (150 mL) were added, the layers were separated, the aqueous layer was re-extracted with EtOAc (2×100 mL), the combined organic layers were washed with saturated aqueous NaHCO₃-solution (2×50 mL), saturated aqueous NaCl-solution (30 mL), dried (MgSO₄), filtered and concentrated in vacuo. The crude product [(3aR,6R,6aR)-2,2-dimethyl-4-oxo-6,6a-dihydro-3aH-furo[3,4-d][1,3]dioxol-6-yl]methyl 4-methylbenzenesulfonate (86, 9.31 g, 27.20 mmol, 98%) was directly used for the next step.

LC-MS (Method D):

Rₜ[min] (UV-signal 220 nm): 1.34

M[g/mol]: 343.0 [M+H−H₂O⁺]

### Example 7.12: Synthesis of (3aR,6R,6aR)-6-(aminomethyl)-2,2-dimethyl-6,6a-dihydro-3aH-furo[3,4-d][1,3]dioxol-4-one (87)

[(3aR,6R,6aR)-2,2-dimethyl-4-oxo-6,6a-dihydro-3aH-furo[3,4-d][1,3]dioxol-6-yl]methyl 4-methylbenzenesulfonate (86, 8.29 g, 24.23 mmol, 1.00 equiv.) was dissolved in DMF (10 mL), NaN₃ (6.47 g, 99.52 mmol, 4.11 equiv.) was added and the mixture was stirred at 70° C. for 6 d. EtOAc (300 mL) and water (100 mL) were added, the layers were separated, and the aqueous layer was re-extracted with EtOAc (3×50 mL). The combined organic layers were washed with saturated aqueous NaCl-solution (50 mL), dried (MgSO₄), filtered and concentrated in vacuo. The crude product was purified by flash chromatography (silica, 0-35% EtOAc in n-heptane) to yield (3aR,6R,6aR)-6-(aminomethyl)-2,2-dimethyl-6,6a-dihydro-3aH-furo[3,4-d][1,3]dioxol-4-one (87, 3.26 g, 15.29 mmol, 63%) as a colorless oil.

LC-MS (Method D):

Rₜ[min] (UV-signal 220 nm): 0.94

M[g/mol]: 214.0 [M+H⁺]

### Example 7.13: Synthesis of (3aR,7R,7aR)-7-hydroxy-2,2-dimethyl-5,6,7,7a-tetrahydro-3aH-[1,3]dioxolo[4,5-c]pyridin-4-one (88)

(3aR,6R,6aR)-6-(aminomethyl)-2,2-dimethyl-6,6a-dihydro-3aH-furo[3,4-d][1,3]dioxol-4-one (87, 2.83 g, 13.27 mmol, 1.00 equiv.) was dissolved in THF (50 mL), 10% Pd/C (0.14 g, 0.13 mmol, 0.01 equiv.) was added and the mixture was hydrogenated in an autoclave at r.t. and 4 bar hydrogen gas for 2 d. Since complete piperidine formation was detected by LC/MS, the reaction mixture was filtered and concentrated in vacuo. The crude product (3aR,7R,7aR)-7-hydroxy-2,2-dimethyl-5,6,7,7a-tetrahydro-3aH-[1,3]dioxolo[4,5-c]pyridin-4-one (88, 2.65 g, quant.) was obtained as a yellow solid and directly used for further reaction.

LC-MS (Method D):

Rₜ[min] (TIC-signal): 0.11

M[g/mol]: 188.2 [M+H⁺]

### Example 7.14: Synthesis of (3aR,7R,7aS)-7-[tert-butyl(dimethyl)silyl]oxy-2,2-dimethyl-5,6,7,7a-tetrahydro-3aH-[1,3]dioxolo[4,5-c]pyridin-4-one (89)

To a solution of (3aR,7R,7aR)-7-hydroxy-2,2-dimethyl-5,6,7,7a-tetrahydro-3aH-[1,3] dioxolo[4,5-c]pyridin-4-one (88, 2.88 g, 15.37 mmol, 1.00 equiv.) in DCM (100 mL) were added imidazole (3.30 g, 48.47 mmol, 3.15 equiv.) and TBSCl (4.64 g, 30.79 mmol, 2.00 equiv.). The reaction mixture was stirred overnight at r.t. until TLC indicated full conversion of the starting material. EtOAc (250 mL) and aqueous citric acid-solution (10%, 100 mL) were added, the layers were separated, and the aqueous layer was re-extracted with EtOAc (20 mL). The combined organic layers were washed with saturated aqueous NaCl-solution (30 mL), dried (MgSO₄), filtered and concentrated in vacuo. The crude product was purified by flash chromatography (silica, 5-80% EtOAc in n-heptane) to yield (3aR,7R,7aS)-7-[tert-butyl(dimethyl)silyl]oxy-2,2-dimethyl-5,6,7,7a-tetrahydro-3aH-[1,3]dioxolo[4,5-c]pyridin-4-one (89, 4.22 g, 14.00 mmol, 91%) as a colorless oil.

LC-MS (Method D):

Rₜ[min] (TIC-signal): 1.38

M[g/mol]: 302.1 [M+H⁺]

### Example 8: Synthesis of Linker Precursor

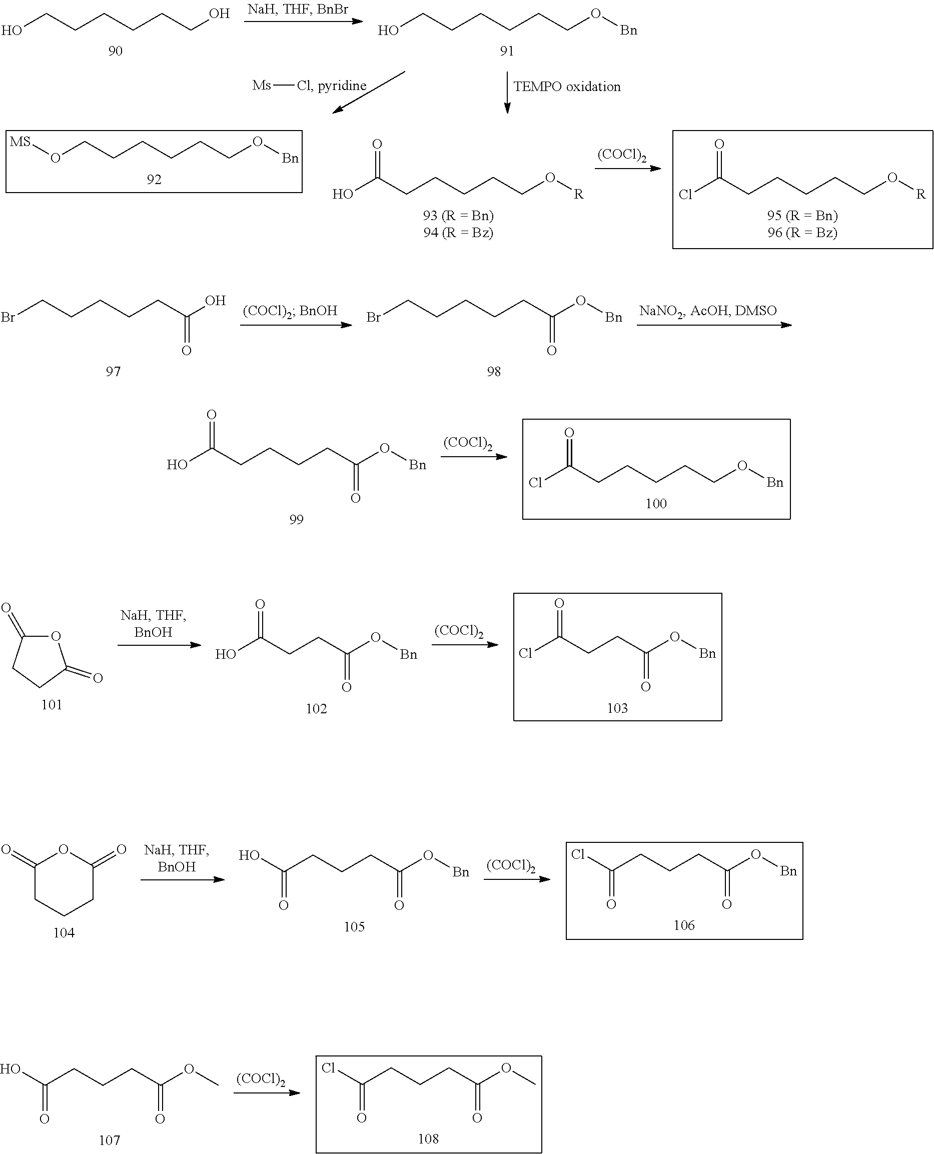

### Example 8.1: Synthesis of 6-benzyloxyhexan-1-ol (91)

A solution of hexane-1,6-diol (90, 9.96 g, 84.28 mmol, 1.00 equiv.) and tetrabutylammonium iodide (934 mg, 2.53 mmol, 0.03 equiv.) in anhydrous THF (100 mL) was cooled to 0° C. and sodium hydride (60% in mineral oil, 3.80 g, 95.01 mmol, 1.13 equiv.) was added in small portions. The reaction mixture was stirred for 10 min at 0° C. and 30 min at r.t. and benzyl bromide (15.86 g, 92.71 mmol, 1.10 equiv.) was added. The reaction mixture was stirred overnight at r.t. and saturated aqueous NH₄Cl-solution (100 mL) and EtOAc (250 mL) were added. The layers were separated, the organic layer was washed with saturated aqueous NaCl-solution (100 mL), dried (MgSO₄), filtered and concentrated in vacuo. The crude product was purified by flash chromatography (silica, 1-38% EtOAc in n-heptane) to yield 6-benzyl-oxyhexan-1-ol (91, 9.08 g, 43.58 mmol, 52%) as a colorless oil.

LC-MS (Method D):

Rₜ[min] (UV-signal 220 nm): 1.27

M[g/mol]: 209.1 [M+H⁺]

### Example 8.2: Synthesis of 6-benzyloxyhexyl methanesulfonate (92)

A solution of 6-benzyloxyhexan-1-ol (91, 9.08 g, 43.58 mmol, 1.00 equiv.) in anhydrous DCM (100 mL) was cooled to 0° C. and pyridine (17.62 mL, 217.88 mmol, 5.00 equiv.) and mesyl chloride (8.47 mL, 108.94 mmol, 2.50 equiv.) were added. The reaction mixture was stirred for 1.5 h at 0° C. until LC/MS indicated full conversion of the starting material. Aqueous 1 N HCl (100 mL) and EtOAc (250 mL) were added, the layers were separated, the organic layer was washed with saturated aqueous NaCl-solution (50 mL), dried (MgSO₄), filtered and concentrated in vacuo. The crude product was purified by flash chromatography (silica, 0-35% EtOAc in n-heptane) to yield 6-benzyloxyhexyl methanesulfonate (92, 10.59 g, 36.98 mmol, 85%) as a colorless oil.

LC-MS (Method D):

Rₜ[min] (UV-signal 220 nm): 1.49

M[g/mol]: 287.1 [M+H⁺]

### Example 8.3: Synthesis of 6-benzyloxyhexanoic acid (93)/6-benzyloxyhexanoic acid (94) (inseparable mixture

To a solution of 6-benzyloxyhexyl methanesulfonate (92, 9.22 g, 44.24 mmol, 1.00 equiv.) and TEMPO (693 mg, 4.44 mmol, 0.10 equiv.) in acetonitrile (50 mL) and aqueous pH 4 buffer solution (50 mL) were added simultaneously a solution of NaClO₂ (technical quality, approx. 80%, 30.01 g, approx. 6.00 equiv.) in water (50 mL) and an aqueous NaOCl-solution (technical quality, approx. 10%, 14.00 mL, approx. 0.51 equiv.). The reaction mixture was stirred overnight at r.t. until LC/MS indicated full conversion of the starting material. EtOAc (200 mL) and saturated aqueous Na₂SO₃-solution (100 mL) were added, the layers were separated, the aqueous layer was acidified by addition of aqueous 1 N HCl (50 mL) and re-extracted with EtOAc (3×50 mL). The combined organic layers were washed with saturated aqueous NaCl-solution (30 mL), dried (MgSO₄), filtered and concentrated in vacuo. The crude product (10.05 g, quant.) was directly used for the next step. LC/MS indicated a partial over-oxidation of the desired product 6-benzyloxyhexanoic acid (93) to 6-benzyloxyhexanoic acid (94) which occur as an inseparable mixture.

LC-MS (Method D):

Rₜ[min] (UV-signal 220 nm): 1.25

M[g/mol]: 223.2 [M+H⁺] (93, main product), 237.1 [M+H⁺] (94, minor product)

### Example 8.4: Synthesis of 6-benzyloxyhexanoyl chloride (95)/6-benzyloxyhexanoyl chloride (96) (inseparable mixture

To a solution of crude mixture of 6-benzyloxyhexanoic acid (93)/6-benzyloxyhexanoic acid (94) (max. 44.24 mmol, 1.00 equiv.) in DCM (50 mL) were added oxalyl chloride (6.00 mL, 67.18 mmol, 1.52 equiv.) and a drop of DMF for catalytic acceleration of the reaction. The reaction mixture was stirred overnight at r.t. until LC/MS indicated full conversion of the starting material (an aliquot of the reaction mixture was added to methanol and the acid chloride was detected as its corresponding methyl ester). The solvent was removed in vacuo and the crude product was used for the next step without purification.

LC-MS (Method D):

Rₜ[min] (UV-signal 220 nm): 1.54

M[g/mol]: 237.2 [M−Cl+OMe+H⁺] (95, main product), 251.1 [M−Cl+OMe+H⁺] (96, minor product)

### Example 8.5: Synthesis of benzyl 6-bromohexanoate (98)

To a solution of 6-bromohexanoic acid (97, 20.19 g, 103.51 mmol, 1.00 equiv.) in DCM (100 mL) were added oxalyl chloride (14.00 mL, 159.94 mmol, 1.55 equiv.) and a drop of DMF for catalytic acceleration of the reaction. The reaction mixture was stirred overnight at r.t. until LC/MS indicated full conversion of the starting material (an aliquot of the reaction mixture was added to methanol and the acid chloride was detected as its corresponding methyl ester). The solvent was removed in vacuo and the crude product was used for the next step without purification.

To a solution of the crude acid chloride (max. 103.51 mmol, 1.00 equiv.) in DCM (100 mL) were added benzyl alcohol (23.30 g, 215.46 mmol, 2.08 equiv.) and pyridine (26.00 mL, 321.46 mmol, 3.11 equiv.). The reaction mixture was stirred overnight at r.t. until LC/MS indicated full conversion of the starting material. Aqueous 1 N HCl (100 mL) and EtOAc (500 mL) were added, the layers were separated. The aqueous layer was re-extracted with EtOAc (3×50 mL), the organic layer was washed with saturated aqueous NaCl-solution (50 mL), dried (MgSO₄), filtered and concentrated in vacuo. The crude product was purified by flash chromatography (silica, 0-35% EtOAc in n-heptane) to yield benzyl 6-bromohexanoate (98, 22.05 g, 77.32 mmol, 75%) as a colorless oil.

LC-MS (Method D):

Rₜ[min] (UV-signal 220 nm): 1.68

M[g/mol]: 302.1/304.1 [M+H₂O+H⁺]

### Example 8.6: Synthesis of 6-benzyloxy-6-oxo-hexanoic acid (99)

To a solution of benzyl 6-bromohexanoate (98, 5.73 g, 20.09 mmol, 1.00 equiv.) in DMSO (40 mL) were added NaNO₂ (5.54 g, 80.30 mmol, 4.00 equiv.) and acetic acid (12 mL). The reaction mixture was stirred for 2 d at 40° C. EtOAc (300 mL) and aqueous 1 N HCl (100 mL) were added, the layers were separated, and the aqueous layer was extracted with EtOAc (3×20 mL). The combined organic layers were washed with saturated aqueous NaCl-solution (3×50 mL), dried (MgSO₄), filtered and concentrated in vacuo. The crude product was purified by flash chromatography (silica, 1-50% EtOAc in n-heptane) to yield 6-benzyloxy-6-oxo-hexanoic acid (99, 2.12 g, 8.97 mmol, 45%) as a colorless oil.

LC-MS (Method D):

Rₜ[min] (UV-signal 220 nm): 1.14

M[g/mol]: 237.1 [M+H⁺]

### Example 8.7: Synthesis of benzyl 6-chloro-6-oxo-hexanoate (100)

To a solution of 6-benzyloxy-6-oxo-hexanoic acid (99, 2.31 g, 9.77 mmol, 1.00 equiv.) in DCM (30 mL) were added oxalyl chloride (1.20 mL, 13.44 mmol, 1.37 equiv.) and a drop of DMF for catalytic acceleration of the reaction. The reaction mixture was stirred overnight at r.t. until LC/MS indicated full conversion of the starting material (an aliquot of the reaction mixture was added to methanol and the acid chloride was detected as its corresponding methyl ester). The solvent was removed in vacuo and the crude product benzyl 6-chloro-6-oxo-hexanoate (100) was used for the next step without purification.

LC-MS (Method D):

Rₜ[min] (UV-signal 220 nm): 1.39

M[g/mol]: 251.1 [M−Cl+OMe+H⁺]

### Example 8.8: Synthesis of 4-benzyloxy-4-oxo-butanoic acid (102)

A solution of benzyl alcohol (9.73 g, 89.93 mmol, 1.00 equiv.) in anhydrous THF (100 mL) was cooled to 0° C. and sodium hydride (60% in mineral oil, 3.74 g, 93.51 mmol, 1.04 equiv.) was added in small portions. The reaction mixture was stirred for 1 h at 0° C. and succinic anhydride (101, 9.00 g, 89.93 mmol, 1.00 equiv.) was added. The reaction mixture was stirred overnight at r.t. and water (300 mL), EtOAc (300 mL) and solid Na₂CO₃ (10.00 g, excess) were added. The layers were separated, the aqueous layer was re-extracted with EtOAc (50 mL), the combined organic layers were discarded and the aqueous layer was acidified to pH 1 by addition of aqueous 1 N HCl. The aqueous layer was extracted with EtOAc (3×50 mL), the combined organic layers were washed with saturated aqueous NaCl-solution (50 mL), dried (MgSO₄), filtered and concentrated in vacuo. The crude product 4-benzyloxy-4-oxo-butanoic acid (102, 12.29 g, 59.02 mmol, 66%) was obtained as a colorless solid which was sufficiently pure for the next conversions.

LC-MS (Method D):

Rₜ[min] (UV-signal 220 nm): 1.06

M[g/mol]: 209.1 [M+H⁺]

### Example 8.9: Synthesis of benzyl 4-chloro-4-oxo-butanoate (103)

To a solution of 4-benzyloxy-4-oxo-butanoic acid (102, 866 mg, 4.16 mmol, 1.00 equiv.) in DCM (10 mL) were added oxalyl chloride (0.72 mL, 8.32 mmol, 2.00 equiv.) and a drop of DMF for catalytic acceleration of the reaction. The reaction mixture was stirred overnight at r.t. until LC/MS indicated full conversion of the starting material (an aliquot of the reaction mixture was added to methanol and the acid chloride was detected as its corresponding methyl ester). The solvent was removed in vacuo and the crude product benzyl 4-chloro-4-oxo-butanoate (103) was used for the next step without purification.

LC-MS (Method D):

Rₜ[min] (UV-signal 220 nm): 1.35

M[g/mol]: 223.1 [M−Cl+OMe+H⁺]

### Example 8.10: Synthesis of 5-benzyloxy-5-oxo-pentanoic acid (105)

A solution of benzyl alcohol (9.49 g, 87.76 mmol, 1.00 equiv.) in anhydrous THF (100 mL) was cooled to 0° C. and sodium hydride (60% in mineral oil, 3.51 g, 87.76 mmol, 1.00 equiv.) was added in small portions. The reaction mixture was stirred for 1 h at 0° C. and glutaric anhydride (104, 10.01 g, 87.76 mmol, 1.00 equiv.) was added. The reaction mixture was stirred overnight at r.t. and water (300 mL); EtOAc (300 mL) and solid Na₂CO₃ (10.00 g, excess) were added. The layers were separated and the aqueous layer was re-extracted with EtOAc (50 mL). The combined organic layers were discarded and the aqueous layer was acidified to pH 1 by addition of aqueous 1 N HCl. The aqueous layer was extracted with EtOAc (3×50 mL), the combined organic layers were washed with saturated aqueous NaCl-solution (50 mL), dried (MgSO₄), filtered and concentrated in vacuo. The crude product 5-benzyloxy-5-oxo-pentanoic acid (105, 9.86 g, 44.37 mmol, 51%) was obtained as a colorless solid which was sufficiently pure for the next conversions.

LC-MS (Method D):

Rₜ[min] (UV-signal 220 nm): 1.11

M[g/mol]: 223.1 [M+H⁺]

### Example 8.11: Synthesis of benzyl 5-chloro-5-oxo-pentanoate (106)

To a solution of 5-benzyloxy-5-oxo-pentanoic acid (105, 930 mg, 4.18 mmol, 1.00 equiv.) in DCM (10 mL) were added oxalyl chloride (0.75 mL, 8.37 mmol, 2.00 equiv.) and a drop of DMF for catalytic acceleration of the reaction. The reaction mixture was stirred overnight at r.t. until LC/MS indicated full conversion of the starting material (an aliquot of the reaction mixture was added to methanol and the acid chloride was detected as its corresponding methyl ester). The solvent was removed in vacuo and the crude benzyl 5-chloro-5-oxo-pentanoate (106) product was used for the next step without purification.

LC-MS (Method D):

Rₜ[min] (UV-signal 220 nm): 1.37

M[g/mol]: 237.1 [M−Cl+OMe+H⁺]

### Example 8.12: Synthesis of methyl 5-chloro-5-oxo-pentanoate (108)

To a solution of commercially available 107 (3.02 g, 18.85 mmol, 1.00 equiv.) in DCM (20 mL) were added oxalyl chloride (3.30 mL, 37.69 mmol, 2.00 equiv.) and a drop of DMF for catalytic acceleration of the reaction. The reaction mixture was stirred overnight at r.t. until LC/MS indicated full conversion of the starting material (an aliquot of the reaction mixture was added to methanol and the acid chloride was detected as its corresponding methyl ester). The solvent was removed in vacuo and the crude product methyl 5-chloro-5-oxo-pentanoate (108) was used for the next step without purification.

LC-MS (Method D):

Rₜ[min] (UV-signal 220 nm): 0.98

M[g/mol]: 175.1 [M−Cl+OMe+H⁺]

### Example 9: Synthesis of Compounds 112, 117, 119, 120, and 121

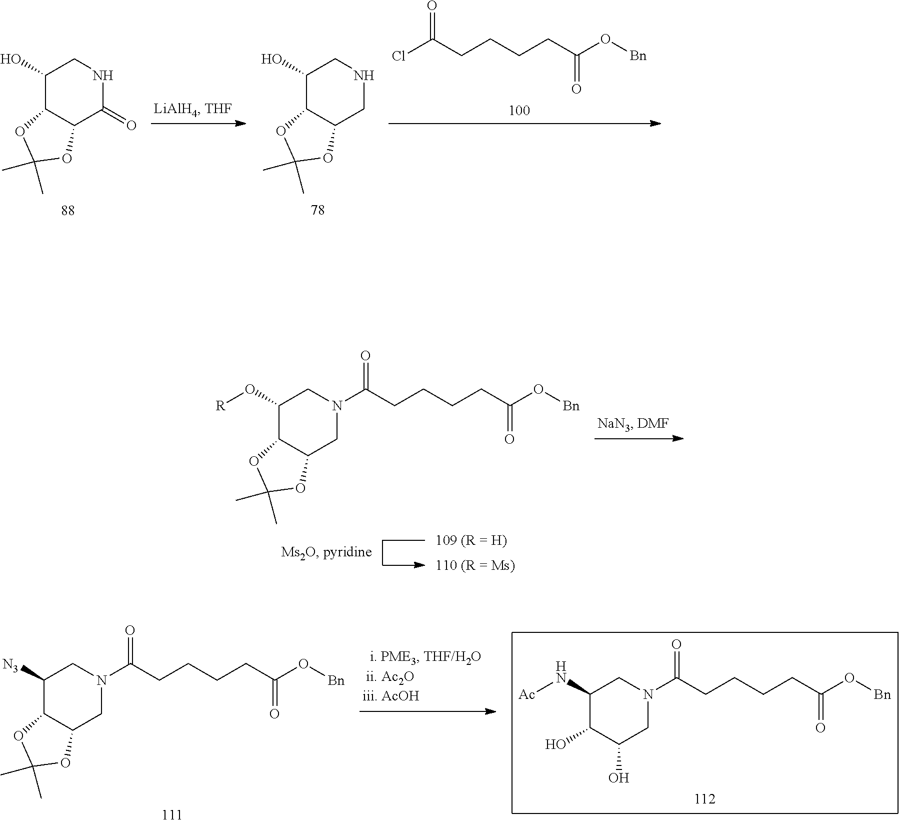

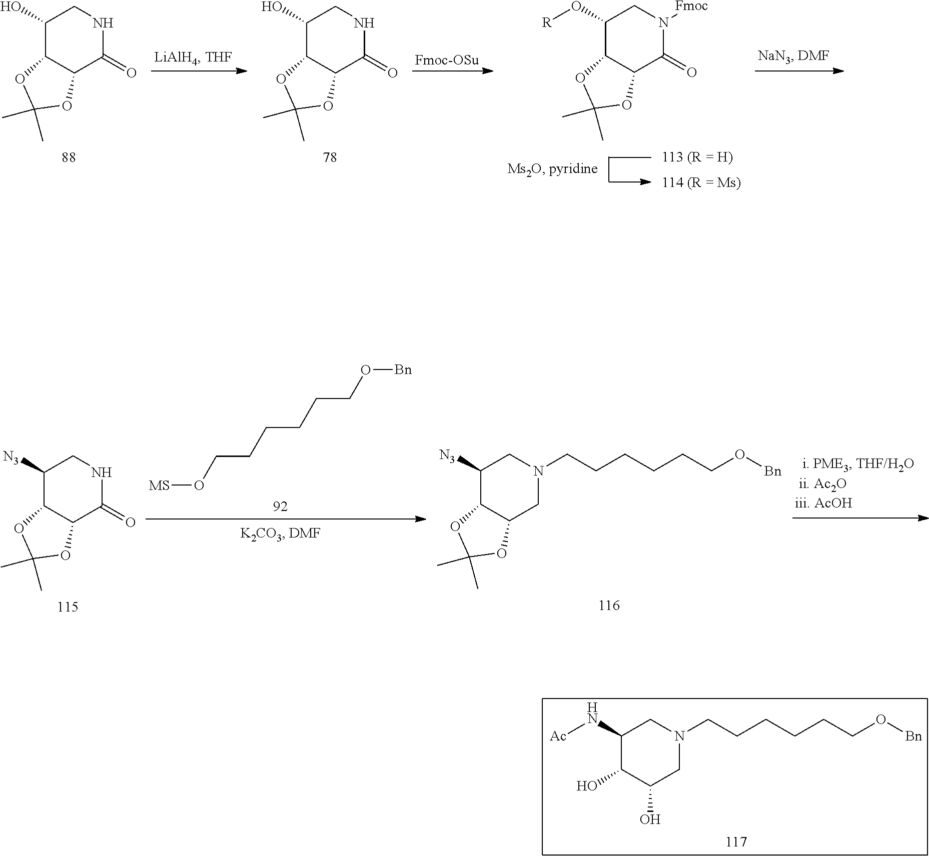

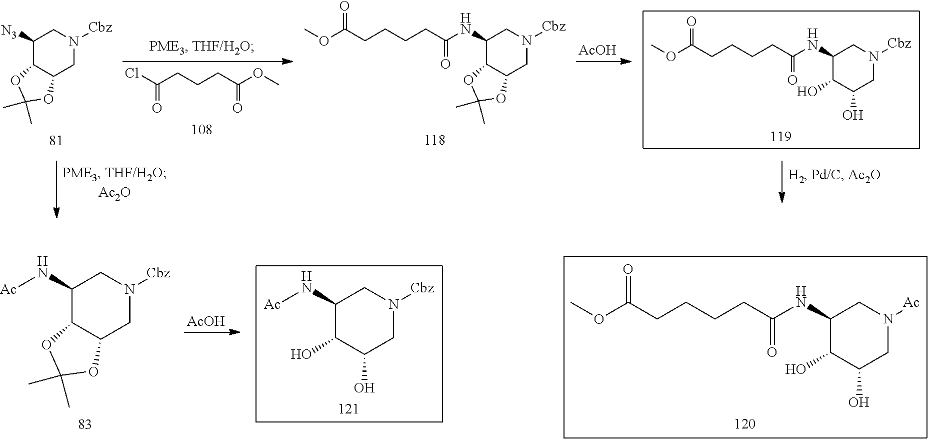

### Example 9.1: Synthesis of benzyl 6-[(3aS,7R,7aR)-7-hydroxy-2,2-dimethyl-4,6,7,7a-tetrahydro-3aH-[1,3]dioxolo[4,5-c]pyridin-5-yl]-6-oxo-hexanoate (109)

To a solution of (3aR,7R,7aR)-7-hydroxy-2,2-dimethyl-5,6,7,7a-tetrahydro-3aH-[1,3]dioxolo [4,5-c]pyridin-4-one (88, 1.20 g, 6.41 mmol, 1.00 equiv.) in anhydrous THF (100 mL) was added LiAlH₄ (15% in toluene/THF, 3.5 M, 5.00 mL, 17.50 mmol, 2.73 equiv.) and the reaction mixture was stirred overnight at r.t. Excess of LiAlH₄ were quenched by careful addition of saturated aqueous NaHCO₃-solution (50 mL) and water (20 mL). The crude product (3aS,7R,7aR)-2,2-dimethyl-3a,4,5,6,7,7a-hexahydro-[1,3]dioxolo[4,5-c]pyridin-7-ol (78) was directly used as THF/saturated aqueous NaHCO₃-solution and a solution of acid chloride benzyl 6-chloro-6-oxo-hexanoate (100, 2.49 g, 9.79 mmol, 1.53 equiv.) in THF (10 mL) was added. The reaction mixture was stirred for 6 h at r.t. EtOAc (200 mL) and water (50 mL) were added and the reaction mixture was filtered over Celite to remove insoluble aluminium salts. The layers were separated, the organic layer was washed with aqueous 2 N NaOH solution (3×30 mL), saturated aqueous NaCl-solution (30 mL), dried (MgSO₄), filtered and concentrated in vacuo. The crude product was purified by flash chromatography (silica, 1-80% EtOAc in n-heptane) to yield benzyl 6-[(3aS,7R,7aR)-7-hydroxy-2,2-dimethyl-4,6,7,7a-tetrahydro-3aH-[1,3]dioxolo[4,5-c]pyridin-5-yl]-6-oxo-hexanoate (109, 1.69 g, 4.32 mmol, 67% over two steps) as a colorless oil.

LC-MS (Method D):

Rₜ[min] (UV-signal 220 nm): 1.09

M[g/mol]: 392.2 [M+H⁺]

### Example 9.2: Synthesis of benzyl 6-[(3aS,7R,7aS)-2,2-dimethyl-7-methylsulfonyloxy-4,6,7,7a-tetrahydro-3aH-[1,3]dioxolo[4,5-c]pyridin-5-yl]-6-oxo-hexanoate (110)

A solution of benzyl 6-[(3aS,7R,7aR)-7-hydroxy-2,2-dimethyl-4,6,7,7a-tetrahydro-3aH-[1,3]dioxolo[4,5-c]pyridin-5-yl]-6-oxo-hexanoate (109, 1.69 g, 4.32 mmol, 1.00 equiv.) in anhydrous DCM (30 mL) was cooled to 0° C. and pyridine (1.10 mL, 13.53 mmol, 3.13 equiv.) and mesyl anhydride (1.17 g, 6.59 mmol, 1.53 equiv.) were added. The reaction mixture was stirred for 3 h at 0° C. until LC/MS indicated full conversion of the starting material. Aqueous 1 N HCl (100 mL) and EtOAc (250 mL) were added, the layers were separated, the organic layer was washed with saturated aqueous NaCl-solution (50 mL), dried (MgSO₄), filtered and concentrated in vacuo. The crude product benzyl 6-[(3aS,7R,7aS)-2,2-dimethyl-7-methylsulfonyloxy-4,6,7,7a-tetrahydro-3aH-[1,3]dioxolo[4,5-c]pyridin-5-yl]-6-oxo-hexanoate (110) was directly used for the next step.

LC-MS (Method D):

Rₜ[min] (UV-signal 220 nm): 1.30

M[g/mol]: 470.1 [M+H⁺]

### Example 9.3: Synthesis of benzyl 6-[(3aS,7S,7aR)-7-azido-2,2-dimethyl-4,6,7,7a-tetrahydro-3aH-[1,3]dioxolo[4,5-c]pyridin-5-yl]-6-oxo-hexanoate (111)

The crude product benzyl 6-[(3aS,7R,7aS)-2,2-dimethyl-7-methylsulfonyloxy-4,6,7,7a-tetrahydro-3aH-[1,3]dioxolo[4,5-c]pyridin-5-yl]-6-oxo-hexanoate (110, max. 4.32 mmol, 1.00 equiv.) was dissolved in DMF (5 mL), NaN₃ (1.14 g, 17.49 mmol, 4.04 equiv.) and 15-crown-5 ether (1.51 g, 6.86 mmol, 1.58 equiv.) were added and the mixture was stirred at 100° C. for 1 d. EtOAc (100 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-80% EtOAc in n-heptane) to yield benzyl 6-[(3aS,7S,7aR)-7-azido-2,2-dimethyl-4,6,7,7a-tetrahydro-3aH-[1,3]dioxolo[4,5-c]pyridin-5-yl]-6-oxo-hexanoate (111, 593 mg, 1.42 mmol, 33% over two steps) as a colorless oil.

LC-MS (Method D):

Rₜ[min] (UV-signal 220 nm): 1.44

M[g/mol]: 417.2 [M+H⁺]

### Example 9.4: Synthesis of benzyl 6-[(3S,4R,5S)-3-acetamido-4,5-dihydroxy-1-piperidyl]-6-oxo-hexanoate (112)

To a solution of benzyl 6-[(3aS,7S,7aR)-7-azido-2,2-dimethyl-4,6,7,7a-tetrahydro-3aH-[1,3]dioxolo[4,5-c]pyridin-5-yl]-6-oxo-hexanoate (111, 95 mg, 0.228 mmol, 1.00 equiv.) in THF (5 mL) and water (0.1 mL) was added PMe₃ (1 N in THF, 0.40 mL, 0.40 mmol, 1.75 equiv.) and the reaction mixture was stirred for 1 h at r.t. Since LC/MS indicated full conversion of the starting material, water (0.9 mL) and acetic acid (5 mL) were added and the reaction mixture was stirred for 3 h at 80° C. for full hydrolysis of the formed iminophosphorane. Acetic anhydride (0.25 mL) was added at r.t. and the reaction mixture was stirred for 1 h. EtOAc (30 mL) and saturated aqueous NaHCO₃-solution (50 mL) were added, the layers were separated, and the aqueous layers was re-extracted with EtOAc (3×10 mL). The combined organic layers were washed with saturated aqueous NaCl-solution (20 mL), dried (MgSO₄), filtered and concentrated in vacuo. The crude product was dissolved in acetic acid (4 mL) and water (1 mL) and heated to 80° C. for 1 h until full acetonide deprotection was monitored by LC/MS. The solvent was removed in vacuo and the crude mixture was purified by HPLC (15 min, 5-90% acetonitrile in water+0.1% TFA) to yield benzyl 6-[(3S,4R,5S)-3-acetamido-4,5-dihydroxy-1-piperidyl]-6-oxo-hexanoate (112, 83 mg, 0.211 mmol, 93%) as a colorless solid.

LC-MS (Method D):

Rₜ[min] (UV-signal 220 nm): 0.90

M[g/mol]: 393.2 [M+H⁺]

¹H NMR (400 MHz, DMSO-d6) δ[ppm]: 7.88/7.69 (d, J=7.5/8.4 Hz, 1H), 7.45-7.35 (m, 5H), 5.12 (s, 2H), 4.14-4.07 (m, 1H), 4.00-3.78 (m, 7H), 3.18-3.05 (m, 1H), 2.40-2.17 (m, 4H), 1.81/1.80 (s, 3H), 1.58-1.43 (m, 4H) (two main conformers due to amide resonance)

### Example 9.5: Synthesis of 9H-fluoren-9-ylmethyl (3aS,7R,7aR)-7-hydroxy-2,2-dimethyl-4,6,7,7a-tetrahydro-3aH-[1,3]dioxolo[4,5-c]pyridine-5-carboxylate (113)

To a solution of (3aR,7R,7aR)-7-hydroxy-2,2-dimethyl-5,6,7,7a-tetrahydro-3aH-[1,3]dioxolo [4,5-c]pyridin-4-one (88, 1.24 g, 6.61 mmol, 1.00 equiv.) in anhydrous THF (100 mL) was added LiAlH₄ (15% in toluene/THF, 3.5 M, 5.00 mL, 17.50 mmol, 2.65 equiv.). The reaction mixture was stirred overnight at r.t. and saturated aqueous NaHCO₃-solution (50 mL) and water (20 mL) were added. The crude product 3aS,7R,7aR)-2,2-dimethyl-3a,4,5,6,7,7a-hexahydro-[1,3]dioxolo[4,5-c]pyridin-7-ol (78) was directly used as THF/aqueous NaHCO₃-solution and a solution of FmocOSu (3.38 g, 10.02 mmol, 1.51 equiv.) in THF (10 mL) was added. The reaction mixture was stirred overnight at r.t. EtOAc (100 mL) and saturated aqueous NaHCO₃-solution (50 mL) were added and the reaction mixture was filtered over Celite to remove insoluble aluminium salts. The layers were separated, the aqueous layer was re-extracted with EtOAc (3×30 mL), the combined organic layers were washed with saturated aqueous NaCl-solution (20 mL), dried (MgSO₄), filtered and concentrated in vacuo. The crude product was purified by flash chromatography (silica, 1-30% EtOAc in n-heptane) to yield 9H-fluoren-9-ylmethyl(3aS,7R,7aR)-7-hydroxy-2,2-dimethyl-4,6,7,7a-tetrahydro-3aH-[1,3]dioxolo[4,5-c] pyridine-5-carboxylate (113, 1.99 g, 5.03 mmol, 76% over two steps) as a colorless oil.

LC-MS (Method D):

Rₜ[min] (UV-signal 220 nm): 1.35

M[g/mol]: 338.1 [M−C₃H₆O+H⁺]

### Example 9.6: Synthesis of 9H-fluoren-9-ylmethyl (3aS,7R,7aS)-2,2-dimethyl-7-methylsulfonyloxy-4,6,7,7a-tetrahydro-3aH-[1,3]dioxolo[4,5-c]pyridine-5-carboxylate (114)

A solution of 9H-fluoren-9-ylmethyl (3aS,7R,7aR)-7-hydroxy-2,2-dimethyl-4,6,7,7a-tetrahydro-3aH-[1,3]dioxolo[4,5-c]pyridine-5-carboxylate (113, 1.99 g, 5.03 mmol, 1.00 equiv.) in anhydrous DCM (50 mL) was cooled to 0° C. and pyridine (1.30 mL, 16.80 mmol, 3.34 equiv.) and mesyl anhydride (1.49 g, 8.54 mmol, 1.70 equiv.) were added. The reaction mixture was stirred for 3 h at 0° C. until LC/MS indicated full conversion of the starting material. Aqueous 1 N HCl (100 mL) and EtOAc (250 mL) were added, the layers were separated, the organic layer was washed with saturated aqueous NaCl-solution (50 mL), dried (MgSO₄), filtered and concentrated in vacuo. The crude product 9H-fluoren-9-ylmethyl (3aS,7R,7aS)-2,2-dimethyl-7-methylsulfonyloxy-4,6,7,7a-tetrahydro-3aH-[1,3]dioxolo[4,5-c]pyridine-5-carbo-xylate (114) was directly used for the next step.

LC-MS (Method D):

Rₜ[min] (UV-signal 220 nm): 1.57

M[g/mol]: 474.0 [M+H⁺]

### Example 9.7: Synthesis of (3aS,7S,7aR)-7-azido-2,2-dimethyl-3a,4,5,6,7,7a-hexahydro-[1,3]dioxolo[4,5-c]pyridine (115)

The crude product 9H-fluoren-9-ylmethyl (3aS,7R,7aS)-2,2-dimethyl-7-methylsulfonyloxy-4,6,7,7a-tetrahydro-3aH-[1,3]dioxolo[4,5-c]pyridine-5-carboxylate (114, max. 5.03 mmol, 1.00 equiv.) was dissolved in DMF (10 mL), NaN₃ (1.32 g, 20.26 mmol, 4.03 equiv.) and 15-crown-5 ether (1.67 g, 7.60 mmol, 1.51 equiv.) were added and the mixture was stirred at 100° C. for 1 d. LC/MS indicated full unintended Fmoc-deprotection and formation of the azide (3aS,7S,7aR)-7-azido-2,2-dimethyl-3a,4,5,6,7,7a-hexahydro-[1,3]dioxolo[4,5-c]pyridine (115). EtOAc (100 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-100% EtOAc in n-heptane) to yield (3aS,7S,7aR)-7-azido-2,2-dimethyl-3a,4,5,6,7,7a-hexahydro-[1,3]dioxolo[4,5-c]pyridine (115) (857 mg, 4.32 mmol, 85%) as a colorless oil.

LC-MS (Method D):

Rₜ[min] (TIC-signal): 0.37

M[g/mol]: 199.1 [M+H⁺]

### Example 9.8: Synthesis of (3aS,7S,7aR)-7-azido-5-(6-benzyloxyhexyl)-2,2-dimethyl-4,6,7,7a-tetrahydro-3aH-[1,3]dioxolo[4,5-c]pyridine (116)

(3aS,7S,7aR)-7-azido-2,2-dimethyl-3a,4,5,6,7,7a-hexahydro-[1,3]dioxolo[4,5-c]pyridine (115, 570 mg, 2.88 mmol, 1.00 equiv.) was dissolved in DMF (5 mL), NaI (665 mg, 4.44 mmol, 1.54 equiv.), K₂CO₃ (2.00 g, 14.45 mmol, 5.02 equiv.) and 6-benzyloxyhexyl methanesulfonate (92, 1.06 g, 3.69 mmol, 1.28 equiv.) were added and the mixture was stirred at r.t. for 3 d. EtOAc (100 mL) and water (50 mL) were added, the layers were separated, and the aqueous layer was re-extracted with EtOAc (3×20 mL). The combined organic layers were washed with saturated aqueous NaCl-solution (30 mL), dried (MgSO₄), filtered and concentrated in vacuo. The crude product was purified by flash chromatography (silica, 0-40% EtOAc in n-heptane) to yield (3aS,7S,7aR)-7-azido-5-(6-benzyloxyhexyl)-2,2-dimethyl-4,6,7,7a-tetrahydro-3aH-[1,3]dioxolo[4,5-c]pyridine (116, 345 mg, 0.88 mmol, 31%) as a colorless oil.

LC-MS (Method D):

Rₜ[min] (UV-signal 220 nm): 1.14

M[g/mol]: 389.2 [M+H⁺]

### Example 9.9: Synthesis of N-[(3S,4R,5S)-1-(6-benzyloxyhexyl)-4,5-dihydroxy-3-piperidyl]acetamide (117)

To a solution of (3aS,7S,7aR)-7-azido-5-(6-benzyloxyhexyl)-2,2-dimethyl-4,6,7,7a-tetrahydro-3aH-[1,3]dioxolo[4,5-c]pyridine (116, 86 mg, 0.221 mmol, 1.00 equiv.) in THF (5 mL) and water (0.1 mL) was added PMe₃ (1 N in THF, 0.40 mL, 0.40 mmol, 1.75 equiv.) and the reaction mixture was stirred for 1 h at r.t. Since LC/MS indicated full conversion of the starting material, the reaction mixture was concentrated in vacuo. The residue was dissolved in EtOAc (5 mL), acetic anhydride (0.20 mL) and pyridine (0.10 mL) were added at r.t. and the reaction mixture was stirred for 1 h. Since LC/MS indicated full formation of the acetamide, the reaction mixture was concentrated in vacuo. The crude product was dissolved in acetic acid (4 mL) and water (1 mL) and heated to 80° C. for 3 h until full acetonide deprotection was monitored by LC/MS. The solvent was removed in vacuo and the crude mixture was purified by HPLC (15 min, 5-90% acetonitrile in water+0.1% TFA) to yield N-[(3S,4R,5S)-1-(6-benzyloxyhexyl)-4,5-dihydroxy-3-piperidyl]acetamide (117, TFA-salt, 65 mg, 0.138 mmol, 62%) as a colorless solid.

LC-MS (Method D):

Rₜ[min] (UV-signal 220 nm): 0.80

M[g/mol]: 365.2 [M+H⁺]

¹H NMR (400 MHz, DMSO-d₆) δ[ppm]: 9.55 (s, br, 1H), 7.93 (d, J=7.9 Hz, 1H), 7.40-7.24 (m, 5H), 5.60-5.35 (m, 2H), 4.44 (s, 2H), 4.12-3.98 (m, 1H), 3.80-3.71 (m, 2H), 3.51-3.37 (m, 2H), 3.21-3.03 (m, 4H), 2.94-2.70 (m, 2H), 1.86 (s, 3H), 1.68-1.49 (m, 4H), 1.41-1.21 (m, 4H).

### Example 9.10: Synthesis of benzyl (3aS,7S,7aR)-7-[(6-methoxy-6-oxo-hexanoyl)amino]-2,2-dimethyl-4,6,7,7a-tetrahydro-3aH-[1,3]dioxolo[4,5-c]pyridine-5-carboxylate (118)

To a solution of benzyl (3aS,7S,7aR)-7-hydroxy-2,2-dimethyl-4,6,7,7a-tetrahydro-3aH-[1,3]dioxolo[4,5-c]pyridine-5-carboxylate (81, 90 mg, 0.27 mmol, 1.00 equiv.) in THF (5 mL) and water (0.1 mL) was added PMe₃ (1 N in THF, 0.41 mL, 0.41 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL) and used as a stock solution for parallel reactions. 8.00 mL of this stock solution (0.11 mmol) were used, acid chlorid methyl 5-chloro-5-oxo-pentanoate (108, 39 mg, 0.22 mmol, 2.00 equiv.) and pyridine (0.05 mL, approx. 6 equiv.) were added at r.t. and the reaction mixture was stirred for 1 h. Since LC/MS indicated full formation of the acetamide, the reaction mixture was concentrated in vacuo. The crude product benzyl (3aS,7S,7aR)-7-[(6-methoxy-6-oxo-hexanoyl)amino]-2,2-dimethyl-4,6,7,7a-tetrahydro-3aH-[1,3]dioxolo[4,5-c]pyridine-5-carboxylate (118) was directly used for the next step.

LC-MS (Method D):

Rₜ[min] (UV-signal 220 nm): 1.21

M[g/mol]: 449.2 [M+H⁺]

### Example 9.11: Synthesis of benzyl (3S,4R,5S)-3,4-dihydroxy-5-[(6-methoxy-6-oxo-hexanoyl)amino]piperidine-1-carboxylate (119)

The crude product benzyl (3aS,7S,7aR)-7-[(6-methoxy-6-oxo-hexanoyl)amino]-2,2-dimethyl-4,6,7,7a-tetrahydro-3aH-[1,3]dioxolo[4,5-c]pyridine-5-carboxylate (118, max. 0.11 mmol, 1.00 equiv.) was dissolved in acetic acid (4 mL) and water (1 mL) and heated to 80° C. for 3 h until full acetonide deprotection was monitored by LC/MS. The solvent was removed in vacuo and the crude mixture was purified by HPLC (15 min, 10-60% acetonitrile in water+0.1% TFA) to yield benzyl (3S,4R,5S)-3,4-dihydroxy-5-[(6-methoxy-6-oxo-hexanoyl)amino] piperidine-1-carboxylate (119, 32 mg, 0.08 mmol, 72% over two steps) as a colorless solid.

LC-MS (Method D):

Rₜ[min] (UV-signal 220 nm): 0.99

M[g/mol]: 409.2 [M+H⁺]

¹H NMR (400 MHz, DMSO-d6) δ[ppm]: 7.69 (m, 1H), 7.41-7.27 (m, 5H), 5.07 (d, J=12.8 Hz, 1H), 5.03 (d, J=12.8 Hz, 1H), 4.74 (d, J=4.5 Hz, 1H), 4.69 (s, br, 1H), 3.84 (m, 1H), 3.78-3.59 (m, 3H), 3.57 (s, 3H), 3.48-3.38/3.24-3.15 (m, 2H), 3.12-3.00/2.84-2.74 (m, 1H), 2.34-2.24 (m, 2H), 2.12-2.04 (m, 2H), 1.54-1.42 (m, 4H) (two main conformers due to amide resonance).

### Example 9.12: Synthesis of methyl 6-[[(3S,4R,5S)-1-acetyl-4,5-dihydroxy-3-piperidyl]amino]-6-oxo-hexanoate (120)

A solution of benzyl (3S,4R,5S)-3,4-dihydroxy-5-[(6-methoxy-6-oxo-hexanoyl)amino]piperi-dine-1-carboxylate (119, 25 mg, 0.061 mmol, 1.00 equiv.) in EtOH (10 mL) was hydrogenated in an H-Cube (10% Pd(OH)₂/C, full hydrogen mode, 60° C., flow 1 mL/min). Full hydrogenation was detected after three cycles. The solvent was removed in vacuo, the crude product was dissolved in EtOAc (10 mL), acetic anhydride (0.02 mL, approx. 4 equiv.) and pyridine (0.02 mL, approx. 4 equiv.) were added at r.t. and the reaction mixture was stirred for 1 h. Since LC/MS indicated full formation of the acetamide, the reaction was stopped by the addition of water (1 mL) and the mixture was concentrated in vacuo. The crude mixture was purified by HPLC (15 min, 2-52% acetonitrile in water+0.1% TFA) to yield methyl 6-[[(3S,4R,5S)-1-acetyl-4,5-dihydroxy-3-piperidyl]amino]-6-oxo-hexanoate (120, 5 mg, 0.016 mmol, 26%) as a colorless solid.

LC-MS (Method D):

Rₜ[min] (UV-signal 220 nm): 0.48

M[g/mol]: 317.2 [M+H⁺]

¹H NMR (400 MHz, DMSO-d₆) δ[ppm]: 7.80/7.59 (d, J=7.5/8.2 Hz, 1H), 4.93-4.47 (m, 2H), 4.09-4.02/3.85-3.78 (m, 1H), 3.78-3.70 (m, 1H), 3.68-3.54 (m, 1H), 3.58 (s, 3H), 3.53-3.08 (m, 4H), 2.34-2.27 (m, 2H), 2.14-2.04 (m, 2H), 1.98/1.91 (s, 3H), 1.55-1.45 (m, 4H) (two main conformers due to amide resonance).

### Example 9.13: Synthesis of benzyl (3S,4R,5S)-3-acetamido-4,5-dihydroxy-piperidine-1-carboxylate (121)

To a solution of benzyl (3aS,7S,7aR)-7-hydroxy-2,2-dimethyl-4,6,7,7a-tetrahydro-3aH-[1,3]dioxolo[4,5-c]pyridine-5-carboxylate (81, 90 mg, 0.27 mmol, 1.00 equiv.) in THF (5 mL) and water (0.1 mL) was added PMe₃ (1 N in THF, 0.41 mL, 0.41 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL) and used as a stock solution for parallel reactions. 8.00 mL of this stock solution (0.11 mmol) were used, acetic anhydride (0.10 mL, approx. 10 equiv.) and pyridine (0.05 mL, approx. 6 equiv.) were added at r.t. and the reaction mixture was stirred for 1 h. Since LC/MS indicated full formation of the acetamide, the reaction mixture was concentrated in vacuo. The crude product was dissolved in acetic acid (4 mL) and water (1 mL) and heated to 80° C. for 3 h until full acetonide deprotection was monitored by LC/MS. The solvent was removed in vacuo and the crude mixture was purified by HPLC (15 min, 1-50% acetonitrile in water+0.1% TFA) to yield benzyl (3S,4R,5S)-3-acetamido-4,5-dihydroxy-piperidine-1-carboxylate (121, 27 mg, 0.087 mmol, 80%) as a colorless solid.

LC-MS (Method D):

Rₜ[min] (UV-signal 220 nm): 0.81

M[g/mol]: 309.2 [M+H⁺]

¹H NMR (400 MHz, DMSO-d₆) δ[ppm]: 7.75 (m, 1H), 7.40-7.27 (m, 5H), 5.14-4.20 (s, br, 2H), 5.08 (d, J=12.8 Hz, 1H), 5.03 (d, J=12.8 Hz, 1H), 3.83 (m, 1H), 3.79-3.57 (m, 3H), 3.52-3.15 (m, 2H), 3.10-2.99/2.85-2.74 (m, 1H), 1.80 (s, 3H), (two main conformers due to amide resonance).

### Example 10: Synthesis of compounds 128, 129, 131, and 132

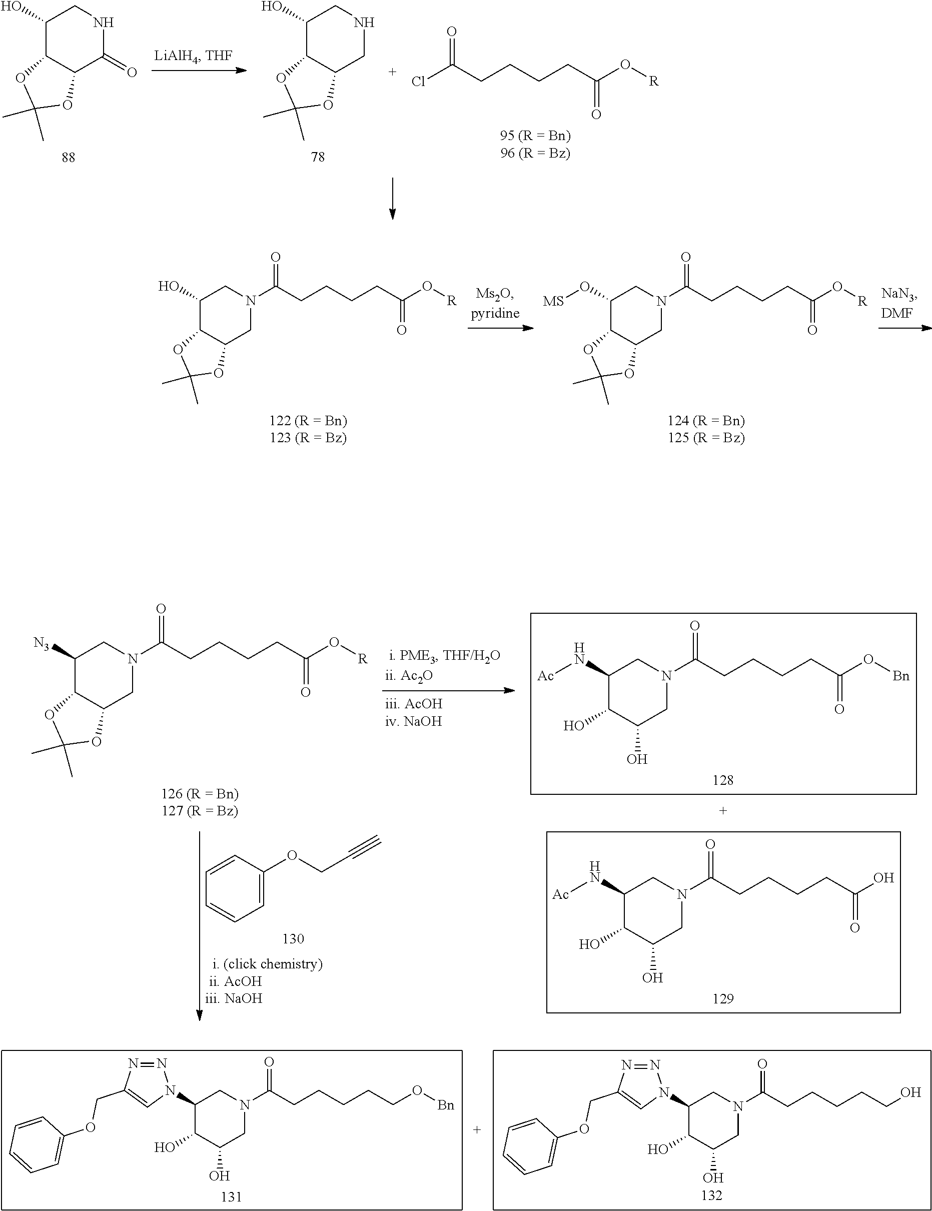

### Example 10.1: Synthesis of 1-[(3aS,7R,7aR)-7-hydroxy-2,2-dimethyl-4,6,7,7a-tetrahydro-3aH-[1,3]dioxolo[4,5-c]pyridin-5-yl]-6-benzyloxy-hexan-1-one (122) and [6-[(3aS,7R,7aR)-7-hydroxy-2,2-dimethyl-4,6,7,7a-tetrahydro-3aH-[1,3]dioxolo[4,5-c]pyridin-5-yl]-6-oxo-hexyl] benzoate (123, inseparable mixture

To a solution of (3aR,7R,7aR)-7-hydroxy-2,2-dimethyl-5,6,7,7a-tetrahydro-3aH-[1,3]dioxolo [4,5-c]pyridin-4-one (88, 0.93 g, 4.97 mmol, 1.00 equiv.) in anhydrous THF (100 mL) was added LiAlH₄ (15% in toluene/THF, 3.5 M, 5.00 mL, 17.50 mmol, 3.52 equiv.). The reaction mixture was stirred overnight at r.t. and saturated aqueous NaHCO₃-solution (50 mL) and water (20 mL) were added. The crude product (3aS,7R,7aR)-2,2-dimethyl-3a,4,5,6,7,7a-hexahydro-[1,3]dioxolo[4,5-c]pyridin-7-ol (78) was directly used as THF/saturated aqueous NaHCO₃-solution and a solution of acid chlorides 6-benzyloxyhexanoyl chloride (95)/6-benzyloxyhexanoyl chloride (96) (inseparable mixture, 2.36 g, 9.81 mmol, 1.97 equiv.) in THF (10 mL) was added. The reaction mixture was stirred for 6 h at r.t. EtOAc (200 mL) and water (50 mL) were added and the reaction mixture was filtered over Celite to remove insoluble aluminium salts. The layers were separated, the organic layer was washed with aqueous 2 N NaOH solution (3×30 mL), saturated aqueous NaCl-solution (30 mL), dried (MgSO₄), filtered and concentrated in vacuo. The crude product was purified by flash chromatography (silica, 5-100% EtOAc in n-heptane) to yield 1-[(3aS,7R,7aR)-7-hydroxy-2,2-dimethyl-4,6,7,7a-tetrahydro-3aH-[1,3]dioxolo[4,5-c]pyridin-5-yl]-6-benzyloxy-hexan-1-one (122) and [6-[(3aS,7R,7aR)-7-hydroxy-2,2-dimethyl-4,6,7,7a-tetrahydro-3aH-[1,3]dioxolo[4,5-c]pyridin-5-yl]-6-oxo-hexyl] benzoate (123, inseparable mixture, 0.73 g, 1.93 mmol, 39% over two steps) as a colorless oil.

LC-MS (Method D):

Rₜ[min] (UV-signal 220 nm): 1.19

M[g/mol]: 378.2 [M+H⁺] (122, main product), 392.1 [M+H⁺] (123, minor product) Example 10.2: Synthesis of [(3aS,7R,7aS)-5-(6-benzyloxyhexanoyl)-2,2-dimethyl-4,6,7,7a-tetrahydro-3aH-[1,3]dioxolo[4,5-c]pyridin-7-yl] methanesulfonate (124) and [6-[(3aS,7R,7aS)-2,2-dimethyl-7-methylsulfonyloxy-4,6,7,7a-tetrahydro-3aH-[1,3]dioxolo[4,5-c]pyridin-5-yl]-6-oxo-hexyl] benzoate (125, inseparable mixture)

A solution of 1-[(3aS,7R,7aR)-7-hydroxy-2,2-dimethyl-4,6,7,7a-tetrahydro-3aH-[1,3]dioxolo [4,5-c]pyridin-5-yl]-6-benzyloxy-hexan-1-one (122) and [6-[(3aS,7R,7aR)-7-hydroxy-2,2-dimethyl-4,6,7,7a-tetrahydro-3aH-[1,3]dioxolo[4,5-c]pyridin-5-yl]-6-oxo-hexyl]benzoate (123, inseparable mixture, 0.73 g, 1.93 mmol, 1.00 equiv.) in anhydrous DCM (10 mL) was cooled to 0° C. and pyridine (0.40 mL, 4.92 mmol, 2.54 equiv.) and mesyl anhydride (0.57 g, 3.27 mmol, 1.69 equiv.) were added. The reaction mixture was stirred for 3 h at 0° C. until LC/MS indicated full conversion of the starting material. Aqueous 1 N HCl (30 mL) and EtOAc (50 mL) were added, the layers were separated, the organic layer was washed with saturated aqueous NaCl-solution (30 mL), dried (MgSO₄), filtered and concentrated in vacuo. The crude product mixture of [(3aS,7R,7aS)-5-(6-benzyloxyhexanoyl)-2,2-dimethyl-4,6,7,7a-tetrahydro-3aH-[1,3]dioxolo[4,5-c]pyridin-7-yl] methanesulfonate (124) and [6-[(3aS,7R,7aS)-2,2-dimethyl-7-methylsulfonyloxy-4,6,7,7a-tetrahydro-3aH-[1,3]dioxolo[4,5-c]pyridin-5-yl]-6-oxo-hexyl] benzoate (125) was directly used for the next step.

LC-MS (Method D):

Rₜ[min] (UV-signal 220 nm): 1.43

M[g/mol]: 456.2 [M+H⁺] (124, main product), 470.2 [M+H⁺] (125, minor product)

### Example 10.3: Synthesis of 1-[(3aS,7S,7aR)-7-azido-2,2-dimethyl-4,6,7,7a-tetrahydro-3aH-[1,3]dioxolo[4,5-c]pyridin-5-yl]-6-benzyloxy-hexan-1-one (126) and [6-[(3aS,7S,7aR)-7-azido-2,2-dimethyl-4,6,7,7a-tetrahydro-3aH-[1,3]dioxolo[4,5-c]pyridin-5-yl]-6-oxo-hexyl]benzoate (127, inseparable mixture

The crude product mixture of [(3aS,7R,7aS)-5-(6-benzyloxyhexanoyl)-2,2-dimethyl-4,6,7,7a-tetrahydro-3aH-[1,3]dioxolo[4,5-c]pyridin-7-yl] methanesulfonate (124) and [6-[(3aS,7R,7aS)-2,2-dimethyl-7-methylsulfonyloxy-4,6,7,7a-tetrahydro-3aH-[1,3]dioxolo[4,5-c]pyridin-5-yl]-6-oxo-hexyl] benzoate (125) (max. 1.93 mmol, 1.00 equiv.) was dissolved in DMF (5 mL), NaN₃ (0.45 g, 6.97 mmol, 4.01 equiv.) and 15-crown-5 ether (0.60 g, 2.73 mmol, 1.57 equiv.) were added and the mixture was stirred at 100° C. for 1 d. EtOAc (50 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-80% EtOAc in n-heptane) to yield 1-[(3aS,7S,7aR)-7-azido-2,2-dimethyl-4,6,7,7a-tetrahydro-3aH-[1,3]dioxolo[4,5-c]pyridin-5-yl]-6-benzyloxy-hexan-1-one (126) and [6-[(3aS,7S,7aR)-7-azido-2,2-dimethyl-4,6,7,7a-tetrahydro-3aH-[1,3]dioxolo [4,5-c]pyridin-5-yl]-6-oxo-hexyl] benzoate (127, inseparable mixture, 189 mg, 0.47 mmol, 27% over two steps) as a colorless oil.

LC-MS (Method D):

Rₜ[min] (UV-signal 220 nm): 1.48

M[g/mol]: 403.2 [M+H⁺] (126, main product), 417.2 [M+H⁺] (127, minor product)

### Example 10.4: Synthesis of N-[(3S,4R,5S)-1-(6-benzyloxyhexanoyl)-4,5-dihydroxy-3-piperidyl]acetamide (128) and N-[(3S,4R,5S)-4,5-dihydroxy-1-(6-hydroxyhexanoyl)-3-piperidyl]acetamide (129)

To a solution of the mixture of 1-[(3aS,7S,7aR)-7-azido-2,2-dimethyl-4,6,7,7a-tetrahydro-3aH-[1,3]dioxolo[4,5-c]pyridin-5-yl]-6-benzyloxy-hexan-1-one (126) and [6-[(3aS,7S,7aR)-7-azido-2,2-dimethyl-4,6,7,7a-tetrahydro-3aH-[1,3]dioxolo[4,5-c]pyridin-5-yl]-6-oxo-hexyl] benzoate (127, 95 mg, 0.24 mmol, 1.00 equiv.) in THF (5 mL) and water (0.1 mL) was added PMe₃ (1 N in THF, 0.41 mL, 0.41 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.09 mL, approx. 4 equiv.) and pyridine (0.05 mL, approx. 2.5 equiv.) were added and the reaction mixture was stirred for 1 h at r.t. Since LC/MS indicated full formation of the acetamide, the reaction mixture was concentrated in vacuo. The crude product was dissolved in methanol (6 mL) and aqueous 1 N HCl (3 mL) and heated to 45° C. for 5 h until full acetonide deprotection was monitored by LC/MS. The solvent was removed in vacuo, the crude mixture was dissolved in methanol (10 mL) and solid NaOH (0.04 g, 1.00 mmol, 4.17 mmol) was added. The reaction mixture was stirred until full transesterification of benzyl ester was monitored by LC/MS. Acetic acid (0.10 mL) was added, the solvent was removed in vacuo and the crude mixture was purified by HPLC (15 min, 1-50% acetonitrile in water+0.1% TFA) to yield N-[(3S,4R,5S)-1-(6-benzyloxyhexanoyl)-4,5-dihydroxy-3-piperidyl]acetamide (128, 15 mg, 0.040 mmol, 17%) and N-[(3S,4R,5S)-4,5-dihydroxy-1-(6-hydroxyhexanoyl)-3-piperidyl]acetamide (129, 15 mg, 0.052 mmol, 22%) as colorless solids. N-[(3S,4R,5S)-1-(6-benzyloxyhexanoyl)-4,5-dihydroxy-3-piperidyl]acetamide (128): LC-MS (Method D):

Rₜ[min] (UV-signal 220 nm): 0.93

M[g/mol]: 379.2 [M+H⁺]

¹H NMR (400 MHz, DMSO-d₆) δ[ppm]: 7.82/7.64 (d, J=7.4/8.2 Hz, 1H), 7.41-7.23 (m, 5H), 4.78/4.73 (d, J=4.9/3.7 Hz, 1H), 4.68/4.65 (d, J=5.1/5.7 Hz, 1H), 4.44 (s, 2H), 4.14-3.24 (m, 8H), 3.16-3.04 (m, 1H), 2.38-2.12 (m, 2H), 1.82/1.81 (s, 3H), 1.59-1.43 (m, 4H), 1.37-1.22 (m, 2H) (two main conformers due to amide resonance)

N-[(3S,4R,5S)-4,5-dihydroxy-1-(6-hydroxyhexanoyl)-3-piperidyl]acetamide (129):

Rₜ[min] (UV-signal 220 nm): 0.18

M[g/mol]: 289.1 [M+H⁺]

¹H NMR (400 MHz, DMSO-d₆) δ[ppm]: 7.82/7.64 (d, J=7.3/7.9 Hz, 1H), 4.69 (s, 2H), 4.41-4.35/4.13-4.06 (m, 1H), 3.82-3.04 (m, 9H), 2.35-2.11 (m, 2H), 1.82/1.81 (s, 3H), 1.59-1.18 (m, 6H) (two main conformers due to amide resonance)

### Example 10.5: Synthesis of 6-benzyloxy-1-[(3S,4R,5S)-3,4-dihydroxy-5-[4-(phenoxy-methyl)triazol-1-yl]-1-piperidyl]hexan-1-one (131) and 1-[(3S,4R,5S)-3,4-dihydroxy-5-[4-(phenoxymethyl)triazol-1-yl]-1-piperidyl]-6-hydroxy-hexan-1-one (132)

To a solution of a mixture of 1-[(3aS,7S,7aR)-7-azido-2,2-dimethyl-4,6,7,7a-tetrahydro-3aH-[1,3]dioxolo[4,5-c]pyridin-5-yl]-6-benzyloxy-hexan-1-one (126) and [6-[(3aS,7S,7aR)-7-azido-2,2-dimethyl-4,6,7,7a-tetrahydro-3aH-[1,3]dioxolo[4,5-c]pyridin-5-yl]-6-oxo-hexyl] benzoate (127, 107 mg, 0.26 mmol, 1.00 equiv.) in methanol (4 mL) were added (prop-2-yn-1-yloxy)benzene (130, 42 mg, 0.32 mmol, 1.20 equiv.), tris-(2-(1-benzyl-1H-1,2,3-triazol-4-yl)ethyl)amine (TBTA, 8 mg, 0.01 mmol, 0.05 equiv.), copper (II) acetate (9 mg, 0.05 mmol, 0.18 equiv.) and sodium ascorbate (529 mg, 2.67 mmol, 10.08 equiv.) and the reaction mixture was stirred for 1 h at r.t. Since LC/MS indicated full conversion of the starting material, EtOAc (40 mL) and water (30 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo.

The crude mixture was dissolved in methanol (10 mL) and solid NaOH (0.04 g, 1.00 mmol, 4.17 mmol) was added. The reaction mixture was stirred until full transesterification of benzyl ester was monitored by LC/MS. Acetic acid (0.10 mL) was added, the solvent was removed in vacuo and the crude mixture was purified by HPLC (15 min, 1-50% acetonitrile in water+0.1% TFA) to yield 6-benzyloxy-1-[(3S,4R,5S)-3,4-dihydroxy-5-[4-(phenoxy-methyl)triazol-1-yl]-1-piperidyl]hexan-1-one (131, 47 mg, 0.094 mmol, 36%) and 1-[(3S,4R,5S)-3,4-di-hydroxy-5-[4-(phenoxymethyl)triazol-1-yl]-1-piperidyl]-6-hydroxy-hexan-1-one (132, 22 mg, 0.054 mmol, 21%) as colorless solids. 6-benzyloxy-1-[(3S,4R,5S)-3,4-dihydroxy-5-[4-(phenoxy-methyl)triazol-1-yl]-1-piperidyl]hexan-1-one (131):

LC-MS (Method D):

Rₜ[min] (UV-signal 220 nm): 1.28

M[g/mol]: 495.3 [M+H⁺]

¹H NMR (400 MHz, DMSO-d₆) δ[ppm]: 8.28/8.27 (s, 1H), 7.38-7.24 (m, 7H), 7.07-7.02 (m, 2H), 6.96 (m, 1H), 5.32-4.81 (m, 2H), 5.13 (s, 2H), 4.72-4.40 (m, 4H), 4.12-3.84 (m, 3H), 3.53-3.25 (m, 3H), 3.07-2.82 (m, 1H), 2.42-2.29 (m, 2H), 1.60-1.44 (m, 4H), 1.39-1.28 (m, 2H) (two main conformers due to amide resonance).

1-[(3S,4R,5S)-3,4-dihydroxy-5-[4-(phenoxymethyl)triazol-1-yl]-1-piperidyl]-6-hydroxy-hexan-1-one (132):

LC-MS (Method D):

Rₜ[min] (UV-signal 220 nm): 0.85

M[g/mol]: 405.3 [M+H⁺]

¹H NMR (400 MHz, DMSO-d₆) δ[ppm]: 8.28/8.28 (s, 1H), 7.97-7.92/7.08-7.02 (m, 2H), 7.66-7.59/6.99-6.93 (m, 1H), 7.53-7.47/7.34-7.27 (m, 2H), 5.20-4.91 (m, 4H), 4.73-4.27 (m, 3H), 4.14-3.81 (m, 3H), 3.54-3.25 (m, 3H), 3.08-2.81 (m, 1H), 2.43-2.29 (m, 2H), 1.57-1.36 (m, 4H), 1.36-1.20 (m, 2H) (two main conformers due to amide resonance).

### Example 11: Synthesis of Compounds 138 and 140

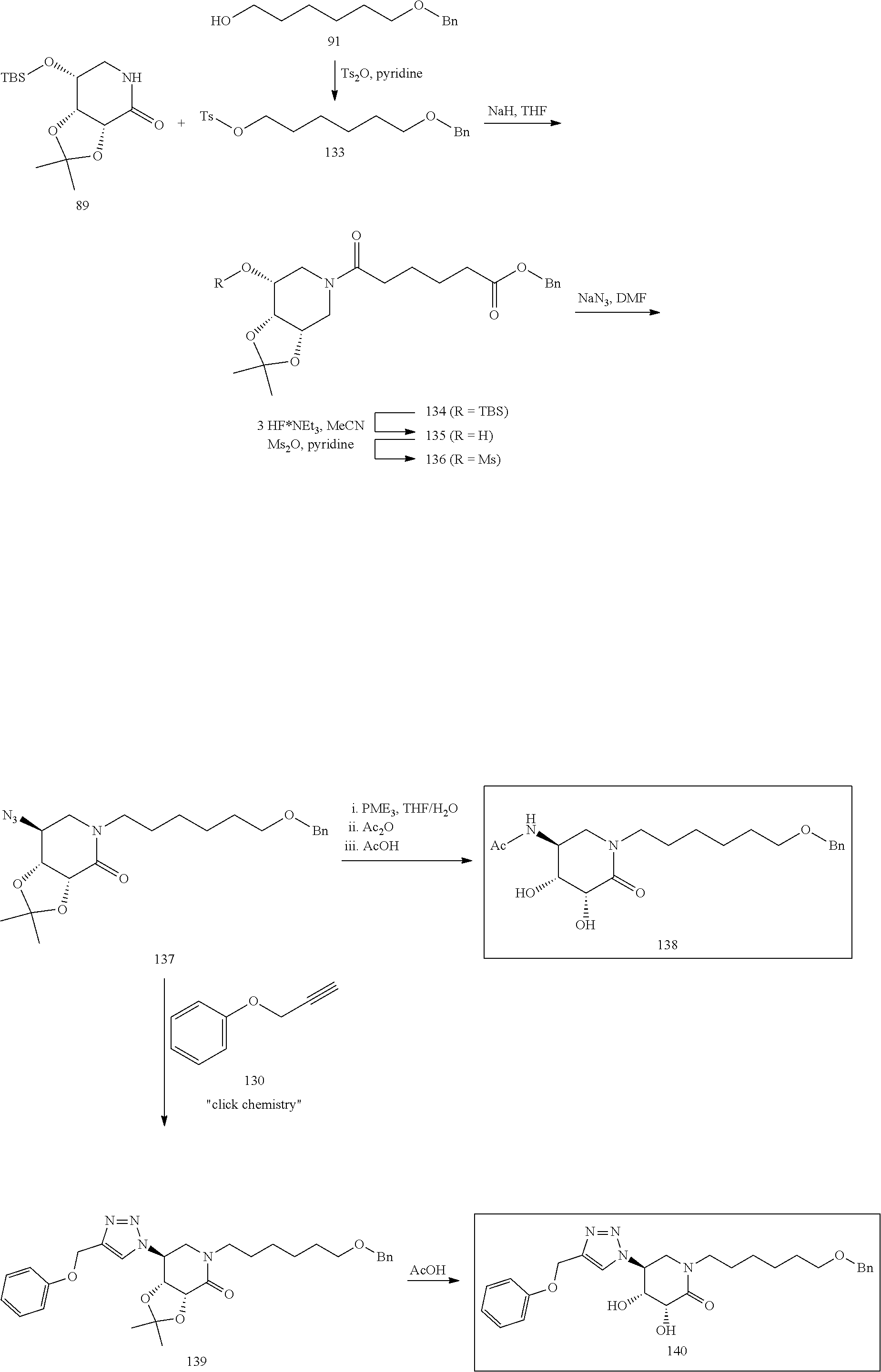

### Example 11.1: Synthesis of 6-benzyloxyhexyl 4-methylbenzenesulfonate (133)

To a solution of 6-benzyloxyhexan-1-ol (91, 1.00 g, 4.80 mmol, 1.00 equiv.) in anhydrous DCM (30 mL) was added pyridine (1.94 mL, 24.00 mmol, 5.00 equiv.) and tosyl anhydride (3.92 g, 12.00 mmol, 2.50 equiv.). The reaction mixture was stirred for 3 h at r.t. until LC/MS indicated full conversion of the starting material. Water (50 mL) was added, the layers were separated, and the aqueous layer was re-extracted with DCM (3×40 mL). The combined organic layers were dried (Na₂SO₄), filtered and concentrated in vacuo. The crude product was purified by flash chromatography (silica, 10% EtOAc in n-heptane) to yield 6-benzyloxyhexyl 4-methylbenzenesulfonate (133, 1.10 g, 3.03 mmol, 63%) as a colorless oil.

LC-MS (Method E):

Rₜ[min] (UV-signal 214 nm): 2.02

M[g/mol]: 363.5 [M+H⁺]

### Example 11.2: Synthesis of (3aR,7R,7aS)-5-(6-benzyloxyhexyl)-7-[tert-butyl(dimethyl)silyl]oxy-2,2-dimethyl-3a,6,7,7a-tetrahydro-[1,3]dioxolo[4,5-c]pyridin-4-one (134)

A solution of (3aR,7R,7aS)-7-[tert-butyl(dimethyl)silyl]oxy-2,2-dimethyl-5,6,7,7a-tetrahydro-3aH-[1,3]dioxolo[4,5-c]pyridin-4-one (89, 1.00 g, 3.32 mmol, 1.00 equiv.) in anhydrous THF (50 mL) was cooled to 0° C. and sodium hydride (60% in mineral oil, 528 mg, 13.20 mmol, 3.98 equiv.) was added in small portions. The reaction mixture was stirred for 10 min at 0° C. and 6-benzyloxyhexyl 4-methylbenzenesulfonate (133, 1.40 g, 3.86 mmol, 1.16 equiv.) was added. The reaction mixture was stirred at 80° C. for 5 h. Water (50 mL) and EtOAc (40 mL) were added, the layers were separated, and the aqueous layer was re-extracted with EtOAc (2×40 mL). The combined organic layers was washed with saturated aqueous NaCl-solution (40 mL), dried (Na₂SO₄), filtered and concentrated in vacuo. The crude product was purified by flash chromatography (silica, 16% EtOAc in DCM) to yield (3aR,7R,7aS)-5-(6-benzyloxyhexyl)-7-[tert-butyl(dimethyl)silyl]oxy-2,2-dimethyl-3a,6,7,7a-tetrahydro-[1,3]dioxolo[4,5-c]pyridin-4-one (134, 723 mg, 1.47 mmol, 44%) as a colorless oil.

LC-MS (Method F):

Rₜ[min] (UV-signal 214 nm): 2.39

M[g/mol]: 491.9 [M+H⁺]

¹H NMR (400 MHz, DMSO-d₆) δ[ppm]: 7.37-7.24 (m, 5H), 4.43 (s, 2H), 4.39-4.33 (m, 2H), 4.15 (m, 1H), 3.45-3.32 (m, 4H), 3.18-3.03 (m, 2H), 1.58-1.37 (m, 4H), 1.37-1.16 (m, 5H), 1.31 (s, 3H), 1.29 (s, 3H), 0.86 (s, 9H), 0.09 (s, 6H).

### Example 11.3: Synthesis of (3aR,7R,7aR)-5-(6-benzyloxyhexyl)-7-hydroxy-2,2-dimethyl-3a,6,7,7a-tetrahydro-[1,3]dioxolo[4,5-c]pyridin-4-one (135)

The reaction was performed in a plastic vial due to the use of HF. To a solution of (3aR,7R,7aS)-5-(6-benzyloxyhexyl)-7-[tert-butyl(dimethyl)silyl]oxy-2,2-dimethyl-3a,6,7,7a-tetrahydro-[1,3]dioxolo[4,5-c]pyridin-4-one (134, 723 mg, 1.47 mmol, 1.00 equiv.) in acetonitrile (10 mL) was added 3 HF·NEt₃ (2.34 g, 14.53 mmol, 9.88 equiv.) and the solution was stirred at r.t. for 16 h until LC/MS indicated full conversion. EtOAc (150 mL) and saturated aqueous NaHCO₃-solution (50 mL) were added, the layers were separated, and the aqueous layer was re-extracted with EtOAc (2×20 mL). The combined organic layers were washed with saturated aqueous NaCl-solution (30 mL), dried (MgSO₄), filtered and concentrated in vacuo. The crude product (3aR,7R,7aR)-5-(6-benzyloxyhexyl)-7-hydroxy-2,2-dimethyl-3a,6,7,7a-tetrahydro-[1,3]dioxo-lo[4,5-c]pyridin-4-one (135, 492 mg, 1.30 mmol, 89%) was obtained as a colorless oil and was sufficiently pure to be used for the next step.

LC-MS (Method D):

Rₜ[min] (UV-signal 220 nm): 0.76

M[g/mol]: 378.2 [M+H⁺]

### Example 11.4: Synthesis of [(3aR,7R,7aS)-5-(6-benzyloxyhexyl)-2,2-dimethyl-4-oxo-3a,6,7,7a-tetrahydro-[1,3]dioxolo[4,5-c]pyridin-7-yl] methanesulfonate (136)

A solution of (3aR,7R,7aR)-5-(6-benzyloxyhexyl)-7-hydroxy-2,2-dimethyl-3a,6,7,7a-tetrahydro-[1,3]dioxolo[4,5-c]pyridin-4-one (135, 492 mg, 1.30 mmol, 1.00 equiv.) in anhydrous DCM (20 mL) was cooled to 0° C. and pyridine (0.30 mL, 3.69 mmol, 2.83 equiv.) and mesyl anhydride (0.40 g, 2.30 mmol, 1.77 equiv.) were added. The reaction mixture was stirred for 3 h at 0° C. until LC/MS indicated full conversion of the starting material. Aqueous 1 N HCl (30 mL) and EtOAc (50 mL) were added, the layers were separated, the organic layer was washed with saturated aqueous NaCl-solution (30 mL), dried (MgSO₄), filtered and concentrated in vacuo. The crude product [(3aR,7R,7aS)-5-(6-benzyloxyhexyl)-2,2-dimethyl-4-oxo-3a,6,7,7a-tetrahydro-[1,3]dioxolo[4,5-c]pyridin-7-yl] methanesulfonate (136) was directly used for the next step.

LC-MS (Method D):

Rₜ[min] (UV-signal 220 nm): 1.42

M[g/mol]: 456.1 [M+H⁺]

### Example 11.5: Synthesis of (3aR,7S,7aR)-7-azido-5-(6-benzyloxyhexyl)-2,2-dimethyl-3a,6,7,7a-tetrahydro-[1,3]dioxolo[4,5-c]pyridin-4-one (137)

The crude product [(3aR,7R,7aS)-5-(6-benzyloxyhexyl)-2,2-dimethyl-4-oxo-3a,6,7,7a-tetrahydro-[1,3]dioxolo[4,5-c]pyridin-7-yl] methanesulfonate (136, max. 1.30 mmol, 1.00 equiv.) was dissolved in DMF (5 mL), NaN₃ (0.34 g, 5.23 mmol, 4.00 equiv.) and 15-crown-5 ether (0.43 g, 1.96 mmol, 1.50 equiv.) were added and the mixture was stirred at 100° C. for 1 d. EtOAc (50 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-40% EtOAc in n-heptane) to yield (3aR,7S,7aR)-7-azido-5-(6-benzyloxyhexyl)-2,2-dimethyl-3a,6,7,7a-tetrahydro-[1,3]dioxolo[4,5-c]pyridin-4-one (137, 135 mg, 0.34 mmol, 26% over two steps) as a colorless oil.

LC-MS (Method D):

Rₜ[min] (UV-signal 220 nm): 1.60

M[g/mol]: 403.2 [M+H⁺]

### Example 11.6: Synthesis of N-[(3S,4R,5R)-1-(6-benzyloxyhexyl)-4,5-dihydroxy-6-oxo-3-piperidyl]acetamide (138)

To a solution of (3aR,7S,7aR)-7-azido-5-(6-benzyloxyhexyl)-2,2-dimethyl-3a,6,7,7a-tetrahydro-[1,3]dioxolo[4,5-c]pyridin-4-one (137, 79 mg, 0.19 mmol, 1.00 equiv.) in THF (5 mL) and water (0.1 mL) was added PMe₃ (1 N in THF, 0.29 mL, 0.29 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.09 mL, approx. 4 equiv.) and pyridine (0.05 mL, approx. 2.5 equiv.) were added and the reaction mixture was stirred for 1 h at r.t. Since LC/MS indicated full formation of the acetamide, the reaction mixture was concentrated in vacuo. The crude product was dissolved in methanol (6 mL) and aqueous 1 N HCl (3 mL) and heated to 45° C. for 5 h until full acetonide deprotection was monitored by LC/MS. The solvent was removed in vacuo, the crude mixture was purified by HPLC (15 min, 10-95% acetonitrile in water+0.1% TFA) to yield N-[(3S,4R,5R)-1-(6-benzyloxyhexyl)-4,5-dihydroxy-6-oxo-3-piperidyl]acet-amide (138, 15 mg, 0.040 mmol, 17%) as a colorless solid.

LC-MS (Method D):

Rₜ[min] (UV-signal 220 nm): 1.04

M[g/mol]: 379.2 [M+H⁺]

¹H NMR (400 MHz, DMSO-d₆) δ[ppm]: 8.09 (d, J=7.7 Hz, 1H), 7.37-7.24 (m, 5H), 5.46-4.53 (m, 2H), 4.44 (s, 2H), 4.04-3.95 (m, 2H), 3.82 (m, 1H), 3.56 (dd, J=12.7, 5.6 Hz, 1H), 3.41 (t, J=6.6 Hz, 2H), 3.39-3.09 (m, 1H), 2.97 (dd, J=12.8, 5.0 Hz, 1H), 1.83 (s, 3H), 1.58-1.17 (m, 8H).

### Example 11.7: Synthesis of (3aR,7S,7aR)-5-(6-benzyloxyhexyl)-2,2-dimethyl-7-[4-(phenoxymethyl)triazol-1-yl]-3a,6,7,7a-tetrahydro-[1,3]dioxolo[4,5-c]pyridin-4-one (139)

To a solution of (3aR,7S,7aR)-7-azido-5-(6-benzyloxyhexyl)-2,2-dimethyl-3a,6,7,7a-tetra-hydro-[1,3]dioxolo[4,5-c]pyridin-4-one (137, 100 mg, 0.25 mmol, 1.00 equiv.) in methanol (4 mL) were added (prop-2-yn-1-yloxy)benzene (130, 39 mg, 0.30 mmol, 1.20 equiv.), tris-(2-(1-benzyl-1H-1,2,3-triazol-4-yl)ethyl)amine (TBTA, 8 mg, 0.01 mmol, 0.05 equiv.), copper (II) acetate (9 mg, 0.05 mmol, 0.18 equiv.) and sodium ascorbate (495 mg, 2.50 mmol, 10.00 equiv.) and the reaction mixture was stirred for 1 h at r.t. Since LC/MS indicated full conversion of the starting material, EtOAc (40 mL) and water (30 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo.

The crude mixture was purified by HPLC (15 min, 10-90% acetonitrile in water+0.1% TFA) to yield a (3aR,7S,7aR)-5-(6-benzyloxyhexyl)-2,2-dimethyl-7-[4-(phenoxymethyl)triazol-1-yl]-3a,6,7,7a-tetrahydro-[1,3]dioxolo[4,5-c]pyridin-4-one (139) containing acetonitrile water mixture which was directly used for acetonide deprotection.

LC-MS (Method D):

Rₜ[min] (UV-signal 220 nm): 1.65

M[g/mol]: 535.2 [M+H⁺]

### Example 11.8: Synthesis of (3R,4R,5S)-1-(6-benzyloxyhexyl)-3,4-dihydroxy-5-[4-(phenoxymethyl)triazol-1-yl]piperidin-2-one (140)

The product (3aR,7S,7aR)-5-(6-benzyloxyhexyl)-2,2-dimethyl-7-[4-(phenoxymethyl)triazol-1-yl]-3a,6,7,7a-tetrahydro-[1,3]dioxolo[4,5-c]pyridin-4-one (139) containing acetonitrile water mixture from the HPLC purification was concentrated in vacuo to remove most of the acetonitrile, then re-dissolved in methanol (6 ML) and aqueous 1 N HCl (3 mL) and heated to 45° C. for 16 h until full acetonide deprotection was monitored by LC/MS. The solvent was removed in vacuo, the crude mixture was purified by HPLC (15 min, 20-90% acetonitrile in water+0.1% TFA) to yield (3R,4R,5S)-1-(6-benzyloxyhexyl)-3,4-dihydroxy-5-[4-(phenoxymethyl)triazol-1-yl]piperidin-2-one (140, 44 mg, 0.089 mmol, 36% over 2 steps) as a colorless solid.

LC-MS (Method D):

Rₜ[min] (UV-signal 220 nm): 1.44

M[g/mol]: 495.2 [M+H⁺]

¹H NMR (400 MHz, DMSO-d₆) δ[ppm]: 8.36 (s, 1H), 7.38-7.23 (m, 7H), 7.07-7.02 (m, 2H), 6.96 (m, 1H), 5.48 (s, br, 2H), 5.14 (s, 2H), 4.96 (m, 1H), 4.44 (s, 2H), 4.25 (dd, J=7.0, 3.7 Hz, 1H), 4.02 (d, J=3.7 Hz, 1H), 3.76 (dd, J=12.8, 6.4 Hz, 1H), 3.69 (dd, J=12.8, 8.3 Hz, 1H), 3.41 (t, J=6.4 Hz, 2H), 3.39-3.19 (m, 2H), 1.58-1.42 (m, 4H), 1.38-1.19 (m, 4H).

### Example 12: Synthesis of Compounds 146, 147, 148, 153, and 154

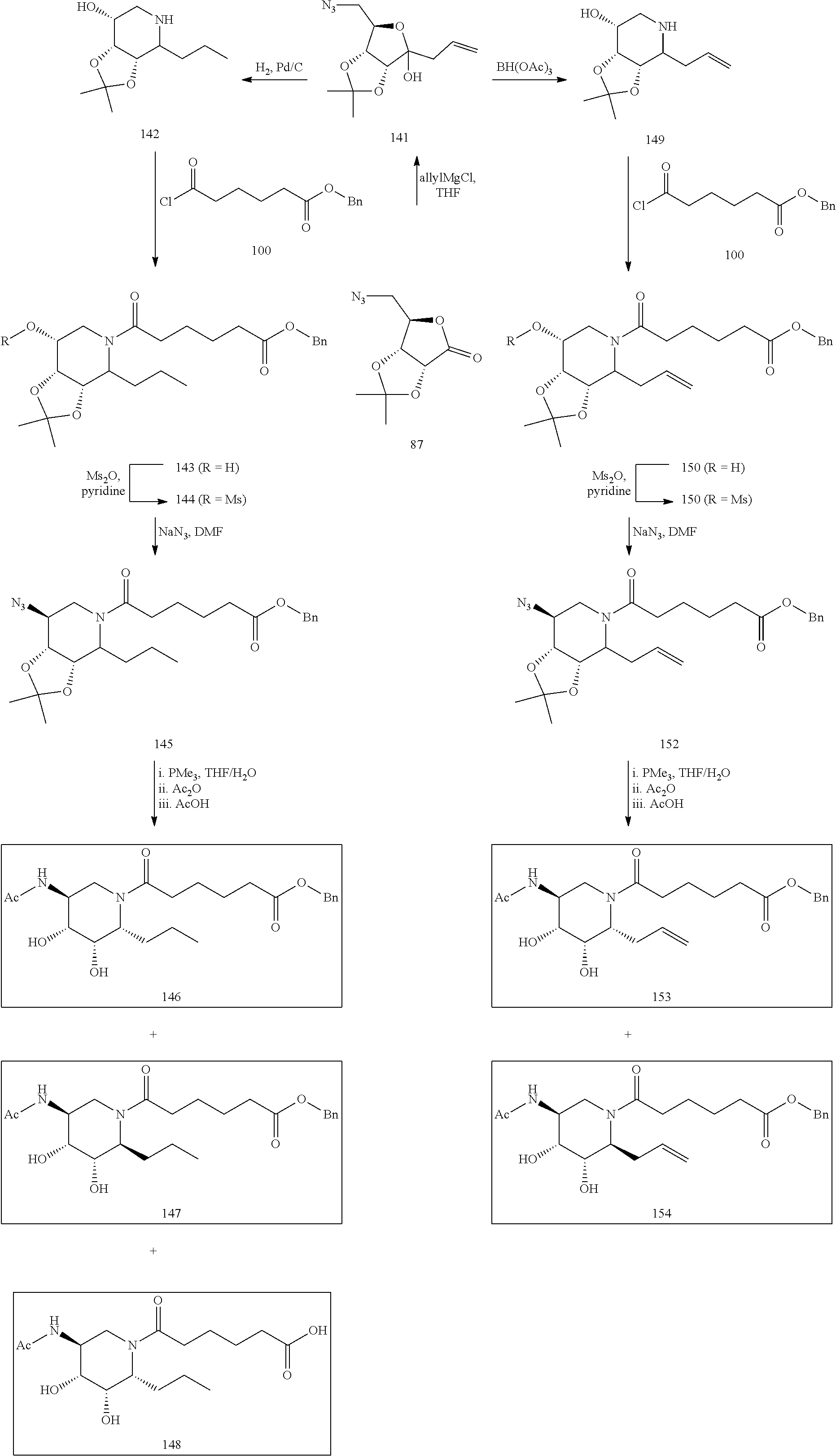

### Example 12.1: Synthesis of (3aR,6R,6aR)-4-allyl-6-(aminomethyl)-2,2-dimethyl-6,6a-dihydro-3aH-furo[3,4-d][1,3]dioxol-4-ol (141)

A solution of (3aR,6R,6aR)-6-(aminomethyl)-2,2-dimethyl-6,6a-dihydro-3aH-furo[3,4-d][1,3]dioxol-4-one (87, 2.22 g, 10.43 mmol, 1.00 equiv.) in THF (50 mL) was cooled to −78° C., allyl magnesium chloride (1.7 M, 9.20 mL, 15.64 equiv. 1.50 equiv.) was added and the reaction was stirred for 30 min until LC/MS indicated full conversion of the starting material. Saturated aqueous NH₄Cl-solution (40 mL), EtOAc (50 mL) and water (50 mL) were added, the layers were separated, and the aqueous layer was re-extracted with EtOAc (3×50 mL). The combined organic layers were washed with saturated aqueous NaCl-solution (30 mL), dried (MgSO₄), filtered and concentrated in vacuo. The crude product was purified by flash chromatography (silica, 1-30% EtOAc in n-heptane) to yield (3aR,6R,6aR)-4-allyl-6-(aminomethyl)-2,2-dimethyl-6,6a-dihydro-3aH-furo[3,4-d][1,3]dioxol-4-ol (141, 2.17 g, 8.49 mmol, 81%) as a colorless oil.

LC-MS (Method D):

Rₜ[min] (UV-signal 220 nm): 1.20

M[g/mol]: 238.1[M−H₂O+H⁺], 210.1 238.1[M−H₂O—N₂+H⁺]

### Example 12.2: Synthesis of (3aS,7R,7aR)-2,2-dimethyl-4-propyl-3a,4,5,6,7,7a-hexahydro-[1,3]dioxolo[4,5-c]pyridin-7-ol (142)

(3aR,6R,6aR)-4-allyl-6-(aminomethyl)-2,2-dimethyl-6,6a-dihydro-3aH-furo[3,4-d][1,3]dioxol-4-ol (141, 325 mg, 1.27 mmol, 1.00 equiv.) was dissolved in THF (20 mL), 10% Pd/C (0.07 g, 0.06 mmol, 0.05 equiv.) was added and the mixture was hydrogenated in an autoclave at r.t. and 4 bar hydrogen gas for 1 d. Since complete piperidine formation was detected by LC/MS, the reaction mixture was filtered and concentrated in vacuo. The crude product (3aS,7R,7aR)-2,2-dimethyl-4-propyl-3a,4,5,6,7,7a-hexahydro-[1,3]dioxolo[4,5-c]pyri-din-7-ol (142) was directly used in solution for the next step.

LC-MS (Method D):

Rₜ[min] (TIC-signal): 0.48

M[g/mol]: 216.1 [M+H⁺]

### Example 12.3: Synthesis of benzyl 6-[(3aS,7R,7aR)-7-hydroxy-2,2-dimethyl-4-propyl-4,6,7,7a-tetrahydro-3aH-[1,3]dioxolo[4,5-c]pyridin-5-yl]-6-oxo-hexanoate (143)

To a solution of crude (3aS,7R,7aR)-2,2-dimethyl-4-propyl-3a,4,5,6,7,7a-hexahydro-[1,3]dioxolo[4,5-c]pyri-din-7-ol (142, max. 1.27 mmol, 1.00 equiv.) in THF (30 mL) and saturated aqueous NaHCO₃-solution (20 mL) was added a solution of acid chloride benzyl 6-chloro-6-oxo-hexanoate (100, 486 mg, 1.91 mmol, 1.51 equiv.) in THF (10 mL) and the reaction mixture was stirred for 16 h at r.t. EtOAc (100 mL) and water (20 mL) were added, the layers were separated, the organic layer was washed with aqueous 2 N NaOH solution (3×30 mL) saturated aqueous NaCl-solution (30 mL), dried (MgSO₄), filtered and concentrated in vacuo. The crude product was purified by flash chromatography (silica, 5-70% EtOAc in n-heptane) to yield benzyl 6-[(3aS,7R,7aR)-7-hydroxy-2,2-dimethyl-4-propyl-4,6,7,7a-tetrahydro-3aH-[1,3]dioxolo[4,5-c]pyridin-5-yl]-6-oxo-hexanoate (143, 392 mg, 0.90 mmol, 72% over two steps) as a colorless oil and an inseparable mixture of epimers at position 4.

LC-MS (Method D):

Rₜ[min] (UV-signal 220 nm): 1.40

M[g/mol]: 434.3 [M+H⁺]

### Example 12.4: Synthesis of benzyl 6-[(3aS,7R,7aS)-2,2-dimethyl-7-methylsulfonyloxy-4-propyl-4,6,7,7a-tetrahydro-3aH-[1,3]dioxolo[4,5-c]pyridin-5-yl]-6-oxo-hexanoate (144)

A solution of benzyl 6-[(3aS,7R,7aR)-7-hydroxy-2,2-dimethyl-4-propyl-4,6,7,7a-tetrahydro-3aH-[1,3]dioxolo[4,5-c]pyridin-5-yl]-6-oxo-hexanoate (143, 392 mg, 0.90 mmol, 1.00 equiv.) in anhydrous DCM (20 mL) was cooled to 0° C. and pyridine (0.25 mL, 3.08 mmol, 3.40 equiv.) and mesyl anhydride (236 mg, 1.36 mmol, 1.50 equiv.) were added. The reaction mixture was stirred for 3 h at 0° C. until LC/MS indicated full conversion of the starting material. Aqueous 1 N HCl (30 mL) and EtOAc (50 mL) were added, the layers were separated, the organic layer was washed with saturated aqueous NaCl-solution (30 mL), dried (MgSO₄), filtered and concentrated in vacuo. The crude product benzyl 6-[(3aS,7R,7aS)-2,2-dimethyl-7-methylsulfonyloxy-4-propyl-4,6,7,7a-tetrahydro-3aH-[1,3]dioxolo[4,5-c]pyridin-5-yl]-6-oxo-hexanoate (144) was directly used for the next step.

LC-MS (Method D):

Rₜ[min] (UV-signal 220 nm): 1.60

M[g/mol]: 512.2 [M+H⁺]

### Example 12.5: Synthesis of benzyl 6-[(3aS,7S,7aR)-7-azido-2,2-dimethyl-4-propyl-4,6,7,7a-tetrahydro-3aH-[1,3]dioxolo[4,5-c]pyridin-5-yl]-6-oxo-hexanoate (145)

The crude product benzyl 6-[(3aS,7R,7aS)-2,2-dimethyl-7-methylsulfonyloxy-4-propyl-4,6,7,7a-tetrahydro-3aH-[1,3]dioxolo[4,5-c]pyridin-5-yl]-6-oxo-hexanoate (144, max. 0.90 mmol, 1.00 equiv.) was dissolved in DMF (5 mL), NaN₃ (0.24 g, 3.62 mmol, 4.00 equiv.) and 15-crown-5 ether (0.30 g, 1.36 mmol, 1.50 equiv.) were added and the mixture was stirred at 100° C. for 1 d. EtOAc (50 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-30% EtOAc in n-heptane) to yield benzyl 6-[(3aS,7S,7aR)-7-azido-2,2-dimethyl-4-propyl-4,6,7,7a-tetrahydro-3aH-[1,3]dioxolo[4,5-c]pyridin-5-yl]-6-oxo-hexanoate (145, 261 mg, 0.57 mmol, 63% over two steps) as a colorless oil.

LC-MS (Method D):

Rₜ[min] (UV-signal 220 nm): 1.73

M[g/mol]: 459.3 [M+H⁺]

### Example 12.6: Synthesis of benzyl 6-[(2R,3S,4R,5S)-5-acetamido-3,4-dihydroxy-2-propyl-1-piperidyl]-6-oxo-hexanoate (146) and benzyl 6-[(2S,3S,4R,5S)-5-acetamido-3,4-dihydroxy-2-propyl-1-piperidyl]-6-oxo-hexanoate (147) and 6-[(2R,3S,4R,5S)-5-acetamido-3,4-dihydroxy-2-propyl-1-piperidyl]-6-oxo-hexanoic acid (148)

To a solution of benzyl 6-[(3aS,7S,7aR)-7-azido-2,2-dimethyl-4-propyl-4,6,7,7a-tetrahydro-3aH-[1,3]dioxolo[4,5-c]pyridin-5-yl]-6-oxo-hexanoate (145, 261 mg, 0.57 mmol, 1.00 equiv.) in THF (5 mL) and water (0.1 mL) was added PMe₃ (1 N in THF, 0.86 mL, 0.86 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.27 mL, approx. 5 equiv.) and pyridine (0.18 mL, approx. 4 equiv.) were added and the reaction mixture was stirred for 1 h at r.t. Since LC/MS indicated full formation of the acetamide, the reaction mixture was concentrated in vacuo. The crude product was dissolved in methanol (3 mL) and purified by HPLC (15 min, 10-95% acetonitrile in water+0.1% TFA) to yield an inseparable mixture of the acetonide protected intermediates as a colorless solid. The solid was dissolved in acetic acid (5 mL) and water (1 mL) and heated at 80° C. for 16 h. The reaction mixture was concentrated in vacuo, the residue was dissolved in methanol (3 mL) and purified by HPLC (15 min, 10-95% acetonitrile in water+0.1% TFA) to yield the two isomers benzyl 6-[(2R,3S,4R,5S)-5-acetamido-3,4-dihydroxy-2-propyl-1-piperidyl]-6-oxo-hexanoate (146, 16 mg, 0.04 mmol, 6% over two steps) and benzyl 6-[(2S,3S,4R,5S)-5-acetamido-3,4-dihydroxy-2-propyl-1-piperidyl]-6-oxo-hexano-ate (147, 18 mg, 0.04 mmol, 7% over two steps) and 6-[(2R,3S,4R,5S)-5-acetamido-3,4-dihydroxy-2-propyl-1-piperidyl]-6-oxo-hexanoic acid (148, 6 mg, 0.02 mmol, 3% over two steps). The assignment of the absolute stereochemistry was done arbitrarily.

Benzyl 6-[(2R,3S,4R,5S)-5-acetamido-3,4-dihydroxy-2-propyl-1-piperidyl]-6-oxo-hexanoate (146):

LC-MS (Method D):

Rₜ[min] (UV-signal 220 nm): 1.09

M[g/mol]: 435.2 [M+H⁺]

¹H NMR (400 MHz, DMSO-d6) δ[ppm]: 7.75 (s, br, 1H), 7.42-7.28 (m, 5H), 5.09 (s, 2H), 4.92-4.35 (m, 3H), 3.85-3.76 (m, 1H), 3.74-3.66 (m, 1H), 3.55 (m, 2H), 3.39-3.19 (m, 1H), 2.40-2.22 (m, 3H), 2.20-2.07 (m, 1H), 1.81 (s, 3H), 1.80-1.46 (m, 6H), 1.25-1.05 (m, 2H), 0.84 (t, J=7.3 Hz, 3H) (main conformer due to amide resonance).

Benzyl 6-[(2S,3S,4R,5S)-5-acetamido-3,4-dihydroxy-2-propyl-1-piperidyl]-6-oxo-hexanoate (147)

LC-MS (Method D):

Rₜ[min] (UV-signal 220 nm): 0.92

M[g/mol]: 435.2 [M+H⁺]

¹H NMR (400 MHz, DMSO-d₆) δ[ppm]: 8.04/7.97 (d, J=7.3/8.3 Hz, 1H), 7.44-7.28 (m, 5H), 5.36-5.26 (m, 2H), 5.09 (s, 2H), 4.04-3.90 (m, 1H), 3.78-3.69 (m, 1H), 3.59-3.46 (m, 1H), 3.23/3.14 (dd, J=12.1, 4.7/12.4, 4.8 Hz, 1H), 2.83-2.63 (m, 1H), 2.48-2.28 (m, 4H), 1.84/1.78 (s, 3H), 1.70-1.50 (m, 4H), 1.49-1.20 (m, 4H), 0.89/0.83 (t, J=7.3/6.8 Hz, 3H) (two main conformers due to amide resonance).

6-[(2R,3S,4R,5S)-5-Acetamido-3,4-dihydroxy-2-propyl-1-piperidyl]-6-oxo-hexanoic acid (148)

LC-MS (Method D):

Rₜ[min] (UV-signal 220 nm): 0.64

M[g/mol]: 345.2 [M+H⁺]

¹H NMR (400 MHz, DMSO-d₆) δ[ppm]: 7.93 (d, J=6.8 Hz, 1H), 5.12-4.67 (m, 2H), 4.58-4.47 (m, 1H), 3.91-3.72 (m, 2H), 3.70-3.61 (m, 1H), 3.57-3.43 (m, 2H), 2.39-1.94 (m, 4H), 1.82 (s, 3H), 1.81-0.98 (m, 8H), 0.93-0.78 (m, 3H) (main conformer due to amide resonance).

### Example 12.7: Synthesis of (3aS,7R,7aR)-4-allyl-2,2-dimethyl-3a,4,5,6,7,7a-hexahydro-[1,3]dioxolo[4,5-c]pyridin-7-ol (149)

(3aR,6R,6aR)-4-allyl-6-(aminomethyl)-2,2-dimethyl-6,6a-dihydro-3aH-furo[3,4-d][1,3]di-oxol-4-ol (141, 325 mg, 1.27 mmol, 1.00 equiv.) was dissolved in THF (30 mL) and water (3 mL), PMe₃ (1 N in THF, 1.91 mL, 1.91 mmol, 1.50 equiv.) was added and the reaction mixture was stirred for 30 min at r.t. Since LC/MS indicated full conversion of the starting material, acetic acid (1 mL) was added and the reaction mixture was stirred for 30 min at r.t. NaBH(OAc)₃ (810 mg, 3.82 mmol, 3.00 equiv.) was added and the reaction mixture was stirred for 30 min at r.t. until LC/MS indicated full reductive amination to the piperidine. The reaction was stopped by the addition of water (30 mL), the THF was removed in vacuo and the aqueous solution was freeze dried. The crude product (3aS,7R,7aR)-4-allyl-2,2-dimethyl-3a,4,5,6,7,7a-hexahydro-[1,3]dioxolo[4,5-c]pyridin-7-ol (149) was directly used for the next step.

LC-MS (Method D):

Rₜ[min] (TIC-signal): 0.47

M[g/mol]: 214.1 [M+H⁺]

### Example 12.8: Synthesis of benzyl 6-[(3aS,7R,7aR)-4-allyl-7-hydroxy-2,2-dimethyl-4,6,7,7a-tetrahydro-3aH-[1,3]dioxolo[4,5-c]pyridin-5-yl]-6-oxo-hexanoate (150)

To a solution of crude (3aS,7R,7aR)-4-allyl-2,2-dimethyl-3a,4,5,6,7,7a-hexahydro-[1,3]dioxolo[4,5-c]pyridin-7-ol (149, max. 1.27 mmol, 1.00 equiv.) in THF (30 mL) and saturated aqueous NaHCO₃-solution (20 mL) was added a solution of acid chloride benzyl 6-chloro-6-oxo-hexanoate (100, 486 mg, 1.91 mmol, 1.51 equiv.) in THF (10 mL) and the reaction mixture was stirred for 16 h at r.t. EtOAc (100 mL) and water (20 mL) were added, the layers were separated, the organic layer was washed with aqueous 2 N NaOH solution (3×30 mL), saturated aqueous NaCl-solution (30 mL), dried (MgSO₄), filtered and concentrated in vacuo. The crude product was purified by flash chromatography (silica, 5-70% EtOAc in n-heptane) to yield benzyl 6-[(3aS,7R,7aR)-4-allyl-7-hydroxy-2,2-dimethyl-4,6,7,7a-tetrahydro-3aH-[1,3]dioxolo[4,5-c]pyridin-5-yl]-6-oxo-hexanoate (150, 204 mg, 0.47 mmol, 37% over two steps) as a colorless oil and an inseparable mixture of epimers at position 4.

LC-MS (Method D):

Rₜ[min] (UV-signal 220 nm): 1.33

M[g/mol]: 432.2 [M+H⁺]

### Example 12.9: Synthesis of benzyl 6-[(3aS,7R,7aS)-4-allyl-2,2-dimethyl-7-methylsulfonyl-oxy-4,6,7,7a-tetrahydro-3aH-[1,3]dioxolo[4,5-c]pyridin-5-yl]-6-oxo-hexanoate (151)

A solution of benzyl 6-[(3aS,7R,7aR)-4-allyl-7-hydroxy-2,2-dimethyl-4,6,7,7a-tetrahydro-3aH-[1,3]dioxolo[4,5-c]pyridin-5-yl]-6-oxo-hexanoate (150, 204 mg, 0.47 mmol, 1.00 equiv.) in anhydrous DCM (20 mL) was cooled to 0° C. and pyridine (0.15 mL, 1.85 mmol, 3.90 equiv.) and mesyl anhydride (124 mg, 0.71 mmol, 1.50 equiv.) were added. The reaction mixture was stirred for 3 h at 0° C. until LC/MS indicated full conversion of the starting material. Aqueous 1 N HCl (30 mL) and EtOAc (50 mL) were added, the layers were separated, the organic layer was washed with saturated aqueous NaCl-solution (30 mL), dried (MgSO₄), filtered and concentrated in vacuo. The crude product benzyl 6-[(3aS,7R,7aS)-4-allyl-2,2-dimethyl-7-methylsulfonyl-oxy-4,6,7,7a-tetrahydro-3aH-[1,3]dioxolo[4,5-c]pyridin-5-yl]-6-oxo-hexanoate (151) was directly used for the next step.

LC-MS (Method D):

Rₜ[min] (UV-signal 220 nm): 1.53

M[g/mol]: 510.2 [M+H⁺]

### Example 12.10: Synthesis of benzyl 6-[(3aS,7S,7aR)-4-allyl-7-azido-2,2-dimethyl-4,6,7,7a-tetrahydro-3aH-[1,3]dioxolo[4,5-c]pyridin-5-yl]-6-oxo-hexanoate (152)

The crude product benzyl 6-[(3aS,7R,7aS)-4-allyl-2,2-dimethyl-7-methylsulfonyl-oxy-4,6,7,7a-tetrahydro-3aH-[1,3]dioxolo[4,5-c]pyridin-5-yl]-6-oxo-hexanoate (151, max. 0.47 mmol, 1.00 equiv.) was dissolved in DMF (5 mL), NaN₃ (0.12 g, 1.89 mmol, 4.00 equiv.) and 15-crown-5 ether (0.16 g, 0.71 mmol, 1.50 equiv.) were added and the mixture was stirred at 100° C. for 1 d. EtOAc (50 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-40% EtOAc in n-heptane) to yield benzyl 6-[(3aS,7S,7aR)-4-allyl-7-azido-2,2-dimethyl-4,6,7,7a-tetrahydro-3aH-[1,3]dioxolo[4,5-c]pyridin-5-yl]-6-oxo-hexanoate (152, 64 mg, 0.14 mmol, 30% over two steps) as a colorless oil.

LC-MS (Method D):

Rₜ[min] (UV-signal 220 nm): 1.72

M[g/mol]: 457.2 [M+H⁺]

### Example 12.11: Synthesis of benzyl 6-[(2R,3S,4R,5S)-5-acetamido-2-allyl-3,4-dihydroxy-1-piperidyl]-6-oxo-hexanoate (153) and benzyl 6-[(2S,3S,4R,5S)-5-acetamido-2-allyl-3,4-dihydroxy-1-piperidyl]-6-oxo-hexanoate (154)

To a solution of benzyl 6-[(3aS,7S,7aR)-4-allyl-7-azido-2,2-dimethyl-4,6,7,7a-tetrahydro-3aH-[1,3]dioxolo[4,5-c]pyridin-5-yl]-6-oxo-hexanoate (152, 64 mg, 0.14 mmol, 1.00 equiv.) in THF (5 mL) and water (0.1 mL) was added PMe₃ (1 N in THF, 0.21 mL, 0.21 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.06 mL, approx. 5 equiv.) and pyridine (0.05 mL, approx. 4 equiv.) were added and the reaction mixture was stirred for 1 h at r.t. Since LC/MS indicated full formation of the acetamide, the reaction mixture was concentrated in vacuo. The crude product was dissolved in methanol (3 mL) and purified by HPLC (15 min, 10-95% acetonitrile in water+0.1% TFA) to yield an inseparable mixture of the acetonide protected intermediates as a colorless solid. The solid was dissolved in acetic acid (5 mL) and water (1 mL) and heated at 80° C. for 16 h. The reaction mixture was concentrated in vacuo, the residue was dissolved in methanol (3 mL) and purified by HPLC (15 min, 30-90% acetonitrile in water+0.1% TFA) to yield the two isomers benzyl 6-[(2R,3S,4R,5S)-5-acetamido-2-allyl-3,4-dihydroxy-1-piperidyl]-6-oxo-hexanoate (153, 6 mg, 0.01 mmol, 10% over two steps) and benzyl 6-[(2S,3S,4R,5S)-5-acetamido-2-allyl-3,4-dihydroxy-1-piperidyl]-6-oxo-hexanoate (154, 6 mg, 0.01 mmol, 10% over two steps). The assignment of the absolute stereochemistry was done arbitrarily.

Benzyl 6-[(2R,3S,4R,5S)-5-acetamido-2-allyl-3,4-dihydroxy-1-piperidyl]-6-oxo-hexanoate (153):

LC-MS (Method D):

Rₜ[min] (UV-signal 220 nm): 1.05 (only one peak for both isomers)

M[g/mol]: 443.1 [M+H⁺]

¹H NMR (400 MHz, DMSO-d6) δ[ppm]: 7.76 (s, br, 1H), 7.46-7.25 (m, 5H), 5.76-5.54 (m, 1H), 5.09 (s, 2H), 5.02-4.49 (m, 5H), 3.89-3.70 (m, 2H), 3.59-3.55 (m, 1H), 2.70-2.43 (m, 2H), 2.38-2.07 (m, 4H), 1.81 (s, 3H), 1.64-1.37 (m, 5H), 1.33-1.19 (m, 1H) (main conformer due to amide resonance).

Benzyl 6-[(2S,3S,4R,5S)-5-acetamido-2-allyl-3,4-dihydroxy-1-piperidyl]-6-oxo-hexanoate (154)

LC-MS (Method D):

Rₜ[min] (UV-signal 220 nm): 1.05 (only one peak for both isomers)

M[g/mol]: 443.1 [M+H⁺]

¹H NMR (400 MHz, DMSO-d6) δ[ppm]: 7.76 (s, br, 1H), 7.42-7.28 (m, 5H), 5.83-5.67 (m, 1H), 5.17-4.83 (m, 5H), 5.09 (s, 2H), 3.85-3.45 (m, 1H), 3.24-2.98 (m, 2H), 2.45-2.27 (m, 4H), 2.08-1.94 (m, 2H), 1.80 (s, 3H), 1.66-1.39 (m, 5H), 1.33-1.17 (m, 1H) (main conformer due to amide resonance).

### Example 13: Syntheses of compounds 160, 161, and 162

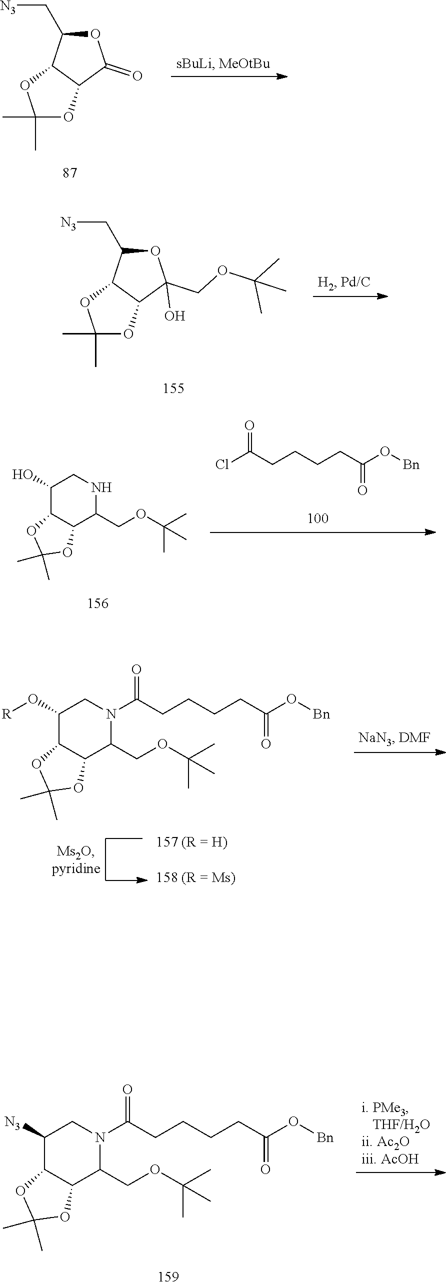

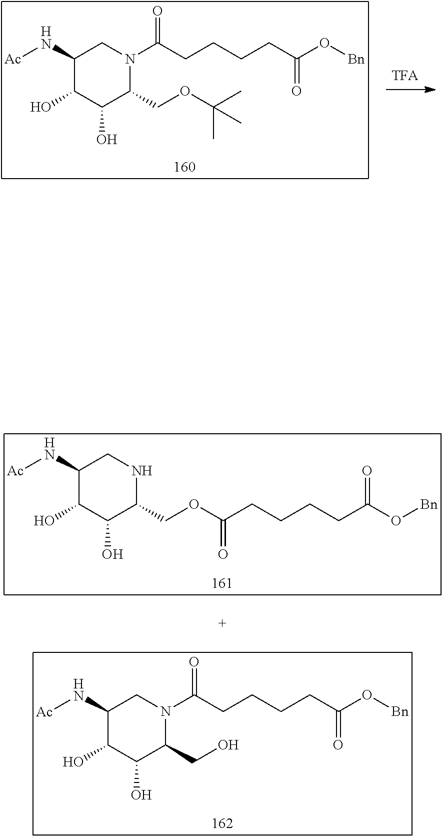 [SMILES: CC(=O)N[C@H]1CN(C(=O)CCCCC(=O)OCc2ccccc2)[C@H](COC(C)(C)C)[C@H](O)[C@@H]1O ; CC(=O)N[C@H]1CN[C@H](COC(=O)CCCCC(=O)OCc2ccccc2)[C@H](O)[C@@H]1O ; CC(=O)N[C@H]1CN(C(=O)CCCCC(=O)OCc2ccccc2)[C@@H](CO)[C@H](O)[C@@H]1O]

### Example 13.1: Synthesis of (3aR,6R,6aR)-6-(azidomethyl)-4-(tert-butoxymethyl)-2,2-dimethyl-6,6a-dihydro-3aH-furo[3,4-d][1,3]dioxol-4-ol (155)

The reaction with sec-butyllithium was performed under an argon atmosphere and in flame-dried glassware. A suspension of KOtBu (2.83 g, 25.18 mmol, 2.20 equiv.) in anhydrous tert-butyl methyl ether (100 mL) was cooled to −78° C., sec-butyllithium (1.3 M in hexane, 17.61 mL, 22.89 mmol, 2.00 equiv.) was added dropwise and the reaction mixture was stirred for 2 h at −78° C. In a separate flask, a solution of (3aR,6R,6aR)-6-(aminomethyl)-2,2-dimethyl-6,6a-dihydro-3aH-furo[3,4-d][1,3]dioxol-4-one (87, 2.44 g, 11.45 mmol, 1.00 equiv.) in anhydrous THF (50 mL) was cooled to −78° C. and the solution of the prepared lithium reagent was added portionwise. After addition of approx. 1.5 equiv., full conversion of starting material was observed by LC/MS and the reaction was stopped by addition of saturated aqueous NH₄C1-solution (50 mL). EtOAc (150 mL) was added, the layers were separated, the organic layer was washed with saturated aqueous NaCl-solution (30 mL), dried (MgSO₄), filtered and concentrated in vacuo. The crude product was purified by flash chromatography (silica, 1-30% EtOAc in n-heptane) to yield (3aR,6R,6aR)-6-(azidomethyl)-4-(tert-butoxymethyl)-2,2-dimethyl-6,6a-dihydro-3aH-furo[3,4-d][1,3]dioxol-4-ol (155, 704 mg, 2.34 mmol, 20%) as a colorless oil and an inseparable mixture of epimers at the semi-ketal.

LC-MS (Method D):

Rₜ[min] ((TIC-signal): 1.33/1.39 (two isomers detected)

M[g/mol]: 256.2 [M−H₂O—N₂+H⁺]

### Example 13.2: Synthesis of (3aS,7R,7aR)-4-(tert-butoxymethyl)-2,2-dimethyl-3a,4,5,6,7,7a-hexahydro-[1,3]dioxolo[4,5-c]pyridin-7-ol (156)

(3aR,6R,6aR)-6-(azidomethyl)-4-(tert-butoxymethyl)-2,2-dimethyl-6,6a-dihydro-3aH-furo[3,4-d] [1,3]dioxol-4-ol (155, 762 mg, 2.53 mmol, 1.00 equiv.) was dissolved in THF (40 mL), 10% Pd/C (0.07 g, 0.06 mmol, 0.03 equiv.) was added and the mixture was hydrogenated in an autoclave at r.t. and 4 bar hydrogen gas for 1 d. Since complete piperidine formation was detected by LC/MS, the reaction mixture was filtered and concentrated in vacuo. The crude product (3aS,7R,7aR)-4-(tert-butoxymethyl)-2,2-dimethyl-3a,4,5,6,7,7a-hexahydro-[1,3]di-oxolo[4,5-c]pyridin-7-ol (156) was directly used in solution for the next step.

LC-MS (Method D):

Rₜ[min] (TIC-signal): 0.64

M[g/mol]: 206.2 [M+H⁺]

### Example 13.3: Synthesis of benzyl 6-[(3aS,7R,7aR)-4-(tert-butoxymethyl)-7-hydroxy-2,2-dimethyl-4,6,7,7a-tetrahydro-3aH-[1,3]dioxolo[4,5-c]pyridin-5-yl]-6-oxo-hexanoate (157)

To a solution of crude (3aS,7R,7aR)-4-(tert-butoxymethyl)-2,2-dimethyl-3a,4,5,6,7,7a-hexahydro-[1,3]dioxolo[4,5-c]pyridin-7-ol (156, max. 2.53 mmol, 1.00 equiv.) in THF (40 mL) and saturated aqueous NaHCO₃-solution (50 mL) was added a solution of acid chloride benzyl 6-chloro-6-oxo-hexanoate (100, 709 mg, 2.78 mmol, 1.10 equiv.) in THF (10 mL) and the reaction mixture was stirred for 16 h at r.t. EtOAc (100 mL) and water (20 mL) were added, the layers were separated, the organic layer was washed with aqueous 2 N NaOH solution (3×30 mL), saturated aqueous NaCl-solution (30 mL), dried (MgSO₄), filtered and concentrated in vacuo. The crude product was purified by flash chromatography (silica, 10-80% EtOAc in n-heptane) to yield benzyl 6-[(3aS,7R,7aR)-4-(tert-butoxymethyl)-7-hydroxy-2,2-dimethyl-4,6,7,7a-tetrahydro-3aH-[1,3]dioxolo[4,5-c]pyridin-5-yl]-6-oxo-hexanoate (157, 881 mg, 1.84 mmol, 73% over two steps) as a colorless oil and an inseparable mixture of epimers at position 4.

LC-MS (Method D):

Rₜ[min] (UV-signal 220 nm): 1.48

M[g/mol]: 478.3 [M+H⁺]

### Example 13.4: Synthesis of benzyl 6-[(3aS,7R,7aS)-4-(tert-butoxymethyl)-2,2-dimethyl-7-methylsulfonyloxy-4,6,7,7a-tetrahydro-3aH-[1,3]dioxolo[4,5-c]pyridin-5-yl]-6-oxo-hexanoate (158)

A solution of benzyl 6-[(3aS,7R,7aR)-4-(tert-butoxymethyl)-7-hydroxy-2,2-dimethyl-4,6,7,7a-tetrahydro-3aH-[1,3]dioxolo[4,5-c]pyridin-5-yl]-6-oxo-hexanoate (157, 881 mg, 1.84 mmol, 1.00 equiv.) in anhydrous DCM (20 mL) was cooled to 0° C. and pyridine (0.50 mL, 6.15 mmol, 3.33 equiv.) and mesyl anhydride (545 mg, 3.07 mmol, 1.66 equiv.) were added. The reaction mixture was stirred for 3 h at 0° C. until LC/MS indicated full conversion of the starting material. Aqueous 1 N HCl (30 mL) and EtOAc (50 mL) were added, the layers were separated, the organic layer was washed with saturated aqueous NaCl-solution (30 mL), dried (MgSO₄), filtered and concentrated in vacuo. The crude product benzyl 6-[(3aS,7R,7aS)-4-(tert-butoxymethyl)-2,2-dimethyl-7-methylsulfonyloxy-4,6,7,7a-tetrahydro-3aH-[1,3]dioxolo[4,5-c]pyridin-5-yl]-6-oxo-hexanoate (158) was directly used for the next step.

LC-MS (Method D):

Rₜ[min] (UV-signal 220 nm): 1.69

M[g/mol]: 556.3 [M+H⁺]

### Example 13.5: Synthesis of benzyl 6-[(3aS,7S,7aR)-4-(tert-butoxymethyl)-2,2,7-trimethyl-4,6,7,7a-tetrahydro-3aH-[1,3]dioxolo[4,5-c]pyridin-5-yl]-6-oxo-hexanoate (159)

The crude product benzyl 6-[(3aS,7R,7aS)-4-(tert-butoxymethyl)-2,2-dimethyl-7-methylsulfonyloxy-4,6,7,7a-tetrahydro-3aH-[1,3]dioxolo[4,5-c]pyridin-5-yl]-6-oxo-hexanoate (158, max. 1.84 mmol, 1.00 equiv.) was dissolved in DMF (10 mL), NaN₃ (0.49 g, 7.50 mmol, 4.41 equiv.) and 15-crown-5 ether (0.63 g, 2.85 mmol, 1.68 equiv.) were added and the mixture was stirred at 100° C. for 2 d. EtOAc (50 mL) and water (50 mL) were added, the layers were separated, and the aqueous layer was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 1-30% EtOAc in n-heptane) to yield benzyl 6-[(3aS,7S,7aR)-4-(tert-butoxymethyl)-2,2,7-trimethyl-4,6,7,7a-tetrahydro-3aH-[1,3]dioxolo[4,5-c]pyridin-5-yl]-6-oxo-hexanoate (159, 284 mg, 0.57 mmol, 31% over two steps) as a colorless oil and an inseparable mixture of epimers at position 4.

LC-MS (Method D):

Rₜ[min] (UV-signal 220 nm): 1.83

M[g/mol]: 503.2 [M+H⁺]

### Example 13.6: Synthesis of benzyl 6-[(2R,3S,4R,5S)-5-acetamido-2-(tert-butoxymethyl)-3,4-dihydroxy-1-piperidyl]-6-oxo-hexanoate (160)

To a solution of benzyl 6-[(3aS,7S,7aR)-4-(tert-butoxymethyl)-2,2,7-trimethyl-4,6,7,7a-tetrahydro-3aH-[1,3]dioxolo[4,5-c]pyridin-5-yl]-6-oxo-hexanoate (159, 284 mg, 0.57 mmol, 1.00 equiv.) in THF (5 mL) and water (0.1 mL) was added PMe₃ (1 N in THF, 0.84 mL, 0.84 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.26 mL, approx. 5 equiv.) and pyridine (0.18 mL, approx. 4 equiv.) were added at r.t. and the reaction mixture was stirred for 1 h. Since LC/MS indicated full formation of the acetamide, the reaction mixture was concentrated in vacuo. The crude product was dissolved in methanol (3 mL) and purified by HPLC (15 min, 10-95% acetonitrile in water+0.1% TFA) to yield an inseparable mixture of the acetonide protected intermediates as a colorless solid. The solid was dissolved in acetic acid (5 mL) and water (1 mL) and heated at 80° C. for 16 h. The reaction mixture was concentrated in vacuo and the residue was dissolved in methanol (3 mL) and purified by HPLC (15 min, 20-70% acetonitrile in water+0.1% TFA) to yield benzyl 6-[(2R,3S,4R,5S)-5-acetamido-2-(tert-butoxymethyl)-3,4-dihydroxy-1-piperidyl]-6-oxo-hexanoate (160, 102 mg, 0.21 mmol, 37% over two steps) as an inseparable mixture of isomers.

LC-MS (Method D):

Rₜ[min] (UV-signal 220 nm): 1.14

M[g/mol]: 479.2 [M+H⁺]

¹H NMR (400 MHz, DMSO-d₆) δ[ppm]: 7.90/7.56 (d, J=6.5/8.3 Hz, 1H), 7.44-7.26 (m, 5H), 5.36-4.44 (m, 3H), 5.08 (s, 2H), 4.21-3.46 (m, 6H), 2.90 (d, J=13.6 Hz, 1H), 2.70-2.55 (m, 1H), 2.44-2.02 (m, 3H), 1.81/1.80 (s, 3H), 1.60-1.42 (m, 4H), 1.06 (s, 9H) (mixture of two isomers).

### Example 13.7: Synthesis of 06-[[(2R,3S,4R,5S)-5-acetamido-3,4-dihydroxy-2-piperidyl]methyl] 01-benzyl hexanedioate (161) and benzyl 6-[(2S,3S,4R,5S)-5-acetamido-3,4-dihydroxy-2-(hydroxymethyl)-1-piperidyl]-6-oxo-hexanoate (162)

Benzyl 6-[(2R,3S,4R,5S)-5-acetamido-2-(tert-butoxymethyl)-3,4-dihydroxy-1-piperidyl]-6-oxo-hexanoate (160, 74 mg, 0.16 mmol, 1.00 equiv.) was dissolved in DCM (2 mL) and TFA (1 mL). After 1 h the reaction mixture was concentrated in vacuo and the residue was purified by HPLC (15 min, 10-90% acetonitrile in water+0.1% TFA) to yield 06-[[(2R,3S,4R,5S)-5-acetamido-3,4-dihydroxy-2-piperidyl]methyl] 01-benzyl hexanedioate (161, TFA-salt, 38 mg, 0.07 mmol, 46%) and benzyl 6-[(2S,3S,4R,5S)-5-acetamido-3,4-dihydroxy-2-(hydroxymethyl)-1-piperidyl]-6-oxo-hexanoate (162, 22 mg, 0.05 mmol, 34%) as colorless solids. The assignment of the absolute stereochemistry was done by full NMR characterisation. For the cis-isomer 06-[[(2R,3S,4R,5S)-5-acetamido-3,4-dihydroxy-2-piperidyl]methyl] 01-benzyl hexanedioate (161), the migration of the acyl linker from the piperidine nitrogen to the proximate primary oxygen was proven by 2D-NMR experiments.

O6-[[(2R,3S,4R,5S)-5-Acetamido-3,4-dihydroxy-2-piperidyl]methyl] 01-benzyl hexanedioate (161):

LC-MS (Method D):

Rₜ[min] (UV-signal 220 nm): 0.82

M[g/mol]: 423.2 [M+H⁺]

¹H NMR (500 MHz, DMSO-d₆) δ[ppm]: 9.06 (s, br, 1H), 8.78 (s, br, 1H), 7.89/7.85 (d, J=8.2/7.6 Hz, 1H), 7.41-7.29 (m, 5H), 5.95/5.79 (s, br, 1H), 5.09 (s, 2H), 4.20 (d, J=4.2 Hz, 2H), 4.11 (m, 1H), 3.96 (m, 1H), 3.53 (m, 2H), 3.17 (m, 1H), 2.66 (m, 1H), 2.41-2.30 (m, 4H), 1.85/1.79 (s, 3H), 1.62-1.54 (m, 4H) (two main conformers due to amide resonance).

¹³C NMR (125 MHz, DMSO-d₆) δ[ppm]: 172.4, 172.2, 169.8, 136.1, 128.3, 127.8, 127.7, 69.9, 66.7, 65.2, 61.3, 56.4, 45.0, 44.6, 33.0, 32.7, 23.7, 23.5, 22.6.

Benzyl 6-[(2S,3S,4R,5S)-5-acetamido-3,4-dihydroxy-2-(hydroxymethyl)-1-piperidyl]-6-oxo-hexanoate (162):

LC-MS (Method D):

Rₜ[min] (UV-signal 220 nm): 0.87

M[g/mol]: 423.2 [M+H⁺]

¹H NMR (400 MHz, DMSO-d₆) δ[ppm]: 7.98-7.80/7.52 (m/d, J=8.2 Hz, 1H), 7.41-7.28 (m, 5H), 5.36-4.80 (m, 2H), 5.08 (s, 2H), 4.56/4.43 (m, 1H), 4.20-3.39 (m, 7H), 3.10/2.92 (m, 1H), 2.42-2.31 (m, 4H), 1.85-1.76 (m 3H), 1.61-1.45 (m, 4H).

(two main conformers due to amide resonance).

### Example 14: Syntheses of Compounds 180 and 181

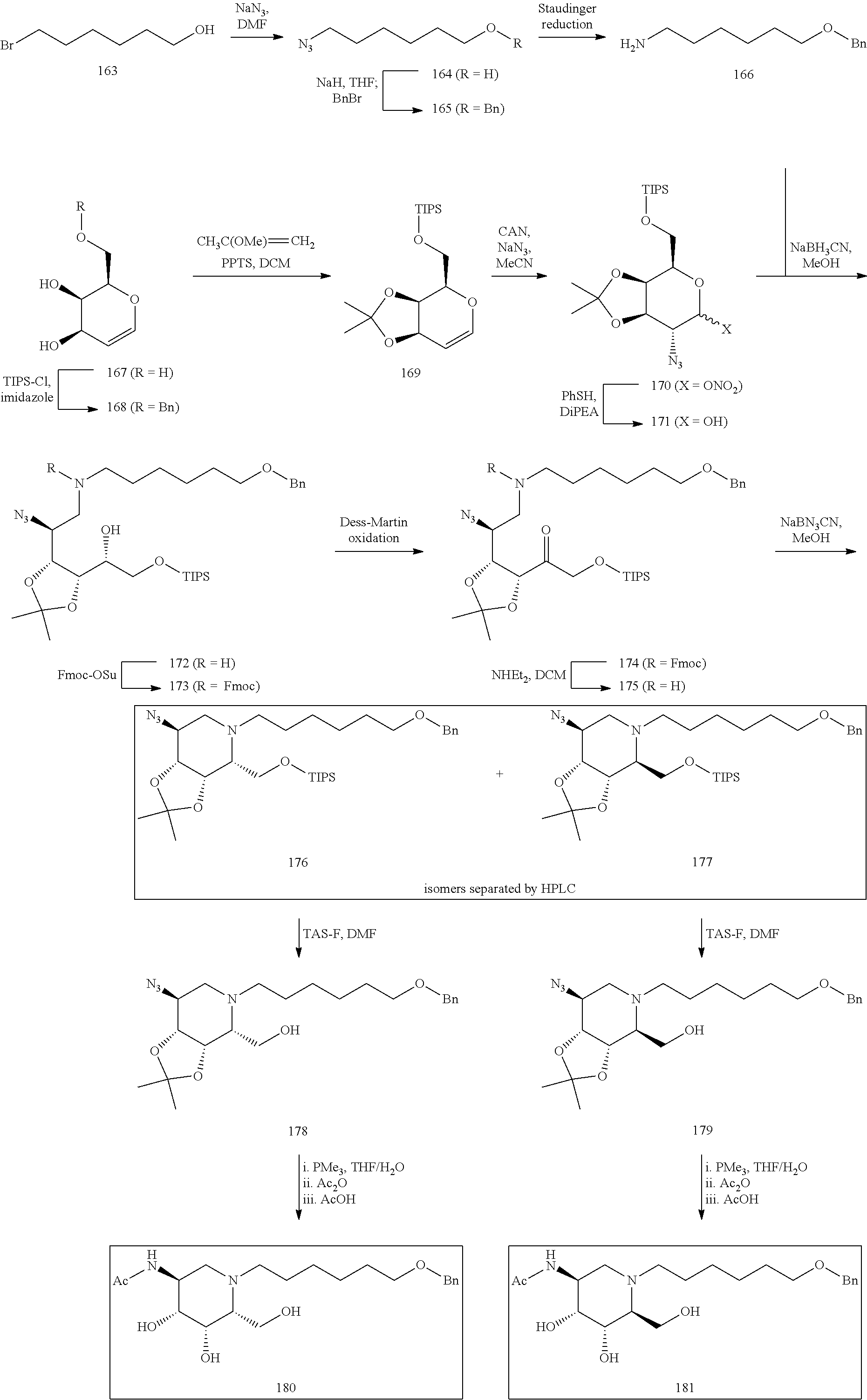

### Example 14.1: Synthesis of 6-azidohexan-1-ol (164)

6-Bromohexan-1-ol (163, 5.10 g, 28.17 mmol, 1.00 equiv.) was dissolved in DMF (60 mL), NaN₃ (4.58 g, 70.41 mmol, 2.50 equiv.) was added and the mixture was stirred at 60° C. for 16 h. EtOAc (100 mL) and water (100 mL) were added, the layers were separated, and the aqueous layer was re-extracted with EtOAc (2×100 mL). The combined organic layers were washed with saturated aqueous NaCl-solution (50 mL), dried (Na₂SO₄), filtered and concentrated in vacuo. The crude product 6-azidohexan-1-ol (164) was obtained as a colorless oil and directly used for the next reaction.

LC-MS (Method F):

Rₜ[min] (UV-signal 214 nm): 1.70

M[g/mol]: 144 [M+H⁺]

### Example 14.2: Synthesis of (((6-azidohexyl)oxy)methyl)benzene (165)

A solution of crude 6-azidohexan-1-ol (164, max. 28.14 mmol, 1.00 equiv.) in anhydrous THF (100 mL) was cooled to 0° C. and sodium hydride (60% in mineral oil, 1.69 g, 42.22 mmol, 1.50 equiv.) was added in small portions. The reaction mixture was stirred for 10 min at 0° C. and benzyl bromide (5.78 g, 33.77 mmol, 1.20 equiv.) was added. The reaction mixture was stirred for 16 h at r.t. EtOAc (100 mL) and water (100 mL) were added, the layers were separated, and the aqueous layer was re-extracted with EtOAc (2×100 mL). The combined organic layers were washed with saturated aqueous NaCl-solution (50 mL), dried (Na₂SO₄), filtered and concentrated in vacuo. The crude product was purified by flash chromatography (silica, 10% EtOAc in n-heptane) to yield (((6-azidohexyl)oxy)methyl)benzene (165, 4.80 g, 20.57 mmol, 73% over two steps) as a colorless oil.

LC-MS (Method F):

Rₜ[min] (UV-signal 214 nm): 2.25

M[g/mol]: 234 [M+H⁺]

### Example 14.3: Synthesis of 6-(benzyloxy)hexan-1-amine (166)

To a solution of (((6-azidohexyl)oxy)methyl)benzene (165, 4.80 g, 20.57 mmol, 1.00 equiv.) in THF (100 mL) was added 2-(diphenylphosphanyl)benzoic acid (7.56 g, 24.69 mmol, 1.20 equiv.) and the reaction mixture was stirred for 16 h at r.t. Aqueous 0.5 N HCl (200 mL) and diethyl ether (50 mL) were added, the layers were separated, and the aqueous layers was re-extracted with diethyl ether (2×50 mL). The aqueous layer was neutralized with saturated aqueous NaHCO₃-solution and was extracted with DCM (3×50 mL). The combined organic layers were washed with saturated aqueous NaCl-solution (50 mL), dried (Na₂SO₄), filtered and concentrated in vacuo. The crude product 6-(benzyloxy)hexan-1-amine (166, 1.90 g, 9.16 mmol, 45%) was obtained as a yellow oil and used for the next step without further purification.

LC-MS (Method F):

Rₜ[min] (UV-signal 214 nm): 1.50

M[g/mol]: 208 [M+H⁺]

### Example 14.4: Synthesis of (2R,3R,4R)-2-(((triisopropylsilyl)oxy)methyl)-3,4-dihydro-2H-pyran-3,4-diol (168)

A solution of (2R,3R,4R)-2-(hydroxymethyl)-3,4-dihydro-2H-pyran-3,4-diol (167, 10.00 g, 68.43 mmol, 1.00 equiv.) in DMF (100 mL) was cooled to 0° C. and imidazole (9.32 g, 136.85 mmol, 2.00 equiv.) and TIPSCl (19.79 g, 102.64 mmol, 1.50 equiv.) were added. The reaction mixture was stirred for 16 h at r.t. EtOAc (500 mL) and water (500 mL) were added, the layers were separated, and the organic layer was washed with saturated aqueous NaCl-solution (100 mL), dried (Na₂SO₄), filtered and concentrated in vacuo. The crude product was purified by flash chromatography (silica, 17% EtOAc in n-heptane) to yield (2R,3R,4R)-2-(((triisopropylsilyl)oxy)methyl)-3,4-dihydro-2H-pyran-3,4-diol (168, 17.00 g, 56.20 mmol, 82%) as a colorless oil.

LC-MS (Method F):

Rₜ[min] (UV-signal 214 nm): 2.11

M[g/mol]: 325 [M+Na⁺]

### Example 14.5: Synthesis of (((3aR,4R,7aR)-2,2-dimethyl-3a,7a-dihydro-4H-[1,3]dioxolo[4,5-c]pyran-4-yl)methoxy)triisopropylsilane (169)

A solution of (2R,3R,4R)-2-(((triisopropylsilyl)oxy)methyl)-3,4-dihydro-2H-pyran-3,4-diol (168, 10.00 g, 33.06 mmol, 1.00 equiv.) in DCM (100 mL) was cooled to 0° C. and 2-methoxypropene (3.58 g, 49.59 mmol, 1.50 equiv.) and PPTS (415 mg, 1.65 mmol, 0.05 equiv.) were added. The reaction mixture was stirred for 30 min at 0° C. and 4 h at r.t. The reaction mixture was concentrated in vacuo, dissolved in diethyl ether (100 mL) and saturated aqueous NaHCO₃-solution (50 mL). The layers were separated, the organic layer was washed with saturated aqueous NaCl-solution (30 mL), dried (Na₂SO₄), filtered and concentrated in vacuo. The crude product was purified by flash chromatography (silica, 10% EtOAc in n-heptane) to yield (((3aR,4R,7aR)-2,2-dimethyl-3a,7a-dihydro-4H-[1,3]dioxolo[4,5-c]pyran-4-yl)methoxy) triisopropylsilane (169, 9.20 g, 26.86 mmol, 81%) as a colorless oil.

LC-MS (Method F):

Rₜ[min] (UV-signal 214 nm): 1.74

M[g/mol]: 365 [M+Na⁺]

### Example 14.6: Synthesis of (3aR,4R,7R,7aR)-7-azido-2,2-dimethyl-4-(((triisopropylsilyl)oxy)methyl)tetrahydro-4H-[1,3]dioxolo[4,5-c]pyran-6-yl nitrate (170)

A solution of ((((3aR,4R,7aR)-2,2-dimethyl-3a,7a-dihydro-4H-[1,3]dioxolo[4,5-c]pyran-4-yl)methoxy) triisopropylsilane (169, 9.00 g, 26.27 mmol, 1.00 equiv.) in acetonitrile (200 mL) was cooled to −20° C. and NaN₃ (2.56 g, 39.41 mmol, 1.50 equiv.) and CAN (43.21 g, 78.82 mmol, 3.00 equiv.) were added. The reaction mixture was stirred for 16 h at −20° C. Diethyl ether (500 mL) was added and the organic layer was washed with water (100 mL), saturated aqueous NaCl-solution (100 mL), dried (Na₂SO₄), filtered and concentrated in vacuo. The crude product was purified by flash chromatography (silica, 10% EtOAc in n-heptane) to yield (3aR,4R,7R,7aR)-7-azido-2,2-dimethyl-4-(((triisopropylsilyl)oxy)methyl)tetrahydro-4H-[1,3] dioxolo[4,5-c]pyran-6-yl nitrate (170, 3.40 g, 7.61 mmol, 29%) as a yellow oil. The ratio of α-isomer and β-isomer was almost 1:1 based on ¹H NMR.

¹H NMR (300 MHz, CDCl₃) δ[ppm] (isomer 1): 6.34 (d, J=3.9 Hz, 1H), 4.34 (m, 2H), 4.21 (t, J=6.4 Hz, 1H), 3.95 (dd, J=9.6, 7.2 Hz, 1H), 3.85 (dd, J=9.6, 6.4 Hz, 1H), 3.78 (m, 1H), 1.52 (s, 3H), 1.35 (s, 3H), 1.04 (m, 21H);

¹H NMR (300 MHz, CDCl₃) δ[ppm] (isomer 2): 5.50 (d, J=8.9 Hz, 1H), 4.30 (dd, J=4.3, 1.5 Hz, 1H), 4.15 (dd, J=6.2, 4.3 Hz, 1H), 3.89-4.03 (m, 3H), 3.56 (dd, J=8.9, 7.3 Hz, 1H), 1.58 (s, 3H), 1.38 (s, 3H), 1.08 (m, 21H).

### Example 14.7: Synthesis of (3aR,4R,7R,7aR)-7-azido-2,2-dimethyl-4-(((triisopropylsilyl)oxy) methyl)tetrahydro-4H-[1,3]dioxolo[4,5-c]pyran-6-ol (171)

A solution of (3aR,4R,7R,7aR)-7-azido-2,2-dimethyl-4-(((triisopropylsilyl)oxy)methyl) tetrahydro-4H-[1,3]dioxolo[4,5-c]pyran-6-yl nitrate (170, 3.40 g, 7.61 mmol, 1.00 equiv.) in acetonitrile (30 mL) was cooled to 0° C. and DIPEA (1.33 mL, 7.61 mmol, 1.00 equiv.) and thiophenol (2.52 g, 22.84 mmol, 3.00 equiv.) were added. The reaction mixture was stirred for 1 h at 0° C. The reaction mixture was concentrated in vacuo and the crude product was purified by flash chromatography (silica, 16% EtOAc in n-heptane) to yield (3aR,4R,7R,7aR)-7-azido-2,2-dimethyl-4-(((triisopropylsilyl)oxy)methyl)tetrahydro-4H-[1,3]dioxolo[4,5-c]pyran-6-ol (171, 3.10 g, quant.) as a colorless oil.

LC-MS (Method F):

Rₜ[min] (UV-signal 214 nm): 2.37, 2.43 (two isomers)

M[g/mol]: 424 [M+Na⁺]

### Example 14.8: Synthesis of (R)-1-((4S,5R)-5-((S)-1-azido-2-((6-(benzyloxy)hexyl)amino) ethyl)-2,2-dimethyl-1,3-dioxolan-4-yl)-2-((triisopropylsilyl)oxy) ethan-1-ol (172)

A mixture of (3aR,4R,7R,7aR)-7-azido-2,2-dimethyl-4-(((triisopropylsilyl)oxy)methyl) tetrahydro-4H-[1,3]dioxolo[4,5-c]pyran-6-ol (171, 1.20 g, 2.99 mmol, 1.00 equiv.) and 6-(benzyloxy)hexan-1-amine (166, 619 mg, 2.99 mmol, 1.00 equiv.) in methanol (40 mL) was stirred at r.t. for 1 h and NaBH₃CN (939 mg, 14.94 mmol, 5.00 equiv.) and acetic acid (1.71 mL, 29.88 mmol, 10.00 equiv.) were added. The reaction mixture was stirred for 48 h at 80° C. DCM (20 mL) and saturated aqueous NaHCO₃-solution (20 mL) were added, the layers were separated, and the aqueous layers was re-extracted with DCM (2×20 mL). The combined organic layers were washed with water (20 mL), saturated aqueous NaCl-solution (20 mL), dried (Na₂SO₄), filtered and concentrated in vacuo. The crude product was purified by flash chromatography (silica, 10% MeOH in DCM) to yield (R)-1-((4S,5R)-5-((S)-1-azido-2-((6-(benzyloxy)hexyl)amino)ethyl)-2,2-dimethyl-1,3-dioxolan-4-yl)-2-((triisopropylsilyl)oxy) ethan-1-ol (172, 510 mg, 0.86 mmol, 29%) as a colorless oil.

LC-MS (Method F):

Rₜ[min] (UV-signal 214 nm): 1.99

M[g/mol]: 593 [M+Na⁺]

### Example 14.9: Synthesis of (9H-fluoren-9-yl)methyl ((S)-2-azido-2-((4R,5S)-5-((R)-1-hydroxy-2-((triisopropylsilyl)oxy)ethyl)-2,2-dimethyl-1,3-dioxolan-4-yl)ethyl)(6-(benzyloxy)hexyl)carbamate (173)

To a solution of (R)-1-((4S,5R)-5-((S)-1-azido-2-((6-(benzyloxy)hexyl)amino)ethyl)-2,2-dimethyl-1,3-dioxolan-4-yl)-2-((triisopropylsilyl)oxy) ethan-1-ol (172, 510 mg, 0.86 mmol, 1.00 equiv.) in THF (8 mL) and water (2 mL) was added NaHCO₃ (361 mg, 4.30 mmol, 5.00 equiv.) and Fmoc-OSu (348 mg, 1.03 mmol, 1.20 equiv.). The reaction mixture was stirred for 16 h at r.t. EtOAc (50 mL) and water (30 mL) were added, the layers were separated, and the organic layer was washed with water (10 mL), saturated aqueous NaCl-solution (10 mL), dried (Na₂SO₄), filtered and concentrated in vacuo. The crude product was purified by preparative TLC (silica, 17% EtOAc in n-heptane) to yield (9H-fluoren-9-yl)methyl((S)-2-azido-2-((4R,5S)-5-((R)-1-hydroxy-2-((triisopropylsilyl)oxy)ethyl)-2,2-dimethyl-1,3-dioxolan-4-yl) ethyl)(6-(benzyloxy)hexyl)carbamate (173, 410 mg, 0.50 mmol, 59%) as a colorless oil.

LC-MS (Method F):

Rₜ[min] (UV-signal 214 nm): 2.16

M[g/mol]: 816 [M+H⁺]

### Example 14.10: Synthesis of (9H-fluoren-9-yl)methyl ((S)-2-azido-2-((4R,5R)-2,2-dimethyl-5-(2-((triisopropylsilyl)oxy)acetyl)-1,3-dioxolan-4-yl)ethyl)(6-(benzyloxy)hexyl)carbamate (174)

To a solution of (9H-fluoren-9-yl)methyl((S)-2-azido-2-((4R,5S)-5-((R)-1-hydroxy-2-((triiso-propylsilyl)oxy)ethyl)-2,2-dimethyl-1,3-dioxolan-4-yl)ethyl)(6-(benzyloxy)hexyl)carbamate (173, 410 mg, 0.50 mmol, 1.00 equiv.) in DMSO (15 mL) was added IBX (704 mg, 2.51 mmol, 5.00 equiv.). The reaction mixture was stirred for 2 h at 60° C. EtOAc (50 mL) and water (30 mL) were added, the layers were separated, and the organic layer was washed with water (10 mL), saturated aqueous NaCl-solution (10 mL), dried (Na₂SO₄), filtered and concentrated in vacuo. The crude product was purified by preparative TLC (silica, 17% EtOAc in n-heptane) to yield (9H-fluoren-9-yl)methyl ((S)-2-azido-2-((4R,5R)-2,2-dimethyl-5-(2-((triisopropyl-silyl)oxy)acetyl)-1,3-dioxolan-4-yl)ethyl)(6-(benzyloxy)hexyl)carbamate (174, 330 mg, 0.41 mmol, 81%) as a colorless oil.

### Example 14.11: Synthesis of 1-((4R,5R)-5-((S)-1-azido-2-((6-(benzyloxy)hexyl)amino)ethyl)-2,2-dimethyl-1,3-dioxolan-4-yl)-2-((triisopropylsilyl)oxy)ethan-1-one (175)

To a solution of (9H-fluoren-9-yl)methyl((S)-2-azido-2-((4R,5R)-2,2-dimethyl-5-(2-((triisopropyl-silyl)oxy)acetyl)-1,3-dioxolan-4-yl)ethyl)(6-(benzyloxy)hexyl)carbamate (174, 330 mg, 0.41 mmol, 1.00 equiv.) in DCM (2 mL) was added NHEt₂ (1.02 mL, 9.74 mmol, 24.00 equiv.). The reaction mixture was stirred for 16 h at r.t. The reaction mixture was concentrated in vacuo and the crude product was purified by preparative TLC (silica, 17% EtOAc in n-heptane) to yield 1-((4R,5R)-5-((S)-1-azido-2-((6-(benzyloxy)hexyl)amino)ethyl)-2,2-dimethyl-1,3-dioxolan-4-yl)-2-((triisopropylsilyl)oxy)ethan-1-one (175, 260 mg, quant.) as a colorless oil which was used for the next step without further purification.

### Example 14.12: Synthesis of (3aS,4R,7S,7aR)-7-azido-5-(6-(benzyloxy)hexyl)-2,2-dimethyl-4-(((triisopropylsilyl)oxy)methyl)hexahydro-[1,3]dioxolo[4,5-c]pyridine (176) and (3aS,4S,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (177)

To a solution of 1-((4R,5R)-5-((S)-1-azido-2-((6-(benzyloxy)hexyl)amino)ethyl)-2,2-dimethyl-1,3-dioxolan-4-yl)-2-((triisopropylsilyl)oxy)ethan-1-one (175, max. 0.41 mmol, 1.00 equiv.) in methanol (4 mL) was added NaBH₃CN (138 mg, 2.20 mmol, 5.41 equiv.) and acetic acid (0.50 mL, 8.73 mmol, 21.50 equiv.). The reaction mixture was stirred for 16 h at r.t. EtOAc (30 mL) and water (10 mL) were added, the layers were separated, and the organic layer was washed with water (10 mL), saturated aqueous NaCl-solution (10 mL), dried (Na₂SO₄), filtered and concentrated in vacuo. The crude product was pre-purified by preparative TLC (silica, 10% EtOAc in n-heptane) followed by HPLC purification (0-100% acetonitrile in water+0.01% TFA) to obtain an inseparable mixture of (3aS,4R,7S,7aR)-7-azido-5-(6-(benzyloxy)hexyl)-2,2-dimethyl-4-(((triisopropylsilyl)oxy)methyl)hexahydro-[1,3]dioxolo[4,5-c]pyridine (176) and (3aS,4S,7S,7aR)-7-azido-5-(6-(benzyloxy)hexyl)-2,2-dimethyl-4-(((triisopropylsilyl)oxy) methyl) hexahydro-[1,3]dioxolo[4,5-c]pyridine (177, 105 mg, 0.18 mmol, 45%) as a yellow oil.

The mixture of (3aS,4R,7S,7aR)-7-azido-5-(6-(benzyloxy)hexyl)-2,2-dimethyl-4-(((triisopro-pylsilyl)oxy)methyl)hexahydro-[1,3]dioxolo[4,5-c]pyridine (176) and its epimer (3aS,4S,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (177) obtained from ChemPartner (114 mg, 0.20 mmol) was purified by HPLC (15 min, 35-95% acetonitrile in water+0.1% TFA) to obtain (3aS,4R,7S,7aR)-7-azido-5-(6-(benzyloxy)hexyl)-2,2-dimethyl-4-(((triisopropylsilyl) oxy)methyl)hexahydro-[1,3]dioxolo[4,5-c]pyridine (176, 58 mg, 0.10 mmol) and (3aS,4S,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (177, 27 mg, 0.05 mmol) as colorless oils which were directly used for the TIPS-deprotection. The absolute stereochemistry was elucidated retrospectively by full NMR characterisation.

(3aS,4R,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (176):

LC-MS (Method D):

Rₜ[min] (UV-signal 220 nm): 1.76

M[g/mol]: 575.3 [M+H⁺](3aS,4S,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (177):

LC-MS (Method D):

Rₜ[min] (UV-signal 220 nm): 1.85

M[g/mol]: 575.3 [M+H⁺]

### Example 14.13: Synthesis of [(3aS,4R,7S,7aR)-7-azido-5-(6-benzyloxyhexyl)-2,2-dimethyl-4,6,7,7a-tetrahydro-3aH-[1,3]dioxolo[4,5-c]pyridin-4-yl]methanol (178)

(3aS,4R,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (176, 58 mg, 0.10 mmol, 1.00 equiv.) was dissolved in DMF (1 mL), TAS-F (1 M in DMF, 0.15 mL, 0.15 mmol, 1.50 equiv.) was added and the mixture was stirred at r.t. for 5 h until full deprotection was detected by LC/MS. The solution was filtered and purified by HPLC (15 min, 10-99% acetonitrile in water+0.1% TFA) to obtain [(3aS,4R,7S,7aR)-7-azido-5-(6-benzyloxyhexyl)-2,2-dimethyl-4,6,7,7a-tetrahydro-3aH-[1,3]dioxolo[4,5-c]pyridin-4-yl]methanol (178, TFA-salt, 29 mg, 0.05 mmol, 54%) as a colorless oil. The absolute stereochemistry was elucidated by full NMR characterisation.

LC-MS (Method D):

Rₜ[min] (UV-signal 220 nm): 1.11

M[g/mol]: 419.2 [M+H⁺]

¹H NMR (600 MHz, DMSO-d₆) δ[ppm]: 7.38-7.24 (m, 5H), 4.49 (m, 1H), 4.45 (s, 2H), 4.23 (dd, J=7.6/5.7 Hz, 1H), 3.99 (s, br, 1H), 3.86 (m, 2H), 3.71 (s, br, 1H), 3.44 (t, J=6.4 Hz, 2H), 3.41 (dd, J=12.9/4.0, 1H), 3.36-3.25 (m, 1H), 3.25-3.17 (m, 1H), 3.13 (m, 1H), 1.68 (s, br, 2H), 1.57 (m, 2H), 1.51 (s, 3H), 1.41-1.26 (m, 4H), 1.33 (s, 3H).

¹³C NMR (150 MHz, DMSO-d₆) δ[ppm]: 138.6, 128.0, 127.2, 127.1, 109.6, 74.6, 72.7 (br), 71.7, 69.3, 60.5, 58.1 (br), 48.8 (br), 28.8, 27.4, 25.6, 25.4, 25.1 (three signals could not be assigned due to extreme line broadening).

### Example 14.14: Synthesis of [(3aS,4S,7S,7aR)-7-azido-5-(6-benzyloxyhexyl)-2,2-dimethyl-4,6,7,7a-tetrahydro-3aH-[1,3]dioxolo[4,5-c]pyridin-4-yl]methanol (179)

(3aS,4S,7S,7aR)-7-azido-5-(6-(benzyloxy)hexyl)-2,2-dimethyl-4-(((triisopropylsilyl)oxy) methyl)hexahydro-[1,3]dioxolo[4,5-c]pyridine (177, 27 mg, 0.05 mmol, 1.00 equiv.) was dissolved in DMF (1 mL), TAS-F (1 M in DMF, 0.08 mL, 0.08 mmol, 1.60 equiv.) was added and the mixture was stirred at r.t. for 5 h until full deprotection was detected by LC/MS. The solution was filtered and purified by HPLC (15 min, 15-65% acetonitrile in water+0.1% TFA) to obtain [(3aS,4S,7S,7aR)-7-azido-5-(6-benzyloxyhexyl)-2,2-dimethyl-4,6,7,7a-tetrahydro-3aH-[1,3]dioxolo[4,5-c]pyridin-4-yl]methanol (179, TFA-salt, 20 mg, 0.04 mmol, 80%) as a colorless oil. The absolute stereochemistry was elucidated by full NMR characterisation.

LC-MS (Method D):

Rₜ[min] (UV-signal 220 nm): 1.13

M[g/mol]: 419.2 [M+H⁺]

¹H NMR (600 MHz, DMSO-d₆) δ[ppm]: 7.37-7.26 (m, 5H), 4.45 (s, 2H), 4.42 (s, br, 1H), 4.25 (t, J=5.8 Hz, 1H), 4.22 (s, br, 1H), 3.98 (m, 1H), 3.76 (dd, J=12.2/4.5 Hz, 1H), 3.64 (s, br, 1H), 3.44 (t, J=6.4 Hz, 2H), 3.37-3.16 (m, 4H), 1.68 (m, 2H), 1.57 (m, 2H), 1.51 (s, 3H), 1.41-1.27 (m, 4H), 1.34 (s, 3H).

¹³C NMR (150 MHz, DMSO-d₆) δ[ppm]: 138.6, 128.0, 127.2, 127.1, 109.0, 74.0 (br), 71.7, 71.5 (br), 69.3, 60.5, 57.4 (br), 54.1 (br), 48.0 (br), 28.8, 27.3, 25.6, 25.5, 25.0, 23.4 (one signal could not be assigned due to extreme line broadening).

### Example 14.15: Synthesis of N-[(3S,4R,5S,6R)-1-(6-benzyloxyhexyl)-4,5-dihydroxy-6-(hydroxymethyl)-3-piperidyl]acetamide (180)

To a solution of [(3aS,4R,7S,7aR)-7-azido-5-(6-benzyloxyhexyl)-2,2-dimethyl-4,6,7,7a-tetrahydro-3aH-[1,3]dioxolo [4,5-c]pyridin-4-yl]methanol (178, 38 mg, 0.09 mmol, 1.00 equiv.) in THF (5 mL) and water (0.1 mL) was added PMe₃ (1 N in THF, 0.14 mL, 0.14 mmol, 1.56 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.09 mL, approx. 10 equiv.) and pyridine (0.07 mL, approx. 10 equiv.) were added and the reaction mixture was stirred for 1 h at r.t. Since LC/MS indicated full formation of the acetamide, the reaction mixture was concentrated in vacuo. The crude product was dissolved in acetic acid (5 mL) and water (1 mL) and heated at 80° C. for 16 h. The reaction mixture was concentrated in vacuo and the residue was dissolved in methanol (3 mL) and NaOMe (10% in MeOH, 0.2 mL) was added since partial formation of O-acetylation was detected by LC/MS. Full and selective saponification was detected by LC/MS after 1 h. Acetic acid (0.2 mL) was added, the sample was filtrated and purified by HPLC (15 min, 20-70% acetonitrile in water+0.1% TFA) to yield N-[(3S,4R,5S,6R)-1-(6-benzyloxyhexyl)-4,5-dihydroxy-6-(hydroxymethyl)-3-piperidyl]acetamide (180, TFA-salt, 28 mg, 0.06 mmol, 60%) as a colorless solid.

LC-MS (Method D):

Rₜ[min] (UV-signal 220 nm): 0.81

M[g/mol]: 395.3 [M+H⁺]

¹H NMR (400 MHz, DMSO-d6) δ[ppm]: 9.41 (s, br, 1H), 7.93 (d, J=7.6 Hz, 1H), 7.41-7.23 (m, 5H), 5.81 (s, br, 1H), 5.29-4.94 (m, 2H), 4.44 (s, 2H), 4.22-3.86 (m, 2H), 3.79-3.64 (m, 2H), 3.63-2.99 (m, 7H), 2.75 (m, 1H), 1.84 (s, 3H), 1.71-1.48 (m, 4H), 1.42-1.16 (m, 4H).

### Example 14.16: Synthesis of N-[(3S,4S,5S,6R)-1-(6-benzyloxyhexyl)-4,5-dihydroxy-6-(hydroxymethyl)-3-piperidyl]acetamide (181)

To a solution of [(3aS,4S,7S,7aR)-7-azido-5-(6-benzyloxyhexyl)-2,2-dimethyl-4,6,7,7a-tetrahydro-3aH-[1,3]dioxolo[4,5-c]pyridin-4-yl]methanol (179, 20 mg, 0.05 mmol, 1.00 equiv.) in THF (5 mL) and water (0.1 mL) was added PMe₃ (1 N in THF, 0.08 mL, 0.08 mmol, 1.60 equiv.) and the reaction mixture was stirred for 1 h at r.t. Since LC/MS indicated full conversion of the starting material, the reaction mixture was concentrated in vacuo. The residue was dissolved in EtOAc (20 mL), acetic anhydride (0.05 mL, approx. 10 equiv.) and pyridine (0.04 mL, approx. 10 equiv.) were added and the reaction mixture was stirred for 1 h at r.t. Since LC/MS indicated full formation of the acetamide, the reaction mixture was concentrated in vacuo. The crude product was dissolved in acetic acid (5 mL) and water (1 mL) and heated at 80° C. for 16 h. The reaction mixture was concentrated in vacuo and the residue was dissolved in methanol (3 mL) and NaOMe (10% in MeOH, 0.2 mL) was added since partial formation of O-acetylation was detected by LC/MS. Full and selective saponification was detected by LC/MS after 1 h. Acetic acid (0.2 mL) was added, the sample was filtrated and purified by HPLC (15 min, 20-70% acetonitrile in water+0.1% TFA) to yield N-[(3S,4S,5S,6R)-1-(6-benzyloxyhexyl)-4,5-dihydroxy-6-(hydroxymethyl)-3-piperidyl]acetamide (181, TFA-salt, 16 mg, 0.03 mmol, 66%) as a colorless solid.

LC-MS (Method D):

Rₜ[min] (UV-signal 220 nm): 0.84

M[g/mol]: 395.3 [M+H⁺]

¹H NMR (400 MHz, DMSO-d₆) δ[ppm]: 9.22/8.92 (s, br, 1H), 8.10/7.88 (d, J=6.6/7.7 Hz, 1H), 7.42-7.24 (m, 5H), 5.90/5.07 (s, br, 1H), 5.66-5.49 (m, 1H), 5.46-5.32 (m, 1H), 4.45 (s, 2H), 4.30-3.60 (m, 5H), 3.51-3.37 (m, 3H), 3.29-2.89 (m, 4H), 1.87/1.82 (s, 3H), 1.79-1.46 (m, 4H), 1.41-1.20 (m, 4H).

### Example 15: Synthesis of Linker Precursor for Trimerization

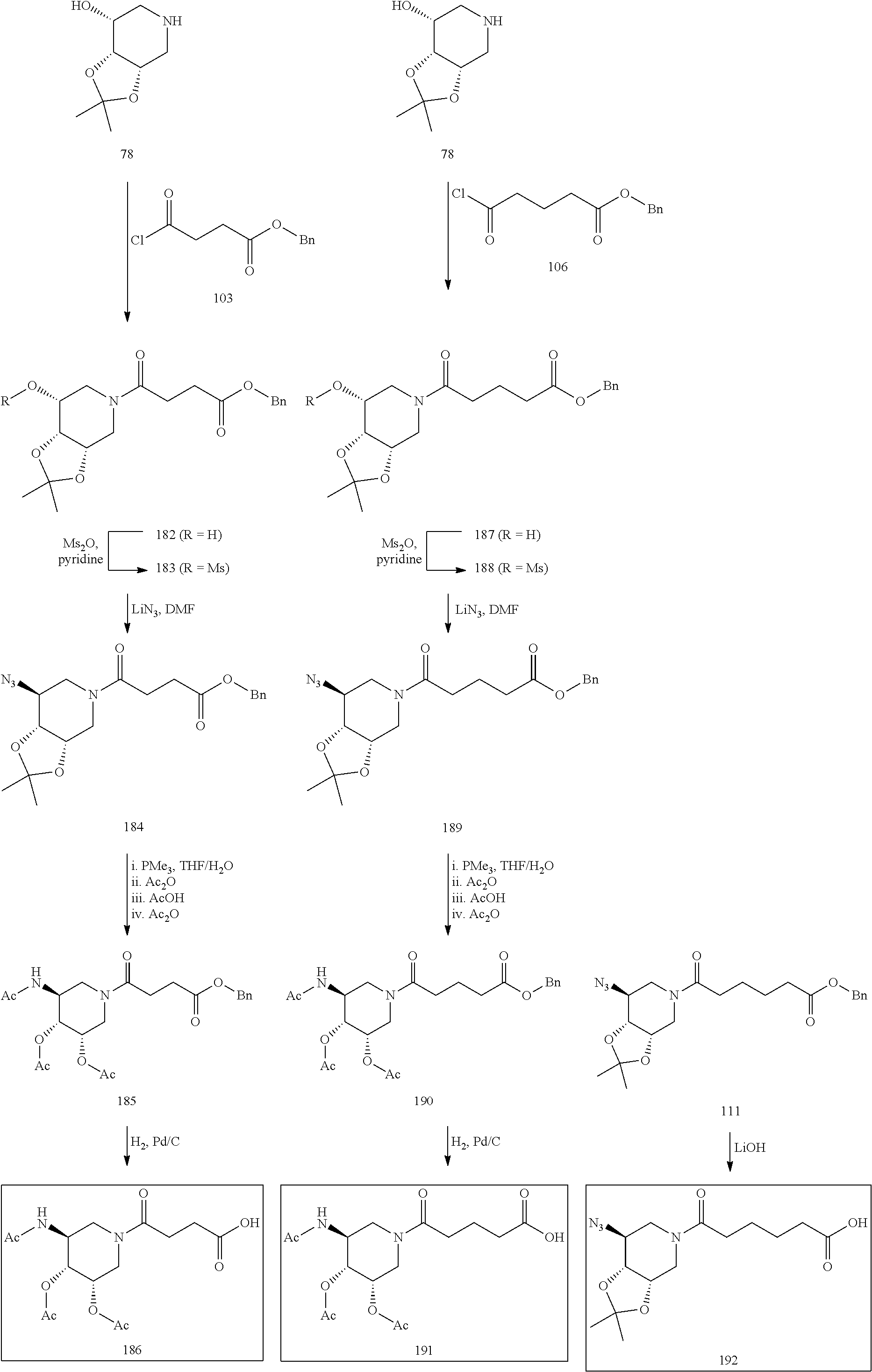

### Example 15.1: Synthesis of benzyl 4-[(3aS,7R,7aR)-7-hydroxy-2,2-dimethyl-4,6,7,7a-tetrahydro-3aH-[1,3]dioxolo[4,5-c]pyridin-5-yl]-4-oxo-butanoate (182)

To a solution of (3aS,7R,7aR)-2,2-dimethyl-3a,4,5,6,7,7a-hexahydro-[1,3]dioxolo[4,5-c]pyridin-7-ol (78) (318 mg, 1.84 mmol, 1.00 equiv.) in THF (50 mL) and saturated aqueous NaHCO₃-solution (10 mL), acid chloride benzyl 4-chloro-4-oxo-butanoate (103, 834 mg, 3.68 mmol, 2.00 equiv.) was added. The reaction mixture was stirred for 1 h at r.t. EtOAc (100 mL) and water (10 mL) were added, the layers were separated, the organic layers were washed with saturated aqueous NaCl-solution (20 mL), dried (MgSO₄), filtered and concentrated in vacuo. The crude product benzyl 4-[(3aS,7R,7aR)-7-hydroxy-2,2-dimethyl-4,6,7,7a-tetrahydro-3aH-[1,3]dioxolo[4,5-c]pyridin-5-yl]-4-oxo-butanoate (182) was directly used for the next step without further purification.

LC-MS (Method D):

Rₜ[min] (UV-signal 220 nm): 1.04

M[g/mol]: 364.1 [M+H⁺]

### Example 15.2: Synthesis of benzyl 4-[(3aS,7R,7aS)-2,2-dimethyl-7-methylsulfonyloxy-4,6,7,7a-tetrahydro-3aH-[1,3]dioxolo[4,5-c]pyridin-5-yl]-4-oxo-butanoate (183)

A solution of benzyl 4-[(3aS,7R,7aR)-7-hydroxy-2,2-dimethyl-4,6,7,7a-tetrahydro-3aH-[1,3]dioxolo[4,5-c]pyridin-5-yl]-4-oxo-butanoate (182, max. 1.84 mmol, 1.00 equiv.) in anhydrous DCM (10 mL) was cooled to 0° C. and pyridine (0.56 mL, 6.95 mmol, 3.77 equiv.) and mesyl anhydride (617 mg, 3.47 mmol, 1.88 equiv.) were added. The reaction mixture was stirred for 1.5 h at 0° C. until LC/MS indicated full conversion of the starting material. Aqueous 1 N HCl (10 mL) and EtOAc (50 mL) were added, the layers were separated, the organic layer was washed with saturated aqueous NaCl-solution (10 mL), dried (MgSO₄), filtered and concentrated in vacuo. The crude product was purified by flash chromatography (silica, 1-80% EtOAc in n-heptane) to yield benzyl 4-[(3aS,7R,7aS)-2,2-dimethyl-7-methylsulfonyloxy-4,6,7,7a-tetrahydro-3aH-[1,3]dioxolo[4,5-c]pyridin-5-yl]-4-oxo-butanoate (183, 445 mg, 1.01 mmol, 55% over two steps) as a colorless oil.

LC-MS (Method D):

Rₜ[min] (UV-signal 220 nm): 1.28

M[g/mol]: 442.1 [M+H⁺]

### Example 15.3: Synthesis of benzyl 4-[(3aS,7S,7aR)-7-azido-2,2-dimethyl-4,6,7,7a-tetrahydro-3aH-[1,3]dioxolo[4,5-c]pyridin-5-yl]-4-oxo-butanoate (184)

Benzyl 4-[(3aS,7R,7aS)-2,2-dimethyl-7-methylsulfonyloxy-4,6,7,7a-tetrahydro-3aH-[1,3]di-oxolo[4,5-c]pyridin-5-yl]-4-oxo-butanoate (183, 445 mg, 1.01 mmol, 1.00 equiv.) was dissolved in DMF (3 mL), LiN₃ (2 M in DMF, 1.26 mL, 2.52 mmol, 2.50 equiv.) was added and the mixture was stirred at 100° C. for 2 d. The reaction was stopped since significant amounts of an elimination product were detected by LC/MS. EtOAc (50 mL) and water (10 mL) were added, the layers were separated, the aqueous layer was re-extracted with EtOAc (3×20 mL), the combined organic layers were washed with saturated aqueous NaCl-solution (30 mL), dried (MgSO₄), filtered and concentrated in vacuo. The crude product was purified by flash chromatography (silica, 1-80% EtOAc in n-heptane) to yield benzyl 4-[(3aS,7S,7aR)-7-azido-2,2-dimethyl-4,6,7,7a-tetrahydro-3aH-[1,3]dioxolo[4,5-c]pyridin-5-yl]-4-oxo-butanoate (184, 72 mg, 0.18 mmol, 18%) as a colorless oil and recovered benzyl 4-[(3aS,7R,7aS)-2,2-dimethyl-7-methylsulfonyloxy-4,6,7,7a-tetrahydro-3aH-[1,3]dioxolo[4,5-c]pyridin-5-yl]-4-oxo-butanoate (183, 196 mg, 0.44 mmol, 44%).

LC-MS (Method D):

Rₜ[min] (UV-signal 220 nm): 1.43

M[g/mol]: 389.0 [M+H⁺]

### Example 15.4: Synthesis of benzyl 4-[(3S,4R,5S)-3-acetamido-4,5-diacetoxy-1-piperidyl]-4-oxo-butanoate (185)

To a solution of benzyl 4-[(3aS,7S,7aR)-7-azido-2,2-dimethyl-4,6,7,7a-tetrahydro-3aH-[1,3]dioxolo[4,5-c]pyridin-5-yl]-4-oxo-butanoate (184, 72 mg, 0.185 mmol, 1.00 equiv.) in THF (5 mL) and water (0.1 mL) was added PMe₃ (1 N in THF, 0.27 mL, 0.27 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (5 mL) and acetic anhydride (0.07 mL, approx. 4 equiv.) and pyridine (0.09 mL, approx. 6 equiv.) were added at r.t. and the reaction mixture was stirred for 1 h. Since LC/MS indicated full formation of the acetamide, the reaction mixture was concentrated in vacuo. The crude product was dissolved in acetic acid (4 mL) and water (1 mL) and heated to 80° C. for 3 h until full acetonide deprotection was monitored by LC/MS. The solvent was removed in vacuo and the residue was dissolved in acetic anhydride (1.00 mL) and pyridine (0.50 mL) and stirred for 1 h at r.t. The solvent was removed in vacuo and the crude mixture was purified by HPLC (15 min, 10-60% acetonitrile in water+0.1% TFA) to yield benzyl 4-[(3S,4R,5S)-3-acetamido-4,5-diacetoxy-1-piperidyl]-4-oxo-butanoate (185, 39 mg, 0.087 mmol, 47%) as a colorless oil.

LC-MS (Method D):

Rₜ[min] (UV-signal 220 nm): 1.05

M[g/mol]: 449.2 [M+H⁺]

### Example 15.5: Synthesis of 4-[(3S,4R,5S)-3-acetamido-4,5-diacetoxy-1-piperidyl]-4-oxo-butanoic acid (186)

A solution of benzyl 4-[(3S,4R,5S)-3-acetamido-4,5-diacetoxy-1-piperidyl]-4-oxo-butanoate (185, 39 mg, 0.089 mmol, 1.00 equiv.) in EtOH (10 mL) was hydrogenated in an H-Cube (10% Pd(OH)₂/C, full hydrogen mode, 60° C., flow 1 mL/min). Full hydrogenation was detected after three cycles. The solvent was removed in vacuo, the crude product 4-[(3S,4R,5S)-3-acetamido-4,5-diacetoxy-1-piperidyl]-4-oxo-butanoic acid (186, 32 mg, 0.089 mmol, quant.) was obtained as a colorless oil and used without further purification.

### Example 15.6: Synthesis of methyl 5-[(3aS,7R,7aR)-7-hydroxy-2,2-dimethyl-4,6,7,7a-tetrahydro-3aH-[1,3]dioxolo[4,5-c]pyridin-5-yl]-5-oxo-pentanoate (187)

To a solution of (3aS,7R,7aR)-2,2-dimethyl-3a,4,5,6,7,7a-hexahydro-[1,3]dioxolo[4,5-c]pyridin-7-ol (78) (318 mg, 1.84 mmol, 1.00 equiv.) in THF (50 mL) and saturated aqueous NaHCO₃-solution (10 mL) acid chloride benzyl 5-chloro-5-oxo-pentanoate (106, 886 mg, 3.68 mmol, 2.00 equiv.) was added. The reaction mixture was stirred for 1 h at r.t. EtOAc (100 mL) and water (10 mL) were added, the layers were separated, the organic layer was washed with saturated aqueous NaCl-solution (20 mL), dried (MgSO₄), filtered and concentrated in vacuo. The crude product methyl 5-[(3aS,7R,7aR)-7-hydroxy-2,2-dimethyl-4,6,7,7a-tetrahydro-3aH-[1,3]dioxolo[4,5-c]pyridin-5-yl]-5-oxo-pentanoate (187) was directly used for the next step without further purification.

LC-MS (Method D):

Rₜ[min] (UV-signal 220 nm): 1.09

M[g/mol]: 378.1 [M+H⁺]

### Example 15.7: Synthesis of methyl 5-[(3aS,7R,7aS)-2,2-dimethyl-7-methylsulfonyloxy-4,6,7,7a-tetrahydro-3aH-[1,3]dioxolo[4,5-c]pyridin-5-yl]-5-oxo-pentanoate (188)

A solution of methyl 5-[(3aS,7R,7aR)-7-hydroxy-2,2-dimethyl-4,6,7,7a-tetrahydro-3aH-[1,3]dioxolo[4,5-c]pyridin-5-yl]-5-oxo-pentanoate (187, max. 1.84 mmol, 1.00 equiv.) in anhydrous DCM (10 mL) was cooled to 0° C. and pyridine (0.56 mL, 6.95 mmol, 3.77 equiv.) and mesyl anhydride (617 mg, 3.47 mmol, 1.88 equiv.) were added. The reaction mixture was stirred for 1.5 h at 0° C. until LC/MS indicated full conversion of the starting material. Aqueous 1 N HCl (10 mL) and EtOAc (50 mL) were added, the layers were separated, the organic layer was washed with saturated aqueous NaCl-solution (10 mL), dried (MgSO₄), filtered and concentrated in vacuo. The crude product was purified by flash chromatography (silica, 1-80% EtOAc in n-heptane) to yield methyl 5-[(3aS,7R,7aS)-2,2-dimethyl-7-methylsulfonyloxy-4,6,7,7a-tetrahydro-3aH-[1,3]dioxolo[4,5-c]pyridin-5-yl]-5-oxo-pentanoate (188, 441 mg, 0.97 mmol, 53% over two steps) as a colorless oil.

LC-MS (Method D):

Rₜ[min] (UV-signal 220 nm): 1.31

M[g/mol]: 456.1 [M+H⁺]

### Example 15.8: Synthesis of benzyl 5-[(3aS,7S,7aR)-7-azido-2,2-dimethyl-4,6,7,7a-tetrahydro-3aH-[1,3]dioxolo[4,5-c]pyridin-5-yl]-5-oxo-pentanoate (189)

Methyl 5-[(3aS,7R,7aS)-2,2-dimethyl-7-methylsulfonyloxy-4,6,7,7a-tetrahydro-3aH-[1,3]di-oxolo[4,5-c]pyridin-5-yl]-5-oxo-pentanoate (188, 528 mg, 1.16 mmol, 1.00 equiv.) was dissolved in DMF (3 mL), LiN₃ (2 M in DMF, 1.45 mL, 2.90 mmol, 2.50 equiv.) was added and the mixture was stirred at 100° C. for 2 d. The reaction was stopped since significant amounts of an elimination product were detected by LC/MS. EtOAc (50 mL) and water (10 mL) were added, the layers were separated, and the aqueous layer was re-extracted with EtOAc (3×20 mL). The combined organic layers were washed with saturated aqueous NaCl-solution (30 mL), dried (MgSO₄), filtered and concentrated in vacuo. The crude product was purified by flash chromatography (silica, 1-80% EtOAc in n-heptane) to yield benzyl 5-[(3aS,7S,7aR)-7-azido-2,2-dimethyl-4,6,7,7a-tetrahydro-3aH-[1,3]dioxolo[4,5-c]pyridin-5-yl]-5-oxo-pentano-ate (189, 60 mg, 0.15 mmol, 13%) as a colorless oil and recovered methyl 5-[(3aS,7R,7aS)-2,2-dimethyl-7-methylsulfonyloxy-4,6,7,7a-tetrahydro-3aH-[1,3]dioxolo[4,5-c]pyridin-5-yl]-5-oxo-pentanoate (188, 248 mg, 0.54 mmol, 47%).

LC-MS (Method D):

Rₜ[min] (UV-signal 220 nm): 1.46

M[g/mol]: 403.0 [M+H⁺]

### Example 15.9: Synthesis of benzyl 5-[(3S,4R,5S)-3-acetamido-4,5-diacetoxy-1-piperidyl]-5-oxo-pentanoate (190)

To a solution of benzyl 5-[(3aS,7S,7aR)-7-azido-2,2-dimethyl-4,6,7,7a-tetrahydro-3aH-[1,3]dioxolo[4,5-c]pyridin-5-yl]-5-oxo-pentanoate (189, 60 mg, 0.149 mmol, 1.00 equiv.) in THF (5 mL) and water (0.1 mL) was added PMe₃ (1 N in THF, 0.22 mL, 0.22 mmol, 1.50 equiv.) and the reaction mixture was stirred for 1 h at r.t. Since LC/MS indicated full conversion of the starting material, the reaction mixture was concentrated in vacuo. The residue was dissolved in EtOAc (5 mL) and acetic anhydride (0.06 mL, approx. 4 equiv.) and pyridine (0.07 mL, approx. 6 equiv.) were added and the reaction mixture was stirred for 1 h at r.t. Since LC/MS indicated full formation of the acetamide, the reaction mixture was concentrated in vacuo. The crude product was dissolved in acetic acid (4 mL) and water (1 mL) and heated to 80° C. for 3 h until full acetonide deprotection was monitored by LC/MS. The solvent was removed in vacuo and the residue was dissolved in acetic anhydride (1.00 mL) and pyridine (0.50 mL) and stirred for 1 h at r.t. The solvent was removed in vacuo and the crude mixture was purified by HPLC (15 min, 10-60% acetonitrile in water+0.1% TFA) to yield benzyl 5-[(3S,4R,5S)-3-acetamido-4,5-diacetoxy-1-piperidyl]-5-oxo-pentanoate (190, 46 mg, 0.099 mmol, 67%) as a colorless oil.

LC-MS (Method D):

Rₜ[min] (UV-signal 220 nm): 1.04

M[g/mol]: 463.2 [M+H⁺]

### Example 15.10: Synthesis of 5-[(3S,4R,5S)-3-acetamido-4,5-diacetoxy-1-piperidyl]-5-oxo-pentanoic acid (191)

A solution of benzyl 5-[(3S,4R,5S)-3-acetamido-4,5-diacetoxy-1-piperidyl]-5-oxo-pentanoate (190, 46 mg, 0.099 mmol, 1.00 equiv.) in EtOH (10 mL) was hydrogenated in an H-Cube (10% Pd(OH)₂/C, full hydrogen mode, 60° C., flow 1 mL/min). Full hydrogenation was detected after three cycles. The solvent was removed in vacuo, the crude product 5-[(3S,4R,5S)-3-acetamido-4,5-diacetoxy-1-piperidyl]-5-oxo-pentanoic acid (191, 26 mg, 0.070 mmol, 70%) was obtained as a colorless oil and used without further purification.

### Example 15.11: Synthesis of 6-[(3aS,7S,7aR)-7-azido-2,2-dimethyl-4,6,7,7a-tetrahydro-3aH-dioxolo[4,5-c]pyridin-5-yl]-6-oxo-hexanoic acid (192)

To a solution of benzyl 6-[(3aS,7S,7aR)-7-azido-2,2-dimethyl-4,6,7,7a-tetrahydro-3aH-[1,3]dioxolo[4,5-c]pyridin-5-yl]-6-oxo-hexanoate (111, 300 mg, 0.72 mmol, 1.00 equiv.) in THF (5 mL) and water (1 mL) was added LiOH·H₂O (173 mg, 4.13 mmol, 5.73 equiv.). The reaction mixture was stirred for 3 d at r.t. EtOAc (30 mL) and aqueous 1 N HCl (50 mL) were added, the layers were separated, and the aqueous layers was re-extracted with EtOAc (3×10 mL). The combined organic layers were washed with saturated aqueous NaCl-solution (20 mL), dried (MgSO₄), filtered and concentrated in vacuo. The crude product was purified by flash chromatography (silica, 15-100% EtOAc in n-heptane) to yield 6-[(3aS,7S,7aR)-7-azido-2,2-dimethyl-4,6,7,7a-tetrahydro-3aH-[1,3]dioxolo[4,5-c]pyridin-5-yl]-6-oxo-hexanoic acid (192, 155 mg, 0.47 mmol, 66%) as a colorless oil.

LC-MS (Method D):

Rₜ[min] (UV-signal 220 nm): 0.89

M[g/mol]: 327.2 [M+H⁺]

### Example 16: Structures and IUPAC-Names of Additional Guanosine-Type Compounds as Potential ASGPR-Binders are Summarized in Table H Below

TABLE H

| Entry | Structure | Compound # | IUPAC-Name |
|---|---|---|---|
| 1 | 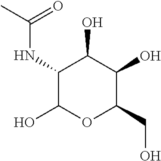 | 193 | N-[(3R,4R,5R,6R)-2,4,5-trihydroxy-6- (hydroxymethyl)-tetrahydropyran-3- yl]acetamide (GalNAc; positive control) |
| 2 | 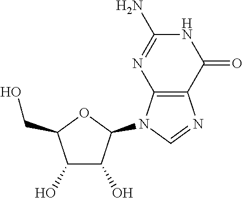 [SMILES: Nc1nc2c(ncn2[C@@H]2O[C@H](CO)[C@@H](O)[C@H]2O)c(=O)[nH]1] | 194 | 2-amino-9-[(2R,3R,4S,5R)-3,4- dihydroxy-5-(hydroxymethyl)- tetrahydrofuran-2-yl]-1H-purin-6-one |
| 3 | 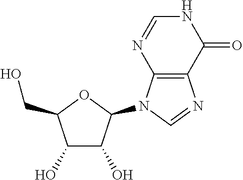 [SMILES: O=c1[nH]cnc2c1ncn2[C@@H]1O[C@H](CO)[C@@H](O)[C@H]1O] | 195 | 9-[(2R,3R,4S,5R)-3,4-dihydroxy-5- (hydroxymethyl)-tetrahydro-furan-2-yl]- 1H-purin-6-one |
| 4 | 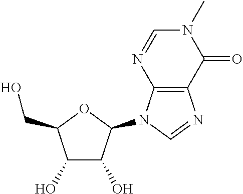 [SMILES: Cn1cnc2c(ncn2[C@@H]2O[C@H](CO)[C@@H](O)[C@H]2O)c1=O] | 196 | 9-[(2R,3R,4S,5R)-3,4-dihydroxy-5- (hydroxymethyl)-tetrahydro-furan-2-yl]- 1-methyl-purin-6-one |
| 5 | 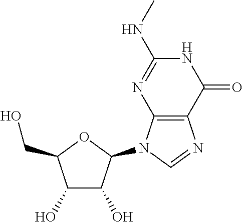 [SMILES: CNc1nc2c(ncn2[C@@H]2O[C@H](CO)[C@@H](O)[C@H]2O)c(=O)[nH]1] | 197 | 9-[(2R,3R,4S,5R)-3,4-dihydroxy-5- (hydroxymethyl)-tetrahydro-furan-2-yl]- 2-(methylamino)-1H-purin-6-one |
| 6 | 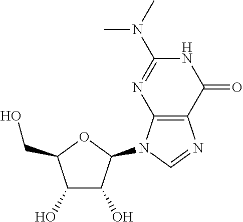 [SMILES: CN(C)c1nc2c(ncn2[C@@H]2O[C@H](CO)[C@@H](O)[C@H]2O)c(=O)[nH]1] | 198 | 9-[(2R,3R,4S,5R)-3,4-dihydroxy-5- (hydroxymethyl)-tetrahydro-furan-2-yl]- 2-(dimethylamino)-1H-purin-6-one |
| 7 | 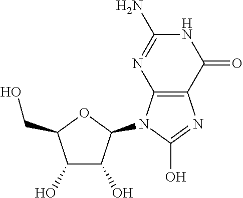 [SMILES: Nc1nc2c(nc(O)n2[C@@H]2O[C@H](CO)[C@@H](O)[C@H]2O)c(=O)[nH]1] | 199 | 2-amino-9-[(2R,3R,4S,5R)-3,4- dihydroxy-5-(hydroxymethyl)- tetrahydrofuran-2-yl]-8-hydroxy-1H- purin-6-one |
| 8 | 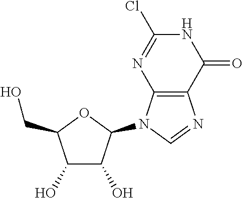 [SMILES: O=c1[nH]c(Cl)nc2c1ncn2[C@@H]1O[C@H](CO)[C@@H](O)[C@H]1O] | 200 | 2-chloro-9-[(2R,3R,4S,5R)-3,4- dihydroxy-5-(hydroxymethyl)- tetrahydrofuran-2-yl]-1H-purin-6-one |
| 9 | 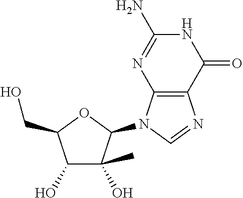 [SMILES: C[C@@]1(O)[C@H](O)[C@@H](CO)O[C@H]1n1cnc2c(=O)[nH]c(N)nc21] | 201 | 2-amino-9-[(2R,3R,4R,5R)-3,4- dihydroxy-5-(hydroxymethyl)-3-methyl- tetrahydrofuran-2-yl]-1H-purin-6-one |
| 10 | 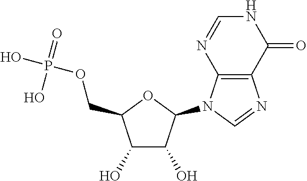 [SMILES: O=c1[nH]cnc2c1ncn2[C@@H]1O[C@H](COP(=O)(O)O)[C@@H](O)[C@H]1O] | 202 | [(2R,3S,4R,5R)-3,4-dihydroxy-5-(6-oxo- 1H-purin-9-yl)tetrahydro-furan-2- yl]methyl dihydrogen phosphate |
| 12 | 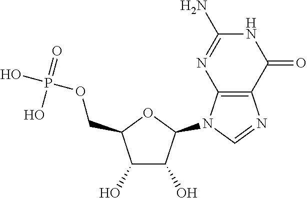 [SMILES: Nc1nc2c(ncn2[C@@H]2O[C@H](COP(=O)(O)O)[C@@H](O)[C@H]2O)c(=O)[nH]1] | 203 | [(2R,3S,4R,5R)-5-(2-amino-6-oxo-1H- purin-9-yl)-3,4-di-hydroxy- tetrahydrofuran-2-yl]methyl dihydrogen phosphate |
| 13 | 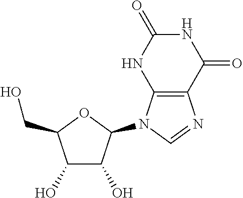 [SMILES: O=c1[nH]c(=O)c2ncn([C@@H]3O[C@H](CO)[C@@H](O)[C@H]3O)c2[nH]1] | 204 | 9-[(2R,3R,4S,5R)-3,4-dihydroxy-5- (hydroxymethyl)-tetrahydro-furan-2-yl]- 3H-purine-2,6-dione |
| 14 | 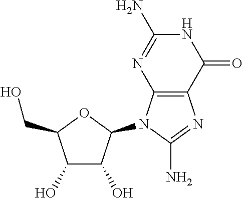 [SMILES: Nc1nc2c(nc(N)n2[C@@H]2O[C@H](CO)[C@@H](O)[C@H]2O)c(=O)[nH]1] | 205 | 2,8-diamino-9-[(2R,3R,4S,5R)-3,4- dihydroxy-5-(hydroxy-methyl)- tetrahydrofuran-2-yl]-1H-purin-6-one |

### Example 17.1: Synthesis of 2-[2-(2-Benzyloxyethoxy)ethoxy]ethyl 4-methylbenzenesulfonate (207)

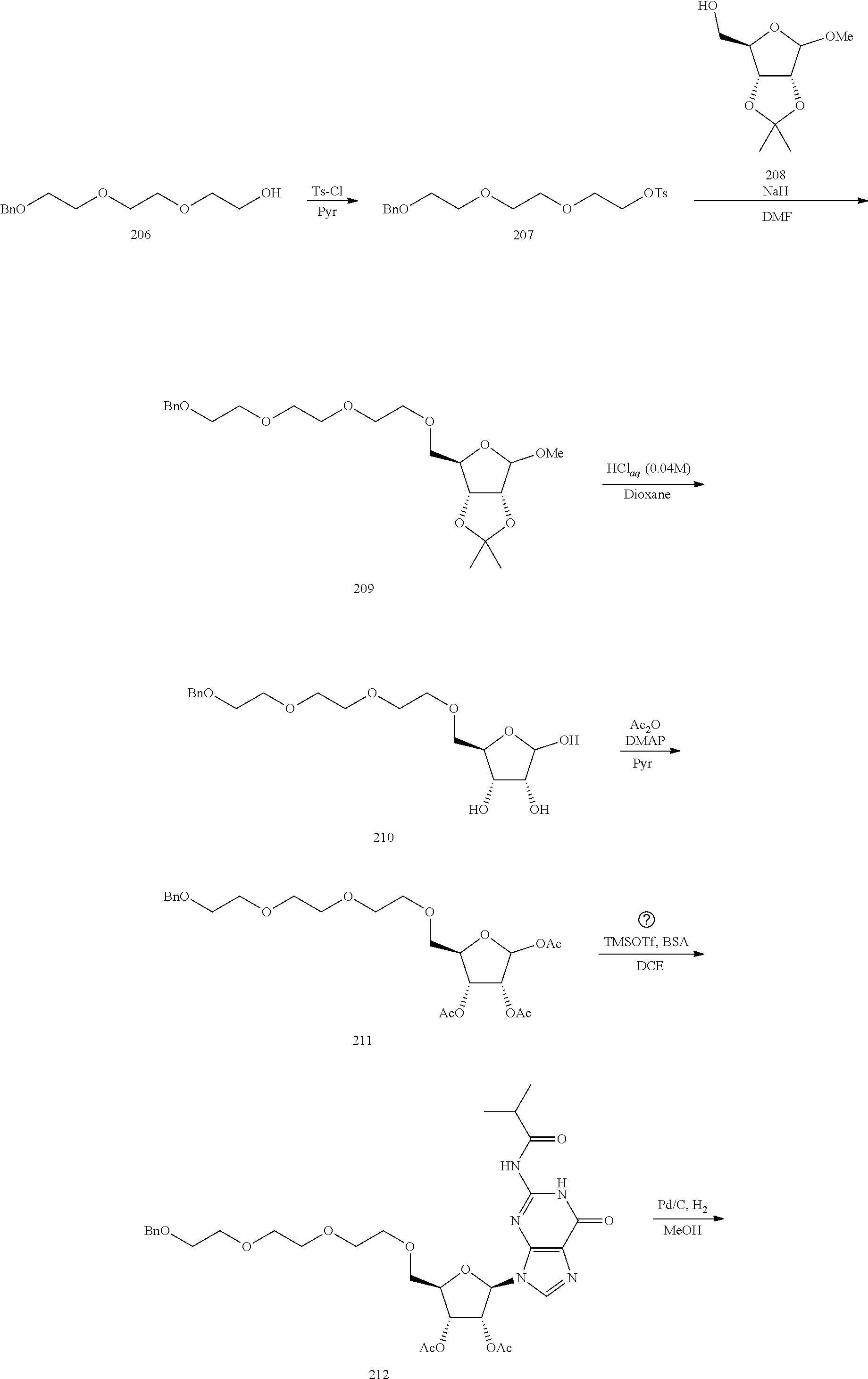

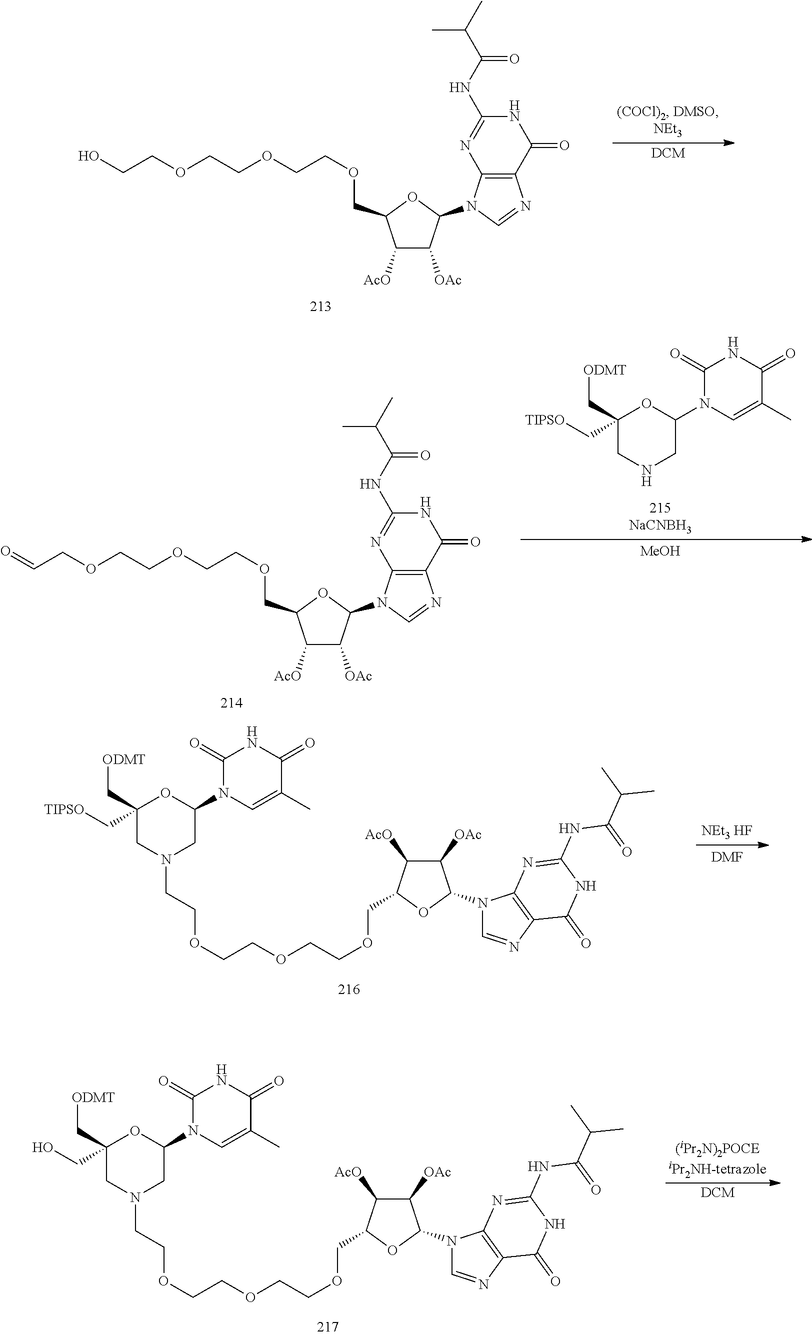

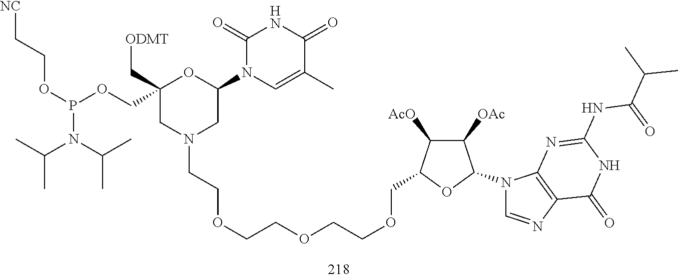

10.0 g (39.53 mmol) 2-[2-(2-benzyloxyethoxy)ethoxy]ethanol (206) were dissolved in 100 ml dry pyridine. At room temperature, 8.37 g (43.49 mmol) p-toluenesulfonyl chloride and a catalytical amount of DMAP were added and the solution was stirred for 14 h. The solvent was removed in vacuo and the residue dissolved in 250 ml methyl-tert. butyl ether. The organic solution was washed twice with 250 ml 10% citric acid solution, H₂O and sat. NaCl-solution. After drying with MgSO₄, the solvent was evaporated and the residue purified on silica (0 to 100% EtOAc in n-heptane), yielding 4.06 g (26.0%) of the title compound 2-[2-(2-benzyloxyethoxy)ethoxy]ethyl 4-methylbenzenesulfonate (207) as colorless liquid.

LC-MS (Method A):

Rₜ[min] (ELSD-signal): 2.38

MS (calc.: 394.1) (m/z)=395.3 [M+H⁺].

### Example 17.2: Synthesis of (3aR,6R,6aR)-6-[2-[2-(2-Benzyloxyethoxy)ethoxy]ethoxy-methyl]-4-methoxy-2,2-dimethyl-3a,4,6,6a-tetrahydrofuro[3,4-d][1,3]dioxole (209)

2.09 g (10.24 mmol) Methyl-2,3-di-O-isoproylidene-D-ribofuranoside (208) were dissolved in 25 ml dry DMF. At 0° C., 294.9 mg (12.29 mmol) sodium hydride were added over a period of 30 min and stirring was continued for another 30 min at room temperature. After cooling again to 0° C., a solution of 4.04 g (10.24 mmol) 2-[2-(2-benzyloxyethoxy)ethoxy]ethyl 4-methyl-benzenesulfonate (207) in 25 ml dry DMF was added and the ice bath was removed. After stirring for 3 d at room temperature, the solvent was evaporated and the residue dissolved in 150 ml methyl-tert. butyl ether. After washing with 250 ml H2O and 3×250 ml sat. NaCl-solution, the organic layer was dried with MgSO₄. Purification on silica (0 to 60% EtOAc in n-heptane) gave 3.84 g (88.0%) of the desired product (3aR,6R,6aR)-6-[2-[2-(2-benzyloxy-ethoxy)ethoxy]ethoxymethyl]-4-methoxy-2,2-dimethyl-3a,4,6,6a-tetrahydrofuro[3,4-d][1,3]dioxole (209) as colorless oil.

LC-MS (Method A):

Rₜ[min] (ELSD-signal): 2.23

MS (calc.: 426.2) (m/z)=395.4 [M-OMe-]

### Example 17.3: Synthesis of (3R,4S,5R)-5-[2-[2-(2-Benzyloxyethoxy)ethoxy]ethoxymethyl]-tetrahydro-furan-2,3,4-triol (210)

3.82 g (8.95 mmol) of the starting material (3aR,6R,6aR)-6-[2-[2-(2-benzyloxyethoxy)-ethoxy]ethoxymethyl]-4-methoxy-2,2-dimethyl-3a,4,6,6a-tetrahydrofuro[3,4-d][1,3]dioxole (209) were dissolved in 56 ml dioxane. After adding 56 ml 0.04% aqueous HCl, the reaction mixture was stirred at 100° C. for 2 h. The reaction solution was cooled to room temperature. The dioxane was removed in vacuo and 230 mg (1.16 mmol) BaCO₃ were added. The heterogenous mixture was vigorously stirred for 1 h, filtered and evaporated. The residue was co-evaporated with ACN, which gave 3.59 g (crude product) of (3R,4S,5R)-5-[2-[2-(2-benzyloxyethoxy)ethoxy]ethoxymethyl]tetrahydro-furan-2,3,4-triol (210) as colorless oil.

LC-MS (Method A):

Rₜ[min] (ELSD-signal): 1.34

MS (calc.: 372.2) (m/z)=417.3 [M−H⁺+FA].

### Example 17.4: Synthesis of [(2R,3R,4R)-4,5-Diacetoxy-2-[2-[2-(2-benzyloxyethoxy)ethoxy]-ethoxymethyl]tetrahydro-furan-3-yl] acetate (211)

To a solution of 3.57 g (9.59 mmol) of the triol (3R,4S,5R)-5-[2-[2-(2-benzyloxyethoxy)-ethoxy]ethoxymethyl]tetrahydro-furan-2,3,4-triol (210) in 66 ml dry pyridine were added 9.89 g (95.86 mmol) acetic anhydride, followed by a catalytical amount of DMAP. After the solution was stirred at room temperature for 18 h, the reaction was quenched by adding 35 ml EtOH. The solvents were removed in vacuo and the residue dissolved in 300 ml methyl-tert. butyl ether. After washing with 250 ml H₂O, 2×250 ml 10% citric acid solution, again 250 ml H₂O and 250 ml sat. NaCl-solution, the organic layer was dried with MgSO₄. Evaporation of the solvent gave 3.80 g (crude product) of the desired acetylated product [(2R,3R,4R)-4,5-diacet-oxy-2-[2-[2-(2-benzyloxyethoxy)ethoxy]ethoxy-methyl]tetrahydro-furan-3-yl] acetate (211) as yellow oil, which was used without further purification.

LC-MS (Method A):

Rₜ[min] (ELSD-signal): 2.06, 2.12 (mixture of diastereomers)

MS (calc.: 498.2) (m/z)=439.3 [M−AcO⁻].

### Example 17.5: Synthesis of [(2R,3R,4R,5R)-4-Acetoxy-2-[2-[2-(2-benzyloxyethoxy)ethoxy]-ethoxymethyl]-5-[2-(2-methylpropanoylamino)-6-oxo-1H-purin-9-yl]tetrahydrofuran-3-yl] acetate (212)

To a solution of 4.27 g (18.93 mmol) N²-Isobutyryl-guanine in 60 ml dry DCE were added 14.59 g (68.15 mmol) BSA and the solution was refluxed for 1 h. The reaction mixture was than cooled to room temperature and a solution of 3.78 g (7.57 mmol) [(2R,3R,4R)-4,5-diacetoxy-2-[2-[2-(2-benzyloxyethoxy)ethoxy]ethoxy-methyl]tetrahydro-furan-3-yl] acetate (211) in 20 ml dry DCE was added, followed by the addition of 7.65 g (34.08 mmol) TMSOTf. The reaction was stirred for 5 h under reflux conditions and left at room temperature overnight. After the addition of 250 ml DCM, the solution was washed with 650 ml sat. NaHCO₃/H₂O (1:1). The precipitates were filtered and the organic layer separated. After extraction of the aqueous phase with 2×250 ml DCM, the combined organic layers were dried with MgSO₄ and evaporated. The obtained crude product was purified by silicagel chromatography (40 to 80% MeOH/EtOAc (1:9) in n-heptane), yielding 2.92 g (58.5%) of the guanosine analog [(2R,3R,4R,5R)-4-acetoxy-2-[2-[2-(2-benzyloxyethoxy)ethoxy]ethoxy-methyl]-5-[2-(2-meth-ylpropanoylamino)-6-oxo-1H-purin-9-yl]tetrahydrofuran-3-yl] acetate (212) as colorless foam.

LC-MS (Method A):

Rₜ[min] (ELSD-signal): 2.06

MS (calc.: 659.3) (m/z)=660.5 [M+H⁺].

### Example 17.6: Synthesis of [(2R,3R,4R,5R)-4-Acetoxy-2-[2-[2-(2-hydroxyethoxy)ethoxy]-ethoxymethyl]-5-[2-(2-methylpropanoylamino)-6-oxo-1H-purin-9-yl]tetrahydrofuran-3-yl]acetate (213)

2.90 g (4.40 mmol) of the benzylether [(2R,3R,4R,5R)-4-acetoxy-2-[2-[2-(2-benzyloxy-ethoxy)ethoxy]ethoxy-methyl]-5-[2-(2-methylpropanoylamino)-6-oxo-1H-purin-9-yl]tetra-hydrofuran-3-yl] acetate (212) were dissolved in 50 ml MeOH. After adding 163.7 mg (154 μmol) Pd/C (10%), the apparatus was flushed with hydrogen and the reaction mixture was kept under 4.6 bar H2 at room temperature for 2 d. The heterogenous mixture was filtered and the filtrate evaporated. The residue was again dissolved in 50 ml MeOH and 327.4 mg (308 μmol) Pd/C (10%) were added. The hydrogenation mixture was set under 4.6 bar H₂ for another 22 h, to achieve complete conversion. The mixture was filtered and the solvent evaporated. After co-evaporation with ACN, 2.44 g (crude) of the alcohol [(2R,3R,4R,5R)-4-acetoxy-2-[2-[2-(2-hydroxyethoxy)ethoxy]ethoxy-methyl]-5-[2-(2-methylpropanoylamino)-6-oxo-1H-purin-9-yl]tetrahydrofuran-3-yl] acetate (213) were obtained as colorless foam, which was used in the next step without further purification.

LC-MS (Method A):

Rₜ[min] (ELSD-signal): 1.42

MS (calc.: 569.2) (m/z)=570.4 [M+H⁺].

### Example 17.7: Synthesis of [(2R,3R,4R,5R)-4-Acetoxy-5-[2-(2-methylpropanoylamino)-6-oxo-1H-purin-9-yl]-2-[2-[2-(2-oxoethoxy)ethoxy]ethoxymethyl]tetrahydrofuran-3-yl] acetate (214)

1.05 ml (2.11 mmol) of a 2 M solution of oxalyl chloride in DCM were diluted with 10 ml dry DCM. At −60° C., a solution of 329.5 mg (4.21 mmol) DMSO in 10 ml dry DCM was added and stirred for 30 min, followed by the addition of a solution of 1.0 g (1.76 mmol) [(2R,3R,4R,5R)-4-acetoxy-2-[2-[2-(2-hydroxyethoxy)ethoxy]ethoxy-methyl]-5-[2-(2-methyl-propanoylamino)-6-oxo-1H-purin-9-yl]tetrahydrofuran-3-yl] acetate (213) in 10 ml dry DCM. Stirring was continued for 30 min at −60° C. when 892.8 mg (8.78 mmol) NEt₃ were added. The cooling bath was removed and the reaction solution allowed to reach room temperature. The solution was washed with 50 ml H₂O and the layers were separated. The aqueous phase was extracted with 2×50 ml DCM and the combined organic layers dried with MgSO₄. Evaporation of the solvent gave 1.06 g (crude) of the desired aldehyde [(2R,3R,4R,5R)-4-acetoxy-5-[2-(2-methylpropanoylamino)-6-oxo-1H-purin-9-yl]-2-[2-[2-(2-oxoethoxy)ethoxy]ethoxymethyl]-tetrahydrofuran-3-yl] acetate (214) as light yellow foam.

LC-MS (Method A):

Rₜ[min] (ELSD-signal): 1.43

MS (calc.: 567.2) (m/z)=568.4 [M+H⁺].

### Example 17.8: Synthesis of [(2R,3R,4R,5R)-4-Acetoxy-2-[2-[2-[2-[(2S,6R)-2-[[bis(4-meth-oxyphenyl)-phenyl-methoxy]methyl]-6-(5-methyl-2,4-dioxo-pyrimidin-1-yl)-2-(triisopropyl-silyloxy-methyl)morpholin-4-yl]ethoxy]ethoxy]ethoxymethyl]-5-[2-(2-methylpropanoyl-amino)-6-oxo-1H-purin-9-yl]tetrahydrofuran-3-yl] acetate (216)

1.03 g (1.81 mmol) of the aldehyde [(2R,3R,4R,5R)-4-acetoxy-5-[2-(2-methylpropanoyl-amino)-6-oxo-1H-purin-9-yl]-2-[2-[2-(2-oxoethoxy)ethoxy]ethoxymethyl]-tetrahydrofuran-3-yl] acetate (214) and 1.32 g (1.81 mmol) of the morpholine 1-[(2R,6S)-6-[[bis(4-methoxy-phenyl)-phenyl-methoxy]methyl]-6-(triisopropylsilyloxymethyl)morpholin-2-yl]-5-methyl-pyrimidine-2,4-dione (215) (Hofmeister et al., WO2019170731) were dissolved in in 25 ml dry MeOH. At room temperature, molecular sieves (3 Å) were added, followed by the addition of 738.4 mg (7.22 mmol) NEt₃ and 1.10 g (18.06 mmol) AcOH. After 1 h, 238.9 mg (3.61 mmol) sodium cyanoboronhydride were added and the reaction solution stirred for 15 h at room temperature. The reaction mixture was filtered and the filtrate brought to pH 7 by the addition of sat. NaHCO₃-solution. The MeOH was evaporated and the remaining aqueous solution diluted with H₂O and extracted with EtOAc. The organic layer was separated and washed with sat. NaCl-solution. After drying with MgSO₄, the solvent was removed in vacuo. After purification on silica (column precondition with n-heptane+1% NEt₃, 0 to 100% MeOH/EtOAc (1:9) in n-heptane), 952 mg (41.1%) of the title compound [(2R,3R,4R,5R)-4-acetoxy-2-[2-[2-[2-[(2S,6R)-2-[[bis(4-methoxyphenyl)-phenyl-methoxy]-methyl]-6-(5-meth-yl-2,4-dioxo-pyrimidin-1-yl)-2-(triisopropylsilyloxymethyl)-morpholin-4-yl]ethoxy]ethoxy]-ethoxymethyl]-5-[2-(2-methylpropanoylamino)-6-oxo-1H-purin-9-yl]tetra-hydrofuran-3-yl]acetate (216) were obtained as colorless foam.

LC-MS (Method A):

Rₜ[min] (ELSD-signal): 3.32

MS (calc.: 1280.6) (m/z)=1281.8 [M+H⁺].

### Example 17.9: Synthesis of [(2R,3R,4R,5R)-4-Acetoxy-2-[2-[2-[2-[(2R,6R)-2-[[bis(4-meth-oxyphenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoylamino)-6-oxo-1H-purin-9-yl]tetrahydrofuran-3-yl] acetate (217)

To a solution of 930 mg (0.73 mmol) of the silylether [(2R,3R,4R,5R)-4-acetoxy-2-[2-[2-[2-[(2S,6R)-2-[[bis(4-methoxyphenyl)-phenyl-methoxy]methyl]-6-(5-methyl-2,4-dioxo-pyr-imidin-1-yl)-2-(triisopropylsilyloxy-methyl)morpholin-4-yl]ethoxy]ethoxy]ethoxymethyl]-5-[2-(2-methylpropanoylamino)-6-oxo-1H-purin-9-yl]tetrahydrofuran-3-yl] acetate (216) in 22 ml DMF were added 1.03 g (10.16 mmol) NEt₃ and 1.87 g (11.61 mmol) NEt₃·3HF. The reaction solution was stirred for 2 h at 75° C., followed by 14 h at room temperature. After another 6 h at 85° C., full conversion was achieved. The reaction solution was cooled to room temperature, diluted with 30 ml EtOAc and poured carefully, under vigorous stirring into 200 ml of sat. NaHCO₃-solution and H₂O (1:1). After 1 h, 150 ml EtOAc were added and the layers were separated. The organic layer was washed with 3×200 ml 10% NaCl-solution and dried with MgSO₄. After evaporation of the solvent, the crude product was purified by silicagel chromatography (0 to 100% EtOAc in n-heptane) yielding 580 mg (71.0%) of the deprotected alcohol [(2R,3R,4R,5R)-4-acetoxy-2-[2-[2-[2-[(2R,6R)-2-[[bis(4-methoxyphenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoylamino)-6-oxo-1H-purin-9-yl]tetra-hydrofuran-3-yl] acetate (217) as colorless foam.

LC-MS (Method A):

Rₜ[min] (ELSD-signal): 2.22

MS (calc.: 1124.5) (m/z)=1125.8 [M+H⁺].

¹H NMR (500 MHz, DMSO-d₆) δ[ppm]: 12.09 (s, 1H), 11.68 (s, 1H), 11.36 (s, 1H), 8.30 (s, 1H), 7.54 (s, 1H), 7.40 (br d, J=7.8 Hz, 2H), 7.19-7.31 (m, 7H), 6.86 (d, J=8.7 Hz, 4H), 6.08 (d, J=7.5 Hz, 1H), 5.84 (m, 1H), 5.78 (m, 1H), 5.45 (br d, J=5.1 Hz, 1H), 4.60 (t, J=5.2 Hz, 1H), 4.34 (m, 1H), 3.49-3.80 (m, 14H), 3.73 (s, 6H), 3.00 (m, 2H), 2.92 (m, 1 H), 2.73-2.83 (m, 2H), 2.53 (m, 2H), 2.27 (m, 1H), 2.09-2.22 (m, 1H), 2.13 (s, 3H), 1.97 (s, 3H), 1.66 (s, 3H), 1.12 (d, J=6.8 Hz, 6H).

### Example 17.10: Synthesis of [(2R,3R,4R,5R)-4-Acetoxy-2-[2-[2-[2-[(2S,6R)-2-[[bis(4-meth-oxyphenyl)-phenyl-methoxy]methyl]-2-[[2-cyanoethoxy-(diisopropylamino)phosphanyl]-oxymethyl]-6-(5-methyl-2,4-dioxo-pyrimidin-1-yl)morpholin-4-yl]ethoxy]ethoxy]ethoxy-methyl]-5-[2-(2-methylpropanoylamino)-6-oxo-1H-purin-9-yl]tetrahydrofuran-3-yl] acetate (218)

Under an argon atmosphere, 558 mg (0.50 mmol) of the alcohol [(2R,3R,4R,5R)-4-acetoxy-2-[2-[2-[2-[(2R,6R)-2-[[bis(4-methoxyphenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoylamino)-6-oxo-1H-purin-9-yl]tetrahydrofuran-3-yl] acetate (217) and 257.4 mg (1.49 mmol) diisopropylammonium tetrazolide were dissolved in 17 ml dry DCM. At room temperature, 231.2 mg (0.74 mmol) 2-cyanoethyl-N,N,N′,N′-tetraisopropylphosphoro-diamidite were added dropwise. After stirring for 16 h, 50 ml H₂O were added, the organic layer was separated and the aqueous layer extracted with 50 ml DCM. The combined organic layers were dried with MgSO₄ and evaporated. The crude product was dissolved in 10 ml EtOAc/diethylether (1:1). After the addition of 40 ml n-pentane, the precipitate was isolated by centrifugation (2 min, 4000 r/min, 15° C.) and decantation of the solvents. The precipitation- and isolation procedure was repeated three times and the obtained crude product dried on a Speedvac, which gave 640 mg (97.4%) of the desired phosphoramidite [(2R,3R,4R,5R)-4-acetoxy-2-[2-[2-[2-[(2S,6R)-2-[[bis(4-methoxyphenyl)-phenyl-methoxy]methyl]-2-[[2-cyano-ethoxy-(diisopropylamino)phosphanyl]oxymethyl]-6-(5-methyl-2,4-dioxo-pyrimidin-1-yl)-morpholin-4-yl]ethoxy]ethoxy]ethoxymethyl]-5-[2-(2-methylpropanoylamino)-6-oxo-1H-purin-9-yl]tetrahydrofuran-3-yl] acetate (218) as colorless foam (mixture of diastereomers).

LC-MS (Method B):

Rₜ[min] (UV-signal 254 nm): 0.81

MS (calc.: 1324.6) (m/z)=940.3 [M−DMT⁺−^{iPr2}N⁻+H₂O+H⁺].

HR-MS (m/z): calc.: 1325.5854, found: 1325.5789 [M+H⁺].

³¹P-NMR (162 MHz) δ[ppm]: 147.58, 147.37.

### Example 18: Synthesis of Targeted Nucleotide Precursor 230 (Pre-lsT2

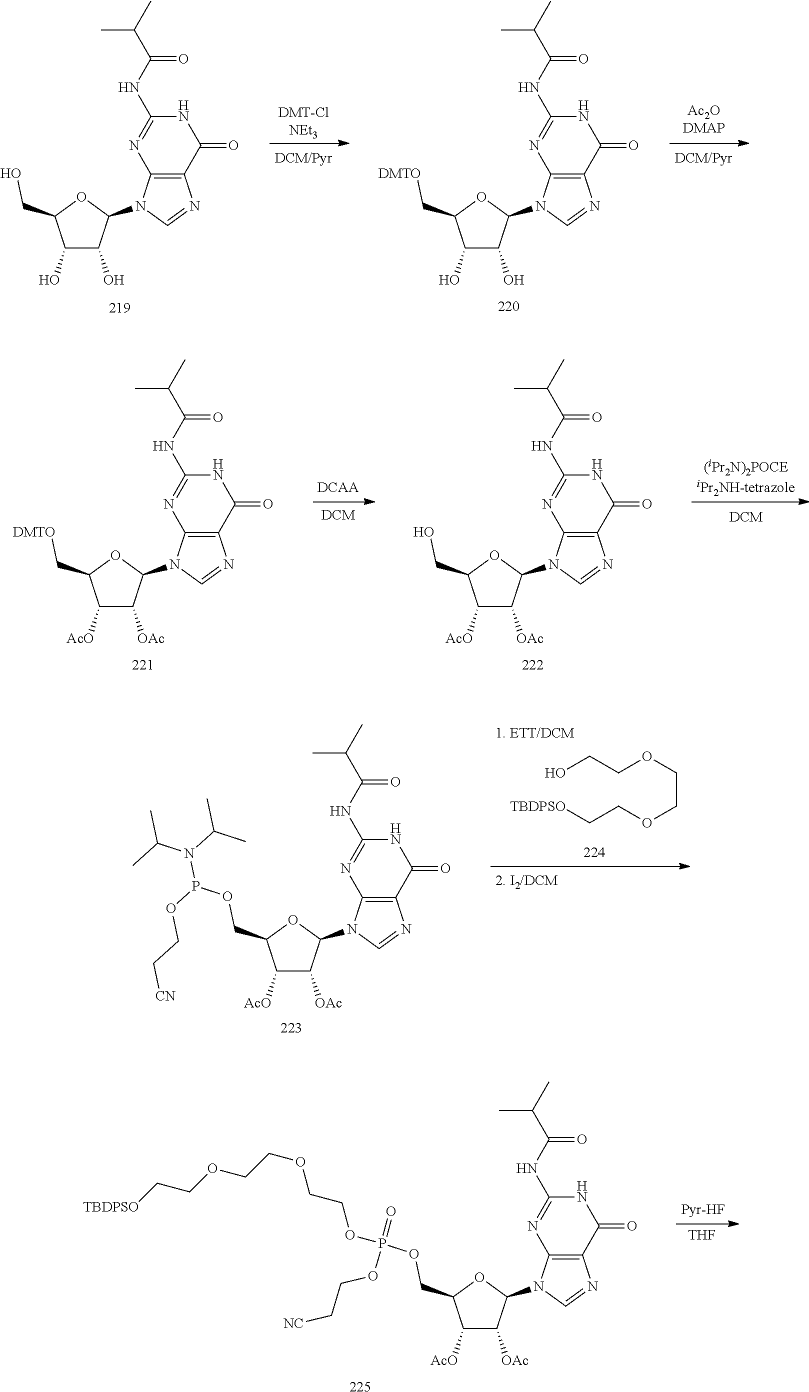

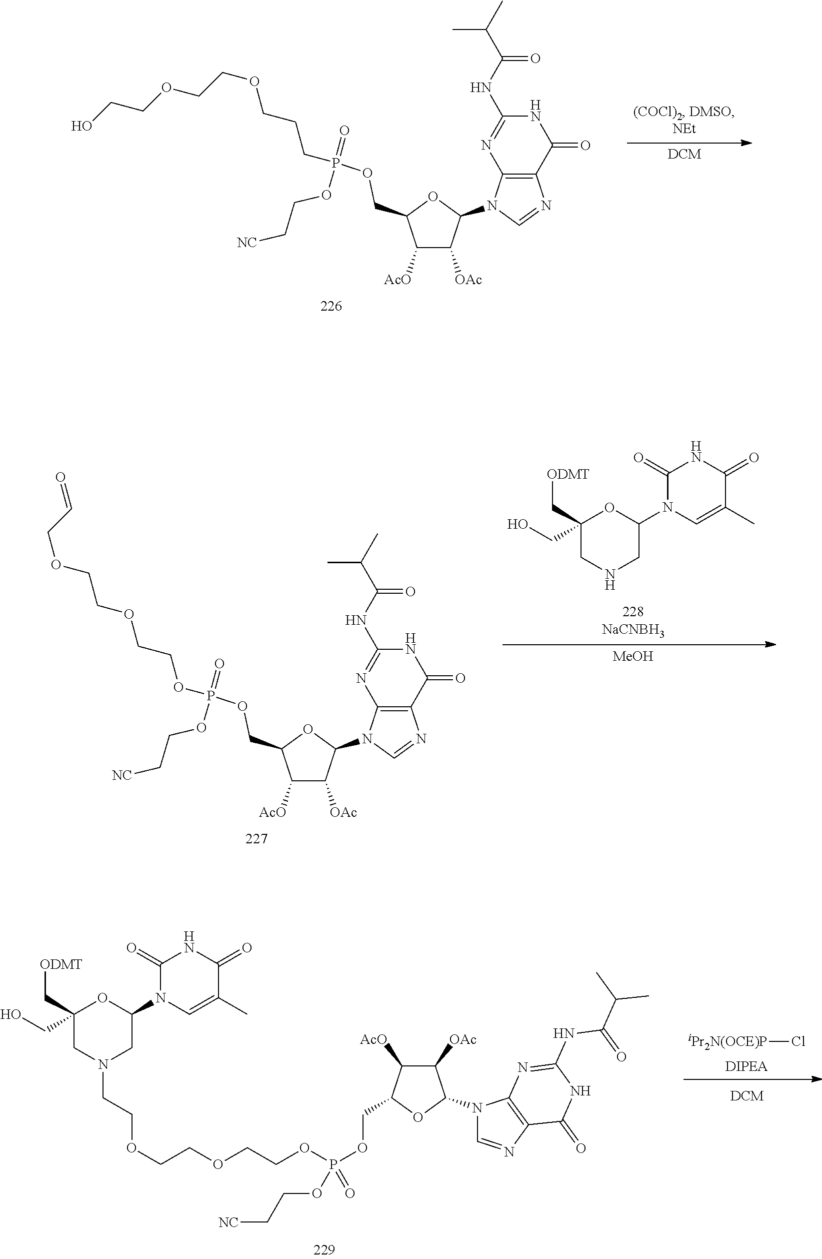

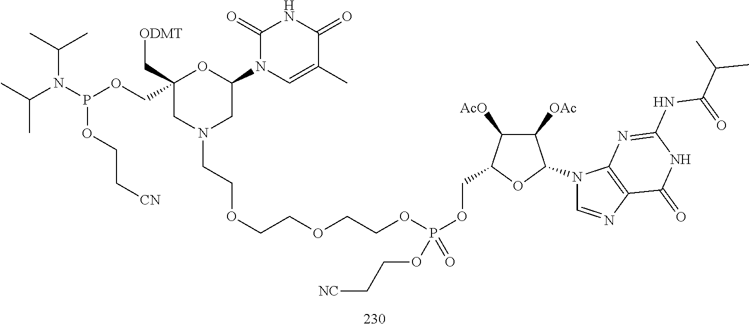

### Example 18.1: Synthesis of N-[9-[(2R,3R,4S,5R)-5-[[Bis(4-methoxyphenyl)-phenyl-meth-oxy]methyl]-3,4-dihydroxy-tetrahydrofuran-2-yl]-6-oxo-1H-purin-2-yl]-2-methyl-propan-amide (220)

10.0 g (27.74 mmol) N-Isobuturylguanosine (219) were co-destilled with 2×50 ml dry pyridine and dissolved in 100 ml dry pyridine. 4.22 g (41.60 mmol) NEt₃ were added at room temperature. After the addition of a solution of 10.55 g (30.51 mmol) DMT-Cl in 75 ml DCM, the reaction solution was stirred for 18 h to achieve complete conversion. 5 ml n-propanol were added to the reaction and stirring was continued for 30 min. The solvents were evaporated and the residue dissolved in EtOAc. After washing with H₂O, 2× citric acid solution (10%), sat. NaHCO₃- and NaCl-solution, the organic layer was dried with MgSO₄ and evaporated. The crude product was dissolved in 100 ml EtOAc and dropped into 600 ml n-heptane. The precipitate was filtered and washed with diethylether/n-heptane (1:1). After drying in vacuo at 45° C., 17.14 g (94.3%) of the DMT-ether N-[9-[(2R,3R,4S,5R)-5-[[bis(4-methoxyphenyl)-phenyl-methoxy]methyl]-3,4-dihydroxy-tetrahydrofuran-2-yl]-6-oxo-1H-purin-2-yl]-2-meth-ylpropanamide (220) were isolated as colorless solid.

LC-MS (Method A):

Rₜ[min] (ELSD-signal): 2.26

MS (calc.: 655.3) (m/z)=656.4 [M+H⁺].

### Example 18.2: Synthesis of [(2R,3R,4R,5R)-4-Acetoxy-2-[[bis(4-methoxyphenyl)-phenyl-methoxy]-methyl]-5-[2-(2-methylpropanoylamino)-6-oxo-1H-purin-9-yl]tetrahydrofuran-3-yl] acetate (221)

5.93 g (57.51 mmol) acetic anhydride were added dropwise to a solution of 17.14 g (26.14 mmol) of the DMT-ether N-[9-[(2R,3R,4S,5R)-5-[[bis(4-methoxyphenyl)-phenyl-methoxy]-methyl]-3,4-dihydroxy-tetrahydrofuran-2-yl]-6-oxo-1H-purin-2-yl]-2-methyl-propanamide (220) in 350 ml DCM/pyridine (4:1) at room temperature. After 10 min, a catalytical amount of DMAP was added and the mixture was stirred for 4 h, to achieve complete conversion. The reaction was quenched by the addition of 10 ml EtOH and the solvent were evaporated. The residue was dissolved in EtOAc and washed with H₂O, 2× citric acid solution (10%), sat. NaHCO₃- and NaCl-solution. The organic layer was dried with MgSO₄ and evaporated, which gave 19.90 g (crude) of the title compound [(2R,3R,4R,5R)-4-acetoxy-2-[[bis(4-methoxy-phenyl)-phenyl-methoxy]-methyl]-5-[2-(2-methylpropanoylamino)-6-oxo-1H-purin-9-yl]-tetrahydrofuran-3-yl] acetate (221) as colorless foam, which was used in the next reaction step without further purification.

LC-MS (Method A):

Rₜ[min] (ELSD-signal): 2.60

MS (calc.: 739.3) (m/z)=740.4 [M+H⁺].

### Example 18.3: Synthesis of [(2R,3R,4R,5R)-4-Acetoxy-2-(hydroxymethyl)-5-[2-(2-methyl-propanoyl-amino)-6-oxo-1H-purin-9-yl]tetrahydrofuran-3-yl] acetate (222)

To a solution of 19.89 g (26.89 mmol) of the starting material [(2R,3R,4R,5R)-4-acetoxy-2-[[bis(4-methoxyphenyl)-phenyl-methoxy]-methyl]-5-[2-(2-methylpropanoylamino)-6-oxo-1H-purin-9-yl]tetrahydrofuran-3-yl] acetate (221) in 180 ml DCM, were added 35.02 g (268.86 mmol) DCAA at room temperature. After 5 min, 200 ml H₂O were added, followed by 50 g NaHCO₃ (portion wise) and stirring was continued for 2 h. The organic layer was separated and the aqueous layer extracted with 2×100 ml DCM. The combined organic extracts were dried with MgSO₄ and evaporated. Purification on silica (25 to 100% EtOAc/MeOH (9:1) in n-heptane) gave 6.61 g (56.2%) of the free alcohol [(2R,3R,4R,5R)-4-acetoxy-2-(hydroxy-methyl)-5-[2-(2-methylpropanoyl-amino)-6-oxo-1H-purin-9-yl]tetrahydrofuran-3-yl] acetate (222) as colorless foam.

LC-MS (Method A):

Rₜ[min] (ELSD-signal): 1.31

MS (calc.: 437.2) (m/z)=438.2 [M+H⁺].

### Example 18.4: Synthesis of [(2R,3R,4R,5R)-4-Acetoxy-2-[[2-cyanoethoxy-(diisopropyl-amino)-phosphanyl]oxymethyl]-5-[2-(2-methylpropanoylamino)-6-oxo-1H-purin-9-yl]tetrahydro-furan-3-yl] acetate (223)

Under an argon atmosphere, 6.10 g (13.95 mmol) of the alcohol [(2R,3R,4R,5R)-4-acetoxy-2-(hydroxymethyl)-5-[2-(2-methylpropanoyl-amino)-6-oxo-1H-purin-9-yl]tetrahydrofuran-3-yl] acetate (222) and 6.27 g (36.26 mmol) diisopropylammonium tetrazolide were dissolved in 100 ml dry DCM. At room temperature, 5.63 g (18.13 mmol) 2-cyanoethyl-N,N,N′,N′-tetraiso-propylphosphoro-diamidite were added dropwise. After stirring for 2 h, 250 ml H₂O were added, the organic layer was separated and the aqueous layer extracted with 2×50 ml DCM. The combined organic layers were dried with MgSO₄ and evaporated. The crude product was dissolved in 100 ml EtOAc/diethylether (1:1) and added drop wise into 400 ml n-pentane. The precipitate was isolated by centrifugation (2 min, 4000 r/min, 15° C.) and decantation of the solvents. The precipitation- and isolation procedure was repeated three times and the obtained crude product dried in vacuo at 40° C., which gave 7.62 g (85.7%) of the desired phosphor-amidite [(2R,3R,4R,5R)-4-acetoxy-2-[[2-cyanoethoxy-(diisopropylamino)-phosphanyl]oxy-methyl]-5-[2-(2-methylpropanoylamino)-6-oxo-1H-purin-9-yl]tetrahydro-furan-3-yl] acetate (223) as colorless foam.

LC-MS (Method A):

Rₜ[min] (UV-signal 254 nm): 0.65

MS (calc.: 637.3) (m/z)=555.1 [M−^{iPr2}N⁻+OH⁻+H⁺].

### Example 18.5: Synthesis of 2-[2-[2-[Tert-butyl(diphenyl)silyl]oxyethoxy]ethoxy]ethanol (224)

8.63 g (56.87 mmol) Triethylene glycol and 1.96 g (28.43 mmol) imidazole were dissolved in 75 ml DCM. At room temperature, a solution of 7.25 g (25.85 mmol) tert-butylchlorodi-phenylsilan in 75 ml DCM was added over a period of 60 min and the solution was stirred for 20 h. After adding 150 ml of H₂O, the layers were separated and the aqueous phase extracted with 2×50 ml DCM. The combined organic layers were dried with MgSO₄ and evaporated. The crude product was purified by silicagel chromatography (0 to 10% MeOH in DCM), yielding 7.52 g (74.9%) of the silylether 2-[2-[2-[tert-butyl(diphenyl)silyl]oxyethoxy]-ethoxy]ethanol (224) as colorless liquid.

LC-MS (Method A):

Rₜ[min] (ELSD-signal): 2.80

MS (calc.: 388.2) (m/z)=411.3 [M+Na⁺].

### Example 18.6: Synthesis of [(2R,3R,4R,5R)-4-Acetoxy-2-[[2-[2-[2-[tert-butyl(diphenyl)-silyl]oxy-ethoxy]ethoxy]ethoxy-(2-cyanoethoxy)phosphoryl]oxymethyl]-5-[2-(2-methyl-propanoyl-amino)-6-oxo-1H-purin-9-yl]tetrahydrofuran-3-yl] acetate (225)

8.05 g (12.62 mmol) of the phosphoamidite [(2R,3R,4R,5R)-4-acetoxy-2-[[2-cyanoethoxy-(diisopropylamino)-phosphanyl]oxymethyl]-5-[2-(2-methylpropanoylamino)-6-oxo-1H-purin-9-yl]tetrahydro-furan-3-yl] acetate (223) and 5.40 g (13.89 mmol) of 2-[2-[2-[tert-butyl(di-phenyl)silyl]oxyethoxy]ethoxy]ethanol (224) were dissolved in 80 ml dry DCM under an argon atmosphere. After adding molecular sieves (3 Å), the mixture was stirred for 15 min, followed by the addition of 3.81 g (27.77 mmol) 5-(ethylthio)-1H-tetrazole. The reaction was stirred at room temperature for 45 min, when 250 ml (25.0 mmol) of a 0.1 M aqueous I₂-solution were added. After the mixture was stirred again for 45 min, 200 ml of a Na₂S203-solution were added, the mixture was filtered and the organic solvents evaporated. The remaining aqueous phase was extracted with EtOAc. The organic layer was separated and washed with sat. NaHCO₃- and NaCl-solution. After the organic solution was dried with MgSO₄, the solvent was evaporated and the crude product purified on silica (0 to 100% EtOAc/MeOH (9:1) in n-heptane), yielding 4.61 g (38.8%) of the title compound [(2R,3R,4R,5R)-4-acetoxy-2-[[2-[2-[2-[tert-butyl(diphenyl)silyl]oxy-ethoxy]ethoxy]ethoxy-(2-cyanoethoxy)phosphoryl]oxymethyl]-5-[2-(2-methylpropanoyl-amino)-6-oxo-1H-purin-9-yl]tetrahydrofuran-3-yl] acetate (225) as color-less foam.

LC-MS (Method A):

Rₜ[min] (ELSD-signal): 2.90, 2.93 (mixture of diastereomers)

MS (calc.: 940.3) (m/z)=941.5 [M+H⁺].

### Example 18.7: Synthesis of [(2R,3R,4R,5R)-4-Acetoxy-2-[[2-cyanoethoxy-[2-[2-(2-hydroxy-ethoxy)-ethoxy]ethoxy]phosphoryl]oxymethyl]-5-[2-(2-methylpropanoylamino)-6-oxo-1H-purin-9-yl]tetrahydrofuran-3-yl] acetate (226)

4.60 g (4.89 mmol) of the silylether [(2R,3R,4R,5R)-4-acetoxy-2-[[2-[2-[2-[tert-butyl-(diphenyl)silyl]oxy-ethoxy]ethoxy]ethoxy-(2-cyanoethoxy)phosphoryl]oxymethyl]-5-[2-(2-methylpropanoyl-amino)-6-oxo-1H-purin-9-yl]tetrahydrofuran-3-yl] acetate (225) were dissolved in 50 ml THF. At room temperature, 7.45 g (48.88 mmol) pyridine-HF were added drop wise and the solution was stirred for 2.5 h to achieve complete deprotection. Under vigorous stirring, 25 g NaHCO₃ were added in portions and stirring was continued for 4 h. The reaction mixture was filtered and the filtrate evaporated. The crude product was purified by silicagel chromatography (0 to 10% MeOH in DCM), which gave 1.80 g (52.4%) of the alcohol [(2R,3R,4R,5R)-4-acetoxy-2-[[2-cyanoethoxy-[2-[2-(2-hydroxyethoxy)-ethoxy]ethoxy]phosphoryl]oxymethyl]-5-[2-(2-methylpropanoylamino)-6-oxo-1H-purin-9-yl]tetrahydrofuran-3-yl] acetate (226) as colorless foam.

LC-MS (Method A):

Rₜ[min] (ELSD-signal): 1.42

MS (calc.: 702.2) (m/z)=703.3 [M+H⁺].

### Example 18.8: Synthesis of [(2R,3R,4R,5R)-4-Acetoxy-2-[[2-cyanoethoxy-[2-[2-(2-oxo-ethoxy)ethoxy]-ethoxy]phosphoryl]oxymethyl]-5-[2-(2-methylpropanoylamino)-6-oxo-1H-purin-9-yl]tetra-hydrofuran-3-yl] acetate (227)

658.1 mg (8.41 mmol) DMSO were dissolved in 50 ml dry DCM. At −60° C., 1.94 ml (3.88 mmol) of a 2 M solution of oxalyl chloride in DCM were added drop wise under an argon atmosphere and the solution was stirred for 15 min. After adding a solution of the alcohol [(2R,3R,4R,5R)-4-acetoxy-2-[[2-cyanoethoxy-[2-[2-(2-hydroxyethoxy)-ethoxy]ethoxy]phosphoryl]oxymethyl]-5-[2-(2-methylpropanoylamino)-6-oxo-1H-purin-9-yl]tetrahydrofuran-3-yl] acetate (226, 1.52 g, 2.16 mmol) in 50 ml DCM, stirring was continued for 20 min, followed by the addition of 3.29 g (32.27 mmol) NEt₃. The cooling bath was removed and the reaction solution allowed to reach room temperature. The solution was washed with 50 ml citric acid solution (5%). The organic layer was separated and dried with MgSO₄. Evaporation of the solvent gave 1.62 g (crude) of the desired aldehyde [(2R,3R,4R,5R)-4-acetoxy-2-[[2-cyanoethoxy-[2-[2-(2-oxoethoxy)ethoxy]-ethoxy]phosphoryl]oxymethyl]-5-[2-(2-methylpro-panoylamino)-6-oxo-1H-purin-9-yl]tetra-hydrofuran-3-yl] acetate (227) as beige foam, which was used without further purification.

LC-MS (Method A):

Rₜ[min] (ELSD-signal): 1.38

MS (calc.: 700.2) (m/z)=701.3 [M+H⁺].

### Example 18.9: Synthesis of 1-[(2R,6R)-6-[[Bis(4-methoxyphenyl)-phenyl-methoxy]methyl]-6-(hydroxymethyl)morpholin-2-yl]-5-methyl-pyrimidine-2,4-dione (228)

3.50 g (4.79 mmol) of the silyl protected morpholine 1-[(2R,6S)-6-[[bis(4-methoxy-phenyl)-phenyl-methoxy]methyl]-6-(triisopropylsilyloxymethyl)morpholin-2-yl]-5-methyl-pyrimi-dine-2,4-dione (215) (Hofmeister et al., WO2019170731) were dissolved in 35 ml DMF. After adding 6.79 g (67.12 mmol) NEt₃ and 12.75 g (76.71 mmol) NEt₃·3HF, the reaction mixture was stirred at 80° C. for 1 h and then allowed to reach room temperature. 500 ml H₂O/sat. NaHCO₃-solution (1:1) were added and the mixture was stirred for 30 min. The aqueous solution was extracted with EtOAc. The organic layer separated, dried with MgSO₄ and evaporated. The crude product was dissolved in 80 ml EtOAc and poured into 320 ml n-heptane. The precipitate was filtered and dried in vacuo at 40° C., yielding 2.27 g (82.5%) of the desired product 1-[(2R,6R)-6-[[bis(4-methoxyphenyl)-phenyl-methoxy]methyl]-6-(hydroxymethyl)-morpholin-2-yl]-5-methyl-pyrimidine-2,4-dione (228) as colorless solid.

LC-MS (Method A):

Rₜ[min] (ELSD-signal): 1.78

MS (calc.: 573.6) (m/z)=572.3 [M−H⁺].

### Example 18.10: Synthesis of [(2R,3R,4R,5R)-4-Acetoxy-2-[[2-[2-[2-[(2R,6R)-2-[[bis(4-methoxyphenyl)-phenyl-methoxy]-methyl]-2-(hydroxymethyl)-6-(5-methyl-2,4-dioxo-pyrimidin-1-yl)-morpholin-4-yl]ethoxy]-ethoxy]ethoxy-(2-cyanoethoxy)phosphoryl]-oxymethyl]-5-[2-(2-methylpropanoylamino)-6-oxo-1H-purin-9-yl]tetrahydrofuran-3-yl]acetate (229)

Following the protocol, described for the synthesis of [(2R,3R,4R,5R)-4-Acetoxy-2-[2-[2-[2-[(2S,6R)-2-[[bis(4-meth-oxyphenyl)-phenyl-methoxy]methyl]-6-(5-methyl-2,4-dioxo-pyr-imidin-1-yl)-2-(triisopropyl-silyloxy-methyl)morpholin-4-yl]ethoxy]ethoxy]ethoxy-methyl]-5-[2-(2-methylpropanoyl-amino)-6-oxo-1H-purin-9-yl]tetrahydrofuran-3-yl] acetate (216), 1.0 g (1.43 mmol) of the aldehyde [(2R,3R,4R,5R)-4-acetoxy-2-[[2-cyanoethoxy-[2-[2-(2-oxoethoxy)ethoxy]-ethoxy]phosphoryl]oxymethyl]-5-[2-(2-methylpropanoylamino)-6-oxo-1H-purin-9-yl]tetra-hydrofuran-3-yl] acetate (227) and 818.8 mg (1.43 mmol) of 1-[(2R,6R)-6-[[bis(4-methoxyphenyl)-phenyl-methoxy]methyl]-6-(hydroxymethyl)morpholin-2-yl]-5-methyl-pyrimidine-2,4-dione (228) underwent a reductive amination reaction, using 377.7 mg (5.71 mmol) of sodium cyanoboronhydride, yielding 1.16 g (64.6%) of the title compound [(2R,3R,4R,5R)-4-acetoxy-2-[[2-[2-[2-[(2R,6R)-2-[[bis(4-methoxyphenyl)-phenyl-methoxy]-methyl]-2-(hydroxymethyl)-6-(5-methyl-2,4-dioxo-pyrimidin-1-yl)-morpholin-4-yl]ethoxy]-ethoxy]ethoxy-(2-cyanoethoxy)phosphoryl]oxymethyl]-5-[2-(2-methylpropanoylamino)-6-oxo-1H-purin-9-yl]tetrahydrofuran-3-yl] acetate (229) after silicagel chromatography (0 to 10% MeOH in DCM) as colorless foam.

LC-MS (Method A):

Rₜ[min] (ELSD-signal): 2.22

MS (calc.: 1257.5) (m/z)=1256.4 [M−H⁺].

¹H NMR (400 MHz, DMSO-d₆) δ[ppm]: 12.11 (s, 1H), 11.57 (s, 1H), 11.36 (s, 1H), 8.24 (d, J=2.7 Hz, 1H), 7.54 (s, 1H), 7.40 (d, J=7.5 Hz, 2H), 7.19-7.31 (m, 7H), 6.86 (d, J=8.8 Hz, 4H), 6.11 (d, J=7.0 Hz, 1H), 5.77-5.88 (m, 2H), 5.50 (m, 1H), 4.58 (m, 1H), 4.28-4.45 (m, 3H), 4.02-4.22 (m, 4H), 3.65-3.80 (m, 1H), 3.73 (s, 6H), 3.65 (m, 1H), 3.44-3.61 (m, 8H), 3.01 (br s, 2H), 2.85-2.95 (m, 3H), 2.72-2.83 (m, 2H), 2.23-2.31 (m, 1H), 2.09-2.21 (s, 4H), 2.07 (s, 2H), 2.01 (s, 3H), 1.67 (s, 3H), 1.13 (d, J=6.8 Hz, 6H).

### Example 18.11: Synthesis of [(2R,3R,4R,5R)-4-Acetoxy-2-[[2-[2-[2-[(2S,6R)-2-[[bis(4-methoxy-phenyl)-phenyl-methoxy]methyl]-2-[[2-cyanoethoxy-(diisopropylamino)-phosphanyl]-oxy-methyl]-6-(5-methyl-2,4-dioxo-pyrimidin-1-yl)morpholin-4-yl]ethoxy]ethoxy]-ethoxy-(2-cyanoethoxy)phosphoryl]oxymethyl]-5-[2-(2-methylpropanoylamino)-6-oxo-1H-purin-9-yl]tetrahydrofuran-3-yl] acetate (230)

271 mg (0.22 mmol) of the alcohol [(2R,3R,4R,5R)-4-acetoxy-2-[[2-[2-[2-[(2R,6R)-2-[[bis(4-methoxyphenyl)-phenyl-methoxy]-methyl]-2-(hydroxymethyl)-6-(5-methyl-2,4-dioxo-pyr-imidin-1-yl)-morpholin-4-yl]ethoxy]-ethoxy]ethoxy-(2-cyanoethoxy)phosphoryl]oxymethyl]-5-[2-(2-methylpropanoylamino)-6-oxo-1H-purin-9-yl]tetrahydrofuran-3-yl] acetate (229) and 46.7 mg (0.36 mmol) DIPEA were dissolved in 3.5 ml dry DCM. Under an argon atmosphere, 78.8 mg (0.32 mmol) 2-cyanoethyl-N,N,diisopropylchlorophsophoramidite were added at 0° C. and the solution was stirred at 0° C. for 2 h. To achieve complete conversion, additional 0.3 equivalents DIPEA and 0.25 equivalents 2-cyanoethyl-N,N,diisopropylchlorophsophor-amidite were added and the solution was stirred for another 45 min. After adding 5 ml diethylether, the reaction solution was dropped into 30 ml n-pentane. The precipitate was isolated by centrifugation (4200 rpm/2 min at 20° C.). The solvents were decanted and the precipitate dissolved in 10 ml EtOAc/diethylether (1:1). After adding 40 ml n-pentane, the precipitate was again collected by centrifugation and decanting of the mother liquor. The precipitation procedure was repeated four times and the final precipitate dried on a Speedvac at 30° C., yielding 314 mg (quant.) of the phosphoramidite [(2R,3R,4R,5R)-4-acet-oxy-2-[[2-[2-[2-[(2S,6R)-2-[[bis(4-methoxyphenyl)-phenyl-methoxy]methyl]-2-[[2-cyano-ethoxy-(diiso-propylamino)phosphanyl]-oxy-methyl]-6-(5-methyl-2,4-dioxo-pyrimidin-1-yl)-morpholin-4-yl]ethoxy]ethoxy]ethoxy-(2-cyanoethoxy)phosphoryl]oxymethyl]-5-[2-(2-methylpropanoyl-amino)-6-oxo-1H-purin-9-yl]tetrahydrofuran-3-yl] acetate (230) as colorless foam (mixture of diastereomers).

LC-MS (Method B):

Rₜ[min] (UV-signal 254 nm): 0.81

MS (calc.: 1457.6) (m/z)=1073.3 [M−DMT⁺−^{iPr2}N⁻+H₂O+H⁺].

³¹P-NMR (162 MHz) δ[ppm]: 147.58, 147.37, 9.40, 8.91.

### Example 19: Synthesis of Targeted Nucleotide Precursor 246 (Pre-lsT3

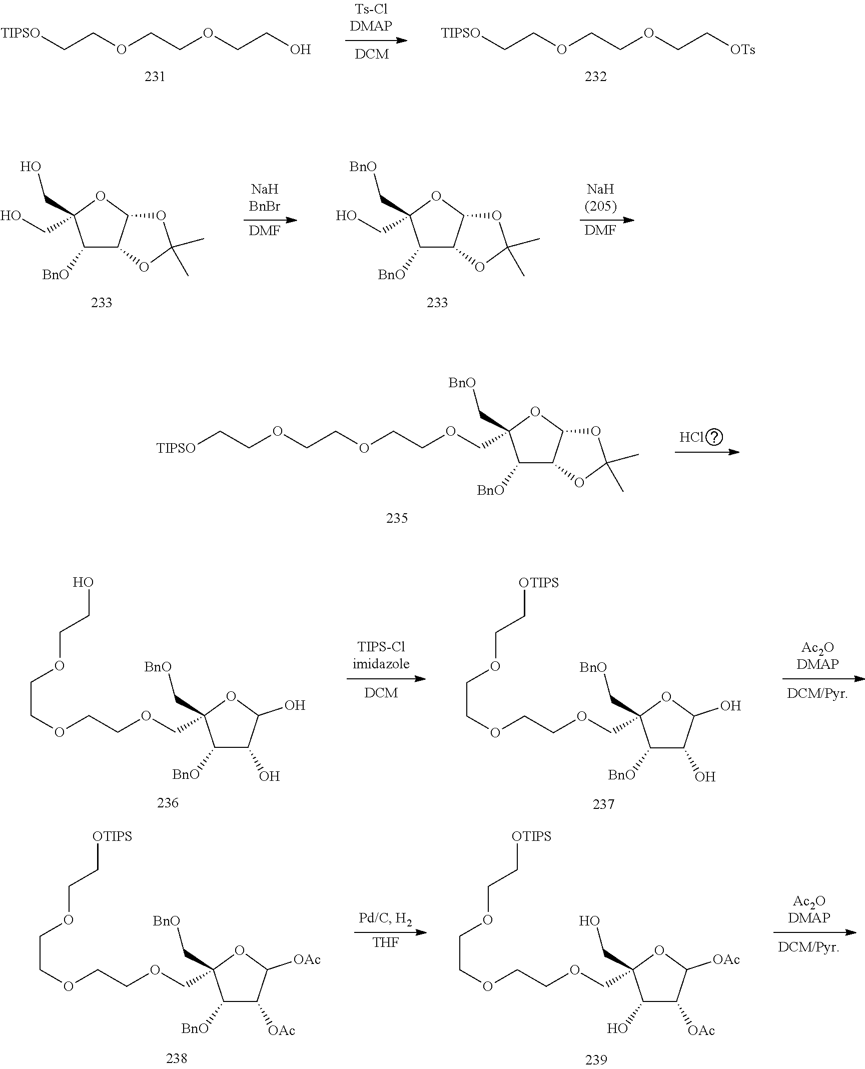

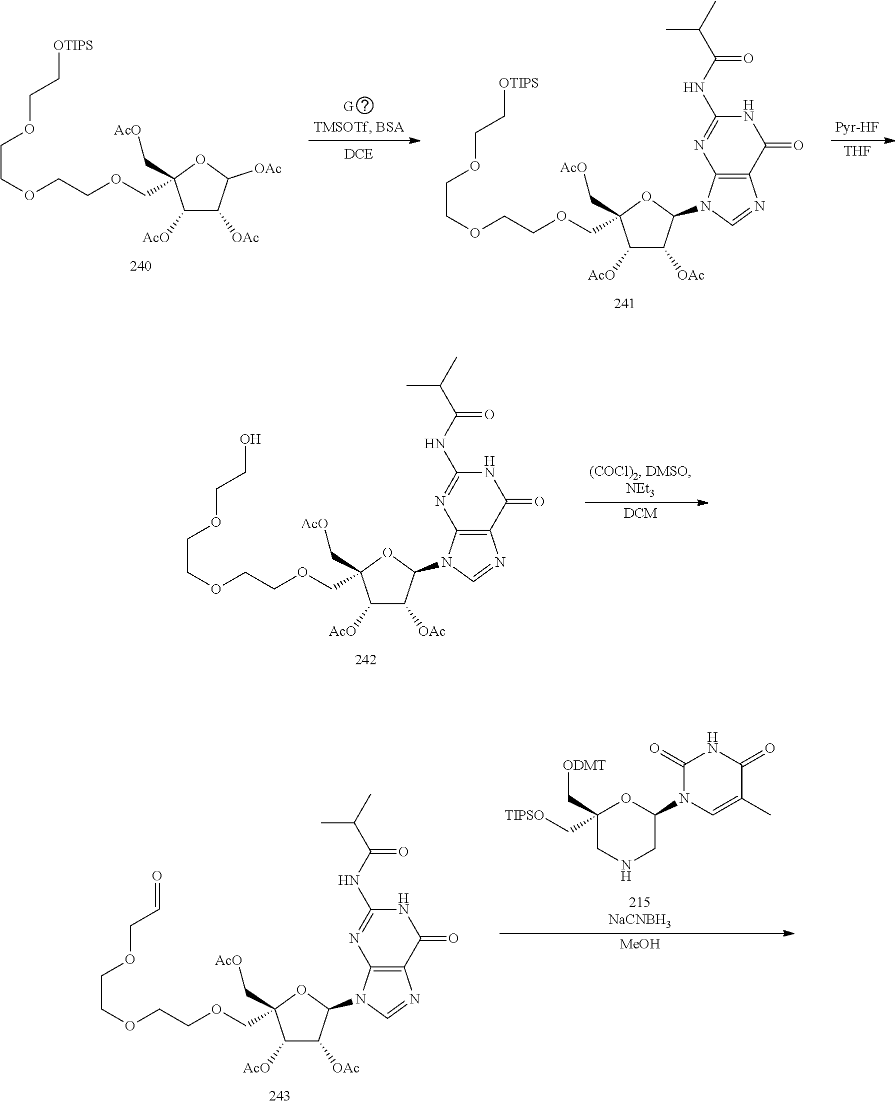

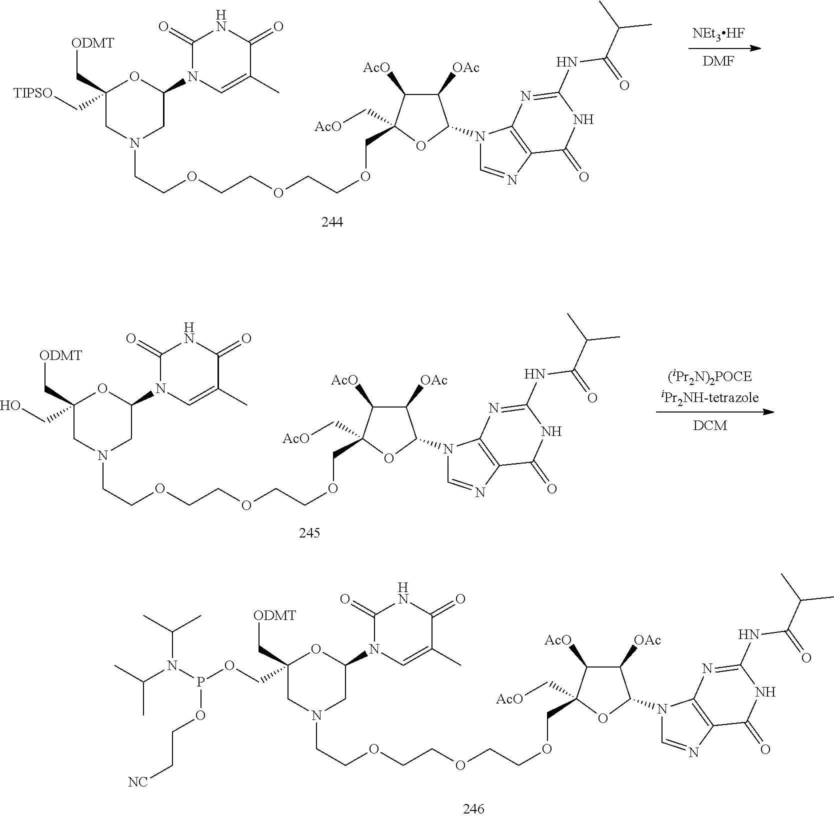

### Example 19.1: Synthesis of [(3aR,5R,6S,6aR)-6-Benzyloxy-5-(benzyloxymethyl)-2,2-dimethyl-6,6a-dihydro-3aH-furo[2,3-d][1,3]dioxol-5-yl]methanol (234)

10.0 g (32.22 mmol) of the starting material [(3aR,6S,6aR)-6-benzyloxy-5-(hydroxymethyl)-2,2-dimethyl-6,6a-dihydro-3aH-furo[2,3-d][1,3]dioxol-5-yl]methanol (233) were dissolved in 50 ml dry DMF. At 0° C., 1.48 g (37.06 mmol) sodium hydride (60%) added in five portions over a period of 60 min. After the addition of 6.47 g (37.06 mmol) benzyl bromide, dissolved in 50 ml DMF, the cooling bath was removed and the reaction stirred for 20 h. To the reaction were added 600 ml H₂O and 300 ml methyl-tert. butylether. The organic layer was separated washed 3× with 5% NaCl-solution. After drying over MgSO₄, the solvents were evaporated and the crude product purified by silicagel chromatography (15 to 65% EtOAc in n-heptane), yielding 7.53 g (58.4%) of the title compound [(3aR,5R,6S,6aR)-6-benzyloxy-5-(benzyloxy-methyl)-2,2-dimethyl-6,6a-dihydro-3aH-furo[2,3-d][1,3]dioxol-5-yl]methanol (234) as color-less oil.

LC-MS (Method A):

Rₜ[min] (ELSD-signal): 2.34

¹H NMR (400 MHz, DMSO-d₆) δ[ppm]: 7.24-7.36 (m, 10H), 5.70 (d, J=3.8 Hz, 1H), 4.75 (dd, J=5.1, 4.0 Hz, 1H), 4.65 (d, J=12.1 Hz, 1H), 4.40-4.51 (m, 3H), 4.26 (dd, J=6.6, 5.1 Hz, 1H), 4.18 (d, J=5.3 Hz, 1H), 3.83 (dd, J=11.9, 5.0 Hz, 1H), 3.59 (dd, J=11.9, 6.7 Hz, 1H), 3.51 (d, J=10.3, 1H), 3.46 (d, J=10.4, 1H), 1.49 (s, 3H), 1.27 (s, 3H).

### Example 19.2: Synthesis of 2-[2-(2-Triisopropylsilyloxyethoxy)ethoxy]ethanol (231)

30.53 g (201.24 mmol) Triethylene glycol and 7.61 g (110.68 mmol) imidazole were dissolved in 150 ml DCM. A solution of 10.0 g (50.31 mmol) TIPS-Cl in 150 ml DCM was added over a period of 1 h and the solution stirred at room temperature. After 18 h, the organic solution was washed with 650 ml H₂O. The organic layer was separated and the aqueous phase extracted with 2×150 ml DCM. The combined organic layers were dried with MgSO₄ and evaporated. Purification on silica (10 to 65% EtOAc in n-heptane) gave 12.83 g (83.2%) of the silylether 2-[2-(2-triisopropylsilyloxyethoxy)ethoxy]ethanol (231) as colorless liquid.

LC-MS (Method A):

Rₜ[min] (MS-signal TIC): 2.76

MS (calc.: 306.2) (m/z)=307.3 [M+H⁺].

### Example 19.3: Synthesis of 2-[2-(2-Triisopropylsilyloxyethoxy)ethoxy]ethyl 4-methyl-benzenesulfonate (232)

To a solution of 12.7 g (41.43 mmol) of the starting material 2-[2-(2-triisopropylsilyloxy-ethoxy)ethoxy]ethanol (231) in 100 ml dry pyridine were added 8.38 g (43.50 mmol) p-toluenesulfonyl chloride and 127.8 mg (1.04 mmol) DMAP at room temperature. After stirring for 1.5 h, 1600 ml of a 2 M HCl and 400 ml methyl-tert. butylether were added. The layers were separated and the organic phase was washed with 2×500 ml H₂O and 350 sat. NaCl-solution. The organic phase was dried with MgSO₄ and evaporated. Purification on silica (10 to 33% EtOAc in n-heptane) gave 10.09 g (52.8%) of the desired tosylate 2-[2-(2-triisopropyl-silyloxyethoxy)ethoxy]ethyl 4-methylbenzenesulfonate (232) as colorless liquid.

LC-MS (Method A):

Rₜ[min] (ELSD-signal): 3.37

MS (calc.: 460.2) (m/z)=461.3 [M+H⁺].

### Example 19.4: Synthesis of 2-[2-[2-[[(3aR,5S,6S,6aR)-6-Benzyloxy-5-(benzyloxymethyl)-2,2-dimethyl-6,6a-dihydro-3aH-furo[2,3-d][1,3]dioxol-5-yl]methoxy]ethoxy]ethoxy]ethoxy-triisopropyl-silane (235)

6.46 g (16.13 mmol) of the primary alcohol [(3aR,5R,6S,6aR)-6-Benzyloxy-5-(benzyloxy-methyl)-2,2-dimethyl-6,6a-dihydro-3aH-furo[2,3-d][1,3]dioxol-5-yl]methanol (234) were dis-solved in 35 ml dry DMF. Under an atmosphere of argon, 871.0 mg (21.78 mmol) sodium hydride (60%) were added and the solution was stirred at room temperature for 1.5 h. The reaction mixture was cooled to 0° C. and a solution of 7.43 g (16.13 mmol) of the tosylate 2-[2-(2-triisopropyl-silyloxyethoxy)ethoxy]ethyl 4-methylbenzenesulfonate (232) in 35 ml dry DMF was added dropwise. The cooling bath was removed and the mixture stirred for 3 h, followed by the addition of 500 ml H₂O. The reaction solution was extracted with 350 ml methyl-tert. butylether, the organic layer was separated and washed with 3×350 ml 5% NaCl-solution. After drying with MgSO₄ and evaporation of the solvent, the crude product was purified by silicagel chromatography (0 to 100% EtOAc in n-heptane), which gave 6.21 g (55.9%) of the title compound 2-[2-[2-[[(3aR,5S,6S,6aR)-6-benzyloxy-5-(benzyloxymethyl)-2,2-dimethyl-6,6a-dihydro-3aH-furo[2,3-d][1,3]dioxol-5-yl]methoxy]ethoxy]ethoxy]ethoxy-tri-isopropyl-silane (235) as colorless oil.

LC-MS (Method A):

Rₜ[min] (ELSD-signal): 3.74

MS (calc.: 688.4) (m/z)=631.4 [M−H₃CC(O)CH₃+H⁺]; 711.5 [M+Na⁺].

### Example 19.5: Synthesis of (3R,4S,5S)-4-Benzyloxy-5-(benzyloxymethyl)-5-[2-[2-(2-hydroxyethoxy)-ethoxy]ethoxymethyl]tetrahydrofuran-2,3-diol (236)

67.49 ml (2.70 mmol) of a 0.04 M HCl were added to 6.20 g (9.00 mmol) of the furanose derivative 2-[2-[2-[[(3aR,5S,6S,6aR)-6-Benzyloxy-5-(benzyloxymethyl)-2,2-dimethyl-6,6a-dihydro-3aH-furo[2,3-d][1,3]dioxol-5-yl]methoxy]ethoxy]ethoxy]ethoxy-triisopropyl-silane (235) in 70 ml dioxane. The reaction solution was stirred for 2 h at 100° C. After the mixture was cooled to room temperature, 50 ml of a sat. NaHCO₃-solution were added and the solution was concentrated in vacuo. The remaining aqueous solution was extracted with 2×150 ml DCM. The combined organic layers were dried with MgSO₄ and evaporated. Purification by silicagel chromatography (0 to 8% MeOH in DCM) gave 4.08 g (91.9%) of the triol (3R,4S,5S)-4-benzyloxy-5-(benzyloxymethyl)-5-[2-[2-(2-hydroxyethoxy)-ethoxy]ethoxy-methyl]tetrahydrofuran-2,3-diol (236) as colorless oil.

LC-MS (Method A):

Rₜ[min] (ELSD-signal): 1.99

MS (calc.: 492.2) (m/z)=475.3 [M-OH⁻].

### Example 19.6: Synthesis of (3R,4S,5S)-4-Benzyloxy-5-(benzyloxymethyl)-5-[2-[2-(2-triiso-propylsilyl-oxyethoxy)ethoxy]ethoxymethyl]tetrahydrofuran-2,3-diol (237)

1.81 g (9.09 mmol) Triisopropylsilyl chloride in 40 ml DCM were added to a solution of 4.07 g (8.26 mmol) (3R,4S,5S)-4-benzyloxy-5-(benzyloxymethyl)-5-[2-[2-(2-hydroxyethoxy)-ethoxy]ethoxymethyl]tetrahydrofuran-2,3-diol (236) and 1.88 g (27.27 mmol) imidazole in 70 ml DCM. The solution was stirred for 30 min at room temperature, when additional 1.59 g (8.26 mmol) triisopropylsilyl chloride were added. After another 60 min at room temperature, the reaction was quenched by the addition of 10 ml MeOH. The organic solution was washed with 100 ml H₂O and the layers were separated. The aqueous phase was extracted with 2×100 ml DCM and the combined organic layers dried with MgSO₄. After evaporation of the solvents, the crude product was purified by silicagel chromatography (20 to 100% EtOAc in n-heptane), yielding 4.86 g (90.5%) of the silylether (3R,4S,5S)-4-benzyloxy-5-(benzyloxymethyl)-5-[2-[2-(2-triisopropylsilyl-oxyethoxy)ethoxy]ethoxymethyl]tetrahydrofuran-2,3-diol (237) as colorless oil.

LC-MS (Method A):

Rₜ[min] (ELSD-signal): 3.46

MS (calc.: 648.4) (m/z)=631.5 [M-OH⁻].

### Example 19.7: Synthesis of [(3R,4S,5S)-2-Acetoxy-4-benzyloxy-5-(benzyloxymethyl)-5-[2-[2-(2-triisopropylsilyloxyethoxy)ethoxy]ethoxymethyl]tetrahydrofuran-3-yl] acetate (238)

The starting material (3R,4S,5S)-4-benzyloxy-5-(benzyloxymethyl)-5-[2-[2-(2-triisopropyl-silyloxyethoxy)ethoxy]ethoxymethyl]tetrahydrofuran-2,3-diol (237, 4.85 g, 7.47 mmol) was dissolved in 100 ml DCM/pyridine (4:1). At room temperature, 3.85 g (37.37 mmol) acetic anhydride in 35 ml DCM were added, followed by 32.6 mg (0.26 mmol) DMAP. The reaction solution was stirred for 1.5 h, to achieve complete conversion. After adding 10 ml EtOH, stirring was continued for 30 min and the solvents were evaporated. The residue was dissolved in 250 ml methyl-tert. butylether and washed with 250 ml H₂O, 2×250 ml citric acid solution (10%) and 2×250 ml sat. NaHCO₃-solution. The organic layer was separated and dried with MgSO₄. Evaporation of the solvent gave 5.40 g (crude) of the title compound [(3R,4S,5S)-2-acetoxy-4-benzyloxy-5-(benzyloxymethyl)-5-[2-[2-(2-triisopropylsilyloxyethoxy)ethoxy]-ethoxymethyl]tetrahydrofuran-3-yl] acetate (238), which was used in the following step without further purification.

LC-MS (Method C):

Rₜ[min] (UV-signal 220 nm): 1.03

MS (calc.: 732.4) (m/z)=750.5 [M+H₂O+H⁺].

### Example 19.8: Synthesis of [(3R,4S,5S)-2-Acetoxy-4-hydroxy-5-(hydroxymethyl)-5-[2-[2-(2-triiso-propylsilyloxyethoxy)ethoxy]ethoxymethyl]tetrahydrofuran-3-yl] acetate (239)

The bisbenzylether [(3R,4S,5S)-2-acetoxy-4-benzyloxy-5-(benzyloxymethyl)-5-[2-[2-(2-tri-isopropylsilyloxyethoxy)ethoxy]ethoxymethyl]tetrahydrofuran-3-yl] acetate (238, 5.40 g, 7.37 mmol, crude) was dissolved in 65 ml THF. After adding 196 mg (0.18 mmol) Pd/C (10%) under argon, the apparatus was set under 4.5 bar H2-pressure and the mixture was stirred for 4 h at room temperature. The catalyst was filtered and the filtrate evaporated, yielding 4.24 g (quant., crude) of the desired diol [(3R,4S,5S)-2-acetoxy-4-hydroxy-5-(hydroxymethyl)-5-[2-[2-(2-triiso-propylsilyloxyethoxy)ethoxy]ethoxymethyl]tetrahydrofuran-3-yl] acetate (239) as colorless oil, which was used in the following reaction without further purification.

LC-MS (Method A):

Rₜ[min] (ELSD-signal): 2.81

MS (calc.: 552.3) (m/z)=575.4 [M+Na⁺].

### Example 19.9: Synthesis of [(2R,3S,4R)-3,4,5-Triacetoxy-2-[2-[2-(2-triisopropylsilyloxy-ethoxy)-ethoxy]ethoxymethyl]tetrahydrofuran-2-yl]methyl acetate (240)

To a solution of 4.23 g (7.35 mmol, crude) of the starting material [(3R,4S,5S)-2-acetoxy-4-hydroxy-5-(hydroxymethyl)-5-[2-[2-(2-triiso-propylsilyloxyethoxy)ethoxy]ethoxymethyl]-tetrahydrofuran-3-yl] acetate (239) in 125 ml DCM/pyridine (4:1) were added 3.79 g (36.73 mmol) acetic anhydride, followed by 38.9 mg (0.31 mmol) DMAP. The solution was stirred at room temperature for 17 h and quenched with the addition of 15 ml EtOH. The solvents were evaporated and the residue dissolved in 250 ml EtOAc. The organic solution was washed with 250 ml H₂O, 2×250 ml citric acid solution (10%) and 2×250 ml sat. NaHCO₃-solution. After drying with MgSO₄ and evaporation, the crude product was purified by silicagel chromatography (0 to 100% EtOAc in n-heptane), yielding 4.24 g (90.6%) of the peracetylated product [(2R,3S,4R)-3,4,5-triacetoxy-2-[2-[2-(2-triisopropylsilyloxyethoxy)-ethoxy]ethoxy-methyl]tetrahydrofuran-2-yl]methyl acetate (240) as colorless oil.

LC-MS (Method A):

Rₜ[min] (ELSD-signal): 3.22

MS (calc.: 636.3) (m/z)=577.4 [M-OAc⁻].

### Example 19.10: Synthesis of [(2R,3S,4R,5R)-3,4-Diacetoxy-5-[2-(2-methylpropanoylamino)-6-oxo-1H-purin-9-yl]-2-[2-[2-(2-triisopropylsilyloxyethoxy)ethoxy]ethoxymethyl]tetrahydro-furan-2-yl]methyl acetate (241)

4.50 g (19.93 mmol) N-Isobutyrylguanine were dissolved in 50 ml dry DCE. Under reflux conditions, 12.80 g (59.78 mmol) BSA were added and the solution was stirred at this temperature for 1 h. After cooling to room temperature, a solution of 4.23 g (6.64 mmol) of the ribose derivative [(2R,3S,4R)-3,4,5-triacetoxy-2-[2-[2-(2-triisopropylsilyloxyethoxy)-ethoxy]-ethoxymethyl]tetrahydrofuran-2-yl]methyl acetate (240) in 15 ml DCE were added, followed by 6.71 g (29.89 mmol) TMSOTf. The reaction mixture was stirred under reflux for 2 h, to achieve complete conversion. The reaction solution was allowed to reach room temperature and dropped under vigorous stirring into 250 ml sat. NaHCO₃-solution/H₂O (1:1). The organic solvent was evaporated and the aqueous mixture extracted with EtOAc. The phases were filtered and separated. The organic layer was washed with sat. NaHCO₃- and sat. NaCl-solution. After drying with MgSO₄, the solvent was evaporated and the crude product purified on silica (10 to 90% MeOH/EtOAc (1:9) in n-heptane), yielding 2.69 g (50.7%) of the guanosine derivative [(2R,3S,4R,5R)-3,4-diacetoxy-5-[2-(2-methylpropanoylamino)-6-oxo-1H-purin-9-yl]-2-[2-[2-(2-triisopropylsilyloxyethoxy)ethoxy]ethoxymethyl]tetrahydrofuran-2-yl]methyl acetate (241) as colorless foam.

LC-MS (Method A):

Rₜ[min] (ELSD-signal): 3.07

MS (calc.: 797.4) (m/z)=798.5 [M+H⁺].

### Example 19.11: Synthesis of [(2R,3S,4R,5R)-3,4-Diacetoxy-2-[2-[2-(2-hydroxyethoxy)-ethoxy]ethoxy-methyl]-5-[2-(2-methylpropanoylamino)-6-oxo-1H-purin-9-yl]tetrahydro-furan-2-yl]methyl acetate (242)

2.75 g (3.45 mmol) of the silylether [(2R,3S,4R,5R)-3,4-diacetoxy-5-[2-(2-methylpropanoyl-amino)-6-oxo-1H-purin-9-yl]-2-[2-[2-(2-triisopropylsilyloxyethoxy)ethoxy]-ethoxymethyl]-tetrahydrofuran-2-yl]methyl acetate (241) were dissolved in 40 ml THF. 5.25 g (34.46 mmol) pyridine-HF (65%) were added and the reaction was stirred at room temperature. After 45 min, 18.0 g NaHCO₃ were added carefully under vigorous stirring. The mixture was filtered and the filtrate evaporated. After purification on silica (0 to 10% MeOH in DCM), 1.82 g (82.4%) of the title compound [(2R,3S,4R,5R)-3,4-diacetoxy-2-[2-[2-(2-hydroxyethoxy)-ethoxy]ethoxy-methyl]-5-[2-(2-methylpropanoylamino)-6-oxo-1H-purin-9-yl]tetrahydro-furan-2-yl]methyl acetate (242) were isolated as colorless foam.

LC-MS (Method A):

Rₜ[min] (ELSD-signal): 1.51

MS (calc.: 641.3) (m/z)=642.3 [M+H⁺].

### Example 19.12: Synthesis of [(2R,3S,4R,5R)-3,4-Diacetoxy-5-[2-(2-methylpropanoylamino)-6-oxo-1H-purin-9-yl]-2-[2-[2-(2-oxoethoxy)ethoxy]ethoxymethyl]tetrahydrofuran-2-yl]-methyl acetate (243)

0.92 ml (1.83 mmol) of a 2 M solution of oxalyl chloride in DCM were dissolved in 8 ml dry DCM. At −60° C., a solution of 286.8 mg (3.67 mmol) DMSO in 8 ml dry DCM was added drop wise under an argon atmosphere and the solution was stirred for 30 min. After adding a solution of 905 mg (1.41 mmol) of the alcohol [(2R,3S,4R,5R)-3,4-diacetoxy-2-[2-[2-(2-hydroxyethoxy)ethoxy]ethoxy-methyl]-5-[2-(2-methylpropanoylamino)-6-oxo-1H-purin-9-yl]tetrahydrofuran-2-yl]methyl acetate (242) in 10 ml DCM, stirring was continued for 30 min at −60° C., followed by the addition of 860.7 mg (8.46 mmol) NEt₃. After 10 min, the cooling bath was removed and the reaction solution allowed to reach room temperature. The solution was washed with 40 ml H₂O and the layers were separated. The aqueous phase was extracted with 2×40 ml DCM and the combined organic layers dried with MgSO₄. Evaporation of the solvent gave 989 mg (crude) of the desired aldehyde [(2R,3S,4R,5R)-3,4-diacetoxy-5-[2-(2-methylpropanoylamino)-6-oxo-1H-purin-9-yl]-2-[2-[2-(2-oxoethoxy)ethoxy]ethoxymethyl]-tetrahydrofuran-2-yl]methyl acetate (243) as light yellow foam, which was used in the next step without further purification.

LC-MS (Method B):

Rₜ[min] (UV-signal 254 nm): 0.57

MS (calc.: 639.2) (m/z)=640.3 [M+H⁺].

### Example 19.13: Synthesis of [(2R,3S,4R,5R)-3,4-Diacetoxy-2-[2-[2-[2-[(2S,6R)-2-[[bis(4-methoxy-phenyl)-phenyl-methoxy]methyl]-6-(5-methyl-2,4-dioxo-pyrimidin-1-yl)-2-(triiso-propyl-silyloxymethyl)morpholin-4-yl]ethoxy]ethoxy]ethoxymethyl]-5-[2-(2-methylpropano-yl-amino)-6-oxo-1H-purin-9-yl]tetrahydrofuran-2-yl]methyl acetate (244)

950.0 mg (1.34 mmol) of the aldehyde [(2R,3S,4R,5R)-3,4-diacetoxy-5-[2-(2-methylpropanoylamino)-6-oxo-1H-purin-9-yl]-2-[2-[2-(2-oxoethoxy)ethoxy]ethoxymethyl]-tetrahydrofuran-2-yl]methyl acetate (243) and 975.8 mg (1.34 mmol) of the morpholine 1-[(2R,6S)-6-[[bis(4-methoxy-phenyl)-phenyl-methoxy]methyl]-6-(triisopropylsilyloxymethyl)-morpholin-2-yl]-5-methyl-pyrimidine-2,4-dione (215) (Hofmeister et al., WO2019170731) were reacted under reductive amination conditions as described for the synthesis of (216). After purification on silica (column precondition with n-heptane+1% NEtO, 0 to 100% MeOH/EtOAc (9:1) in n-heptane), 773 mg (42.7%) of the title compound [(2R,3S,4R,5R)-3,4-diacetoxy-2-[2-[2-[2-[(2S,6R)-2-[[bis(4-methoxy-phenyl)-phenyl-methoxy]methyl]-6-(5-methyl-2,4-dioxo-pyrimidin-1-yl)-2-(triisopropyl-silyloxymethyl)morpholin-4-yl]ethoxy]-ethoxy]ethoxymethyl]-5-[2-(2-methylpropanoyl-amino)-6-oxo-1H-purin-9-yl]tetrahydro-furan-2-yl]methyl acetate (244) were isolated as colorless foam.

LC-MS (Method A):

Rₜ[min] (ELSD-signal): 3.40

MS (calc.: 1352.6) (m/z)=1352.0 [M−H⁺].

### Example 19.14: Synthesis of [(2R,3S,4R,5R)-3,4-Diacetoxy-2-[2-[2-[2-[(2R,6R)-2-[[bis(4-methoxy-phenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropano-ylamino)-6-oxo-1H-purin-9-yl]tetrahydrofuran-2-yl]methyl acetate (245)

750 mg (0.55 mmol) of the silylether [(2R,3S,4R,5R)-3,4-diacetoxy-2-[2-[2-[2-[(2S,6R)-2-[[bis(4-methoxy-phenyl)-phenyl-methoxy]methyl]-6-(5-methyl-2,4-dioxo-pyrimidin-1-yl)-2-(triisopropyl-silyloxymethyl)morpholin-4-yl]ethoxy]ethoxy]ethoxymethyl]-5-[2-(2-methyl-propanoyl-amino)-6-oxo-1H-purin-9-yl]tetrahydrofuran-2-yl]methyl acetate (244) were deprotected, following the protocol described for the synthesis of (217). After silicagel chromatography (0 to 100% EtOAc in n-heptane), 410.0 mg (61.8%) of the desired product [(2R,3S,4R,5R)-3,4-diacetoxy-2-[2-[2-[2-[(2R,6R)-2-[[bis(4-methoxy-phenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoylamino)-6-oxo-1H-purin-9-yl]tetrahydrofuran-2-yl]methyl acetate (245) were isolated as colorless foam.

LC-MS (Method A):

Rₜ[min] (ELSD-signal): 2.30

MS (calc.: 1196.5) (m/z)=1195.6 [M−H⁺].

¹H NMR (500 MHz, DMSO-d₆) δ[ppm]: 12.12 (s, 1H), 11.62 (s, 1H), 11.37 (s, 1H), 8.26 (s, 1H), 7.55 (s, 1H), 7.40 (d, J=7.4 Hz, 2H), 7.19-7.31 (m, 7H), 6.86 (d, J=8.8 Hz, 4H), 6.12 (d, J=7.3 Hz, 1H), 5.96 (dd, J=7.4, 5.8 Hz, 1H), 5.84 (dd, J=9.7, 2.9 Hz, 1H), 5.64 (d, J=5.6 Hz, 1H), 4.61 (br t, J=5.1 Hz, 1H), 4.39 (d, J=11.7 Hz, 1H), 4.26 (d, J=11.6 Hz, 1H), 3.76 (m, 1H), 3.73 (s, 6H), 3.40-3.68 (m, 12H), 3.08 (m, 1H), 3.00 (m, 2H), 2.91 (br d, J=9.3 Hz, 1H), 2.70-2.87 (m, 2H), 2.52 (m, 2H), 2.26 (m, 1H), 2.16 (m, 1H), 2.14 (s, 3H), 2.06 (s, 3H), 1.99 (s, 3H), 1.67 (s, 3H), 1.12 (d, J=6.9 Hz, 6H).

### Example 19.15: Synthesis of [(2R,3S,4R,5R)-3,4-Diacetoxy-2-[2-[2-[2-[(2S,6R)-2-[[bis(4-methoxy-phenyl)-phenyl-methoxy]methyl]-2-[[2-cyanoethoxy-(diisopropylamino)phosphanyl]oxy-methyl]-6-(5-methyl-2,4-dioxo-pyrimidin-1-yl)morpholin-4-yl]ethoxy]ethoxy]-ethoxymethyl]-5-[2-(2-methylpropanoylamino)-6-oxo-1H-purin-9-yl]tetrahydrofuran-2-yl]methyl acetate (246)

Following the protocol, described for the synthesis of (218), 385 mg (0.32 mmol) of the starting material [(2R,3S,4R,5R)-3,4-diacetoxy-2-[2-[2-[2-[(2R,6R)-2-[[bis(4-methoxy-phenyl)-phen-yl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoylamino)-6-oxo-1H-purin-9-yl]tetra-hydrofuran-2-yl]methyl acetate (245) were phosphitylated and delivered 438 mg (97.5%) of the phosphoamidite [(2R,3S,4R,5R)-3,4-diacetoxy-2-[2-[2-[2-[(2S,6R)-2-[[bis(4-methoxy-phenyl)-phenyl-methoxy]methyl]-2-[[2-cyanoethoxy-(diisopropylamino)phosphanyl]oxy-methyl]-6-(5-methyl-2,4-dioxo-pyrimidin-1-yl)morpholin-4-yl]ethoxy]ethoxy]ethoxymethyl]-5-[2-(2-methylpropanoylamino)-6-oxo-1H-purin-9-yl]tetrahydrofuran-2-yl]methyl acetate (246) as colorless foam (mixture of diastereomers).

LC-MS (Method B):

Rₜ[min] (UV-signal 254 nm): 0.83

MS (calc.: 1396.6) (m/z)=1012.4 [M−DMT⁺−^{iPr2}N⁻+H₂O+H⁺].

³¹P-NMR (162 MHz) δ[ppm]: 147.53, 147.36.

### Example 20: Synthesis of Targeted Nucleotide Precursor 249 (Pre-lpT1

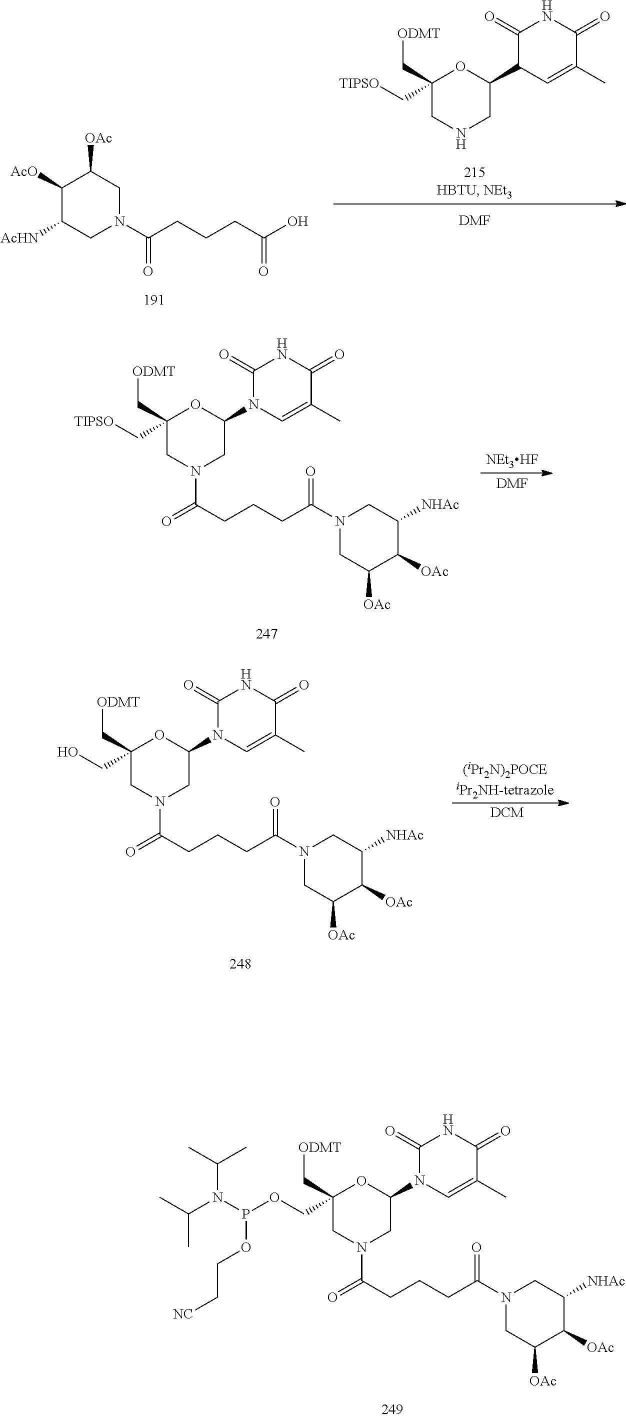

### Example 20.1: Synthesis of [(3S,4R,5S)-5-Acetamido-4-acetoxy-1-[5-[(2S,6R)-2-[[bis(4-methoxy-phenyl)-phenyl-methoxy]methyl]-6-(5-methyl-2,4-dioxo-pyrimidin-1-yl)-2-(tri-isopropylsilyl-oxymethyl)morpholin-4-yl]-5-oxo-pentanoyl]-3-piperidyl] acetate (247)

300.0 mg (0.41 mmol) of the morpholine 1-[(2R,6S)-6-[[bis(4-methoxy-phenyl)-phenyl-methoxy]methyl]-6-(triisopropylsilyloxymethyl)-morpholin-2-yl]-5-methyl-pyrimidine-2,4-dione (215) (Hofmeister et al., WO 2019/170731) and 153.0 mg (0.41 mmol) of the carboxylic acid 5-[(3S,4R,5S)-3-acetamido-4,5-diacetoxy-1-piperidyl]-5-oxo-pentanoic acid (191) were dissolved in 4 ml dry DCM. After the addition of 162.6 mg (1.23 mmol) NEt₃ and 238.6 mg (0.62 mmol) HBTU, the reaction mixture was stirred at room temperature. After 4 h, the reaction solution was diluted with 20 ml DCM and washed with 20 ml sat. NaHCO₃- and 20 ml sat. NaCl-solution. The organic layer was separated and dried with MgSO₄. After evaporation of the solvent, the crude product was purified by silicagel chromatography (0 to 5% MeOH in DCM), which gave 385 mg (86.4%) of the title compound [(3S,4R,5S)-5-acetamido-4-acetoxy-1-[5-[(2S,6R)-2-[[bis(4-methoxy-phenyl)-phenyl-methoxy]methyl]-6-(5-methyl-2,4-dioxo-pyrimidin-1-yl)-2-(triisopropylsilyl-oxymethyl)morpholin-4-yl]-5-oxo-pentanoyl]-3-piperid-yl] acetate (247) compound as colorless foam (mixture of peptide bond isomers).

LC-MS (Method A):

Rₜ[min] (ELSD-signal): 3.10

MS (calc.: 1084.5) (m/z)=1083.2 [M−H⁺].

### Example 20.2: Synthesis of [(3S,4R,5S)-5-Acetamido-4-acetoxy-1-[5-[(2R,6R)-2-[[bis(4-methoxy-phenyl)-phenyl-methoxy]methyl]-2-(hydroxymethyl)-6-(5-methyl-2,4-dioxo-pyrimidin-1-yl)-morpholin-4-yl]-5-oxo-pentanoyl]-3-piperidyl] acetate (248)

To a solution of 380 mg (0.35 mmol) of the silylether [(3S,4R,5S)-5-acetamido-4-acetoxy-1-[5-[(2S,6R)-2-[[bis(4-methoxy-phenyl)-phenyl-methoxy]methyl]-6-(5-methyl-2,4-dioxo-pyrimidin-1-yl)-2-(triisopropylsilyl-oxymethyl)morpholin-4-yl]-5-oxo-pentanoyl]-3-piperid-yl] acetate (247) in 75 ml DMF were added 537.3 mg (5.26 mmol) NEt₃ and 291.2 mg (1.75 mmol) NEt₃·3 HF. The solution was stirred at 75° C. for 4 h to achieve complete conversion. After the solution was cooled to room temperature, the reaction was poured carefully into 20 ml sat. NaHCO₃-solution/H₂O (1:1). The mixture was filtered and the filtrate evaporated. The residue was dissolved in DCM and dried with MgSO₄. After evaporation, the crude product was purified on silica (0 to 5% MeOH in DCM), yielding 147 mg (45.2%) of the alcohol [(3S,4R,5S)-5-acetamido-4-acetoxy-1-[5-[(2R,6R)-2-[[bis(4-methoxy-phenyl)-phenyl-meth-oxy]methyl]-2-(hydroxymethyl)-6-(5-methyl-2,4-dioxo-pyrimidin-1-yl)-morpholin-4-yl]-5-oxo-pentanoyl]-3-piperidyl] acetate (248) as colorless foam (mixture of peptide bond isomers).

LC-MS (Method A):

Rₜ[min] (ELSD-signal): 2.23

MS (calc.: 927.4) (m/z)=926.5 [M−H⁺].

### Example 20.3: Synthesis of [(3S,4R,5S)-5-Acetamido-4-acetoxy-1-[5-[(2S,6R)-2-[[bis(4-methoxy-phenyl)-phenyl-methoxy]methyl]-2-[[2-cyanoethoxy-(diisopropylamino)phosphanyl]oxy-methyl]-6-(5-methyl-2,4-dioxo-pyrimidin-1-yl)morpholin-4-yl]-5-oxo-pentanoyl]-3-piperidyl] acetate (249)

145 mg (0.16 mmol) of the starting material [(3S,4R,5S)-5-acetamido-4-acetoxy-1-[5-[(2R,6R)-2-[[bis(4-methoxy-phenyl)-phenyl-methoxy]methyl]-2-(hydroxymethyl)-6-(5-meth-yl-2,4-dioxo-pyrimidin-1-yl)-morpholin-4-yl]-5-oxo-pentanoyl]-3-piperidyl] acetate (248) were dissolved in 30 ml dry DCM. Under argon, 60.7 mg (0.20 mmol) 2-cyanoethyl-N,N,N′,N′-tetraisopropylphosphoro-diamidite and 14.1 mg (0.08 mmol) diisopropylammonium tetrazolide were added and the solution was stirred overnight at room temperature. After adding additional 60.7 mg (0.20 mmol) of the phosphitylating reagent, stirring was continued for 4 h, followed by the addition of the same amount of phosphitylating reagent. The reaction solution was stirred again overnight. After the mixture was washed with H₂O, the organic layer was separated and dried over MgSO₄. Evaporation of the solvent and chromatography on silica (0 to 100% DCM/EtOAc/MeOH (5:5:1) in DCM/EtOAc (1:1)) gave 130 mg (73.7%) of the desired phosphoamidite [(3S,4R,5S)-5-acetamido-4-acetoxy-1-[5-[(2S,6R)-2-[[bis(4-methoxy-phenyl)-phenyl-methoxy]methyl]-2-[[2-cyanoethoxy-(diisoprop-ylamino)phosphanyl]oxy-methyl]-6-(5-methyl-2,4-dioxo-pyrimidin-1-yl)morpholin-4-yl]-5-oxo-pentanoyl]-3-piperidyl] acetate (249) as colorless foam (mixture of peptide bond isomers and diastereomers).

LC-MS (Method A):

Rₜ[min] (UV-signal 254 nm): 2.22

MS (calc.: 1127.5) (m/z)=1043.6 [M−^{iPr2}N⁻+OH⁻−H⁺].

³¹P-NMR (162 MHz) δ[ppm]: 148.03, 147.94, 147.84, 147.00, 146.95, 146.77, 146.66.

Targeted (218, 230, 246, 249, 250 and 251) and non-targeted (252) precursor compounds as building blocks for the automated oligonucleotide syntheses are shown in Table J below.

TABLE J

| | | | | Abbreviation in an |
|---|---|---|---|---|
| Entry | No. | Structure | Abbreviation | oligonucleotide sequence |
| 1 | 218 | 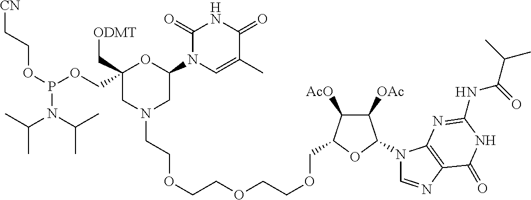 | pre-lsT1 | lsT1 |
| 2 | 230 | 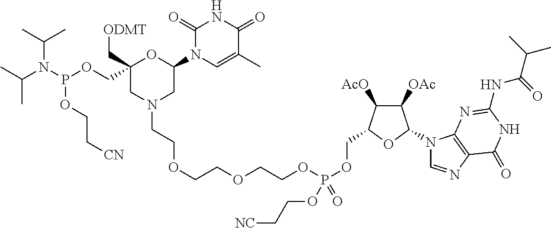 | pre-lsT2 | lsT2 |
| 3 | 246 | 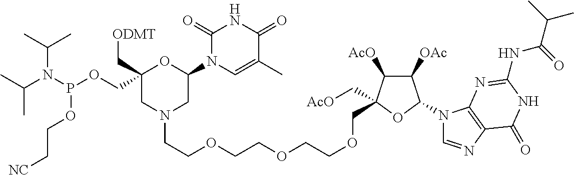 | pre-lsT3 | lsT3 |
| 4 | 249 | 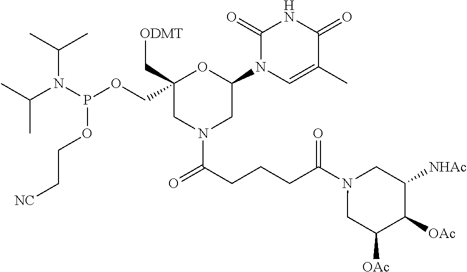 | pre-lpT1 | lpT1 |
| 5 | 250 | 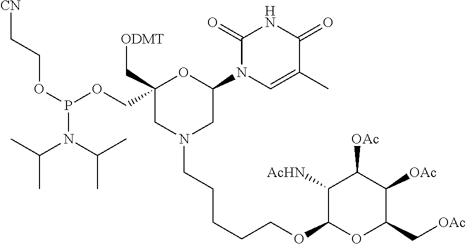 | pre-lgT3 (control compound) | lgT3 |
| 6 | 251 | 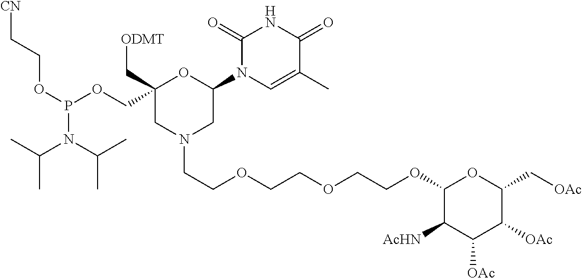 | pre-lgT7 (control compound) | lgT7 |
| 7 | 252 | 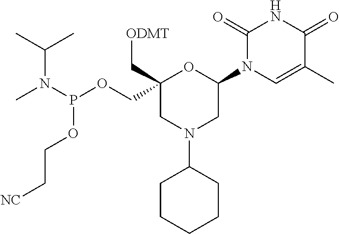 | pre-IT4 | IT4 |

### Example 21A: Synthetic Scheme of Targeted Nucleosides 254 and 258

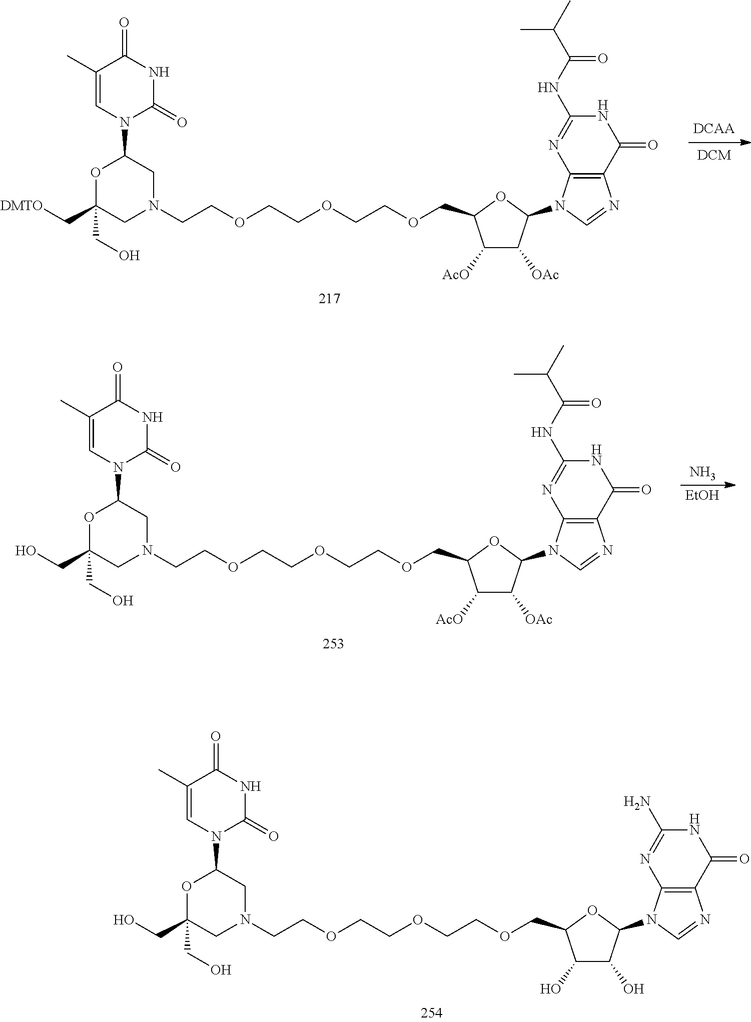

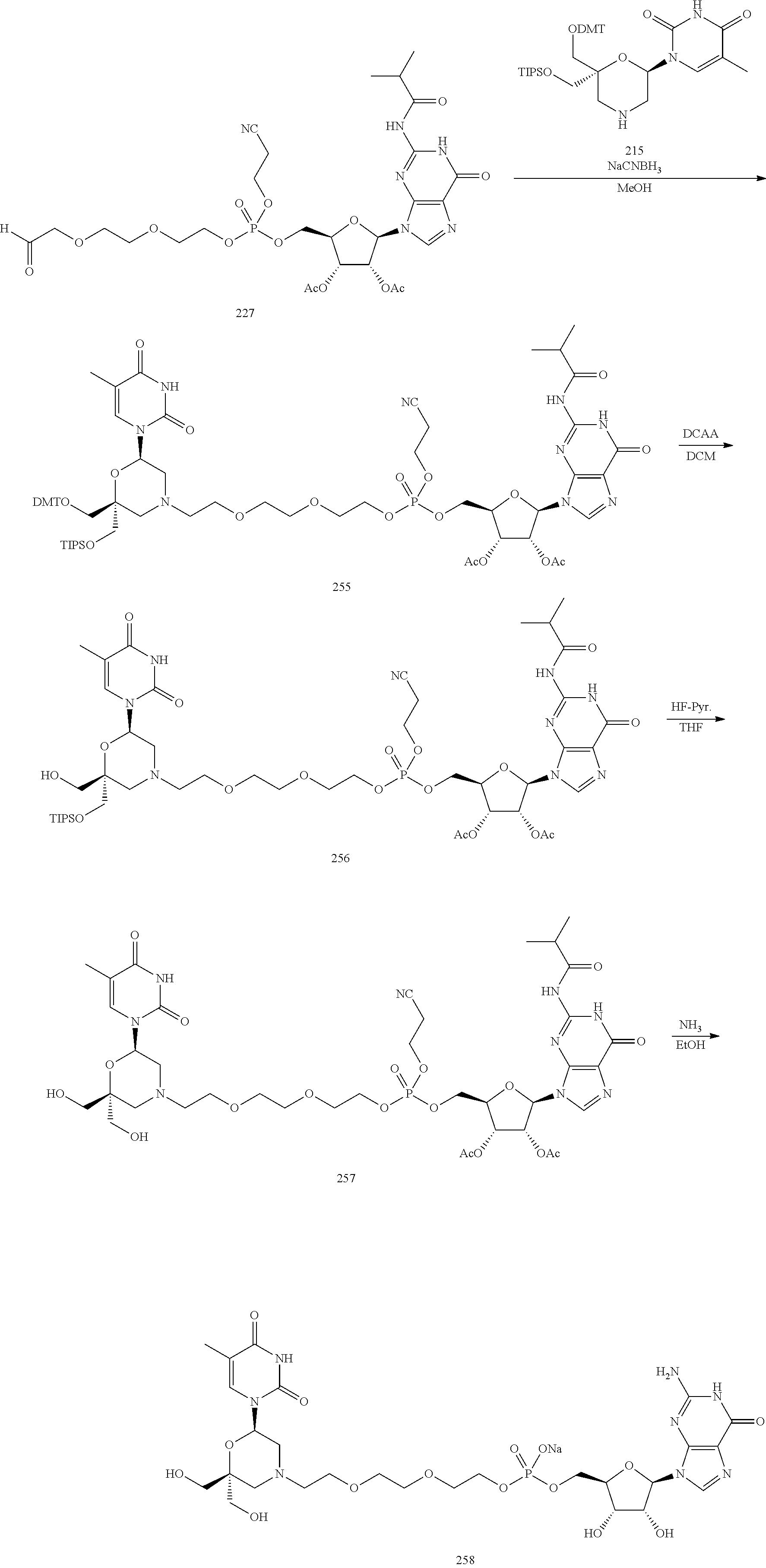

### Example 21B: Synthetic Scheme of Targeted Nucleoside 260

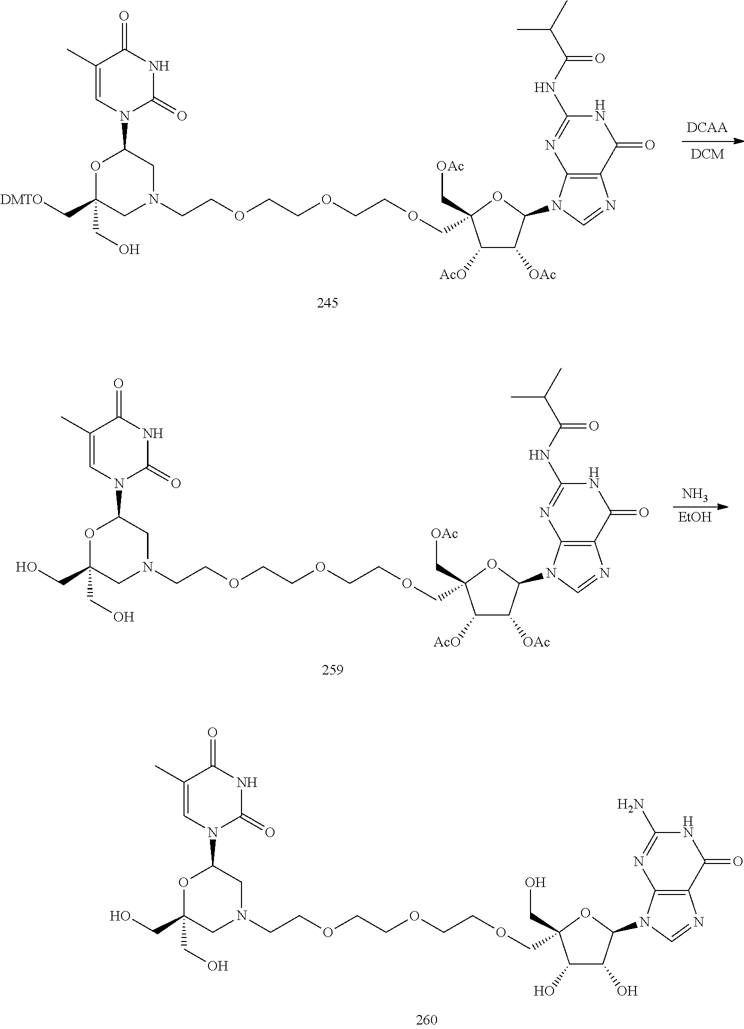

### Example 21.1: Synthesis of [(2R,3R,4R,5R)-4-Acetoxy-2-[2-[2-[2-[(6R)-2,2-bis-(hydroxymethyl)-6-(5-methyl-2,4-dioxo-pyrimidin-1-yl)morpholin-4-yl]ethoxy]ethoxy]-ethoxymethyl]-5-[2-(2-methylpropanoylamino)-6-oxo-1H-purin-9-yl]tetrahydrofuran-3-yl]acetate (253)

259 mg (0.23 mmol) of the DMT-ether [(2R,3R,4R,5R)-4-Acetoxy-2-[2-[2-[2-[(2R,6R)-2-[[bis(4-meth-oxyphenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoyl-amino)-6-oxo-1H-purin-9-yl]tetrahydrofuran-3-yl] acetate (217) were dissolved in 25 ml DCM and 898 mg (6.9 mmol, 30 eq) DCAA were added at room temperature. After 3 min, 35 ml sat. NaHCO₃-solution and H₂O (1:1) were added and the mixture was stirred for 1 h. The organic layer was separated and the aqueous was extracted with DCM (2×10 ml). The combined organic layers were dried with MgSO₄ and the solvent was evaporated. The crude product was purified on silica (0 to 10% MeOH in DCM), yielding 172 mg (90.9%) of the title compound [(2R,3R,4R,5R)-4-acetoxy-2-[2-[2-[2-[(6R)-2,2-bis(hydroxymethyl)-6-(5-methyl-2,4-dioxo-pyrimidin-1-yl)morpholin-4-yl]ethoxy]ethoxy]-ethoxymethyl]-5-[2-(2-methylpropanoylamino)-6-oxo-1H-purin-9-yl]tetrahydrofuran-3-yl]acetate (253) as colorless foam.

LC-MS (Method A):

Rₜ[min] (ELSD-signal): 1.27

MS (calc.: 822.3) (m/z)=823.5 [M+H⁺].

### Example 21.2: Synthesis of 1-[(2R)-4-[2-[2-[2-[[(2R,3S,4R,5R)-5-(2-Amino-6-oxo-1H-purin-9-yl)-3,4-dihydroxy-tetrahydrofuran-2-yl]methoxy]ethoxy]ethoxy]ethyl]-6,6-bis-(hydroxymethyl)-morpholin-2-yl]-5-methyl-pyrimidine-2,4-dione (254)

165 mg (0.20 mmol) of the starting material [(2R,3R,4R,5R)-4-acetoxy-2-[2-[2-[2-[(6R)-2,2-bis(hydroxymethyl)-6-(5-methyl-2,4-dioxo-pyrimidin-1-yl)morpholin-4-yl]ethoxy]ethoxy]-ethoxymethyl]-5-[2-(2-methylpropanoylamino)-6-oxo-1H-purin-9-yl]tetrahydrofuran-3-yl] acetate (253) were dissolved in 6 ml of a 35 M aqueous NH₃-solution and 2 ml EtOH. After stirring at 55° C. for 3 h. the solvents were evaporated in vacuo and the crude product purified by prep. HPLC (Waters XBridge® BEH C18 OBD™ Prep Column 130 Å, 5 μm, 10 mm×100 mm), using and ACN/H₂O-gradient, yielding 88 mg (65.4%) of the title compound 1-[(2R)-4-[2-[2-[2-[[(2R,3S,4R,5R)-5-(2-amino-6-oxo-1H-purin-9-yl)-3,4-dihydroxy-tetrahydrofuran-2-yl]methoxy]ethoxy]ethoxy]ethyl]-6,6-bis(hydroxymethyl)-morpholin-2-yl]-5-methyl-pyrimi-dine-2,4-dione (254) as colorless solid.

LC-MS (Method A):

Rₜ[min] (ELSD-signal): 0.72

MS (calc.: 668.3) (m/z)=669.4 [M+H⁺].

¹H NMR (400 MHz, DMSO-d₆) δ[ppm]: 11.31 (br s, 1H), 10.59 (br s, 1H), 7.91 (s, 1H), 7.60 (d, J=1.1 Hz, 1H), 6.45 (br s, 2H), 5.80 (dd, J=9.9, 2.7 Hz, 1H), 5.70 (d, J=5.7 Hz, 1H), 5.39 (d, J=6.0 Hz, 1H), 5.17 (br d, J=4.4 Hz, 1H), 4.62 (t, J=5.8 Hz, 1H), 4.56 (t, J=5.6 Hz, 1H), 4.41 (dd, J=10.9, 5.5 Hz, 1H), 4.07 (m, 1H), 3.96 (dd, J=7.7, 3.9 Hz, 1H), 3.72 (dd, J=11.3, 4.9 Hz, 1H), 3.61 (dd, J=10.9, 3.8 Hz, 1H), 3.49-3.58 (m, 13H), 3.36-3.45 (m, 3H), 2.89 (m, 1H), 2.74 (d, J=11.7 Hz, 1H), 2.15 (d, J=11.7 Hz, 1H), 2.03 (t, J=10.5 Hz, 1H), 1.78 (d, J=0.9 Hz, 3H).

### Example 21.3: Synthesis of [(2R,3R,4R,5R)-4-Acetoxy-2-[[2-[2-[2-[(2S,6R)-2-[[bis(4-methoxyphenyl)-phenyl-methoxy]methyl]-6-(5-methyl-2,4-dioxo-pyrimidin-1-yl)-2-(triisopropylsilyloxy-methyl)morpholin-4-yl]ethoxy]ethoxy]ethoxy-(2-cyanoethoxy)phosphoryl]oxymethyl]-5-[2-(2-methylpropanoylamino)-6-oxo-1H-purin-9-yl]tetrahydrofuran-3-yl] acetate (255)

270 mg (0.39 mmol) of the aldehyde [(2R,3R,4R,5R)-4-Acetoxy-2-[[2-cyanoethoxy-[2-[2-(2-oxo-ethoxy)ethoxy]-ethoxy]phosphoryl]oxymethyl]-5-[2-(2-methylpropanoylamino)-6-oxo-1H-purin-9-yl]tetra-hydrofuran-3-yl] acetate (217) and 295 mg (0.40 mmol) of the morpho-line 1-[(2R,6S)-6-[[bis(4-methoxy-phenyl)-phenyl-methoxy]methyl]-6-(triisopropyl-silyloxymeth-yl)-morpholin-2-yl]-5-methyl-pyrimidine-2,4-dione (215) (Hofmeister et al., WO2019170731) were dissolved in in 10 ml dry MeOH. At room temperature, molecular sieves (3 Å) were added, followed by the addition of 158 mg (1.54 mmol) NEt₃ and 234 mg (3.85 mmol) AcOH. After 1 h, 102 mg (1.54 mmol) sodium cyanoboronhydride were added and the reaction solution stirred for 17 h at room temperature. The reaction mixture was filtered and the filtrate brought to pH 7 by the addition of sat. NaHCO₃-solution. The MeOH was evaporated and the remaining aqueous solution diluted with H₂O and extracted with EtOAc. The organic layer was separated and washed with sat. NaCl-solution and H₂O. After drying with MgSO₄, the solvent was removed in vacuo. After purification on silica (DCM/MeOH 19:1), 285 mg (52.3%) of the title compound [(2R,3R,4R,5R)-4-acetoxy-2-[[2-[2-[2-[(2S,6R)-2-[[bis(4-methoxyphenyl)-phenyl-methoxy]-methyl]-6-(5-methyl-2,4-dioxo-pyrimidin-1-yl)-2-(triisopropylsilyloxy-methyl)-morpholin-4-yl]ethoxy]ethoxy]ethoxy-(2-cyanoethoxy)phosphoryl]oxymethyl]-5-[2-(2-methylpropanoyl-amino)-6-oxo-1H-purin-9-yl]tetrahydrofuran-3-yl] acetate (255) were ob-tained as colorless foam.

LC-MS (Method A):

Rₜ[min] (ELSD-signal): 3.31, 3.32 (mixture of diastereomers)

MS (calc.: 1413.6) (m/z)=1414.5 [M+H⁺].

### Example 21.4: Synthesis of [(2R,3R,4R,5R)-4-Acetoxy-2-[[2-cyanoethoxy-[2-[2-[2-[(2S,6R)-2-(hydroxymethyl)-6-(5-methyl-2,4-dioxo-pyrimidin-1-yl)-2-(triisopropylsilyloxymethyl)-morpholin-4-yl]ethoxy]ethoxy]ethoxy]phosphoryl]oxymethyl]-5-[2-(2-methylpropanoyl-amino)-6-oxo-1H-purin-9-yl]tetrahydrofuran-3-yl] acetate (256)

To a solution of 205 mg (0.14 mmol) of the starting material [(2R,3R,4R,5R)-4-acetoxy-2-[[2-[2-[2-[(2S,6R)-2-[[bis(4-methoxyphenyl)-phenyl-methoxy]methyl]-6-(5-methyl-2,4-dioxo-pyrimidin-1-yl)-2-(triisopropylsilyloxy-methyl)morpholin-4-yl]ethoxy]ethoxy]ethoxy-(2-cyanoethoxy)phosphoryl]oxymethyl]-5-[2-(2-methylpropanoylamino)-6-oxo-1H-purin-9-yl]-tetrahydrofuran-3-yl] acetate (255) in 15 ml DCM, were added 472 mg (3.62 mmol, 25 eq) DCAA at room temperature. After stirring for 3 min, 30 ml sat. NaHCO₃-solution/H₂O (1:1) were added and stirring was continued for 1 h. The organic phase was separated and the aqueous layer extracted with 25 ml DCM. The combined organic layers were dried with MgSO₄ and evaporated. Silicagel chromatography (0 to 10% MeOH in DCM) of the crude product gave 105 mg (65.1%) of the title compound [(2R,3R,4R,5R)-4-acetoxy-2-[[2-cyanoethoxy-[2-[2-[2-[(2S,6R)-2-(hydroxymethyl)-6-(5-methyl-2,4-dioxo-pyrimidin-1-yl)-2-(triisopropylsilyloxy-methyl)-morpholin-4-yl]ethoxy]ethoxy]ethoxy]phosphoryl]oxymethyl]-5-[2-(2-methylpro-panoyl-amino)-6-oxo-1H-purin-9-yl]tetrahydrofuran-3-yl] acetate (256) as colorless foam.

LC-MS (Method A):

Rₜ[min] (UV-signal, 254 nm): 2.35, 2.36 (mixture of diastereomers)

MS (calc.: 1111.5) (m/z)=1112.5 [M+H⁺], 557.0 (z=2: ½[M+2H⁺]).

### Example 21.5: Synthesis of [(2R,3R,4R,5R)-4-Acetoxy-2-[[2-[2-[2-[(6R)-2,2-bis(hydroxy-methyl)-6-(5-methyl-2,4-dioxo-pyrimidin-1-yl)morpholin-4-yl]ethoxy]ethoxy]ethoxy-(2-cyanoethoxy)-phosphoryl]oxymethyl]-5-[2-(2-methylpropanoylamino)-6-oxo-1H-purin-9-yl]tetrahydro-furan-3-yl] acetate (257)

99 mg (89.0 μmol) of the silylether [(2R,3R,4R,5R)-4-acetoxy-2-[[2-cyanoethoxy-[2-[2-[2-[(2S,6R)-2-(hydroxymethyl)-6-(5-methyl-2,4-dioxo-pyrimidin-1-yl)-2-(triisopropylsilyloxy-methyl)-morpholin-4-yl]ethoxy]ethoxy]ethoxy]phosphoryl]oxymethyl]-5-[2-(2-methylpro-panoyl-amino)-6-oxo-1H-purin-9-yl]tetrahydrofuran-3-yl] acetate (256) were dissolved in 2 ml THF. After adding 204 mg (1.34 mmol, 15 eq) HF-pyridine at room temperature, the solution was stirred for 1.5 h. 700 mg NaHCO₃ (solid) were added and stirring was continued for 1 h. The reaction was filtered and the filtrate evaporated in vacuo. Purification of the crude product on silica (0 to 20% MeOH in DCM) yielded 78 mg (91.3%) of the title compound [(2R,3R,4R,5R)-4-acetoxy-2-[[2-[2-[2-[(6R)-2,2-bis(hydroxymethyl)-6-(5-methyl-2,4-dioxo-pyrimidin-1-yl)morpholin-4-yl]ethoxy]ethoxy]ethoxy-(2-cyanoethoxy)-phosphoryl]oxy-methyl]-5-[2-(2-methylpropanoylamino)-6-oxo-1H-purin-9-yl]tetrahydro-furan-3-yl] acetate (257) as colorless foam.

LC-MS (Method A):

Rₜ[min] (UV-signal, 254 nm): 1.27

MS (calc.: 955.3) (m/z)=956.4 [M+H⁺], 478.9 (z=2: ½[M+2H⁺]).

### Example 21.6: Synthesis of [(2R,3S,4R,5R)-5-(2-Amino-6-oxo-1H-purin-9-yl)-3,4-di-hydroxy-tetrahydrofuran-2-yl]methyl 2-[2-[2-[(6R)-2,2-bis(hydroxymethyl)-6-(5-methyl-2,4-dioxo-pyrimidin-1-yl)morpholin-4-yl]ethoxy]ethoxy]ethyl hydrogen phosphate-sodium salt (258)

Following the protocol, described for the synthesis (254), 72 mg (75 μmol) of the starting material [(2R,3R,4R,5R)-4-acetoxy-2-[[2-[2-[2-[(6R)-2,2-bis(hydroxymethyl)-6-(5-methyl-2,4-dioxo-pyrimidin-1-yl)morpholin-4-yl]ethoxy]ethoxy]ethoxy-(2-cyanoethoxy)-phos-phor-yl]oxymethyl]-5-[2-(2-methylpropanoylamino)-6-oxo-1H-purin-9-yl]tetrahydro-furan-3-yl] acetate (257) gave 39 mg (66.5%) of the title compound [(2R,3S,4R,5R)-5-(2-amino-6-oxo-1H-purin-9-yl)-3,4-dihydroxy-tetrahydrofuran-2-yl]methyl 2-[2-[2-[(6R)-2,2-bis-(hydroxy-methyl)-6-(5-methyl-2,4-dioxo-pyrimidin-1-yl)morpholin-4-yl]ethoxy]ethoxy]-ethyl hydrogen phosphate-sodium salt (258) as colorless solid.

LC-MS (Method A):

Rₜ[min] (UV-signal, 254 nm): 0.63

MS (calc.: 748.2) (m/z)=749.4 [M+H⁺], 375.3 (z=2: ½[M+2H⁺]).

¹H NMR (400 MHz, DMSO-d6) δ[ppm]: 11.33 (s, 1H), 10.59 (br s, 1H), 7.92 (s, 1H), 7.58-7.61 (m, 1H), 6.57 (br s, 2H), 5.81 (dd, J=9.9, 2.5 Hz, 1H), 5.68 (d, J=6.0 Hz, 1H), 5.45 (br d, J=3.7 Hz, 1H), 5.32 (d, J=5.9 Hz, 1H), 4.83-5.05 (m, 2H), 4.51 (dd, J=11.0, 5.3 Hz, 1H), 4.19 (m, 1H), 3.95 (m, 1H), 3.87 (m, 1H), 3.66-3.82 (m, 4H), 3.40-3.63 (m, 10H), 3.25-3.39 (m, 1H), 2.85 (br d, J=11.5 Hz, 2H), 2.40-2.58 (m, 2H), 2.12 (d, J=12.0 Hz, 1H), 2.04 (t, J=10.6 Hz, 1H), 1.78 (s, 3H).

### Example 21.7: Synthesis of [(2R,3S,4R,5R)-3,4-Diacetoxy-2-[2-[2-[2-[(6R)-2,2-bis(hydroxy-methyl)-6-(5-methyl-2,4-dioxo-pyrimidin-1-yl)morpholin-4-yl]ethoxy]ethoxy]ethoxymethyl]-5-[2-(2-methylpropanoylamino)-6-oxo-1H-purin-9-yl]tetrahydrofuran-2-yl]methyl acetate (259)

60 mg (45 μmol) of the DMT-ether [(2R,3S,4R,5R)-3,4-Diacetoxy-2-[2-[2-[2-[(2R,6R)-2-[[bis(4-methoxy-phenyl)-phenyl-methoxy]methyl]-2-(hydroxymethyl)-6-(5-methyl-2,4-dioxo-pyrimidin-1-yl)-morpholin-4-yl]ethoxy]ethoxy]ethoxymethyl]-5-[2-(2-methylpropanoyl-amino)-6-oxo-1H-purin-9-yl]tetrahydrofuran-2-yl]methyl acetate (245) were cleaved under the acidic conditions, described in the protocol for the synthesis of (253). After the working-up procedure, the crude product was dissolved in warm EtOAc and precipitated by the addition of n-pentane. After centrifugation, the mother liquor was decarded. The precipitation procedure was repeated three times. After drying the precipitate in vacuo at 45° C., 42 mg (quant.) of the title compound [(2R,3S,4R,5R)-3,4-diacetoxy-2-[2-[2-[2-[(6R)-2,2-bis(hydroxymethyl)-6-(5-methyl-2,4-dioxo-pyrimidin-1-yl)morpholin-4-yl]ethoxy]-ethoxy]ethoxymethyl]-5-[2-(2-methylpropanoylamino)-6-oxo-1H-purin-9-yl]tetrahydrofuran-2-yl]methyl acetate (259) were isolated as colorless solid.

LC-MS (Method A):

Rₜ[min] (UV-signal, 254 nm): 1.32

MS (calc.: 894.4) (m/z)=895.4 [M+H⁺], 448.4 (z=2: ½[M+2H⁺]).

### Example 21.8: Synthesis of 1-[(2R)-4-[2-[2-[2-[[(2S,3S,4R,5R)-5-(2-Amino-6-oxo-1H-purin-9-yl)-3,4-dihydroxy-2-(hydroxymethyl)tetrahydrofuran-2-yl]methoxy]ethoxy]ethoxy]ethyl]-6,6-bis(hydroxymethyl)morpholin-2-yl]-5-methyl-pyrimidine-2,4-dione (260)

Following the protocol, described for the synthesis (254), 28 mg (31 μmol) of the starting material [(2R,3S,4R,5R)-3,4-diacetoxy-2-[2-[2-[2-[(6R)-2,2-bis(hydroxymethyl)-6-(5-methyl-2,4-dioxo-pyrimidin-1-yl)morpholin-4-yl]ethoxy]ethoxy]ethoxymethyl]-5-[2-(2-methylpro-panoylamino)-6-oxo-1H-purin-9-yl]tetrahydrofuran-2-yl]methyl acetate (259) gave 16 mg (74.7%) of the title compound 1-[(2R)-4-[2-[2-[2-[[(2S,3S,4R,5R)-5-(2-amino-6-oxo-1H-purin-9-yl)-3,4-dihydroxy-2-(hydroxymethyl)tetrahydrofuran-2-yl]methoxy]ethoxy]ethoxy]-ethyl]-6,6-bis(hydroxymethyl)morpholin-2-yl]-5-methyl-pyrimidine-2,4-dione (260) as color-less solid.

LC-MS (Method A):

Rₜ[min] (UV-signal, 254 nm): 0.74

MS (calc.: 698.3) (m/z)=699.3 [M+H⁺], 350.3 (z=2: ½[M+2H⁺]).

¹H NMR (400 MHz, DMSO-d₆) δ[ppm]: 10.17-11.17 (m, 2H), 7.93 (s, 1H), 7.60 (d, J=1.0 Hz, 1H), 6.42 (br s, 2H), 5.79 (dd, J=9.9, 2.7 Hz, 1H), 5.71 (d, J=7.7 Hz, 1H), 5.28 (br d, J=7.1 Hz, 1H), 4.99-5.11 (m, 2H), 4.59-4.71 (m, 2H), 4.56 (m, 1H), 4.11 (br s, 1H), 3.71 (dd, J=11.1, 4.2 Hz, 1H), 3.45-3.64 (m, 16H), 3.36-3.43 (m, 3H), 2.88 (m, 1H), 2.73 (br d, J=11.7 Hz, 1H), 2.14 (d, J=11.7 Hz, 1H), 2.01 (t, J=10.4 Hz, 1H), 1.79 (d, J=0.9 Hz, 3H).

### Example 22A: Synthesis of Trimeric Targeted Nucleotides 261 and 262

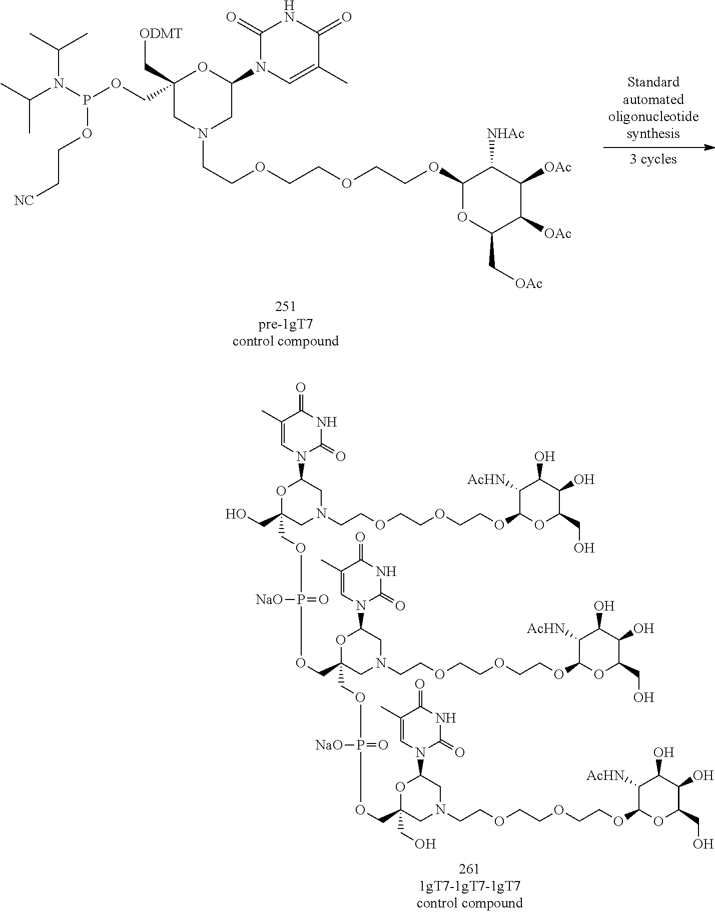

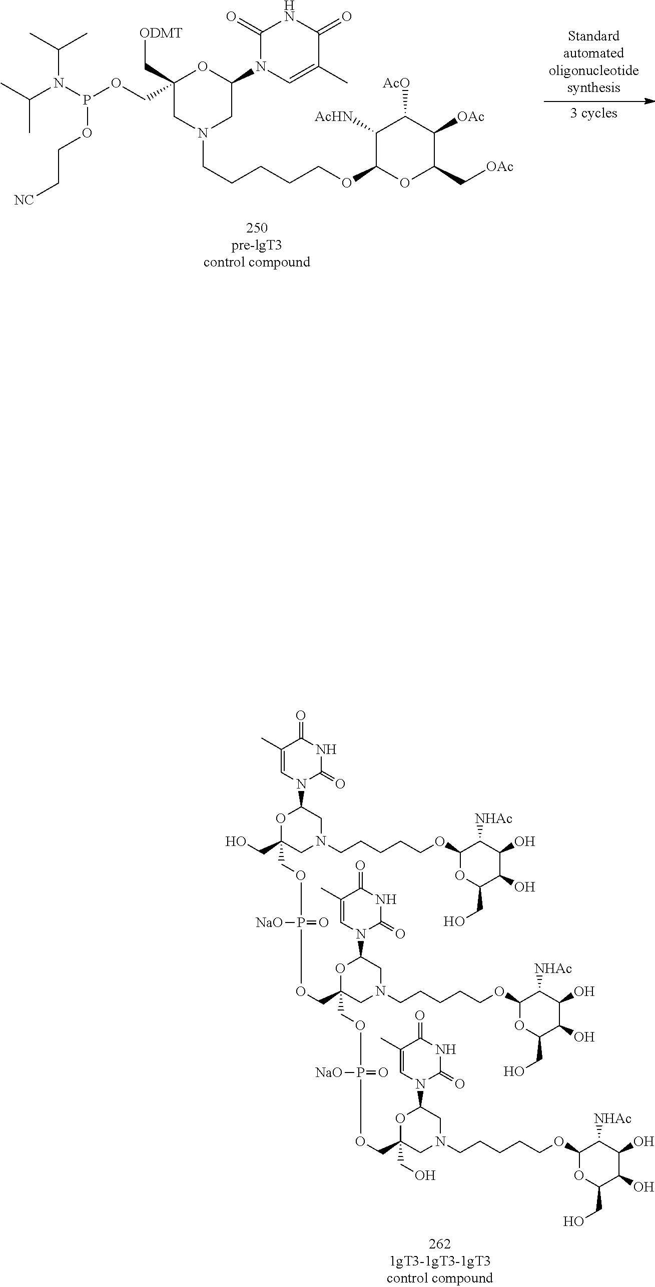

The syntheses of the starting building blocks pre-lgT7 (232) and pre-lgT3 (233) are described by Hofmeister et al. WO 2019/170731.

### Example 22B: Synthesis Trimeric Targeted Nucleotides 263, 264 and 265

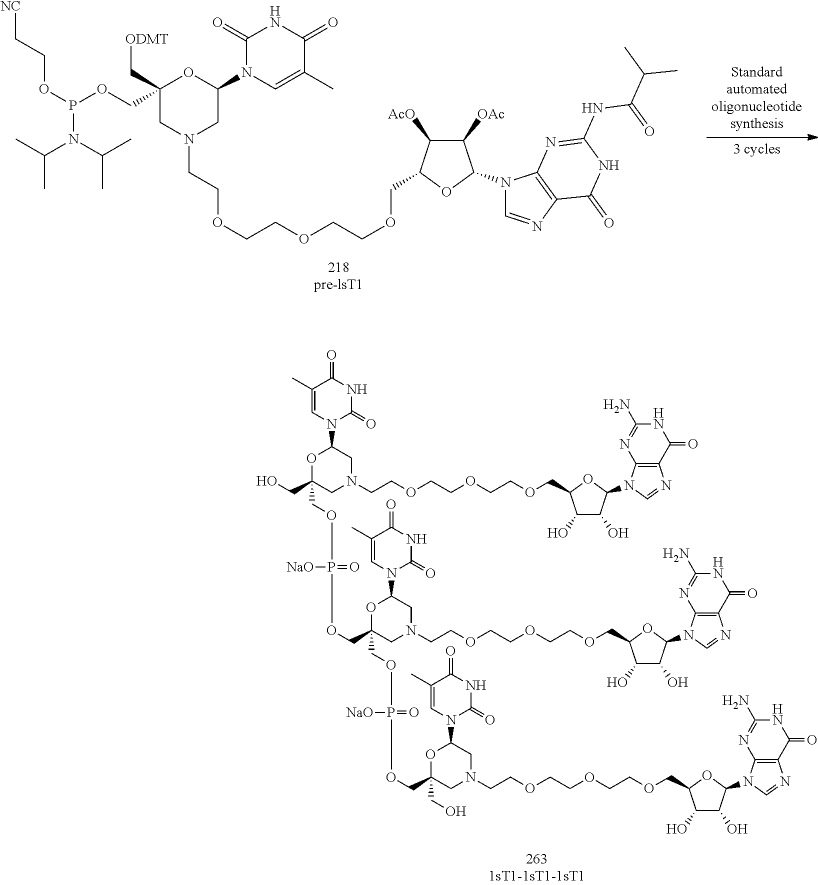

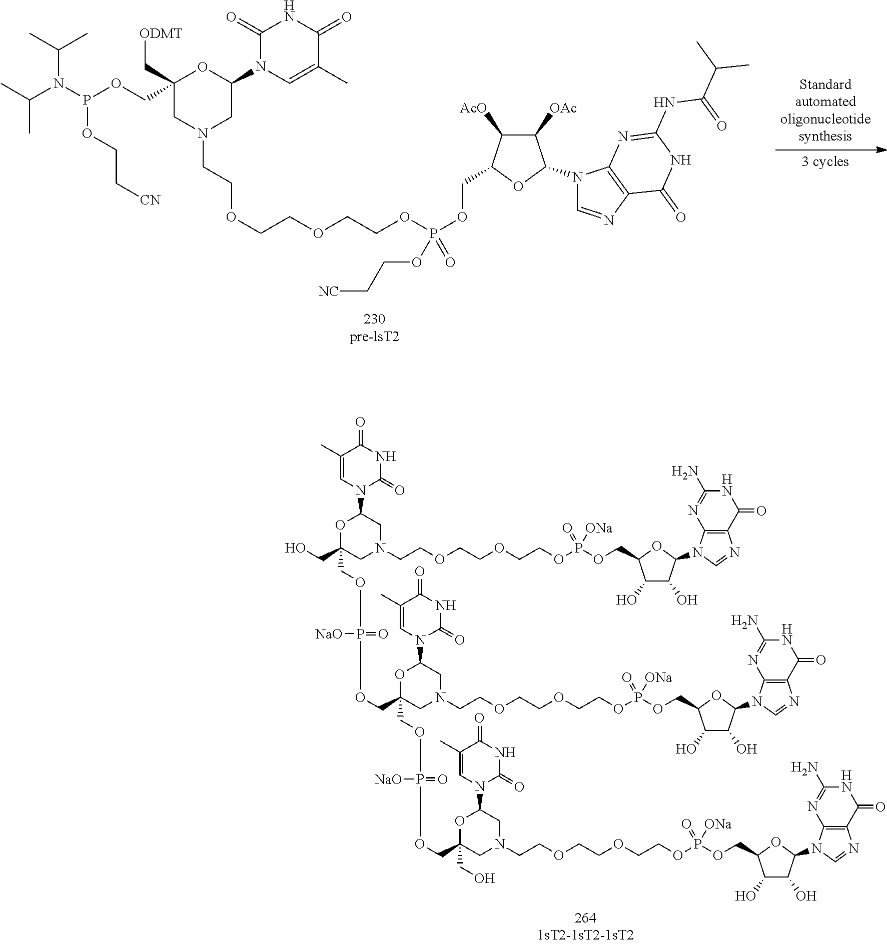

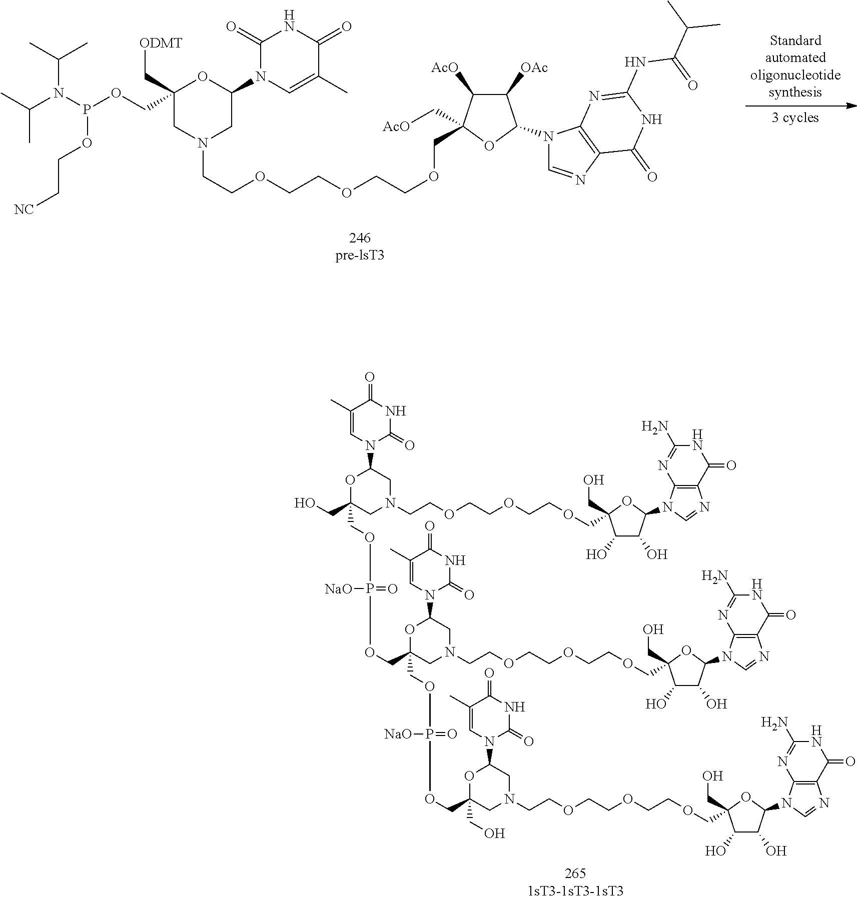

### General Procedure for the Trimerization of the Targeted Nucleotide Precursors

The phosphoamidite building blocks 250, 251, 218, 230 and 246 were coupled, following standard protocols for the automated oligonucleotide syntheses on a universal support (AM chemicals LLC). After three coupling steps and cleavage from the solid support material, the crude products were purified by preparative HPLC on an Agilent 1100 series prep HPLC, (Waters XBridge® BEH C18 OBD™ Prep Column 130 Å, 5 μm, 10 mm×100 mm), using a ACN/H₂O (0.1 M triethylammonium acetate) gradient. After lyophilization of the product fractions, the material was dissolved in 0.25 M NaCl-solution and desalted on an ÄKTA-purifier by SEC-chromatography (GE, HiPrep 26/10 Desalting, Sephadex™ G-25 Fine, cross-linked dextran, 90 μm). The product fraction were collected. Final lyophilization gave the trimeric building blocks as colorless foams.

### Example 22.1: (261) (lgT7-lgT7-lgT7

4×2 μM-scale syntheses using the targeted nucleotide precursor 251 as coupling building block gave 2.89 mg (18.2%) (261) (lgT7-lgT7-lgT7).

MS (calc.: 1942.7) m/z=1943.0.

³¹P NMR (400 MHz D₂O) δ[ppm]: 0.17, 0.09.

### Example 22.2: (262) (lgT3-lgT3-lgT3

2×10 μM-scale syntheses using the targeted nucleotide precursor 250 as coupling building block gave 6.15 mg (16.6%) (262) (lgT3-lgT3-lgT3).

MS (calc.: 1804.7) m/z=1804.7.

### Example 22.3: (263) (lsT1-lsT1-lsT1

4×2 μM-scale syntheses using the targeted nucleotide precursor 218 as coupling building block gave 6.21 mg (35.7%) (263) (lsT1-lsT1-lsT1).

MS (calc.: 2128.7) m/z=2129.3.

³¹P NMR (400 MHz D₂O) δ[ppm]: 0.32, 0.25.

### Example 22.4: (264) (lsT2-lsT2-lsT2

5×2 μM-scale syntheses using the targeted nucleotide precursor 230 as coupling building block gave 7.26 mg (29.3%) (264) (lsT2-lsT2-lsT2).

MS (calc.: 2368.6) m/z=4738.6, 7108.8.

³¹P NMR (400 MHz D₂O) δ[ppm]: 0.40, 0.38, 0.38, 0.13, 0.06.

### Example 22.5: (265) (lsT3-lsT3-lsT3

4×2 μM-scale syntheses using the targeted nucleotide precursor 246 as coupling building block gave 1.14 mg (6.3%) (265) (lsT3-lsT3-lsT3).

MS (calc.: 2218.8) m/z=2219.0.

### Example 23: Synthesis of Trimeric ASGPR-Binder 267

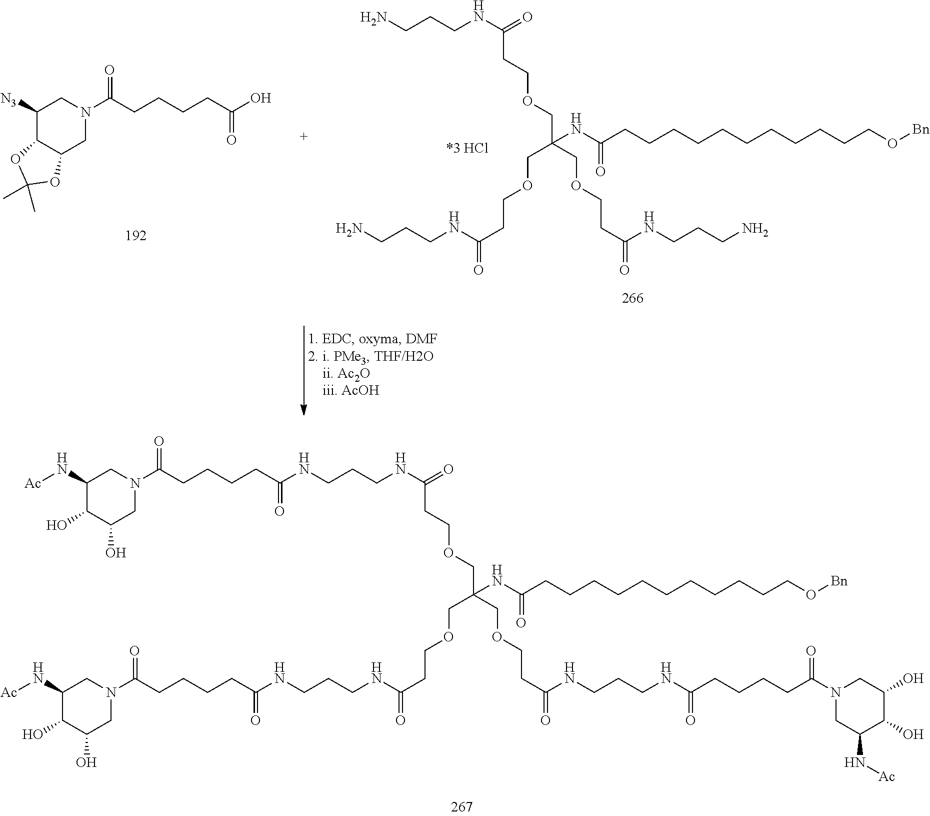 [SMILES: CC1(C)O[C@@H]2[C@@H](N=[N+]=[N-])CN(C(=O)CCCCC(=O)O)C[C@@H]2O1 ; NCCCNC(=O)CCOCC(COCCC(=O)NCCCN)(COCCC(=O)NCCCN)NC(=O)CCCCCCCCCCCOCc1ccccc1 ; CC(=O)N[C@H]1CN(C(=O)CCCCC(=O)NCCCNC(=O)CCOCC(COCCC(=O)NCCCNC(=O)CCCCC(=O)N2C[C@H](NC(C)=O)[C@@H](O)[C@@H](O)C2)(COCCC(=O)NCCCNC(=O)CCCCC(=O)N2C[C@H](NC(C)=O)[C@@H](O)[C@@H](O)C2)NC(=O)CCCCCCCCCCCOCc2ccccc2)C[C@H](O)[C@@H]1O]

### Example 23.1: Synthesis of N,N′,N′-((3S,3′S,3″S,4R,4′R,4″R,5S,5′S,5″S)-(17-(12-(benzyloxy)dodecanamido)-6,12,22,28-tetraoxo-17-((3-oxo-3-((3-(6-oxopentanamido) propyl)amino)propoxy)methyl)-15,19-dioxa-7,11,23,27-tetraazatritriacontanedioyl)tris(4,5-dihydroxypiperidine-1,3-diyl))triacetamide (267)

To a solution of 6-[(3aS,7S,7aR)-7-azido-2,2-dimethyl-4,6,7,7a-tetrahydro-3aH-[1,3]dioxolo [4,5-c]pyridin-5-yl]-6-oxo-hexanoic acid (192, 155 mg, 0.475 mmol, 3.34 equiv.) in DMF (5 mL) were added EDC·HCl (108 mg, 0.563 mmol, 3.96 equiv.), Oxyma pure (75 mg, 0.53 mmol, 3.71 equiv.), N-methyl morpholine (0.10 mL, 0.90 mmol, 6.34 equiv.) and 3,3′-((2-((3-((3-aminopropyl)amino)-3-oxopropoxy)methyl)-2-(12-(benzyloxy)dodecanamido)propane-1,3-diyl)bis(oxy))bis(N-(3-aminopropyl)propanamide) (266, HCl-salt, 113 mg, 0.142 mmol, 1.00 equiv.). The reaction mixture was stirred for overnight at r.t. until LC/MS indicated full conversion of the starting material. EtOAc (50 mL) and aqueous 1 N HCl (25 mL) were added, the layers were separated, the organic layer was washed with 2 N aqueous NaOH-solution (25 mL), saturated aqueous NaCl-solution (20 mL), dried (MgSO₄), filtered and concentrated in vacuo. To a solution of the crude product in THF (5 mL) and water (0.1 mL), PMe₃ (1 N in THF, 0.70 mL, 0.70 mmol, 4.93 equiv.) was added and the reaction mixture was stirred for 1 h at r.t. Since LC/MS indicated full conversion of the starting material, the reaction mixture was concentrated in vacuo. The residue was dissolved in EtOAc (5 mL) and acetic anhydride (0.2 mL, approx. 15 equiv.) and pyridine (0.1 mL, approx. 9 equiv.) were added and the reaction mixture was stirred for 1 h at r.t. Since LC/MS indicated full formation of the acetamide, the reaction mixture was concentrated in vacuo. The crude product was dissolved in acetic acid (4 mL) and water (1 mL) and heated to 80° C. for 3 h until full acetonide deprotection was monitored by LC/MS. The solvent was removed in vacuo and the crude mixture was purified by HPLC (15 min, 5-65% acetonitrile in water+0.1% TFA) to yield CP138 (70 mg, 0.042 mmol, 30% over 2 steps) as a colorless solid.

LC-MS (Method D):

Rₜ[min] (UV-signal 220 nm): 1.06

M[g/mol]: 824.1 [M+H²⁺]

¹H NMR (400 MHz, DMSO-d₆) δ[ppm]: 7.87-7.78 (m, 4H), 7.75 (m, 3H), 7.65 (m, 2H), 7.38-7.23 (m, 5H), 6.97 (s, 1H), 4.43 (s, 2H), 4.08 (dd, J=12.6, 4.4 Hz, 2H), 3.98-3.29 (m, 29H), 3.19-2.96 (m, 17H), 2.37-2.12 (m, 13H), 2.11-1.99 (m, 9H), 1.83/1.81 (s, 9H), 1.59-1.36 (m, 23H), 1.35-1.13 (m, 16H).

### Example 24: Synthesis of Trimeric ASGPR-Binder 268

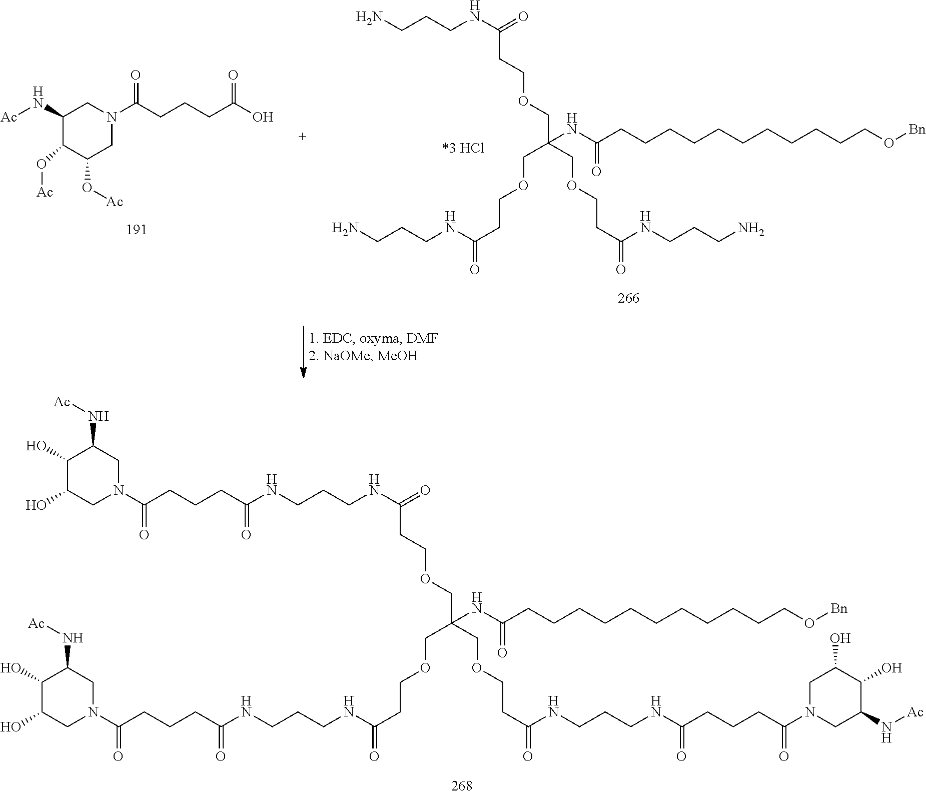 [SMILES: CC(=O)N[C@H]1CN(C(=O)CCCC(=O)O)C[C@H](OC(C)=O)[C@@H]1OC(C)=O ; NCCCNC(=O)CCOCC(COCCC(=O)NCCCN)(COCCC(=O)NCCCN)NC(=O)CCCCCCCCCCCOCc1ccccc1 ; CC(=O)N[C@H]1CN(C(=O)CCCC(=O)NCCCNC(=O)CCOCC(COCCC(=O)NCCCNC(=O)CCCC(=O)N2C[C@H](NC(C)=O)[C@@H](O)[C@@H](O)C2)(COCCC(=O)NCCCNC(=O)CCCC(=O)N2C[C@H](NC(C)=O)[C@@H](O)[C@@H](O)C2)NC(=O)CCCCCCCCCCCOCc2ccccc2)C[C@H](O)[C@@H]1O]

### Example 24.1: Synthesis of N,N′,N′-((3S,3′S,3″S,4R,4′R,4″R,5S,5′S,5″S)-(16-(12-(benzyloxy) dodecanamido)-5,11,21,27-tetraoxo-16-((3-oxo-3-((3-(5-oxopentanamido)propyl)amino) propoxy)methyl)-14,18-dioxa-6,10,22,26-tetraazahentriacontanedioyl)tris(4,5-dihydroxy-piperidine-1,3-diyl))triacetamide (268)

To a solution of 5-[(3S,4R,5S)-3-acetamido-4,5-diacetoxy-1-piperidyl]-5-oxo-pentanoic acid (191, 25 mg, 0.066 mmol, 4.0 equiv.) in DMF (1 mL) were added EDC·HCl (13 mg, 0.066 mmol, 4.0 equiv.), Oxyma pure (12 mg, 0.083 mmol, 5.0 equiv.), N-methyl morpholine (0.02 mL, 0.17 mmol, 10.0 equiv.) and 3,3′-((2-((3-((3-aminopropyl)amino)-3-oxopropoxy)methyl)-2-(12-(benzyloxy)dodecanamido)propane-1,3-diyl)bis(oxy))bis(N-(3-aminopropyl)propanamide) (266, HCl-salt, 15 mg, 0.017 mmol, 1.0 equiv.). The reaction mixture was stirred overnight at r.t. until LC/MS indicated full conversion of the starting material. The crude mixture was filtered and purified by HPLC (15 min, 10-70% acetonitrile in water+0.1% TFA). The product containing fractions were freeze-dried, dissolved in MeOH (3 mL) and NaOMe (100 mg, excess) was added. After 1 h, full acetyl deprotection was monitored. The crude mixture was filtered and purified by HPLC (15 min, 5-65% acetonitrile in water+0.1% TFA) to yield N,N′,N′-((3S,3′S,3″S,4R,4′R,4″R,5S,5′S,5″S)-(16-(12-(benzyloxy)dodecanamido)-5,11,21,27-tetraoxo-16-((3-oxo-3-((3-(5-oxopentan-amido)pro-pyl)amino)propoxy)methyl)-14,18-dioxa-6,10,22,26-tetraazahentriacontanedioyl) tris(4,5-di-hydroxy-piperidine-1,3-diyl))triacetamide (268, 14 mg, 0.009 mmol, 53%) as a colorless solid.

¹H NMR (400 MHz, DMSO-d₆) δ[ppm]: 7.88-7.78 (m, 4H), 7.74 (m, 3H), 7.66 (m, 2H), 7.37-7.23 (m, 5H), 6.97 (s, 1H), 4.43 (s, 2H), 4.07 (dd, J=12.5, 4.2 Hz, 2H), 3.88-3.26 (m, 29H), 3.19-2.97 (m, 17H), 2.59-2.51 (m, 1H), 2.36-2.14 (m, 13H), 2.12-2.00 (m, 8H), 1.83/1.81 (s, 9H), 1.75-1.64 (m, 6H), 1.59-1.13 (m, 27H).

### Example 25: Synthesis of Trimeric ASGPR-Binder 269

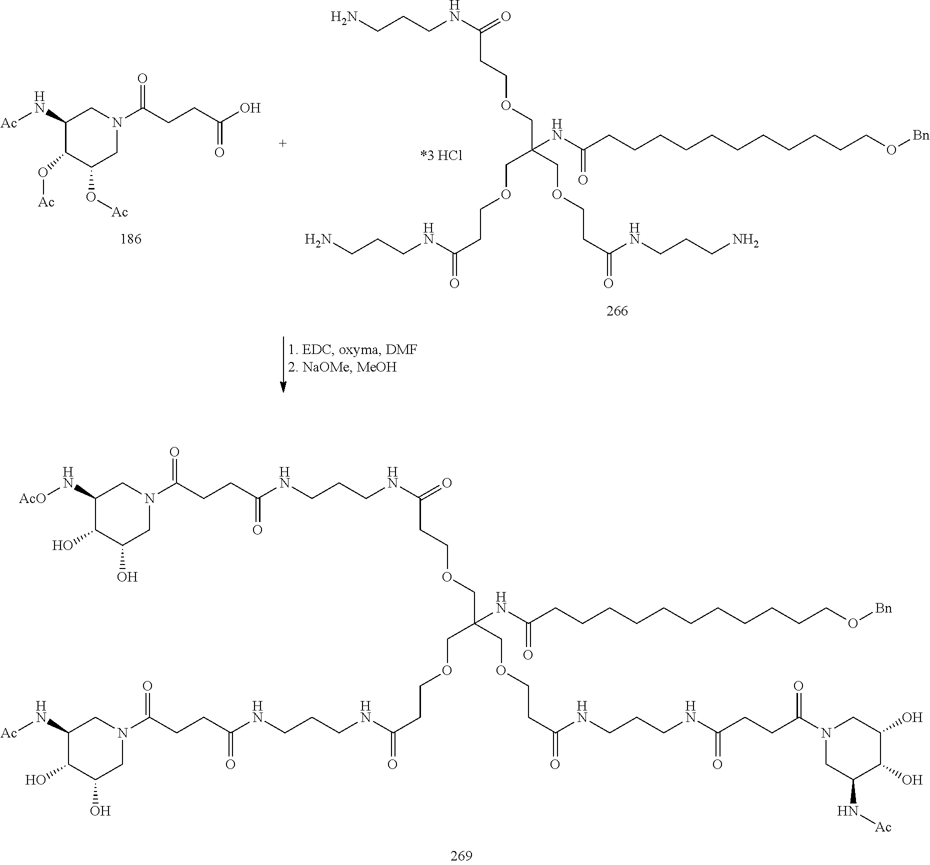 [SMILES: CC(=O)N[C@H]1CN(C(=O)CCC(=O)O)C[C@H](OC(C)=O)[C@@H]1OC(C)=O ; NCCCNC(=O)CCOCC(COCCC(=O)NCCCN)(COCCC(=O)NCCCN)NC(=O)CCCCCCCCCCCOCc1ccccc1 ; CC(=O)N[C@H]1CN(C(=O)CCC(=O)NCCCNC(=O)CCOCC(COCCC(=O)NCCCNC(=O)CCC(=O)N2C[C@H](NOC(C)=O)[C@@H](O)[C@@H](O)C2)(COCCC(=O)NCCCNC(=O)CCC(=O)N2C[C@H](NC(C)=O)[C@@H](O)[C@@H](O)C2)NC(=O)CCCCCCCCCCCOCc2ccccc2)C[C@H](O)[C@@H]1O]

### Example 25.1: Synthesis of N,N′,N′-((3S,3′S,3″S,4R,4′R,4″R,5S,5′S,5″S)-(15-(12-(benzyloxy) dodecanamido)-4,10,20,26-tetraoxo-15-((3-oxo-3-((3-(4-oxobutanamido)propyl)amino)pro-poxy)methyl)-13,17-dioxa-5,9,21,25-tetraazanonacosanedioyl)tris(4,5-dihydroxypiperidine-1,3-diyl))triacetamide (269)

To a solution of 4-[(3S,4R,5S)-3-acetamido-4,5-diacetoxy-1-piperidyl]-4-oxo-butanoic acid (186, 21 mg, 0.058 mmol, 4.0 equiv.) in DMF (1 mL) were added EDC·HCl (11 mg, 0.058 mmol, 4.0 equiv.), Oxyma pure (10 mg, 0.072 mmol, 5.0 equiv.), N-methyl morpholine (0.02 mL, 0.17 mmol, 10.0 equiv.) and 3,3′-((2-((3-((3-aminopropyl)amino)-3-oxopropoxy)methyl)-2-(12-(benzyloxy)dodecanamido)propane-1,3-diyl)bis(oxy))bis(N-(3-aminopropyl)propan-amide) (266, HCl-salt, 13 mg, 0.014 mmol, 1.0 equiv.). The reaction mixture was stirred overnight at r.t. until LC/MS indicated full conversion of the starting material. The crude mixture was filtered and purified by HPLC (15 min, 10-70% acetonitrile in water+0.1% TFA). The product containing fractions were freeze-dried, dissolved in MeOH (3 mL) and NaOMe (100 mg, excess) was added. After 1 h, full acetyl deprotection was monitored. The crude mixture was filtered and purified by HPLC (15 min, 5-65% acetonitrile in water+0.1% TFA) to yield N,N′,N′-((3S,3′S,3″S,4R,4′R,4″R,5S,5′S,5″S)-(15-(12-(benzyloxy) dodecanamido)-4,10,20,26-tetraoxo-15-((3-oxo-3-((3-(4-oxobutanamido)propyl)amino)pro-poxy)methyl)-13,17-dioxa-5,9,21,25-tetraazanonacosanedioyl)tris(4,5-dihydroxypiperidine-1,3-diyl))triacet-amide (269, 2 mg, 0.001 mmol, 9%) as a colorless solid.

¹H NMR (400 MHz, DMSO-d₆) δ[ppm]: 7.89 (d, J=7.8 Hz, 1H), 7.86-7.76 (m, 6H), 7.62 (d, J=8.3 Hz, 2H), 7.38-7.23 (m, 5H), 6.97 (s, 1H), 4.43 (s, 2H), 4.00 (dd, J=13.0, 4.1 Hz, 2H), 3.85-3.37 (m, 29H), 3.26-3.13 (m, 5H), 3.03 (m, 12H) 2.70-2.53 (m, 4H), 2.47-2.23 (m, 15H), 2.10-1.96 (m, 3H), 1.84/1.81 (s, 9H), 1.59-1.13 (m, 27H).

LC-MS:

### Method A:

Flow rate: 1 ml/min
Column: Waters Aquity UPLC BEH C18 1.7 μm 2.1×50 mm; 55° C.
Gradient: H₂O (0.05% FA)/ACN (0.035% FA): 98:2 (0.0 to 0.2 min) to 2:98 (0.2 to 3.8 min) to (3.8 to 4.3 min) to 98:2 (4.3 to 4.5 min).

### Method B:

Flow rate: 1.1 ml/min
Column: Phenomenex Luna C18, 3.0 μm, 2.0×10 mm; 30° C.
Gradient: H₂O (0.05% TFA)/ACN: 93:7 (0.0 min) to 5:95 (1.0 min to 1.45 min) to 93:7 (1.5 min).

### Method C:

Flow rate: 1.1 ml/min
Column: Phenomenex Luna C18, 3.0 μm, 2.0×10 mm; 30° C.
Gradient: H₂O (0.05% TFA)/ACN: 20:80 (0.0 min) to 5:95 (0.6 min to 1.45 min) to 80:20 (1.5 min).

### Method D:

Flow rate: 1 ml/min
Column: YMC J'Sphere ODS H80, 4 μm, 20×2.1 mm, 30° C.
Gradient: H₂O (0.05% TFA)/ACN: 96:4 (0.0 min) to 5:95 (2.4 min) to 96:4 (2.45 min).

### Method E:

Flow rate: 1.8 ml/min
Column: Sunfire C18, 3.5 μm, 50×4.6 mm, 50° C.
Gradient: H₂O (10 mM NH₄HCO₃)/ACN: 95:5 (0.0 min) to 5:95 (2.4 min) to 95:5 (2.45 min).

### Method F:

Flow rate: 1.5 ml/min
Column: Xbridge-C18, 2.5 μm, 30×4.6 mm, 30° C.
Gradient: H₂O (2.5 mM TFA)/ACN: 90:10 (0.0 min) to 5:95 (2.4 min) to 90:10 (2.45 min).

### Example 26.1: ASGPR-Binding Affinities of Compounds of the Present Disclosure

### Methods

### Fluorescent Polarization (FP) ASGPR Binding Assay to Determine the Binding Affinities of ASGPR-Targeted Small Molecules:

The technique of fluorescence polarization (FP) is based on the observation that when a fluorescently labeled molecule is excited by polarized light, it emits light with a degree of polarization that is inversely proportional to the rate of molecular rotation. This property of fluorescence can be used to measure the interaction of a small labeled ligand with a larger protein and provides a basis for direct and competition binding assays.

The assay was performed in 384-well microplates (GREINER bio-one, 781076). Membrane prepared from Wistar rat liver was used as a source of ASGPR and Cy5 fluorophore-labeled trimeric GauNAc-tool compound was used as a tracer.

In the FP binding experiments, 15 μltest sample was mixed with 15 μl of 65 ag liver membrane. After 10 minutes incubation at room temperature, 10 μl of the Cy5-labeled trimeric GalNAc-tool compound (final concentration: 1 nM) were added. The FP signals were recorded after 30 minutes incubation at room temperature at Ex. 612 nm/Em. 670 with PheraStar FXS (BMG Biotech). The optimal FP buffer was 50 mM Tris, pH 7.4; 3 mM CaCl₂; 0.04% Triton X100; 0,05% BSA.

ASGPR-binding data of selected monomeric exemplary compounds of formulae (III) and (V) are shown in Table K below.

TABLE K

| Entry | Structure | Compound # | IC₅₀ [μM] |
|---|---|---|---|
| 1 | 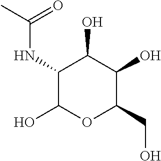 | 193 (control compound) | 545 |
| 2 | 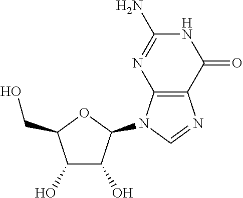 [SMILES: Nc1nc2c(ncn2[C@@H]2O[C@H](CO)[C@@H](O)[C@H]2O)c(=O)[nH]1] | 194 | 513 |
| 3 | 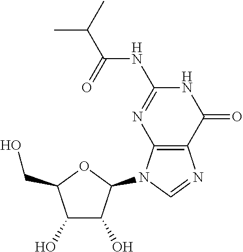 [SMILES: CC(C)C(=O)Nc1nc2c(ncn2[C@@H]2O[C@H](CO)[C@@H](O)[C@H]2O)c(=O)[nH]1] | 219 | 129 |
| 4 | 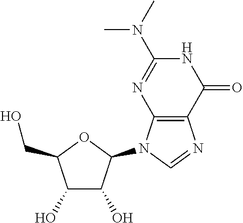 [SMILES: CN(C)c1nc2c(ncn2[C@@H]2O[C@H](CO)[C@@H](O)[C@H]2O)c(=O)[nH]1] | 198 | 1940 |
| 5 | 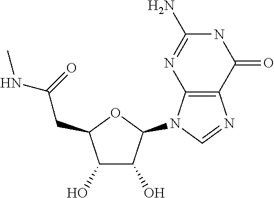 [SMILES: CNC(=O)C[C@H]1O[C@@H](n2cnc3c(=O)[nH]c(N)nc32)[C@H](O)[C@@H]1O] | 71 | 605 |
| 6 | 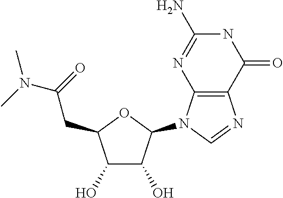 [SMILES: CN(C)C(=O)C[C@H]1O[C@@H](n2cnc3c(=O)[nH]c(N)nc32)[C@H](O)[C@@H]1O] | 72 | 89 |
| 7 | 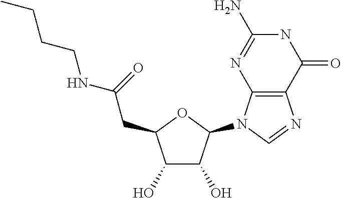 [SMILES: CCCCNC(=O)C[C@H]1O[C@@H](n2cnc3c(=O)[nH]c(N)nc32)[C@H](O)[C@@H]1O] | 73 | 1920 |
| 8 | 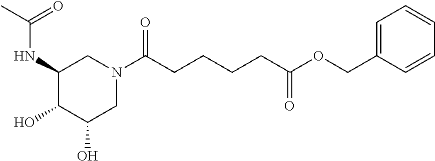 [SMILES: CC(=O)N[C@H]1CN(C(=O)CCCCC(=O)OCc2ccccc2)C[C@H](O)[C@@H]1O] | 112 | 344 |
| 9 | 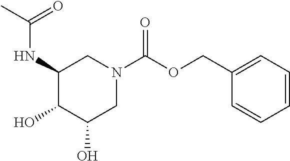 [SMILES: CC(=O)N[C@H]1CN(C(=O)OCc2ccccc2)C[C@H](O)[C@@H]1O] | 121 | 563 |
| 10 | 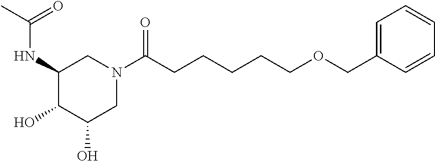 [SMILES: CC(=O)N[C@H]1CN(C(=O)CCCCCOCc2ccccc2)C[C@H](O)[C@@H]1O] | 128 | 451 |
| 11 | 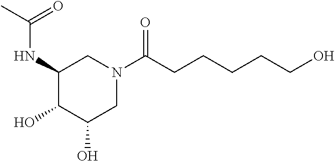 [SMILES: CC(=O)N[C@H]1CN(C(=O)CCCCCO)C[C@H](O)[C@@H]1O] | 129 | 585 |
| 12 | 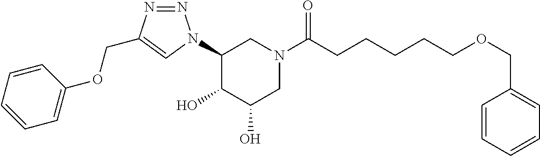 [SMILES: O=C(CCCCCOCc1ccccc1)N1C[C@H](O)[C@H](O)[C@@H](n2cc(COc3ccccc3)nn2)C1] | 131 | 1770 |
| 13 | 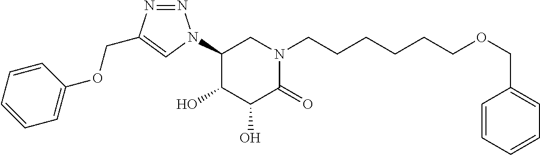 [SMILES: O=C1[C@H](O)[C@H](O)[C@@H](n2cc(COc3ccccc3)nn2)CN1CCCCCCOCc1ccccc1] | 140 | 658 |
| 14 | 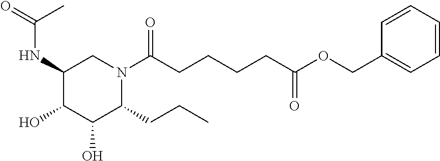 [SMILES: CCC[C@@H]1[C@H](O)[C@H](O)[C@@H](NC(C)=O)CN1C(=O)CCCCC(=O)OCc1ccccc1] | 146 | 488 |
| 15 | 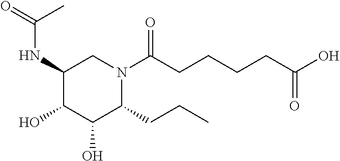 [SMILES: CCC[C@@H]1[C@H](O)[C@H](O)[C@@H](NC(C)=O)CN1C(=O)CCCCC(=O)O] | 148 | 370 |
| 16 | 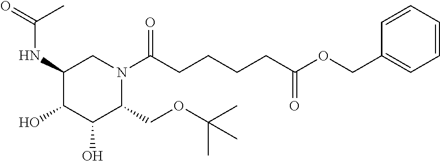 [SMILES: CC(=O)N[C@H]1CN(C(=O)CCCCC(=O)OCc2ccccc2)[C@H](COC(C)(C)C)[C@H](O)[C@@H]1O] | 160 | 1050 |
| 17 | 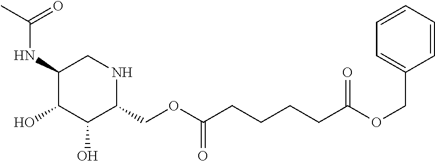 [SMILES: CC(=O)N[C@H]1CN[C@H](COC(=O)CCCCC(=O)OCc2ccccc2)[C@H](O)[C@@H]1O] | 161 | 161 |
| 18 | 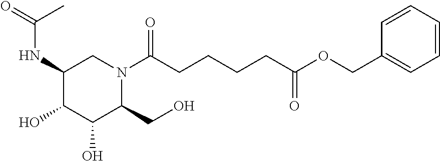 [SMILES: CC(=O)N[C@H]1CN(C(=O)CCCCC(=O)OCc2ccccc2)[C@@H](CO)[C@H](O)[C@@H]1O] | 162 | 576 |
| 19 | 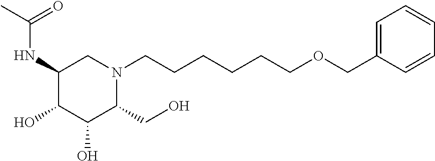 [SMILES: CC(=O)N[C@H]1CN(CCCCCCOCc2ccccc2)[C@H](CO)[C@H](O)[C@@H]1O] | 180 | 628 |

ASGPR-binding data of selected targeted nucleosides of formula (V) are shown in Table L below.

TABLE L

| Entry | Structure | Compound # | IC₅₀ [μM] |
|---|---|---|---|
| 1 | 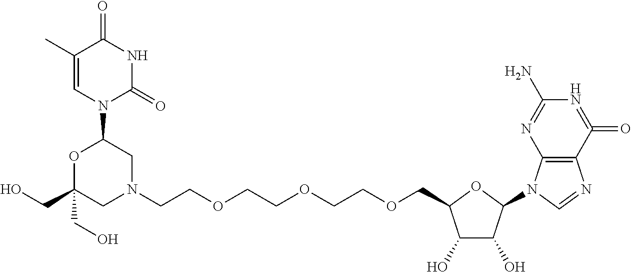 | 254 | 1240 |
| 2 | 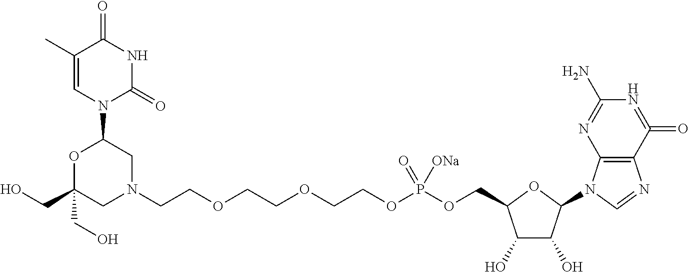 | 258 | 1850 |

ASGPR-binding data of trimeric targeted oligonucleotides of formula (VI) comprising cell-targeting moieties of formulae (IVA) and/or (IVB), and trimeric exemplary compounds comprising a cell-targeting moiety of formula (II) are shown in Table M below:

TABLE M

| | | Com- | IC₅₀ |
|---|---|---|---|
| Entry | Structure | pound # | [μM] |
| 1 | 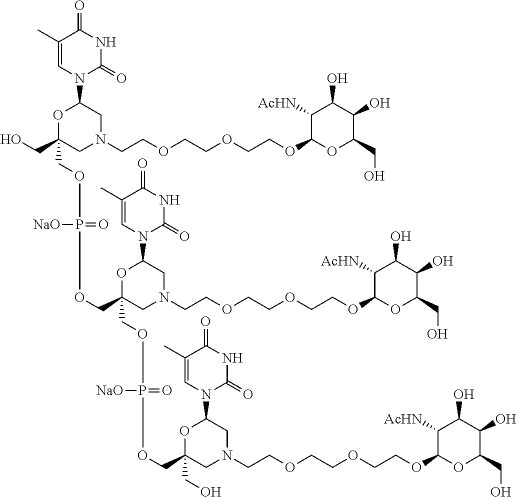 lgT7-lgT7-lgT7 | 261 (control com- pound) | 0.058 |
| 2 | 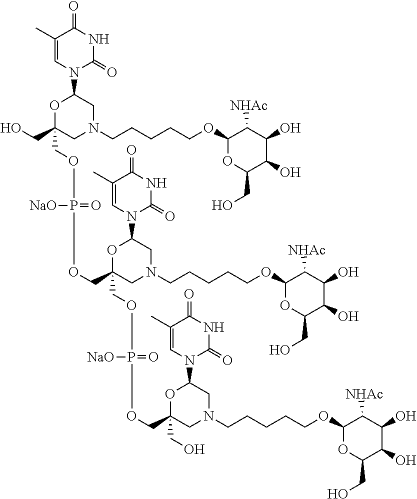 lgT3-lgT3-lgT3 | 262 (control com- pound) | 0.048 |
| 3 | 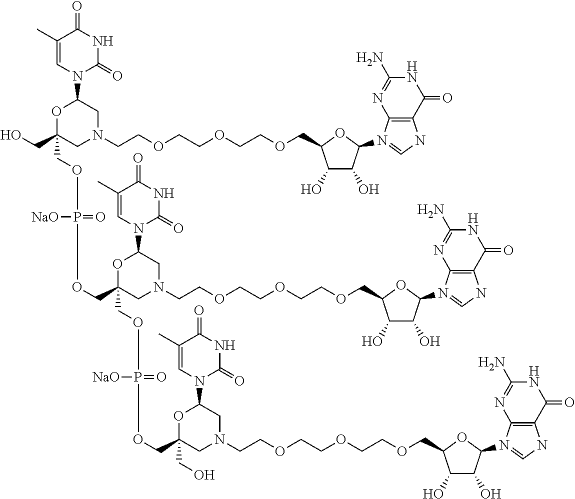 lgT1-lgT1-lgT1 | 263 | 1.64 |
| 4 | 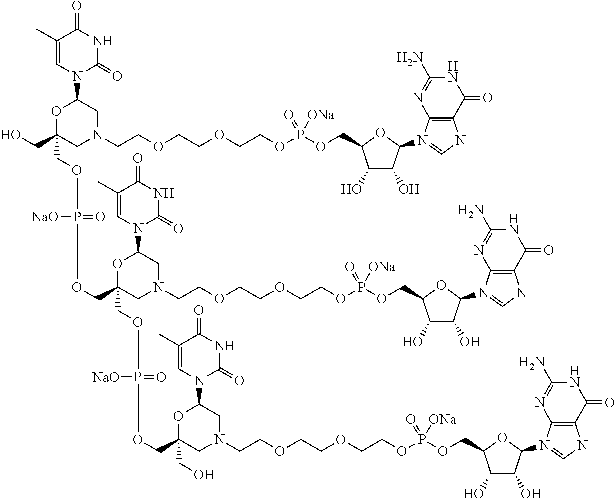 lgT2-lgT2-lgT2 | 264 | 14.4 |
| 5 | 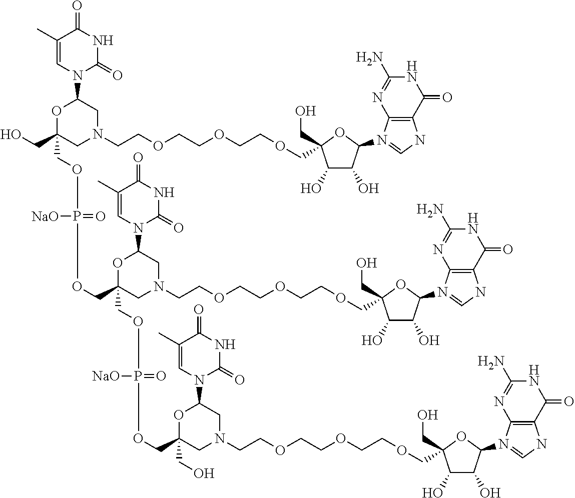 lgT3-lgT3-lgT3 | 265 | 16.2 |
| 6 | 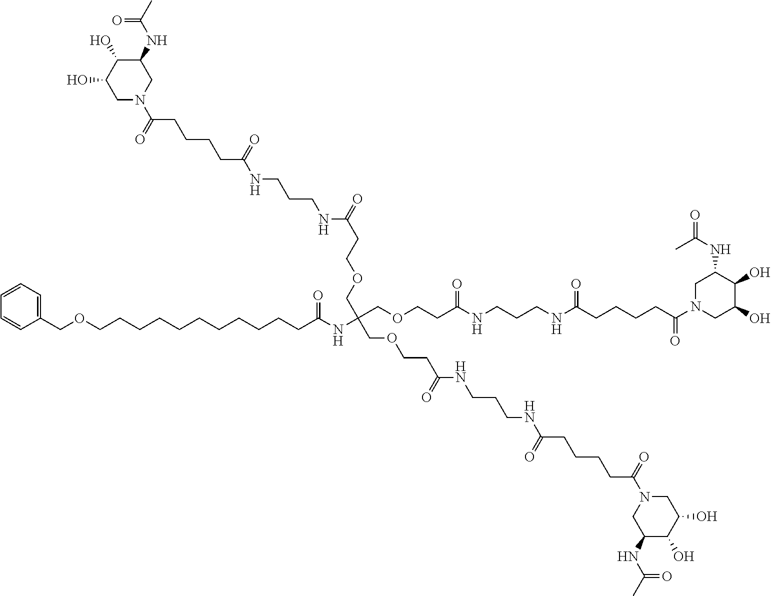 [SMILES: CC(=O)N[C@H]1CN(C(=O)CCCCC(=O)NCCCNC(=O)CCOCC(COCCC(=O)NCCCNC(=O)CCCCC(=O)N2C[C@H](NC(C)=O)[C@@H](O)[C@@H](O)C2)(COCCC(=O)NCCCNC(=O)CCCCC(=O)N2C[C@H](NC(C)=O)[C@@H](O)[C@@H](O)C2)NC(=O)CCCCCCCCCCCOCc2ccccc2)C[C@H](O)[C@@H]1O] | 267 | 0.563 |
| 7 | 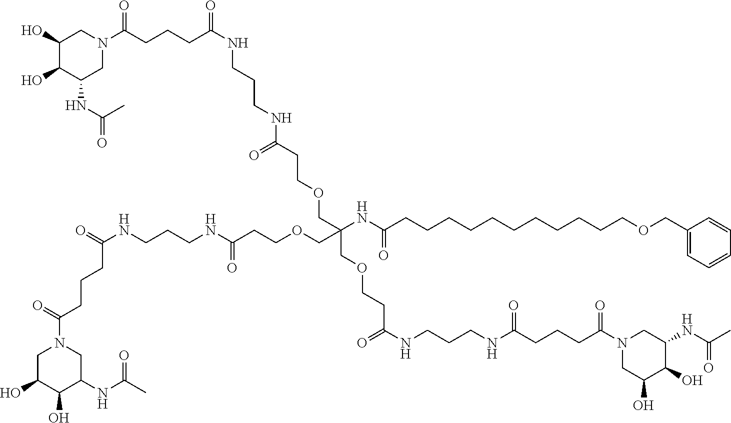 [SMILES: CC(=O)NC1CN(C(=O)CCCC(=O)NCCCNC(=O)CCOCC(COCCC(=O)NCCCNC(=O)CCCC(=O)N2C[C@H](NC(C)=O)[C@@H](O)[C@@H](O)C2)(COCCC(=O)NCCCNC(=O)CCCC(=O)N2C[C@H](NC(C)=O)[C@@H](O)[C@@H](O)C2)NC(=O)CCCCCCCCCCCOCc2ccccc2)C[C@H](O)[C@@H]1O] | 268 | 0.335 |
| 8 | 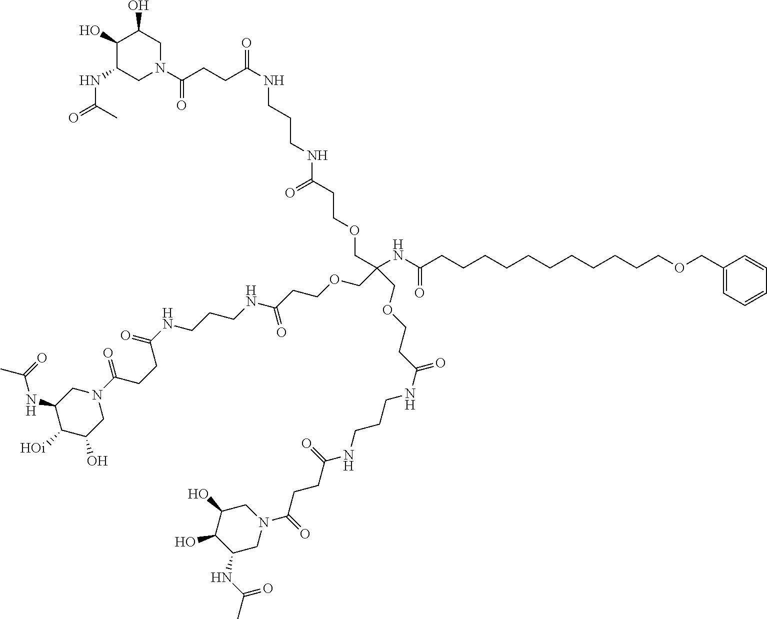 | 269 | 0.598 |

### Example 27: Oligonucleotide Synthesis and siRNA Preparation

### Methods

### Oligonucleotide Synthesis

All oligonucleotides were synthesized on an ABI 394 synthesizer. Commercially available (Sigma Aldrich) DNA-, RNA-, 2′-OMe-RNA, and 2′-deoxy-F-RNA-phosphoramidites with standard protecting groups as 5′-O-dimethoxytrityl-thymidine-3′-O—(N,N-diisopropyl-2-cyanoethyl-phosphoramidite, 5′-O-dimethoxytrityl-2′-O-tert-butyldimethylsilyl-uracile-3′-O—(N,N-diisopropyl-2-cyanoethyl)-phosphoramidite, 5′-O-dimethoxytrityl-2′-O-tert-butyldi-methylsilyl-N4-cytidine-3′-O—(N,N-diisopropyl-2-cyanoethyl)-phosphoramidite, 5′-O-di-methoxytrityl-2′-O-tert-butyldimethylsilyl-N6-benzyl-adeno sine-3′-O—(N,N-diisopropyl-2-cyanoethyl)-phosphoramidite, 5′-O-dimethoxytrityl-2′-O-tert-butyldimethylsilyl-N2-iso-butyryl-guanosine-3′-O—(N,N-diisopropyl-2-cyanoethyl)-phosphoramidite, 5′-O-dimethoxy-trityl-2′-O-methyl-uracile-3′-O—(N,N-diisopropyl-2-cyanoethyl)-phosphoramidite, 5′-O-di-methoxytrityl-2′-O-methyl-N4-cytidine-3′-O—(N,N-diisopropyl-2-cyanoethyl)-phosphoramidite, 5′-O-dimethoxytrityl-2′-O-methyl-N6-benzyl-adenosine-3′-O—(N,N-diiso-propyl-2-cyanoethyl)-phosphoramidite, 5′-O-dimethoxytrityl-2′-O-methyl-N2-isobutyryl-guanosine-3′-O—(N,N-diisopropyl-2-cyanoethyl)-phosphoramidite, 5′-O-dimethoxytrityl-2′-deoxy-fluoro-uracile-3′-O—(N,N-diisopropyl-2-cyanoethyl)-phosphoramidite, 5′-O-di-methoxytrityl-2′-deoxy-fluoro-N4-cytidine-3′-O—(N,N-diisopropyl-2-cyanoethyl)-phosphoramidite, 5′-O-dimethoxytrityl-2′-deoxy-fluoro-N6-benzyl-adenosine-3′-O—(N,N-diisopropyl-2-cyanoethyl)-phosphoramidite and 5′-O-dimethoxytrityl-2′-deoxy-fluoro-N2-isobutyryl-guanosine-3′-O—(N,N-diisopropyl-2-cyanoethyl)-phosphoramidite as well as the corresponding solid support materials (CPG-500 Å, loading 40 μmol/g, ChemGenes) were used for automated oligonucleotide synthesis. For 3′-end cholesterol conjugates, solid support 3′-Cholesterol SynBase™ CPG1000 (link technologies) 32 μmol/g was used.

Phosphoramidite building blocks were used as 0.1 M solutions in acetonitrile and activated with 5-(bis-3,5-trifluoromethylphenyl)-1H-tetrazole (activator 42, 0.25 M in acetonitrile, Sigma Aldrich). Reaction times of 200 s were used for standard phosphoramidite couplings. In case of herein described targeted and non-targeted nucleotide precursors (see Tables A, B and H), coupling times of 300 s were applied. As capping reagents, acetic anhydride in THF (capA for ABI, Sigma Aldrich) and N-methylimidazole in THF (capB for ABI, Sigma Aldrich) were used. As oxidizing reagent, iodine in THF/pyridine/water (0.02 M; oxidizer for ABI, Sigma Aldrich) was used. Alternatively, PS-oxidation was achieved with a 0.05 M solution of 3-((N,N-dimethyl-aminomethylidene)amino)-3H-1,2,4-dithiazole-5-thione (DDTT) in pyridine/acetonitrile (1:1). Deprotection of the DMT-protecting group was done using dichloroacetic acid in DCM (DCA deblock, Sigma Aldrich). Final cleavage from solid support and deprotection (acyl- and cyanoethyl-protecting groups) was achieved with NH₃ (32% aqueous solution/ethanol, v/v 3:1). Treatment with NMP/NEt₃/NEt₃·3 HF (3:1.5:2) was applied for TBDMS-deprotection.

Oligonucleotides with herein described morpholino building blocks at the 3′-end were synthesized on universal linker-solid support (CPG-500 Å, loading 39 μmol/g, AM Chemicals LLC) and the corresponding phosphoramidites, shown in Table A.

Crude products were analyzed by HPLC and purification of the single strands was performed by ion exchange or preparative HPLC-methods.

Ion exchange: ÄKTA purifier (Thermo Fisher Scientific DNAPac PA200 semi prep ion exchange column, 8 μm particles, width 22 mm×length 250 mm).
Buffer A: 1.50 L H₂O, 2.107 g NaClO₄, 438 mg EDTA, 1,818 g TRIS, 540.54 g urea, pH 7.4.
Buffer B: 1.50 L H₂O, 105.34 g NaClO₄, 438 mg EDTA, 1.818 g TRIS, 540.54 g urea, pH 7.4.

Isolation of the oligonucleotides was achieved by precipitation induced by the addition of 4 volumes of ethanol and storing at −20° C.

Preparative HPLC: Agilent 1100 series prep HPLC (Waters XBridge® BEH C18 OBD™ Prep Column 130 Å, 5 μm, 10 mm×100 mm).

Eluent: Triethylammonium acetate (0.1 M) in acetonitrile/water.

After lyophilization, the products were dissolved in 1.0 ml 2.5 M NaCl solution and 4.0 ml H₂O. The corresponding Na⁺-salts were isolated after precipitation by adding 20 ml ethanol and storing at −20° C. for 18 h. Sequences of the sense and antisense strands are shown in Table N.

Final analysis of the single strands was done by LC/MS-TOF methods. Results are shown in Table P.

For double strand formation, equimolar amounts of sense- and antisense strands were mixed in 1×PBS-buffer, heated to 85° C. for 10 min, and then slowly cooled down to room temperature. SiRNA double strand compositions are shown in Table O. Final analysis of the siRNA was done by LC/MS-TOF methods. Results are shown in Table Q.

Following standard protocols for automated oligonucleotide syntheses, the precursor building blocks of the nucleotides of the present disclosure were used for the syntheses of the single stranded sense strands, which are listed in Table N.

Hybridization with the antisense strands listed in Table N gave the final double stranded siRNAs, which are listed in Table O, containing the nucleotide building blocks of the present disclosure.

### Results

Oligonucleotide Sequences exemplified herein are shown in Table N (SEQ=SEQ ID NO); ss=sense strand; as =antisense strand):

TABLE N
Single strand oligo sequences (5′ → 3′)

| | Sense | | | Antisense | |
|---|---|---|---|---|---|
| ss-# | strand sequence | SEQ | as-# | strand sequence | SEQ |
| ss1-0 | lgT3-lgT3-lgT3-fA- | 1 | as1-0 | fA*fU*mA-fC-mG-fA- | 8 |
| (neg. | mU-fC-mG-fU-mA-fC- | | (neg. | mC-fG-mG-fU-mA-fC- | |
| ctrl.) | mG-fU-mA-fC-mC-fG- | | ctrl.) | mG-fU-mA-fC-mG-fA- | |
| | mU-fC-mG-fU*mA*fU | | | mU*dT*dT | |
| ss1-1 | lgT3-lgT3-lgT3-fA- | 2 | as1-1 | mU*fU*mA-fU-mA-fG- | 9 |
| (pos. | mA-fC-mA-fG-mU-fG- | | | mA-fG-mC-fA-mA-mG- | |
| ctrl. 1) | mU-fU-fC-fU-mU-fG- | | | mA-fA-mC-fA-mC-fU- | |
| | mC-fU-mC-fU-mA-fU- | | | mG-fU-mU*mU*mU | |
| | mA-fA-lT4-lT4 | | | | |
| ss1-2 | lgT7-lgT7-lgT7-fA- | 3 | | | |
| (pos. | mA-fC-mA-fG-mU-fG- | | | | |
| ctrl. 2) | mU-fU-fC-fU-mU-fG- | | | | |
| | mC-fU-mC-fU-mA-fU- | | | | |
| | mA-fA-lT4-lT4 | | | | |
| ss1-3 | lpT1-lpT1-lpT1-fA- | 4 | | | |
| | mA-fC-mA-fG-mU-fG- | | | | |
| | mU-fU-fC-fU-mU-fG- | | | | |
| | mC-fU-mC-fU-mA-fU- | | | | |
| | mA-fA-lT4-lT4 | | | | |
| ss1-4 | lsT1-lsT1-lsT1-fA- | 5 | | | |
| | mA-fC-mA-fG-mU-fG- | | | | |
| | mU-fU-fC-fU-mU-fG- | | | | |
| | mC-fU-mC-fU-mA-fU- | | | | |
| | mA-fA-lT4-lT4 | | | | |
| ss1-5 | lsT2-lsT2-lsT2-fA- | 6 | | | |
| | mA-fC-mA-fG-mU-fG- | | | | |
| | mU-fU-fC-fU-mU-fG- | | | | |
| | mC-fU-mC-fU-mA-fU- | | | | |
| | mA-fA-lT4-lT4 | | | | |
| ss1-6 | lsT3-lsT3-lsT3-fA- | 7 | | | |
| | mA-fC-mA-fG-mU-fG- | | | | |
| | mU-fU-fC-fU-mU-fG- | | | | |
| | mC-fU-mC-fU-mA-fU- | | | | |
| | mA-fA-lT4-lT4 | | | | |

TABLE O
siRNAs

| siRNA-# | ss-# | as-# | Ligand |
|---|---|---|---|
| siRNA1-0 | ss1-0 | as1-0 | lgT3 |
| neg. ctrl. | | | |
| siRNA1-1 | ss1-1 | as1-1 | lgT3 |
| pos. ctrl._1 | | | |
| siRNA1-2 | ss1-2 | as1-1 | lgT7 |
| pos. ctrl._2 | | | |
| siRNA1-3 | ss1-3 | as1-1 | lpT1 |
| siRNA1-4 | ss1-4 | as1-1 | lsT1 |
| siRNA1-5 | ss1-5 | as1-1 | lsT2 |
| siRNA1-6 | ss1-6 | as1-1 | lsT3 |

Oligonucleotide analytics is shown below.

TABLE P
Single strand analytics - sense strands
(ss) and antisense strands (as)

| Sense strand | | | | Antisense strand | | | |
|---|---|---|---|---|---|---|---|
| | MW | MW | | | MW | MW | |
| ss-# | (calc.) | (found) | Z | as-# | (calc.) | (found) | z |
| ss1-0 | 8044.6 | 8043.4 | 1 | as1-0 | 9603.53 | 6903.0 | 1 |
| ss1-1 | 9467.6 | 9465.3 | 1 | as1-1 | 7595.96 | 7595.0 | 1 |
| ss1-2 | 9605.7 | 9603.6 | 1 | | | | |
| ss1-3 | 9410.5 | 9408.6 | 1 | | | | |
| ss1-4 | 9791.8 | 9789.8 | 1 | | | | |
| ss1-5 | 10031.7 | 5015.0 | 2 | | | | |
| ss1-6 | 9881.8 | 9879.3 | 1 | | | | |

TABLE Q
Double strand analytics

| | MW (ss) | MW (as) | MW [siRNA] | MW [siRNA] | |
|---|---|---|---|---|---|
| siRNA-# | found | found | calc. | found | z |
| siRNA1-0 | 8044.7 | 6903.1 | 14948.1 | 2490.3 | 6 |
| siRNA1-1 | 9467.0 | 7595.1 | 17063.5 | 2842.8 | 6 |
| siRNA1-2 | 9605.1 | 7595.1 | 17201.6 | 2865.8 | 6 |
| siRNA1-3 | 9409.9 | 7595.2 | 17006.4 | 2833.3 | 6 |
| siRNA1-4 | 9791.1 | 7595.2 | 17387.7 | 2897.0 | 6 |
| siRNA1-5 | 10031.0 | 7595.2 | 17627.7 | 2937.0 | 6 |
| siRNA1-6 | 9881.1 | 7595.2 | 17477.8 | 2911.9 | 6 |

### Example 28: In Vitro Inhibition of a Target Gene Expression with Modified siRNAs

### Methods

IC₅₀ measurements

For IC₅₀ measurements in primary fresh mouse hepatocytes, 40,000 cells in Collagen-I coated 96-well plates were incubated for 48 hours under free uptake conditions with the siRNAs at concentrations ranging from 1 μM to 1 pM using 10-fold dilution steps. The half maximal inhibitory concentration (IC₅₀) for each siRNA was calculated by applying a Biostat-Speed statistical calculation tool. Results were obtained using the 4-parameter logistic model according to Ratkovsky and Reedy (1986, Biometrics, Vol. 42: 575-582). The adjustment was obtained by non-linear regression using the Levenberg-Marquardt algorithm in SAS v9.1.3 software.

mRNA Expression Analysis

48 hours after free siRNA uptake, the cellular RNA was harvested by using Promega's SV96 total RNA isolation system (cat. no. Z3500) according to the manufacturer's protocol including a DNase step during the procedure.

For cDNA synthesis, the Reverse Transcriptase kit (cat. no. N8080234) was used from ThermoFisher. cDNA synthesis from 30 ng RNA was performed using 1.2 μl 10×RT buffer, 2.64 μl MgCl₂ (25 mM), 2.4 μl dNTPs (10 mM), 0.6 μl random hexamers (50 PM), 0.6 μl Oligo(dT)16 (SEQ ID NO: 10) (50 μM), 0.24 μl RNase inhibitor (20 U/μl) and 0.3 μl Multiscribe (50 U/μl) in a total volume of 12 μl. Samples were incubated at 25° C. for 10 minutes and 42° C. for 60 minutes. The reaction was stopped by heating to 95° C. for 5 minutes.

Mouse TTR mRNA levels were quantified by qPCR using the ThermoFisher TaqMan Universal PCR Master Mix (cat. no. 4305719) and the TaqMan Gene Expression assay Mm00443267_ml. PCR was performed in technical duplicates with the ABI Prism 7900 under the following PCR conditions: 2 minutes at 50° C., 10 minutes at 95° C., 40 cycles with 95° C. for 15 seconds and 1 minute at 60° C. PCR was set up as a simplex PCR detecting the target gene in one reaction and the housekeeping gene (mouse RPL37A) for normalization in a second reaction. The final volume for the PCR reaction was 12.5 μl in a 1×PCR master mix, RPL37A primers were used in a final concentration of 50 nM and the probe of 200 nM. The ΔΔCt method was applied to calculate relative expression levels of the target transcripts. Percentage of target gene expression was calculated by normalization based on the levels of LV2 non-silencing siRNA control sequence.

### IFNα Determination

IFNα2a protein concentration was quantified in the supernatant of human PBMCs using an IFNα2a isoform-specific assay (cat. no. K151VHK) based on MesoScale's U-PLEX platform and according to the supplier's protocol.

### Cytotoxicity

Cytotoxicity of mouse TTR siRNAs was measured 72 hours after incubation with 40,000 primary fresh mouse hepatocytes under free uptake conditions by determining the ratio of cellular viability/toxicity in each sample. Cell viability was measured by determination of the intracellular ATP content using the CellTiter-Glo assay (Promega, cat. no. G7570) according to the manufacturer's protocol. Cell toxicity was measured in the supernatant using the LDH assay (Sigma, cat. no. 11644793001) according to the manufacturer's protocol.

### Results

The in vitro knock-down results in primary mouse hepatocytes of the compounds siRNA1-1 to siRNA1-6 are summarized in Table R.

TABLE R
IC₅₀ data of siRNAs in primary mouse hepatocytes

| | Cell targeting | IC₅₀ | |
|---|---|---|---|
| siRNA-# | ligand | [nm] | Imax [%] |
| siRNA1-0 | neg. ctrl. | n.a. | — |
| siRNA1-1 | lgT3 | 3.53 | 98.2 |
| siRNA1-2 | lgT7 | 2.71 | 98.3 |
| siRNA1-3 | lpT1 | 8.47 | 98.0 |
| siRNA1-4 | lsT1 | 4.25 | 98.2 |
| siRNA1-5 | lsT2 | 7.18 | 98.7 |
| siRNA1-6 | lsT3 | 26.80 | 97.5 |

Table R shows that all siRNAs with novel ASGPR-binders (siRNAs 1-3, 1-4, 1-5, and 1-6) show in-vitro knock-down of the target gene. Compared to the reference GalNAc-conjugated siRNAs (siRNA1-1 and 1-2), which show IC₅₀-values of 3.53 nM and 2.71 nM respectively, siRNA1-3 with the trimeric piperidine-based ASGPR-binder still shows excellent potency (8.47 nM). Additionally, siRNA1-4 and siRNA1-5 with trimeric guanosine-derived ASGPR-binders show comparable IC₅₀-values of 4.25 and 7.18 nM, respectively. siRNA1-6, which comprises trimeric guanosine-derived lsT3, seems relatively less potent compared to the GalNAc-analogs and shows an IC₅₀-value of 26.80 nM.

No obvious adverse effects were observed in in vitro assays on cytotoxicity in mouse hepatocytes and immune stimulation in human PBMCs.

### Example 29: In Vitro Stabilities of Modified siRNAs in 50% Mouse Serum

### Methods

### Nuclease Stability Assays

The siRNAs were tested for nuclease stability in 50% mouse serum. For this purpose, 160 μL of 2.5 μM siRNA in 1×DPBS (Life Technologies, cat. no. 14190-094) and 160 μL mouse serum (Sigma, cat. no. M5905) were incubated at 37° C. for 0, 24, 48, 72, 96 and 168 hours. At each time point, 21 μL of the reaction was taken out and quenched with 23 μL stop solution (for 3,000 μL stop solution: 1123 μL Tissue & Cell Lysis Solution (Epicentre, cat. no. MTC096H), 183 μL 20 mg/mL Proteinase K (Qiagen, cat. no. 19133), 1694 μL water) at 65° C. for 30 minutes. Prior to HPLC analysis on an Agilent Technologies 1260 Infinity II instrument using a 1260 DAD detector, 33 μL of RNase-free water was added to each sample. 50 μL of the solution was analyzed by HPLC using a DNAPac PA200 analytical column (Thermo Scientific, cat. no. 063000), and the following gradient:

| Time (min) | Flow (mL/min) | % Buffer A* | % Buffer B** |
|---|---|---|---|
| 0 | 1 | 75 | 25 |
| 20 | 1 | 35 | 65 |

*Buffer A: 20 mM sodium phosphate (Sigma, cat. No. 342483), pH 11;
**Buffer B: 20 mM sodium phosphate (Sigma, cat. No. 342483), 1M sodium bromide (Sigma, cat. No. 02119), pH 11.

### Results

The stabilities of the tested siRNAs (siRNA1-1 to siRNA1-6) are listed in Table S:

TABLE S
SiRNA-stabilities in 50% mouse serum

| siRNA-# | Cell targeting ligand | T_{1/2} [h] |
|---|---|---|
| siRNA1-1 | lgT3 | 72 |
| siRNA1-2 | lgT7 | 96 |
| siRNA1-3 | lpT1 | 96 |
| siRNA1-4 | lsT1 | 72 < T_{1/2} < 96 |
| siRNA1-5 | lsT2 | 96 |
| siRNA1-6 | lsT3 | 72 < T_{1/2} < 96 |

The siRNAs, containing novel ASGPR-binders (siRNA1-3, 1-4, 1-5, and 1-6) show comparable serum half-lifes as the siRNAs linked with reference GalNAc-ligands (siRNA1-1 and 1-2).

### Example 30: In Vivo Inhibition of Target Gene Expression with Modified siRNAs

### Methods

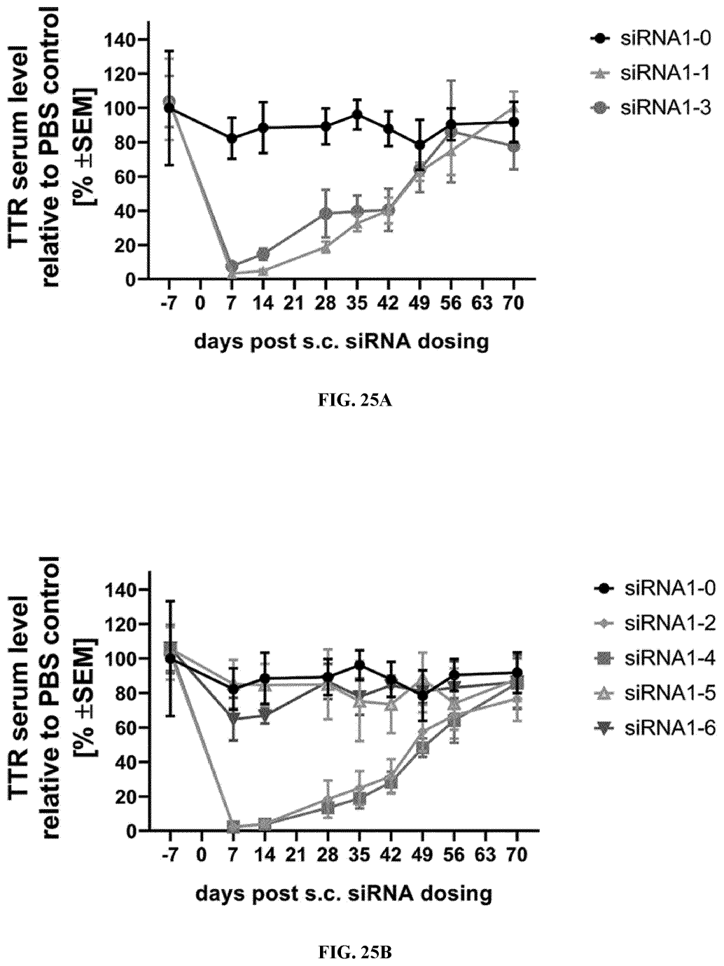

C57BL/6N mice (female 20-22 g; Charles River, Germany) were treated subcutaneously with a single dose of 2.5 mg/kg of the siRNA or PBS (mock control) in groups of n=6. Sequences of the administered compounds are listed in Tables N and O. Blood samples were drawn pre- and post-dosing as indicated in FIG. 25.A and FIG. 25.B. SiRNA target TTR was quantified from serum by a commercially available ELISA assay (Alpco Diagnostics, Cat. no.: 41-PALMS-E01).

### Results

A comparison of in vivo inhibition of target gene expression by both targeting siRNAs (siRNA1-1 and siRNA1-3) shows that siRNA1-3 with a piperidine-derived ASGPR-binder shows comparable in-vivo knock-down activity relative to the GalNAc-derived control compound siRNA1-1 with a comparable linker length between the ASGPR-binding unit and the morpholine-scaffold (FIG. 25A).

Very unexpectedly, the in-vivo potencies of the siRNAs with guanosine-type ASGPR-binders are very different. Whereas siRNA1-5 and 1-6 do not show any in-vivo knock-down activity, siRNA1-4 shows very high potency (FIG. 25B). Compared to the GalNAc-type control siRNA1-2 with the same linker between the morpholine-scaffold and the ASGPR-binding unit, the guanosine-derived siRNA1-4 shows the same in-vivo potency as the GalNAc-analog siRNA1-2. This result could not be derived from the in vitro data shown in Table R, where siRNAs 1-2, 1-4, and 1-5 showed similar potencies and only siRNA1-6 had a comparatively lower in vitro knock-down activity.

As can be seen in Table M, the trimers of lsT2 and lsT3, which correspond to siRNA1-5 and 1-6, show significantly lower ASGPR-binding than the lsT1-trimer, which is linked to siRNA1-4.

1. A compound of formula (I) 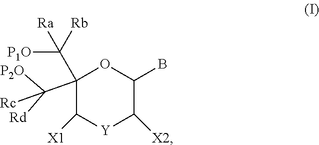
or a pharmaceutically acceptable salt thereof,
wherein:
B is a heterocyclic nucleobase;
P₁ and P₂ are each, independently, H, a reactive phosphorous group, or a protecting group;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy, or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is a cell targeting moiety of formula (II) or a protected derivative thereof: 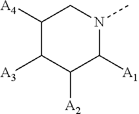
wherein:
R3 targets a mammalian (optionally human) asialoglycoprotein receptor (ASGPR),
A₁, A₂ and A₃ are, independently, H, hydroxy, alkoxy, acyloxy, aryloxy, aroyloxy, alkoxycarbonyl, aryloxycarbonyl, oxo (═O), or a (C1-C20) alkyl group, unsubstituted or optionally substituted by one or more groups selected from OH, a (C3-C8) cycloalkyl group, a (C3-C14) heterocycle, a (C6-C14) aryl group, a (C5-C14) heteroaryl group, —O—Z5, —N(Z5)(Z6), —S—Z5, —CN, —C(=M)-O—Z5, —O—C(=M)-Z5, —C(=M)-N(Z5)(Z6), and —N(Z5)-C(=M)-Z6, wherein:
M is O or S,
each of Z5 and Z6 is, independently, H, a (C1-C6) alkyl group, or a (C6-C14) aryl group, wherein both alkyl and aryl groups are either unsubstituted or optionally substituted by one or more groups selected from halogen, amino, hydroxy, thiol, cyano, alkyl, alkoxy, aryloxy, acyloxy, aroyloxy, carboxy, alkoxycarbonyl, aryloxycarbonyl and arylalkoxycarbonyl;
A4 is —N(R4)₂, —NH—C(═O)—R4, or 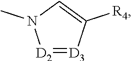
wherein:
D2 and D3 are N, O, or S;
R4 is H or a (C1-C20) alkyl group, unsubstituted or optionally substituted by one or more groups selected from halogen, amino, hydroxy, thiol, cyano, alkyl, alkoxycarbonyl, aryloxycarbonyl, alkoxy, aryloxy, acyloxy, aroyloxy and carboxy; and
each of X1, X2, Ra, Rb, Rc, and Rd independently is H or a —(C1-C6) alkyl group.

2. A compound of formula (I) 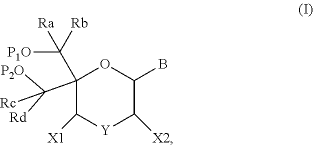
or a pharmaceutically acceptable salt thereof,
wherein:
B is a heterocyclic nucleobase;
P₁ and P₂ are each, independently, H, a reactive phosphorous group, or a protecting group;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy, or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is a cell targeting moiety of formula (IVA) or (IVB) or a protected derivative thereof: 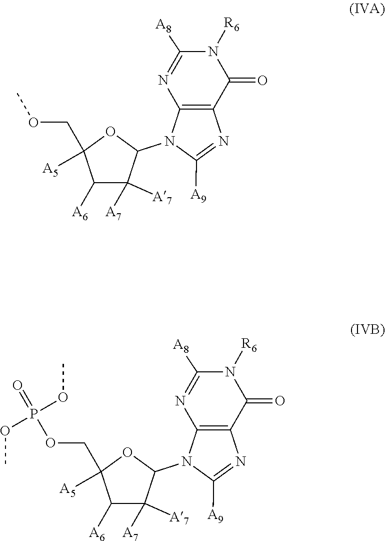
wherein:
R3 targets a mammalian (optionally human) asialoglycoprotein receptor (ASGPR),
R6 is H or a (C1-C6) alkyl group, unsubstituted or optionally substituted by one or more groups selected from halogen, amino, hydroxy, thiol, alkyl, alkoxy, aryloxy, carboxy, alkoxycarbonyl, and aryloxycarbonyl;
A₅, A₆, A₇, and A′₇ are independently H, hydroxy, alkoxy, acyloxy, aryloxy, aroyloxy, alkoxycarbonyl, aryloxycarbonyl, amino, or a (C1-C20) alkyl group unsubstituted or optionally substituted by one or more groups selected from OH, a (C3-C8) cycloalkyl group, a (C3-C14) heterocycle, a (C6-C14) aryl group, a (C5-C14) heteroaryl group, —O—Z7, —N(Z7)(Z8), —S—Z7, —CN, —C(=Q)-O—Z7, —O—C(=Q)-Z7, —C(=Q)-N(Z7)(Z8), and —N(Z7)-C(=Q)-Z8, wherein:
Q is O or S,
each of Z7 and Z8 independently is H, a (C1-C6) alkyl group, or a (C6-C14) aryl group, both groups unsubstituted or optionally substituted by one or more groups selected from a halogen atom and a (C1-C6) alkyl group;
each of A₈ and A₉ independently is H, halogen, OH (or its tautomeric oxo (═O)), —N(R7)₂, —NHR7, or —NH—C(═O)—R7, wherein R7 is hydrogen or a (C1-C20) alkyl group, unsubstituted or optionally substituted by one or more groups selected from a halogen atom, alkoxy, aryloxy, a (C1-C6) alkyl group, a (C3-C8) cycloalkyl group, a (C3-C14) heterocycle, a (C6-C14) aryl group, a (C5-C14) heteroaryl group; and
each of X1, X2, Ra, Rb, Rc, and Rd independently is H or a —(C1-C6) alkyl group.

3. The compound of formula (I) of claim 1, wherein:
(i) L is a C1-C10 hydrocarbon chain;
(ii) L is a C1-C10 hydrocarbon chain terminated by —(CO)—; or
(iii) Y is NR1, wherein R1 is -L-R3, wherein L is a C1-C10 hydrocarbon chain interrupted by one or more —O—.

4-5. (canceled)

6. The compound of formula (I) of claim 1, wherein:
(i) A1 is H, oxo (═O), or a (C1-C6) alkyl group, or a (C1-C6)-alkenyl group, both optionally substituted by hydroxy, alkoxy, or aryloxy;
(ii) A1 is a (C1-C6) alkyl group optionally substituted by —O—C(=M)-Z5, wherein M is O and Z5 is a (C1-C6) alkyl group optionally substituted by an alkoxycarbonyl or arylalkoxycarbonyl group;
(iii) A2 and A3 are hydroxy or acyloxy;
(iv) A4 is —NH—C(═O)—R4, wherein R4 is a (C1-C6) alkyl group optionally substituted by a carboxy, alkoxycarbonyl, or aryloxycarbonyl group; or
(v) A4 is 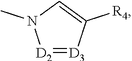
wherein D2 and D3 are N, and R4 is a (C1-C6) alkyl group, optionally substituted by an alkoxy or aryloxy group.

7-10. (canceled)

11. The compound of formula (I) of claim 2, wherein:
(i) A6 and A7 are hydroxy or acyloxy;
(ii) A′7 is H or a (C1-C6) alkyl group;
(iii) A5 is H or a (C1-C6) alkyl group, optionally substituted by one or more hydroxy or acyloxy groups;
(iv) A8 is H, halogen, or OH or its tautomeric oxo (═O);
(v) A8 is selected in the group consisting of —N(R7)₂, —NHR7 or —NH—C(═O)—R7, wherein R7 is H or a (C1-C6) alkyl group;
(vi) A9 is H, OH or its tautomeric oxo (═O), or NH2; or
(vii) R6 is a H or a (C1-C6) alkyl group.

12-17. (canceled)

18. A compound of formula (III) 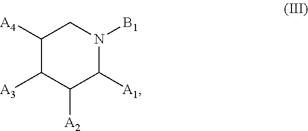
or a pharmaceutically acceptable salt thereof,
wherein:
A1, A2 and A3 are, independently, H, hydroxy, alkoxy, acyloxy, aryloxy, aroyloxy, alkoxycarbonyl, aryloxycarbonyl, oxo (═O), or a (C1-C20) alkyl or alkenyl group, unsubstituted or optionally substituted by one or more groups selected from halogen, hydroxy, a (C3-C8) cycloalkyl group, a (C3-C14) heterocycle, a (C6-C14) aryl group, a (C5-C14) heteroaryl group, —O—Z5, —N(Z5)(Z6), —S—Z5, —CN, —C(=M)-O—Z5, —O—C(=M)-Z5, —C(=M)-N(Z5)(Z6), and —N(Z5)-C(=M)-Z6,
wherein:
M is O or S,
each of Z5 and Z6 is, independently, H, a (C1-C6) alkyl group, unsubstituted or optionally substituted by one or more groups selected from halogen, amino, hydroxy, alkoxy, aryloxy, carboxy, alkoxycarbonyl, aryloxycarbonyl, and carbonyloxy;
A4 is —N(R4)2, —N—C(═O)—R4, or 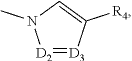
wherein:
D2 and D3 are N, O, or S;
R4 is H or a (C1-C20) alkyl group, unsubstituted or optionally substituted by one or more groups selected from halogen, amino, hydroxy, thiol, cyano, alkyl, alkoxycarbonyl, aryloxycarbonyl, alkoxy, acyloxy, aryloxy, aroyloxy and carboxy;
B1 is H, benzyl ester, -L-R5, or —(CO)-L-R5, wherein:
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —(CO)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxyalkyl, hydroxy, or haloalkyl, the C2-C25 hydrocarbon chain being optionally substituted with one or more -L′-R5, wherein L′ is a C2-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; and
R5 is H, OH, benzyl, benzyloxy, or a nucleoside, nucleoside analog, nucleotide or nucleotide analog.

19. The compound of claim 18, wherein:
(i) L is a C1-C6 hydrocarbon chain, optionally terminated by —(CO)—, and R5 is H, OH, benzyl, or benzyloxy;
(ii) A1 is H, (═O), or a (C1-C6) alkyl or alkenyl group optionally substituted by hydroxy, alkoxy, or aryloxy;
(iii) A1 is a (C1-C6) alkyl group optionally substituted by —O—C(=M)-Z5, wherein M is O and Z5 is a (C1-C6) alkyl group optionally substituted by an alkoxycarbonyl or arylalkoxycarbonyl group;
(iv) A2 and A3 are hydroxy;
(v) A4 is —NH—C(═O)—R4, wherein R4 is a (C1-C6) alkyl group optionally substituted by a carboxy, alkoxycarbonyl, or aryloxycarbonyl group;
(vi) A4 is 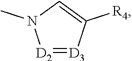
wherein D2 and D3 are N, and R4 is a (C1-C6) alkyl group, optionally substituted by an alkoxy or aryloxy group; or
(vii) B1 is H or a benzyl ester group.

20-25. (canceled)

26. A compound of formula (V) 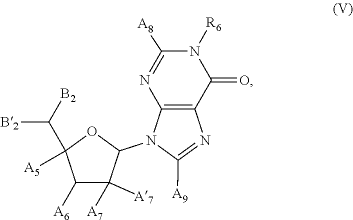
or a pharmaceutically acceptable salt thereof,
wherein:
R6 is H or a (C1-C6) alkyl group, unsubstituted or optionally substituted by one or more groups selected from halogen, amino, hydroxy, thiol, alkyl, alkoxy, aryloxy, carboxy, alkoxycarbonyl, and aryloxycarbonyl;
each of A5, A6, A7, and A′7 independently is H, hydroxy, alkoxy, acyloxy, aryloxy, aroyloxy, amino, or a (C1-C20) alkyl group unsubstituted or optionally substituted by one or more groups selected from halogen, OH, a (C3-C8) cycloalkyl group, a (C3-C14) heterocycle, a (C6-C14) aryl group, a (C5-C14) heteroaryl group, —O—Z7, —N(Z7)(Z8), —S—Z7, —CN, —C(=Q)-O—Z7, —O—C(=Q)-Z7, —C(=Q)-N(Z7)(Z8), and —N(Z7)-C(=Q)-Z8, wherein:
Q is O or S,
each of Z7 and Z8 independently is H or a (C1-C6) alkyl group, unsubstituted or optionally substituted by one or more groups selected from a halogen atom and a (C1-C6) alkyl group;
each of A8 and A9 independently is H, halogen, OH (or its tautomeric oxo (═O)), —N(R7)2, —NHR7, or —N—C(═O)—R7, wherein R7 is hydrogen or a (C1-C20) alkyl group, unsubstituted or optionally substituted by one or more groups selected from a halogen atom, alkoxy, aryloxy, a (C1-C6) alkyl group, a (C3-C8) cycloalkyl group, a (C3-C14) heterocycle, a (C6-C14) aryl group, a (C5-C14) heteroaryl group;
each of B2 and B′2 independently is —H, —R8, —OH, —OR8, —COOH, —C(O)—NR8R′8, —NH2, —NHR8, —NH—C(O)—R8, —O—P(O)(OH)₂, —O—P(O)(OR8)(OR′8) or a (C1-C6) alkyl optionally substituted by —OH, wherein R8 and R′8 are independently H or -L-R9, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxyl, or haloalkyl, the C1-C25 hydrocarbon chain being optionally substituted with one or more -L′-R9, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re), —O—C(O)—(Re), —C(O)—O—(Re), or —O—C(O)—O—;
R9 is H, OH, benzyl, benzyloxy, or a nucleoside, a nucleoside analog, or a nucleotide or a nucleotide analog, and
wherein when B₂ is CH₂OH, B₂′ is OH, A₅ is H, A₆ is OH, A₇ is H, A₇′ is OH, A₉ is H, and R₆ is H, A₈ is not NH₂.

27. The compound of claim 26, wherein:
(i) each of B2 and B′2 independently is H, OH, —NH₂, or —COOH;
(ii) B2 is —NH—C(O)—R8, —C(O)—NR8R′8, or —C(O)—NHR8, wherein R8 and R′8 are independently H or -L-R9, wherein L is a C1-C6 hydrocarbon chain optionally terminated by —C(O), optionally wherein R9 is H, OH or a nucleoside analog;
(iii) A5 is H or a (C1-C6) alkyl group, optionally substituted by one or more hydroxy;
(iv) A6 and A7 are hydroxy;
(v) A′7 is H or a (C1-C6) alkyl group;
(vi) A8 is H, halogen, or hydroxy or its corresponding oxo (═O) tautomer;
(vii) A8 is selected in the group consisting of —N(R7)2, —NHR7 or —NH—C(═O)—R7, wherein R7 is H or a (C1-C6) alkyl group;
(viii) A9 is H, OH or its corresponding oxo (═O) tautomer, or NH₂; or
(ix) R6 is a H or a (C1-C6) alkyl group.

28-36. (canceled)

37. An oligonucleotide comprising one or more compounds of formula (VI): 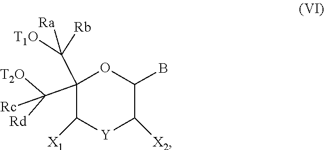
or a pharmaceutically acceptable salt thereof,
wherein
B is a heterocyclic nucleobase;
one of T₁ and T₂ is an internucleoside linking group linking the compound of formula (VI) to the oligomeric compound and the other of T₁ and T₂ is H, a protecting group, a phosphorus moiety, or an internucleoside linking group linking the compound of formula (VI) to the oligomeric compound;
Y is NR1 or N—C(═O)—R1, wherein R1 is -L-R3, wherein
L is a C1-C25 hydrocarbon chain optionally interrupted or terminated by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—; each of Re and Rf, independently, being hydrogen, alkyl, alkenyl, alkynyl, alkoxy, aryloxy, hydroxylalkyl, hydroxyl, or haloalkyl, the C1-C25 hydrocarbon chain being optionally substituted with one or more -L′-R3, wherein L′ is a C1-C25 hydrocarbon chain optionally interrupted by one or more —O—, —C(O)—, —N(Re)—, —N(Re)—C(O)—O—, —O—C(O)—N(Re)—, —N(Re)—C(O)—N(Rf)—, —C(O)—N(Re)—, —N(Re)—C(O)—, —O—C(O)—, —C(O)—O—, or —O—C(O)—O—;
R3 is an ASGPR-binding cell targeting moiety of formula (II), (IVA) or (IVB); and
each of X1, X2, Ra, Rb, Rc, and Rd independently is H or a —(C1-C6) alkyl group.

38. The oligonucleotide of claim 37, wherein Y is selected in the group consisting of NR1 and N—C(═O)—R1, and L is a C1-C10 hydrocarbon chain.

39. The oligonucleotide of claim 37, wherein Y is selected in the group consisting of NR1 and N—C(═O)—R1, and L is a C1-C10 hydrocarbon chain optionally terminated by —C(O)—.

40. The oligonucleotide of claim 37, wherein Y is NR1 and L is a C2-C10 hydrocarbon chain optionally interrupted by one or more —O—.

41. The oligonucleotide of claim 37, wherein the oligonucleotide is single-stranded, such as an antisense oligonucleotide that targets a human mRNA, or is double-stranded.

42. The oligonucleotide of claim 41, wherein the oligonucleotide is a double-stranded interfering RNA that targets a human mRNA and comprises a sense strand and an antisense strand.

43. The oligonucleotide of claim 41, wherein the oligonucleotide has an overhang at the 5′ or 3′ end of the sense or antisense strand, and/or wherein a compound of formula (VI) is located in the 5′ or 3′ end of the sense strand.

44. (canceled)

45. The oligonucleotide of claim 43, wherein a compound of formula (VI) is located in the overhang.

46. A method of delivering an oligonucleotide to liver (hepatic) cells in a human subject in need thereof, comprising administering to the subject the oligonucleotide of claim 37, such as through intravenous or subcutaneous injection or injection through the hepatic portal vein.

47. A method of treating a human subject in need thereof, comprising administering to the subject the oligonucleotide of claim 37.

48. A method of delivering a therapeutic agent to liver (hepatic) cells in a human subject in need thereof, comprising administering to the subject a therapeutic moiety conjugated to the compound of claim 1.
